(19) United States
(12) Patent Application Publication
(10) Pub. No.: US 2024/0270797 A1
(43) Pub. Date: Aug. 15, 2024

(54) CORONAVIRUS NEUTRALIZING COMPOSITIONS AND ASSOCIATED METHODS

(71) Applicants: CZ Biohub SF, LLC, San Francisco, CA (US); The Board of Trustees of the Leland Stanford Junior University, Stanford, CA (US)

(72) Inventors: Payton Anders-Benner Weidenbacher, Menlo Park, CA (US); Peter S. Kim, Stanford, CA (US); Eric Waltari, San Francisco, CA (US)

(73) Assignees: CZ Biohub SF, LLC, San Francisco, CA (US); The Board of Trustees of the Leland Stanford Junior University, Stanford, CA (US)

(21) Appl. No.: 18/568,971

(22) PCT Filed: Jun. 22, 2022
(86) PCT No.: PCT/US2022/034582

§ 371 Date: Dec. 11, 2023

Related U.S. Application Data
(60) Provisional application No. 63/214,114, filed on Jun. 23, 2021.
(60) Provisional application No. 63/302,473, filed on Jan. 24, 2022.

(51) Int. Cl. C07K 14/005; A61K 38/00; A61P 31/14

(52) U.S. Cl. CPC C07K 14/005; A61P 31/14; A61K 38/00; C07K 2317/565; C07K 2317/76; C07K 2319/30; C12N 2770/20022; C12N 2770/20033

(57) ABSTRACT

Provided are fusion proteins and modified proteins comprising a neutralizing polypeptide and an antibody (e.g., a non-neutralizing antibody) that specifically binds to an epitope in a conserved region of one or more coronavirus spike proteins. The fusion proteins and modified proteins are able to specifically bind to and neutralize a broad spectrum of coronaviruses, including SARS-CoV-2 and all known SARS-COV-2 variants of concern (e.g., the Delta and Omicron variants). Also provided are various compositions of such proteins, methods of their use, nucleic acids encoding such proteins or domains thereof, constructs, expression cassettes, and vectors containing such nucleic acids, and host cells capable of expressing these proteins or domains thereof. Additionally provided are prophylactic and therapeutic methods employing the fusion proteins and/or modified proteins of the disclosure.

### CROSS-REFERENCES TO REPLATED APPLICATIONS

This application claims priority to U.S. Provisional Application No. 63/214,114, filed on Jun. 23, 2021 and U.S. Provisional Application No. 63/302,473, filed on Jan. 24, 2022. The entire disclosure of each of the aforementioned provisional applications is herein incorporated by reference for all purposes.

### REFERENCE TO A SEQUENCE LISTING

The official copy of the sequence listing is submitted electronically via EFS-Web as an ASCII formatted sequence listing with a file named 1316301_seqlist.txt, created on Jun. 22, 2022, and having a size of 394 KB, and is filed concurrently with the specification. The sequence listing contained in this ASCII formatted document is part of the specification and is herein incorporated by reference in its entirety.

### BACKGROUND

Coronaviruses (CoV) are a large family of viruses that cause human illness ranging from the common cold to more severe diseases, such as Middle East Respiratory Syndrome (MERS) and Severe Acute Respiratory Syndrome (SARS). Coronaviruses are zoonotic, meaning they can be transmitted between animals and humans. Coronaviruses are large, enveloped, single-stranded RNA viruses having a characteristic crown, or corona, around the virions, due to the surface of the virus particle being covered in well-separated, petal-shaped glycoprotein “spikes,” having a diameter of 80-160 nm, that project from the virions. Spike glycoprotein is a Class I viral fusion protein located on an outer envelope of the virion. Spike protein plays an important role in viral infection by interacting with host cell receptors via a receptor binding domain (RBD).

Severe Acute Respiratory Syndrome Coronavirus 2 (SARS-COV-2) is the strain of coronavirus that causes coronavirus disease 2019 (COVID-19), a respiratory illness. SARS-COV-2 is highly infectious and primarily spreads between people through close contact and via respiratory droplets and aerosols. Both SARS-COV-1 and SARS-COV-2 can enter eukaryotic cells via endosomes or plasma membrane fusion. In both routes, spike proteins on the virion surface bind to the membrane-bound protein Angiotensin-converting enzyme 2 (ACE2) and mediate attachment to the membrane of and entry into a host cell. For MERS-COV, the viral receptor for cell entry is dipeptidyl peptidase-4 (DPP4, CD26).

Research evidence suggests that SARS-COV and MERS-COV originated in bats, and it is likely that SARS-COV-2 did as well. Viral mutation and zoonotic transfer is anticipated to lead to future pandemics and large-scale outbreaks of disease caused by novel coronaviruses. To date, there are a limited number of active pharmaceutical agents that have demonstrated any clinical effect in treating COVID-19 or other coronavirus infections in patients. For example, COVID-19 therapeutics are limited to small molecules remdesivir (Gilead Sciences); paxlovid (Pfizer); molnupiravir; dexamethasone (a corticosteroid) alone or in combination with tocilizumab (Actemra), a recombinant humanized anti-interleukin-6 receptor monoclonal antibody, and a small number of monoclonal antibodies targeting epitopes on the spike protein, sotrovimab (GlaxoSmithKline); regdanvimab (Celltrion); cilgavimab plus tixagevimab (AstraZeneca); bamlanivimab plus etesevimab (AIIa) (Eli Lilly); and casirivimab plus imdevimab (AIIa) (Regeneron), the latter three combinations comprising pairs of monoclonal antibodies directed against non-overlapping epitopes of the spike protein receptor binding domain.

Further, numerous SARS-COV-2 variants have been identified, including the Alpha, Beta, Gamma, Delta, and Omicron strains, some of which reduce the effectiveness of available therapeutics. The onset of the Omicron variant, in particular, has drastically reduced the utility of six of the seven clinically available monoclonal antibodies (mAbs) against SARS-COV-2. Of the eight clinically available mAbs, all of which target the RBD, only sotrovimab retained activity suitable for use against Omicron (Cameroni et al., 2021, doi: 10.1038/d41586-038254). Omicron has a much larger mutational profile than previous variants of concern (VOCs), with 36 total mutations, 15 of which are in the receptor binding domain (RBD) and 10 of which fall in the binding interface between the RBD and ACE2. Treatment of Omicron infections, as well as those caused by any future novel strains, will require development of broad-spectrum therapeutic agents. In view of the limited therapeutic options currently available, and the future threat of novel, wide-spread disease from new SARS-COV-2 variants and new coronaviruses, there is an urgent and ongoing need for therapeutic agents capable of treating infections arising from known coronaviruses such as SARS-COV-2 and any of its variants, as well as novel coronaviruses that will arise in the future.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the creation by the inventors of unique fusion proteins that have therapeutic benefit for treatment of infection by existing and future coronaviruses.

In one aspect, provided herein are fusion proteins and modified proteins comprising a neutralizing polypeptide that binds to a first coronavirus spike protein, a peptide linker and/or a non-peptide linker, and an antibody that specifically binds an epitope in a conserved region of a second coronavirus spike protein. In some embodiments, the neutralizing polypeptide binds to the first coronavirus spike protein through the receptor binding domain (RBD) on the spike protein. In some embodiments, the neutralizing polypeptide is a coronavirus receptor polypeptide. In some embodiments, the coronavirus receptor polypeptide comprises an ACE2 receptor ectodomain polypeptide or a DPP4 receptor ectodomain polypeptide. In some embodiments, the neutralizing polypeptide is a neutralizing antibody. In some embodiments, the antibody that specifically binds an epitope in a conserved region of a coronavirus spike protein is a non-neutralizing antibody. In some embodiments, the conserved region comprises 90% or greater conservation across related coronaviruses. In some embodiments, the related coronaviruses comprise spike proteins with amino acid sequences having 40% or greater amino acid sequence identity to SEQ ID NO:337. In some embodiments, the antibody that specifically binds an epitope in a conserved region of a coronavirus spike protein specifically binds an epitope in a conserved region of SARS-COV-1 spike protein, SARS-COV-2 spike protein, or MERS-COV spike protein. In some embodiments, the first coronavirus spike protein and the second coronavirus spike protein are both a SARS-COV-1 spike protein, a SARS-COV-2 spike protein, or a MERS-COV spike protein. In some embodiments, the first coronavirus spike protein and the second coronavirus spike protein are the same protein. In some embodiments, the neutralizing polypeptide and the antibody that specifically binds an epitope in a conserved region of a coronavirus spike protein do not bind competitively to their respective binding sites. In some embodiments, the first coronavirus spike protein and the second coronavirus spike protein are different coronavirus spike proteins selected from the group consisting of a SARS-COV-1 spike protein, a SARS-COV-2 spike protein, and a MERS-COV spike protein.

In some embodiments of the fusion proteins and modified proteins provided herein, the ACE2 receptor ectodomain polypeptide comprises an amino acid sequence having at least 90% sequence identity to the amino acid sequence as set forth in SEQ ID NO:270 or SEQ ID NO:271. In some embodiments, the DPP4 receptor ectodomain polypeptide comprises an amino acid sequence having at least 90% sequence identity to the amino acid sequence as set forth in SEQ ID NO:273 or SEQ ID NO:274.

In some embodiments of the fusion proteins and modified proteins provided herein, the antibody that specifically binds an epitope in a conserved region of a coronavirus spike protein comprises: a heavy chain variable region comprising (i) a CDRH1 comprising any of SEQ ID NOs: 153-170; (ii) a CDRH2 comprising any of SEQ ID NOs: 171-188; (iii) a CDRH3 comprising any of SEQ ID NOs: 189-214; and a light chain variable region comprising (i) a CDRL1 comprising any of SEQ ID NOs: 215-232; (ii) a CDRL2 compring any of SEQ ID NOs: 233-241; (iii) a CDRL3 comprising any of SEQ ID NOs: 242-268.

In some embodiments of the fusion proteins and modified proteins provided herein, the antibody that specifically binds an epitope in a conserved region of a coronavirus spike protein comprises a heavy chain variable region having at least 90% sequence identity to any of SEQ ID NOs: 1-7, 11-15, 17-23, 26, 29-32, 34, 35, 37, and 38 and a light chain variable region having at least 90% sequence identity to any of SEQ ID NOs: 77-83, 87-91, 93-99, 102, 105-108, 110, 111, 113, and 114. In some embodiments, the conserved region is a region of a SARS-COV-2 spike protein

In some embodiments of the fusion proteins and modified proteins provided herein, the peptide linker comprises at least 25 amino acids. In some embodiments, the peptide linker comprises an amino acid sequence with at least 90% sequence identity to any of SEQ ID NOs: 294-299.

In some embodiments, the fusion proteins provided herein have about 1000-fold increased neutralization potency for SARS-COV-2 relative to the cleaved fusion protein domains. In some embodiments, the fusion proteins have about 44-fold increased neutralization potency for SARS-CoV-2 and/or about 13-fold increased neutralization potency for SARS-COV-1 relative to bivalent ACE2. In some embodiments, the fusion proteins have about 376-fold increased neutralization potency for SARS-COV-2 and/or about 1162-fold increased neutralization potency for SARS-COV-1 relative to monovalent ACE2.

Also provided herein are recombinant nucleic acids encoding the fusion proteins and modified proteins provided herein. Also provided are DNA constructs comprising a promoter operably linked to the recombinant nucleic acids, vectors comprising the DNA constructs, and host cells comprising the recombinant nucleic acids, DNA constructs, and/or vectors. In some embodiments, the host cells are eukaryotic cells.

Also provided herein are methods of producing a fusion protein and/or a modified protein described herein comprising culturing any of the host cells described herein under conditions sufficient for the production of the fusion protein and/or modified protein by the host cell.

Also provided herein are pharmaceutical preparations comprising any of the fusion proteins or modified proteins described herein and a pharmaceutically acceptable carrier.

Also provided are methods for treating a subject infected with a SARS-COV-2 virus, having symptoms suggestive of a SARS-COV-2 infection, exposed to a SARS-COV-2 virus, or at risk of exposure to SARS-COV-2 virus, the method comprising administering to the subject a therapeutically effective amount of a pharmaceutical preparation described herein. In some embodiments, the subject has a confirmed SARS-COV-2 infection. In some embodiments, the subject is human. In some embodiments, the pharmaceutical preparation is administered intravenously. In some embodiments, the pharmaceutical preparation is administered at least once per day.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows that the receptor binding domain (RBD) and N-terminal domain (NTD) of human coronavirus (hCoV) spike proteins are both highly sequence divergent between different coronaviruses compared to other regions on the spike trimer, according to aspects of this disclosure. The top panel shows a structural representation of the trimeric SARS-COV-2 spike protein (PDB ID: 6VXX) shaded according to its sequence conservation compared with 6 total human coronavirus proteins (SARS-COV-2, SARS-COV-1, MERS, 229E, NL63, and OC43, listed in bottom panel) Darker colors indicated more variable sequences while lighter colors indicate more conserved sequences (as shown in key, middle panel). The location of the receptor binding domain (RBD) and N-terminal domain (NTD) are indicated by arrows on a single protomer.

FIG. 2 shows a list of antibody sequences which were converted into scFv format for study in the yeast library described herein, according to aspects of this disclosure.

FIGS. 3A-3C show binding of a yeast library displaying scFv fragments produced from “non-RBD” antibodies to SARS-COV-2 spike protein bait (FIG. 3A), SARS-COV-1 spike protein bait (FIG. 3B), and SARS-COV-2 spike protein RBD bait (FIG. 3C), according to aspects of this disclosure. Staining of the yeast library was tested with a range of bait protein concentrations: 500 nM tetramer, 250 nM tetramer, 125 nM tetramer, or 62.5 nM tetramer. To determine the percentage of antigen positive yeast, the percentage which were both c-myc positive and antigen positive was divided by the sum of the percentages which were either singly c-myc positive or both c-myc and antigen positive and multiplied by 100.

FIG. 4 shows a yeast library sort using the SARS-COV-1 spike protein on the FACSAria IIu, according to aspects of this disclosure. A representative 10,000 events are shown from the sort. The x axis is FITC tagged for c-myc expression, a surrogate for full-length scFv expression and the y axis is a stain of Alexa Flour 647, a surrogate for antigen positivity. The sort was done with 125 nM SARS-COV-1 spike protein. Yeast which were positive for both FITC and Alexa Flour 647 (upper right quadrant, labeled “Q2”) expressed scFvs which bound to SARS-COV-1 spike protein. Yeast were sorted into 1 of 2 gates, either a “hi” gate for all the highest antigen positive clones, or a “low” gate, for all other antigen positive clones. Yeast which did not fall into either one of these two populations were not sorted.

FIG. 5 shows data from biolayer interferometry (BLI) binding assays of scFv proteins identified in the yeast library sort to spike proteins, according to aspects of this disclosure. Binding is shown between the scFvs (scFv names are listed on the left) to either SARS-COV-2 spike (left column of left panel, labeled “SARS-COV-2 2P”), SARS-COV-1 spike (right column of left panel, labeled “SARS-COV-1 2P”), or anti-his octet biosensors (right panel). The values plotted are the “response” or the nm shift after 2 min association phase. Heatmap shading scale is shown on the right side of each panel.

FIG. 6 shows BLI binding curves of IgG antibodies produced from the scFvs identified from the sorts depicted in FIG. 4 and FIG. 5 to either SARS-COV-2 spike, SARS-COV-1 spike, or MERS spike, according to aspects of this disclosure. Purified IgGs were tested for binding to SARS-COV-2 spike, SARS-COV-1 spike, and MERS spike at 100 nM using biolayer interferometry. A dotted line on the x axis depicts the transition from the association phase (2 min) to the dissociation phase (Imin) of the binding.

FIGS. 7A-7B show BLI competition plots depicting the competition groups of the IgG antibodies used in FIG. 6, according to aspects of this disclosure. BLI biosensors loaded with SARS-COV-2 (FIG. 7A) or SARS-COV-1 (FIG. 7B) spike were associated with the antibodies denoted on the x axis until saturation (5 min). These biosensors were then exposed to the antibodies shown on the y axis and the response (nm shift after 2 min) was determined. If the antibodies compete with one another for binding the response would be very low because the antibody which initially was associated would block the binding of the second antibody. If the antibodies do not compete for binding the response will be approximately as high as if the first antibody was not associated. The rows of the plots were normalized such that zero was set to be the antibody competing with itself and 1 was set to be the antibody binding to a biosensor tip which contained only SARS-COV-2 (FIG. 7A) or SARS-COV-1 (FIG. 7B) spike and no other loaded antibody. Therefore, values of ˜1 suggest the antibodies don't compete, where values of ˜0 suggest the antibodies do compete.

FIG. 8 shows that the IgG antibodies described above do not neutralize SARS-COV-2 or SARS-COV-1, according to aspects of this disclosure. A single point neutralization assay for SARS-COV-2 (top panel) or SARS-COV-1 (bottom panel) by the IgGs listed on the x axis was conducted at 100 nM. Values shown are normalized to 100% for virus only (i.e., no antibody added) and 0% for cells only (i.e., no virus added). Values close to 100% indicate that no neutralization occurred, whereas values close to 0% suggest neutralization.

FIGS. 9A-9C show the development, testing, and predicted spike binding properties of scFv-ACE2 fusion proteins, according to aspects of this disclosure. FIG. 9A shows the bivalent scFv-ACE2 fusion construct that was designed utilizing three of the scFvs from above (CV10, CV27, COVA2-14). The non-neutralizing scFv (“Non-neut-scFv”) comprising a heavy chain sequence (“HC”) and a light chain sequence (“LC”) is linked to ACE2 using a linker containing a hexa-his tag (“H”) and a TEV protease cleavage site (“T”) in the linker (FIG. 9A, top panel) Also shown are the products produced by TEV protease cleavage, along with the approximate size of the cleaved fragments (FIG. 9A, bottom panel). FIG. 9B is an SDS-PAGE gel depicting the purified scFv-ACE2 fusions with and without TEV digestion. Arrows denote, Full-length scFv-ACE2 fusions (top), cleaved ACE2 (middle), and cleaved scFv (bottom) FIG. 9C is a schematic depiction of the interaction between the scFv-ACE2 fusion (with and without TEV cleavage) and a coronavirus spike protein.

FIGS. 10A-10B show that uncleaved scFv-ACE2 fusions bind spike proteins more strongly than TEV-cleaved scFv-ACE2 fusion proteins, according to aspects of this disclosure. FIG. 10A shows a BLI binding study comparing the binding of uncleaved and TEV cleaved scFv-ACE2 fusions to either SARS-COV-2 spike (left panel) or SARS-COV-1 spike (right panel). The dotted line at 2 mins depicts the transition from association to dissociation phases. The darker color lines are the uncleaved samples which show greater association (higher nm shift) and also slower dissociation. FIG. 10B shows a BLI binding competition experiment, comparing the competition between uncleaved and TEV cleaved scFv-ACE2 fusions competing with either human-Fc-ACE2 (hFc-ACE2) or CB6 (an antibody which binds to the RBD at the ACE2 binding site) on either SARS-COV-2 spike (top panel) or SARS-COV-1 spike (bottom panel). Spike protein was associated to saturating (5 min) with scFv-ACE2 fusions with and without TEV cleavage. These spike-scFv-ACE2 complexes were then associated with either hFc-ACE2 or CB6. The values were normalized to either the hFc-ACE2 or CB6 binding alone, where this value was set to 1. If the initial association with the scFv-ACE2 complex prevented binding of the hFc-ACE2 or CB6 the normalized nm shift would be expected to be much lower than 1 (lighter colors), suggesting that hFc-ACE2 or CB6 were unable to bind in the presence of the scFv-ACE2 fusions. If the binding was not perturbed by the initial association with the scFv-ACE2 complexes, then the normalized nm shift would be expected to be much closer to 1 (darker colors).

FIGS. 11A-11B show a Western blot analysis of cellular supernatant from a neutralization experiment of the scFv-ACE2 fusions against SARS-COV-2 (FIG. 11A) or SARS-CoV-1 (FIG. 11B) pseudotyped lentivirus, according to aspects of this disclosure. The scFv-ACE2 fusions were tested in their uncleaved and TEV cleaved forms for their neutralizing potency against pseudotyped lentivirus. To ensure that the constructs remained intact over the course of the assay the media was changed on day 1 to remove virus and the cellular supernatants were tested via western blot analysis against the hexa-his tag. In the uncleaved form the his tag is on the scFv-ACE2 fusion (MW ˜100 kDa) and in the cleaved form the his tag is on the scFv component (MW ˜31 kda).

FIGS. 12A-12B show that uncleaved scFv-ACE2 fusion proteins are able to neutralize SARS-COV-2 (FIG. 12A) and SARS-COV-1 (FIG. 12B) more effectively than TEV-cleaved fusion proteins, according to aspects of this disclosure. Values are normalized to a “virus alone” set of wells which is considered 100% infectivity, and a “cells alone” set of wells which is considered 0% infectivity. The x axis shows the concentration of the samples tested and the y axis shows the percent infectivity. Values closer to 0% indicate that the samples are neutralizing. Open symbols are the uncleaved scFv-ACE2 fusions whereas closed symbols have been TEV digested.

FIGS. 13A-13C show the design and characterization of IgG-ACE2 fusion constructs, according to aspects of this disclosure. Sequences from the three scFv fragments tested above (CV10, CV27, and COVA2-14) were expressed as IgG1 constructs. FIG. 13A is a schematic depiction of the constructs. VH: heavy chain variable region; VL: light chain variable region, CH1, CH2, CH3: heavy chain constant region; CL: light chain constant region; H: hexa-his tag; T: TEV protease cleavage site. ACE2 is linked to the carboxy-terminus (C-terminus) of the light chain. FIG. 13B shows a schematic depiction of the assembled IgG-ACE2 fusion antibody, with an ACE2 domain linked onto each of the two light chains. FIG. 13C is an SDS-PAGE gel depicting the purified IgG-ACE2 fusions with and without TEV digestion.

FIGS. 14A-14C show a comparison of spike protein binding between cleaved and uncleaved IgG-ACE2 fusion proteins, according to aspects of this disclosure. Binding of the uncleaved and TEV cleaved CV10-ACE2 fusion at 15 nM (FIG. 14A), CV27-ACE2 fusion at 100 nM (FIG. 14B), and COVA2-14-ACE2 at 100 nM (FIG. 14C) to SARS-COV-2 spike (left column), SARS-COV-1 spike (middle column), and SARS-COV-2 RBD (right column) was measured. Uncleaved protein binding lines are in the darker black color, and cleaved protein binding lines are in the lighter gray color. The association phase was conducted for 6 min, and then the dissociation phase for 2 min. A dotted line at 6 min depicts the transition from association to dissociation phases.

FIGS. 15A-15B show a western blot analysis of cellular supernatant from a neutralization experiment of the IgG-ACE2 fusions against SARS-COV-2 (FIG. 15A) or SARS-CoV-1 (FIG. 15B) pseudotyped lentivirus, according to aspects of this disclosure. The IgG-ACE2 fusions were tested in their uncleaved and TEV cleaved forms for their neutralizing potency against pseudotyped lentivirus. To ensure that the constructs remained intact over the course of the assay the media was changed on day 1 to remove virus and the cellular supernatants were tested via western blot analysis against the human-Fc tag. In the uncleaved form the hFc tag is on the IgG-ACE2 fusion (MW ˜300 kDa) and in the cleaved form the Fc tag is on the IgG component (MW ˜150 kDa). Also shown (right) is the uncleaved and TEV cleaved forms of hFc-ACE2, a control carried in the neutralization assay.

FIGS. 16A-16B show that IgG-ACE2 fusion proteins are able to neutralize SARS-CoV-2 (FIG. 16A) and SARS-COV-1 (FIG. 16B) more effectively than uncleaved and TEV-cleaved hFc-ACE2 fusion proteins, according to aspects of this disclosure. Values are normalized to a “virus alone” set of wells which is considered 100% infectivity, and a “cells alone” set of wells which is considered 0% infectivity. The x axis shows the concentration of the samples tested and the y axis shows the percent infectivity. Values closer to 0% indicate that the samples are neutralizing. Shaded symbols are the uncleaved IgG-ACE2 fusions whereas open symbols are uncleaved hFc-ACE2 and closed symbols are hFc-ACE2 which has been TEV digested.

FIGS. 17A-17B show a depiction of a bispecific construct targeting two distinct non-neutralizing epitopes on SARS-COV-2 and SARS-COV-1, according to aspects of this disclosure. This construct avoids antigenic escape at a single epitope by targeting two distinct, non-overlapping epitopes. The construct is produced by creating a bump and hole hFc domain and a CrossMAb to promote correct pairing of the heavy and light chains. This construct is made with a CV27 component and either a COVA2-14 or CV10 component. The linker and ACE2 fusion protein is only on the LC of the CV27. Constructs are shown with (FIG. 17A) and without (FIG. 17B) a TEV cleavage site (white oval) in the linker.

FIG. 18 shows cleavage of the CrossMAb constructs, according to aspects of this disclosure. The CrossMAb constructs pre- and post-TEV cleavage are shown on a Coomassie stained gel electrophoresis gel along with an IgG-ACE2 fusion (lane 2) or IgG alone (lane 3). Given that there is only a single ACE2 fusion per IgG in these CrossMAb constructs, the expected MW of these constructs in the uncleaved form (lanes 4 and 6) is ˜225 kDa. TEV-cleaved constructs are shown in lanes 5 and 7, with two lower molecular weight fragments that correspond to the MW of the CrossMAb IgG (approximately 150 kDa) and ACE2 (approximately 75 kDa).

FIG. 19 shows that uncleaved CrossMAb-ACE2 fusions are able to neutralize SARS-CoV-2 more effectively than TEV-cleaved CrossMAb-ACE2 fusion proteins, according to aspects of this disclosure. Values are normalized to a “virus alone” set of wells which is considered 100% infectivity, and a “cells alone” set of wells which is considered 0% infectivity. The x axis shows the concentration of the samples tested and the y axis shows the percent infectivity. Values closer to 0% indicate that the samples are neutralizing. Open symbols are the uncleaved CrossMAb-ACE2 fusions whereas closed symbols have been TEV digested.

FIG. 20 shows cleavage of scFv-ACE2 fusion proteins, according to aspects of this disclosure. Depicted is a Coomassie stained SDS-PAGE gel showing purified scFv-ACE2 fusions (as schematically depicted in FIG. 9A) with and without TEV digestion. Arrows denote, Full-length scFv-ACE2 fusions (top), cleaved ACE2 (middle), and cleaved scFv (bottom).

FIG. 21 shows that uncleaved scFv-ACE2 fusions compete with human-Fc-ACE2 for binding to spike proteins more strongly than TEV-cleaved scFv-ACE2 fusion proteins, according to aspects of this disclosure. Depicted is a BLI binding competition experiment, comparing the competition between uncleaved and TEV cleaved scFv-ACE2 fusions competing with human-Fc-ACE2 on either SARS-COV-2 spike (top panel) or SARS-COV-1 spike (bottom panel). Spike protein was associated to saturating (5 min) with scFv-ACE2 fusions with and without TEV cleavage. These spike-scFv-ACE2 complexes were then associated with hFc-ACE2. The values were normalized to 0 (no binding) and 100 (hFc-ACE2 binding alone, i.e., no competitor). If the initial association with the scFv-ACE2 complex prevented binding of the hFc-ACE2, the normalized nm shift would be expected to be much lower than 100 (lighter colors), suggesting that hFc-ACE2 was unable to bind in the presence of the scFv-ACE2 fusions. If the binding was not perturbed by the initial association with the scFv-ACE2 complexes, then the normalized nm shift would be expected to be much closer to 100 (darker colors).

FIG. 22 shows that uncleaved scFv-ACE2 fusions bind spike proteins more strongly than TEV-cleaved scFv-ACE2 fusion proteins, according to aspects of this disclosure. Depicted is a BLI binding study comparing the binding of uncleaved and TEV cleaved scFv-ACE2 fusions to either SARS-COV-2 spike (left panel) or SARS-COV-1 spike (right panel). The dotted line at 1 min depicts the transition from association to dissociation phases. The dashed lines are TEV-cleaved samples, and the solid lines are uncleaved samples, which show greater association (higher nm shift) and also slower dissociation.

FIG. 23 shows that scFv-ACE2 fusion proteins show broad spectrum neutralization, according to aspects of this disclosure. Depicted are pseudoviral 50% neutralization values (NT₅₀) for five scFv-ACE2 fusion constructs (bottom axis) against a range of SARS-COV-2 variants of concern with and without TEV cleavage. Uncleaved scFv-ACE2 fusions show much more robust neutralizing activity against all variants of concern compared to the TEV cleaved scFv-ACE2, suggesting that the linkage between the neutralizing component and the conserved-binding component is required for neutralizing activity. NT₅₀ values shown are the average of two independent experiments.

FIGS. 24A and 24B show a depiction of bispecific constructs targeting two distinct non-neutralizing epitopes on SARS-COV-2 and SARS-COV-1 fused to the ectodomain of ACE2, according to aspects of this disclosure. This construct avoids the risk of antigenic escape at a single epitope by targeting two distinct, non-overlapping epitopes. The construct is produced by creating a bump and hole hFc domain and a CrossMAb to promote correct pairing of the heavy and light chains. This construct is made with a COV2-2449 component and a CV10 component. The linker and ACE2 fusion protein is only on the LC of the COV2-2449 component. Constructs are shown with (FIG. 24A) and without (FIG. 24B) a TEV cleavage site (white oval) in the linker.

FIG. 25 shows cleavage of a CV10-COV2-2449-ACE2 CrossMAb, according to aspects of this disclosure. Depicted is a Coomassie stained SDS-PAGE gel showing the CrossMAb before or after TEV cleavage and with or without 2-mercaptoethanol (BME), as depicted above the gel. Bands are identified as 1: full-length CrossMAb; 2: cleaved CrossMAb IgG; 3: COV2-2449-LC-ACE2 fusion; 4: ACE2 (reduced ACE2 shows double banding); 5: HC; 6: cleaved COV2-2449-LC with linker; and 7: CV10 LC.

FIG. 26 shows that uncleaved CV10-COV2-2449-ACE2 CrossMAbs compete with human-Fc-ACE2 for binding to SARS-COV-2 spike protein more strongly than TEV-cleaved CV10-COV2-2449-ACE2 CrossMAbs, according to aspects of this disclosure. Depicted is a BLI binding competition experiment, comparing the competition between uncleaved and TEV cleaved CV10-COV2-2449-ACE2 CrossMAbs competing with human-Fc-ACE2 on SARS-CoV-2 spike. Spike protein was associated to saturating (5 min) with CV10-COV2-2449-ACE2 CrossMAbs with and without TEV cleavage. These spike-CV10-COV2-2449-ACE2 CrossMAb complexes were then associated with hFc-ACE2. The values were normalized to 0 (no binding) and 1.0 (hFc-ACE2 binding alone, i.e., no competitor). If the initial association with the CV10-COV2-2449-ACE2 CrossMAb prevented binding of the hFc-ACE2, the normalized nm shift would be expected to be much lower than 1.0 (lighter colors), suggesting that hFc-ACE2 was unable to bind in the presence of the CV10-COV2-2449-ACE2 CrossMAbs. If the binding was not perturbed by the initial association with the CV10-COV2-2449-ACE2 CrossMAbs, then the normalized nm shift would be expected to be much closer to 1.0 (darker colors).

FIG. 27 shows that CV10-COV2-2449-ACE2 CrossMAbs show broad spectrum neutralization, according to aspects of this disclosure. The plot shows pseudoviral 50% neutralization values (NT₅₀) for the CV10-COV2-2449-ACE2 CrossMAb against a range of SARS-COV-2 variants of concern with and without TEV cleavage. Uncleaved CV10-COV2-2449-ACE2 fusions show much more robust neutralizing activity against all variants of concern compared to the TEV cleaved form, suggesting that the linkage between the neutralizing component (ACE2) and the conserved-binding component (CrossMAb) is required for neutralizing activity. NT₅₀ values shown are the average of two independent experiments.

FIG. 28 shows two bispecific CrossMAb antibodies, according to aspects of this disclosure, which would be able to bind on one arm to a conserved, non-neutralizing site outside the SARS-COV-2 spike RBD using the antibodies described herein, and on the other arm to the SARS-COV-2 spike RBD. Top and bottom panels depict two different arrangements of the bispecific antibodies-both utilizing the same technology, but with a different binding specificity on each arm, as labelled.

FIG. 29 shows additional approaches for the development of antibodies which simultaneously target at least one highly conserved, non-neutralizing epitope and an RBD-based epitope, according to aspects of this disclosure. Such antibodies could use a CrossMAb (top panel), similar to those discussed here, with a fusion between the LC of one arm (depicted here) or both arms and a neutralizing scFv encoding an antibody that binds to the RBD. The bottom panel shows another approach, a bispecific antibody where both heavy chains and light chains of the antibodies are identical, but fused to the C-terminus of the antibody LC (depicted here) or HC is a linker and an scFv for a neutralizing RBD-directed antibody. In alternate configurations, the scFv binding specificity and the IgG binding specificity could be swapped (not shown).

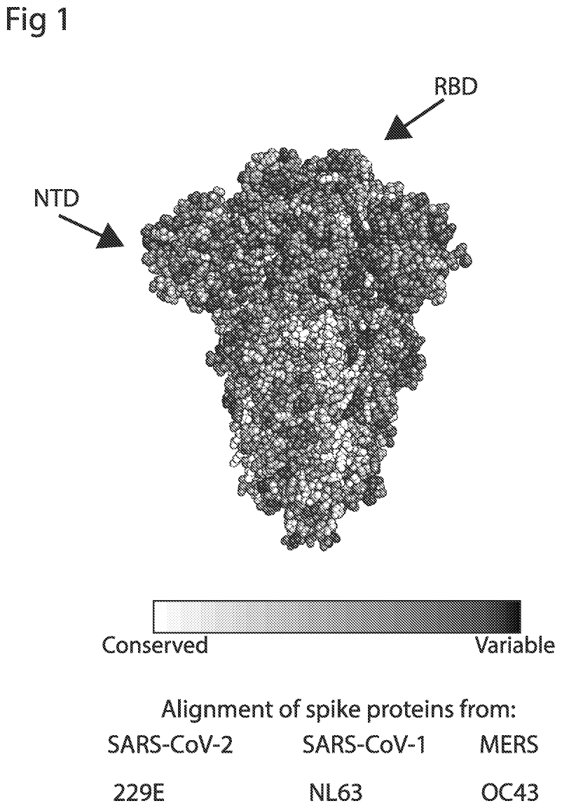

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

### I. Terminology

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95 X, 0.96 X, 0.97 X, 0.98 X, 0.99 X, 1.01 X, 1.02 X, 1.03 X, 1.04 X, and 1.05 X. Thus, expressions “about X” or “approximately X” are intended to teach and provide written support for a claim limitation of, for example, “0.98 X.” Alternatively, in biological systems, the terms “about” and “approximately” may mean values that are within an order of magnitude, preferably within 5-fold, and more preferably within 2-fold of a given value Numerical quantities given herein are approximate unless stated otherwise, meaning that the term “about” or “approximately” can be inferred when not expressly stated. When “about” is applied to the beginning of a numerical range, it applies to both ends of the range.

“Virus” is used in both the plural and singular senses. “Virion” refers to a single virus. For example, the expression “coronavirus virion” refers to a coronavirus particle.

Coronaviruses are a group of enveloped, single-stranded RNA viruses that cause diseases in mammals and birds. Coronavirus hosts include bats, pigs, dogs, cats, mice, rats, cows, rabbits, chickens and turkeys. In humans, coronaviruses cause mild to severe respiratory tract infections. Coronaviruses vary significantly in risk factor. Some can kill more than 30% of infected subjects. The following strains of human coronaviruses are currently known: Human coronavirus 229E (HCoV-229E); Human coronavirus OC43 (HCoV-OC43); Severe acute respiratory syndrome coronavirus (SARS-COV or SARS-COV-1); Human coronavirus NL63 (HCoV-NL63, New Haven coronavirus); Human coronavirus HKU1 (HCoV-HKU1); Middle East respiratory syndrome-related coronavirus (MERS-COV), also known as novel coronavirus 2012 and HCoV-EMC; and Severe acute respiratory syndrome coronavirus 2 (SARS-COV-2), also known as 2019-nCOV or “novel coronavirus 2019.” Several variants of SARS-COV-2 have been identified, including the Alpha, Beta, Gamma, Delta, and Omicron strains (see Garcia-Beltran, et al., 2021, medRxiv preprint Serv. Heal. Sci., doi: 10.1101/2021.02.14.21251704 and Alkhatib et al., 2021, Microbiology Spectrum 9(3)), all of which have mutations relative to the original SARS-COV-2 isolate (see list of shared and unique mutations at covariants. org/shared-mutations). The coronaviruses HCoV-229E,-NL63, -OC43, and HKU1 continually circulate in the human population and cause respiratory infections in adults and children world-wide.

Spike protein (or “S protein”) is a coronavirus surface protein that is able to mediate receptor binding and membrane fusion between a coronavirus virion and its host cell. Characteristic spikes on the surface of coronavirus virions are formed by ectodomains of homotrimers of Spike protein. In comparison to trimeric glycoproteins found on other human-pathogenic enveloped RNA viruses, coronavirus Spike protein is considerably larger, and totals nearly 450 kDa per trimer. Ectodomains of coronavirus Spike proteins contain an N-terminal domain named S1, which is responsible for binding of receptors on the host cell surface, and a C-terminal S2 domain responsible for fusion. S1 domain of SARS-COV-2 Spike protein is able to bind to Angiotensin-converting enzyme 2 (ACE2) of host cells. The region of SARS-COV-2 Spike protein S1 domain that recognizes ACE2 is a 25 kDa domain called the receptor binding domain (RBD) (Walls et al., 2020, “Structure, Function, and antigenicity of the SARS-COV-2 Spike Glycoprotein,” Cell 181(2):281-292.e6). Analysis of sera from COVID-19 patients demonstrates that antibodies are elicited against the Spike protein and can inhibit viral entry into the host cell (Brouwer et al., 2020, “Potent neutralizing antibodies from COVID-19 patients define multiple targets of vulnerability,” Science, 369(6504): 643-650). The first Cryo-EM structure of SARS-COV-2 Spike protein is described in Wrapp et al., 2020, “Cryo-EM structure of the 2019-nCOV spike in the prefusion conformation,” Science 367 (6483): 1260-1263.

The terms “protein,” “peptide,” and “polypeptide” are used interchangeably to refer to a polymer of amino acid residues. The terms apply to naturally occurring amino acid polymers and non-natural amino acid polymers, as well as to amino acid polymers in which one (or more) amino acid residue is an artificial chemical mimetic of a corresponding naturally occurring amino acid. The terms encompass amino acid chains of any length, including full-length proteins, wherein the amino acid residues are linked by covalent peptide bonds.

A “domain” of a protein or a polypeptide refers to a region of the protein or polypeptide defined by structural and/or functional properties. Exemplary function properties include enzymatic activity and/or the ability to bind to or be bound by another protein or non-protein entity. For example, coronavirus Spike protein contains S1 and S2 domains.

The term “oligomer” and related terms, when used in reference to polypeptides or proteins, refer to complexes formed by two or more polypeptide or protein monomers, which can also be referred to as “subunits” or “chains.” For example, a trimer is an oligomer formed by three polypeptide subunits.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of a naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The terms “fusion protein,” “fusion polypeptide,” and the related terms relate to polypeptide molecules, including artificial or engineered polypeptide molecules, that include two or more amino acid sequences previously found in separate polypeptide molecule, that are joined or linked in a fusion protein amino acid sequence to form a single polypeptide. For example, a fusion protein can be an engineered recombinant protein containing amino acid sequence from at least two unrelated proteins that have been joined together, via a peptide bond, to make a single protein. In this context, proteins are considered unrelated, if their amino acid sequences are not normally found joined together via a peptide bond in their natural environment, for example, inside a cell. For example, the present disclosure describes fusion proteins that include an amino acid sequence of a coronavirus receptor polypeptide and an amino acid sequence of an antibody, which are unrelated proteins. The amino acid sequences of a fusion protein are encoded by corresponding nucleic acid sequences that are joined “in frame,” so that they are transcribed and translated to produce a single polypeptide. The amino acid sequences of a fusion protein can be contiguous or separated by one or more spacer, linker or hinge sequences. Fusion proteins can include additional amino acid sequences, such as, for example, signal sequences, tag sequences, and/or linker sequences.

The term “antibody” and the related terms refer to an immunoglobulin or its fragment that binds to a particular spatial and polar organization of another molecule. Immunoglobulins include various classes and isotypes, such as IgA, IgD, IgE, IgG1, IgG2a, IgG2b and IgG3, IgG4, IgM, etc. . . . An antibody can be monoclonal or recombinant, and can be prepared by laboratory techniques, such as by preparing continuous hybrid cell lines and collecting the secreted protein, or by cloning and expressing nucleotide sequences or their mutagenized versions coding at least for the amino acid sequences required for binding. Antibodies as referenced herein may have sequences derived from non-human antibodies, human sequence, chimeric sequences, and wholly synthetic sequences. The term “antibody” encompasses natural, artificially modified, and artificially generated antibody forms, such as humanized, human, single-chain, chimeric, synthetic, recombinant, hybrid, mutated, grafted, and in vitro generated antibodies and their fragments. The term “antibody” also includes composite forms including but not limited to fusion proteins containing an immunoglobulin moiety. “Antibody” also refers to non-quaternary antibody structures (such as camelids and camelid derivatives) and antigen-binding fragments of antibodies, minibodies, bispecific antibodies, nanobodies (also referred to as VHH fragments), and diabodies. See, Siontorou C G. 2013, “Nanobodies as novel agents for disease diagnosis and therapy,” Int J Nanomedicine 8:4215-4227. Antibody fragments may include Fab, Fv, F(ab′)2, Fab′, scFv, dsFv, ds-scFv, Fd, dAb, Fc, and the like. A natural antibody digested by papain yields three fragments: two Fab fragments and one Fc fragment. The Fc fragment is dimeric and contains two CH2 and two CH3 heavy chain domains. CH3 domains interact to form a homodimer. See Yang et al., 2018, “Engineering of Fc Fragments with Optimized Physicochemical Properties Implying Improvement of Clinical Potentials for Fc-Based Therapeutics” Frontiers in Immunology 8:1860. Fc domains in antibodies may also be optimized to alter antibody characteristics of interest (e.g., bioavailabilty, serum half-life). See, e.g., Ko et al., 2014, Nature 514:642-645 and Zalevsky et al., 2010, Nat. Biotech. 28:157-159. In addition, aggregates, polymers and conjugates of immunoglobulins or their fragments can be used where appropriate. Additional details of antibodies useful in the context of this disclosure are provided below.

As used herein, the term antibody encompasses, but is not limited to, whole immunoglobulin (i.e., an intact antibody) of any class. A natural immuoglobulin G (IgG) antibody molecule is a tetramer that contains two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Within light and heavy chains, the variable and constant regions are joined by a “J” region of about 12 or more amino acids, with the heavy chain also including a “D” region of about 10 more amino acids See generally, Fundamental Immunology, Paul, W., ed, 3rd ed. Raven Press, N Y, 1993, SH. 9 (incorporated by reference in its entirety for all purposes). Antibody sequences and structural information is widely available. See, e.g., Lima et al., 2020, “The ABCD database: a repository for chemically defined antibodies” Nucleic Acids Research 48:D261-D264. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. As used herein, the term antibody also encompasses an antibody fragment, for example, an antigen binding fragment. Antigen binding fragments comprise at least one antigen binding domain. One example of an antigen binding domain is an antigen binding domain formed by a VH-VL dimer. Antibodies and antigen binding fragments can be described by the antigen to which they specifically bind.

Within each light or heavy chain variable region, there are three short segments (averaging 10 amino acids in length) called the complementarity determining regions (“CDRs”). The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. Each VH and VL generally comprises three CDRs and four FRs, arranged in the following order (from N-terminus to C-terminus): FR1-CDR1-FR2-CDR2-FR3-CDR3-FR4. The CDRs are involved in antigen binding, and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (VH) may be designated as CDRH1, 2, 3, while CDR sequences on the light chain (VL) may be designated as CDRL1, 2, 3.

A “Fc fragment” contains two heavy chain fragments comprising the CH2 and CH3 domains of an antibody. The two heavy chain fragments are held together by two or more disulfide bonds and by hydrophobic interactions of the CH3 domains. A Fc domain introduced into a fusion protein may promote dimerization.

A “Fab fragment” is comprised of one light chain, and the CH1 and variable regions of one heavy chain and can specifically recognize a target epitope, such as an epitope of a Spike protein. A Fab domain introduced into a fusion protein results in binding of the fusion protein to the target.

A “single-chain variable fragment” or “scFv fragment” is a fusion protein comprising the variable regions of a heavy chain and a light chain from an antibody. The heavy chain and light chain portions may be connected by a linker peptide. An scFv fragment may retain the binding specificity of the antibody from which it is derived.

A “neutralizing polypeptide” is a polypeptide that, when present at physiologically and/or pharmaceutically acceptable concentrations, is capable of keeping an infectious agent, such as a virus, from infecting a cell by neutralizing or inhibiting one or more parts of the life cycle of the infectious agent. A common type of neutralizing polypeptide is a neutralizing antibody, however other polypeptides that can bind specifically to an infectious agent can also be neutralizing (e.g., polypeptides based on the receptor bound by a virus). Typically, neutralizing antibodies can neutralize or inhibit multiple different strains and, as such, can provide protective immunity against heterogeneous and evolving infection agents. For coronaviruses, neutralizing antibodies typically specifically bind to the receptor binding domain (RBD) of the spike protein and typically act to disrupt or prevent interaction of the virus spike with its receptor such that virus entry into the target cell is prevented or reduced. As such, neutralizing antibodies can act to prevent or reduce the incidence of coronavirus infection. Because of the neutralizing ability of neutralizing antibodies, coronaviruses face evolutionary pressure to decrease or eliminate binding of neutralizing antibodies through mutation. Coronaviruses that have epitopes that can be bound by neutralizing antibodies will not propagate as effectively in hosts expressing the neutralizing antibodies relative to coronaviruses that have mutations in the epitopes that reduce or prevent binding of neutralizing antibodies Therefore, neutralizing antibody epitopes are highly variable among different coronaviruses and variant coronaviruses, and this variability makes it difficult to use neutralizing antibodies to treat patients infected with different types or strains of coronavirus.

The term “non-neutralizing antibody”, as used herein, refers to an antibody that, when present at physiologically and/or pharmaceutically acceptable concentrations, has little to no ability of keeping an infectious agent, such as a virus, from infecting a cell by neutralizing or inhibiting one or more parts of the life cycle of the infectious agent.

### II. Introduction

Provided in this disclosure are fusion proteins and modified proteins that specifically bind to one or more coronavirus spike proteins, various compositions of such fusion proteins and/or and modified proteins, and methods of their use. As detailed below, the fusion proteins take advantage of antibodies that bind to highly conserved epitopes in coronavirus spike proteins and, as such, are able to bind to a broad spectrum of coronaviruses, including all known SARS-COV-2 variants of concern (VOCs).

This disclosure further provides for nucleic acids encoding such fusion proteins and modified proteins or domains thereof, as well as constructs, expression cassettes, and vectors containing such nucleic acids, and host cells capable of expressing the fusion proteins, the modified proteins, and/or domains thereof. Additionally, this disclosure provides for prophylactic and therapeutic methods employing the fusion proteins and modified proteins of the disclosure.

III. Fusion proteins and modified proteins

Provided herein are fusion proteins and modified proteins that specifically bind to one or more coronavirus spike proteins. The provided fusion proteins comprise multiple domains: a neutralizing polypeptide that binds to a first coronavirus spike protein (e.g., to the RBD), a peptide linker, and an antibody that specifically binds an epitope in a conserved region of a second coronavirus spike protein. The provided modified proteins comprise multiple domains: a neutralizing polypeptide that binds to a RBD of a first coronavirus spike protein, a non-peptide linker, and an antibody that specifically binds an epitope in a conserved region of a second coronavirus spike protein. In some embodiments, the neutralizing polypeptide is a coronavirus receptor polypeptide. In some embodiments, the neutralizing polypeptide is a neutralizing antibody.

In some embodiments, the binding of the neutralizing polypeptide to a RBD of a coronavirus spike protein facilitates neutralization of the virus, while the antibody that specifically binds an epitope in a conserved region of a coronavirus spike protein enhances the binding affinity of the fusion protein or modified protein to one or more types or strains of coronavirus. As demonstrated in the Examples herein, the combination of the antibody that specifically binds an epitope in a conserved region of a coronavirus spike protein (e.g., a non-neutralizing antibody) with the neutralizing polypeptide (e.g., a coronavirus receptor polypeptide) results in the fusion proteins and modified proteins having increased binding affinity for coronavirus RBD and spike protein relative to the binding affinity of the neutralizing polypeptide portion alone, thus resulting in improved virus neutralization. In some embodiments, the fusion proteins or modified proteins provided herein can be used as therapeutic agents to treat subjects infected with a coronavirus or to prevent coronavirus infection (e.g., in a subject at high risk of coronavirus exposure) for known coronaviruses and for new coronaviruses that evolve in the future. As described above, the fusion proteins and modified proteins are able to bind to different coronaviruses (e.g., SARS-COV-2 and its variants, SARS-COV, MERS-COV) because the epitopes bound by the antibody that specifically binds an epitope in a conserved region of a coronavirus spike protein are conserved across coronavirus types and strains. Additionally, various neutralizing polypeptides (e.g., coronavirus receptor polypeptides used by different types of coronaviruses, neutralizing antibodies binding different coronaviruses or different variants, etc.) may be linked to the that specifically binds an epitope in a conserved region of a coronavirus spike protein antibodies that specifically bind an epitope in a conserved region of a coronavirus spike protein, facilitating easy, rapid design of therapeutic fusion proteins and/or modified proteins for known coronaviruses such as SARS-COV-2, as well as novel coronaviruses that will arise in the future.

### A. Protein Domain Configurations

The domains of the fusion proteins and modified proteins provided herein may be present in a variety of configurations, which may be selected to optimize binding of and/or neutralization by the fusion protein or modified protein for one or more coronavirus spike protein targets. Exemplary, non-limiting embodiments are provided below. Descriptions are given, along with specific examples. Domains listed in order are intended to show fusion proteins or modified proteins from the amino-terminus (N-terminus) to the carboxy-terminus (C-terminus). However, it will be appreciated that additional configurations are possible (for example, either the N-terminus or C-terminus of a given domain may be joined to the next domain).

In some embodiments, the fusion proteins and modified proteins comprise a single neutralizing polypeptide (e.g., a coronavirus receptor polypeptide or a neutralizing antibody) and a single antibody that specifically binds an epitope in a conserved region of a coronavirus spike protein. In some embodiments, a fusion protein or modified protein comprises a single ACE2 polypeptide that binds to a RBD of a coronavirus spike protein and a single antibody that specifically binds an epitope in a conserved region of a coronavirus spike protein (e.g., ACE2-linker-antibody or antibody-linker-ACE2). As another example, a fusion protein or modified protein can comprise a single DPP4 polypeptide that binds to a RBD of a coronavirus spike protein and a single antibody that specifically binds an epitope in a conserved region of a coronavirus spike protein (e.g., DPP4-linker-antibody or antibody-linker-DPP4). The antibody that specifically binds an epitope in a conserved region of a coronavirus spike protein and the neutralizing polypeptide may be linked in any orientation. For example, the N-terminus or C-terminus of the neutralizing polypeptide may be linked to the N-terminus or C-terminus of the antibody that specifically binds an epitope in a conserved region of a coronavirus spike protein. In some embodiments, the neutralizing polypeptide or antibody that specifically binds an epitope in a conserved region of a coronavirus spike protein may be linked internally to another domain of the fusion protein or modified protein (e.g., the neutralizing polypeptide may be flanked by two portions of the antibody that specifically binds an epitope in a conserved region of a coronavirus spike protein).

In some embodiments, the fusion proteins or modified proteins comprise at least one neutralizing polypeptide (e.g., a neutralizing antibody or a coronavirus receptor polypeptide). In some embodiments, the fusion proteins or modified proteins comprise more than one neutralizing polypeptide. In some embodiments, each of the neutralizing polypeptides in a fusion protein or modified protein bind to the same coronavirus spike protein. In some embodiments, the neutralizing polypeptides bind to spike proteins of different coronaviruses. For example, a single fusion protein or modified protein provided herein can comprise an ACE2 receptor polypeptide (i.e., for binding to SARS-COV-2 and/or SARS-COV-1 spike protein) and a DPP4 receptor polypeptide (i.e., for binding to MERS-COV spike protein) along with an antibody that specifically binds an epitope in a conserved region of a coronavirus spike protein (e.g., ACE2-linker-antibody-linker-DPP4 or DPP4-linker-antibody-linker-DPP4) Such configurations may allow a given fusion protein or modified protein to bind to and/or neutralize different coronaviruses (e.g., SARS-COV-2, SARS-COV-1, and/or MERS-COV). As another example, a single fusion protein or modified protein provided herein can comprise two or more ACE2 receptor polypeptides along with an antibody that specifically binds an epitope in a conserved region of a coronavirus spike protein (e.g., ACE2-linker-antibody-linker-ACE2 or ACE2-ACE2-antibody) and/or two or more DPP4 receptor polypeptides (e.g., DPP4-linker-antibody-linker-DPP4 or DPP4-DPP4-antibody). Each of the neutralizing polypeptides may be linked to the other domains of the fusion protein or modified protein in any order and orientation. For example, the amino-terminus (N-terminus) or carboxy-terminus (C-terminus) of a neutralizing polypeptide may be linked to an N-terminus or C-terminus of the antibody that specifically binds an epitope in a conserved region of a coronavirus spike protein or portion thereof or to the N-terminus or C-terminus of another neutralizing polypeptide of the fusion protein or modified protein. As another example, a fusion protein or modified protein may have the N-terminus or C-terminus of a first neutralizing polypeptide linked to an N-terminus of the antibody that specifically binds an epitope in a conserved region of a coronavirus spike protein or portion thereof and the N-terminus or C-terminus of a second neutralizing polypeptide linked to a C-terminus of the antibody that specifically binds an epitope in a conserved region of a coronavirus spike protein or a portion thereof. In some embodiments, multiple neutralizing polypeptides may be linked internally to another domain of the fusion protein or modified protein. For example, one or more neutralizing polypeptides may be flanked by two portions of the antibody that specifically binds an epitope in a conserved region of a coronavirus spike protein.

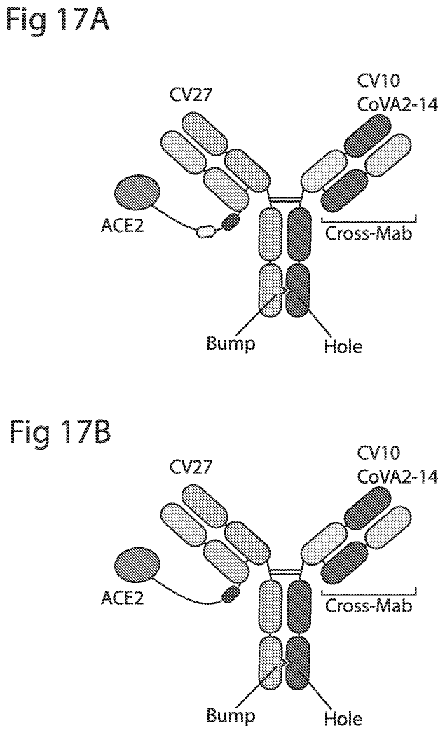

In some embodiments, the fusion proteins or modified proteins comprise at least one antibody that specifically binds an epitope in a conserved region of a coronavirus spike protein. In some embodiments, the fusion proteins or modified proteins comprise more than one antibody that specifically binds an epitope in a conserved region of a coronavirus spike protein. In some embodiments, such antibodies can specifically bind to the same coronavirus spike protein (e.g., all bind to a particular conserved epitope on the coronavirus spike protein or to different conserved epitopes on the same coronavirus spike protein). In some embodiments, such antibodies can specifically bind to different coronavirus spike proteins. In some embodiments of fusion proteins or modified proteins comprising more than one antibody that specifically binds an epitope in a conserved region of a coronavirus spike protein, the antibodies are linked via linkers (e.g., as discussed in the Linkers section below). In some embodiments, the antibodies are joined via dimerization domains (e.g., Fc domains). For example, a fusion protein or modified protein provided herein may comprise two antibodies that each specifically bind an epitope in a conserved region of a coronavirus spike protein, a first antibody that specifically binds to a conserved epitope on SARS-COV-2 spike protein and a second antibody that specifically binds to a conserved epitope on MERS-COV spike protein (e.g., ACE2-linker-first antibody-linker-second antibody or first antibody-linker-ACE2-linker-second antibody). As another example, a fusion protein or modified protein provided herein may comprise two antibodies that each specifically bind to a conserved epitope on a coronavirus spike protein (i.e., two of the same antibody or two different antibodies that specifically bind the same epitope) (e.g., ACE2-linker-antibody-linker-antibody or antibody-linker-ACE2-linker-antibody). Each of the antibodies may be linked to the other domains of the fusion protein or modified protein in any order and orientation. For example, an amino-terminus (N-terminus) or carboxy-terminus (C-terminus) of an antibody that specifically binds an epitope in a conserved region of a coronavirus spike protein may be linked to an N-terminus or C-terminus of another antibody that specifically binds an epitope in a conserved region of a coronavirus spike protein or to the N-terminus or C-terminus of a neutralizing polypeptide. As another example, a fusion protein or modified protein may have an N-terminus or C-terminus of a first antibody that specifically binds an epitope in a conserved region of a coronavirus spike protein linked to the N-terminus of a neutralizing polypeptide and an N-terminus or C-terminus of a second antibody that specifically binds an epitope in a conserved region of a coronavirus spike protein linked to the C-terminus of the neutralizing polypeptide. In some embodiments, the antibodies that each specifically bind an epitope in a conserved region of a coronavirus spike protein may be linked internally to another domain of the fusion protein or modified protein. For example, one or more antibodies that each specifically bind an epitope in a conserved region of a coronavirus spike protein may be flanked by two portions of a neutralizing polypeptide. Exemplary fusion proteins comprising more than one antibody that each specifically bind an epitope in a conserved region of a coronavirus spike protein are shown in FIGS. 17A-17B, FIGS. 24A-24B, and FIG. 28, top panel.

In some embodiments, the fusion proteins or modified proteins comprise more than one neutralizing polypeptide and more than one antibody that each specifically bind an epitope in a conserved region of a coronavirus spike protein, according to the descriptions and exemplary embodiments above.

Also provided are compositions comprising a dimer of fusion proteins or modified proteins as described herein. In some embodiments, the fusion proteins or modified proteins are expressed in a host cell and form a dimer that can be isolated as a composition. In some embodiments, the fusion protein or modified protein monomers in the dimer are the same fusion protein or modified protein. In some embodiments, the fusion protein or modified protein monomers in the dimer are different fusion proteins or modified proteins. In some embodiments, the fusion proteins or modified proteins comprise dimerization domains to facilitate dimerization. In some embodiments, the fusion proteins or modified proteins are synthesized and linked chemically to form dimers. Methods of producing the fusion proteins or modified proteins are discussed in more detail below.

### B. Neutralizing Polypeptides

In some embodiments of the fusion proteins and modified proteins provided herein, the neutralizing polypeptide is a coronavirus receptor polypeptide. In some embodiments, the coronavirus receptor polypeptide comprises an ACE2 receptor ectodomain polypeptide. Full-length human ACE2 is 805 amino acids in length (SEQ ID NO:269), of which amino acids 1-17 is a signal peptide that is cleaved from the mature protein. See NCBI Reference Sequence NP_001358344.1; see also UniProtKB Reference Q9BYF1. The ACE2 ectodomain is composed of a N-terminal peptidase domain (aa 18-614) and a C-terminal dimerization domain, also referred to as a “collectrin” domain (aa 615-740). Recent studies have revealed the structural basis of the high-affinity ACE2-spike interaction through the spike receptor binding domain (RBD) (Lan, J., et al., Nature, 581:215-220 (2020), Song, W., et al., PLoS Pathog. 14:e1007236, and Yan, R., et al., Science, 367(6485): 1444-1448 (2020)). In the context of this disclosure, the ACE2 ectodomain polypeptide can comprise amino acids 18-614 of SEQ ID NO:269 (SEQ ID NO:270) or variants thereof that are slightly longer and/or shorter at either end, such as, for example, a polypeptide comprising amino acids 19-615 of SEQ ID NO:269 (SEQ ID NO:271), as used in the Examples of this application.

In some embodiments, the coronavirus receptor polypeptide comprises an ACE2 receptor ectodomain polypeptide that is at least 80% identical (for example, at least 80%, 81%, 82%, 83%, 84%, 85%, 86%, 87%, 88%, 89%, 90%, 91%, 92%, 93%, 94%, 95%, 96%, 97%, 98% or 99% identical) to the amino acid sequence of SEQ ID NO:270 or SEQ ID NO:271 (i.e., the wild-type human amino acid sequence). In some embodiments, the ACE2 receptor ectodomain polypeptide comprises one or more mutations (i.e., relative to the wild-type human sequence). In some embodiments, the one or more mutations are able to increase binding affinity of the ACE2 receptor ectodomain polypeptide for the RBD of a coronavirus spike protein. In some embodiments, the ACE2 receptor ectodomain polypeptide comprises amino acid substitions at one or more of the following residues (the positions listed are relative to SEQ ID NO:269): the arginine at position 273 (R273), the histidine at position 378 (H378), the glutamate at position 402 (E402), the histidine at position 374 (H374), and the histidine at position 345 (H345). In some embodiments, the ACE2 receptor ectodomain polypeptide comprises one or more of the following amino acid substitions: R273A (i.e., the arginine residue at position 273 (relative to SEQ ID NO:269) is substituted for an alanine residue), H378A, E402A, H374N, and H345L. Exemplary ACE2 receptor ectodomain polypeptides comprising one or more such mutations are described in Liu, P., et al., Int. J. Biol. Macromol., 165:1626-1633 (2020); Glasgow, A., et al., Proc. Nat. Acad. Sci., 117(45):28046-28055 (2020); and Chan, K. K., et al., Science 369:1261-1265 (2020).

In some embodiments of the fusion proteins and modified proteins provided herein, the neutralizing polypeptide comprises a coronavirus receptor polypeptide that comprises a DPP4 receptor ectodomain polypeptide. Full-length human DPP4 is 766 amino acids in length (SEQ ID NO:272) and comprises an ectodomain at amino acids 29-766. See NCBI Reference Sequence NP_001366534.1; see also UniProtKB Reference P27487. Structural studies have examined the high-affinity binding of MERS-COV spike protein to DPP4 through the MERS-COV spike RBD. See, e.g., Wang, N., et al., Cell Research 23:986-993 (2013). Recent studies have also shown that DPP4 binds to the RBD of SARS-COV-2 spike protein. See Li, Y., et al., iScience 23:101160 (2020). In the context of this disclosure, the DPP4 ectodomain polypeptide can comprise amino acids 29-766 of SEQ ID NO:272 (SEQ ID NO:273) or variants thereof that are slightly longer and/or shorter at one or both ends, such as, for example, a polypeptide comprising amino acids 39-766 of SEQ ID NO:272 (SEQ ID NO:274), as used in the Examples of this application.

In some embodiments, the coronavirus receptor polypeptide comprises a DPP4 receptor ectodomain polypeptide that is at least 80% identical (for example, at least 80%, 81%, 82%, 83%, 84%, 85%, 86%, 87%, 88%, 89%, 90%, 91%, 92%, 93%, 94%, 95%, 96%, 97%, 98% or 99% identical) to the amino acid sequence of SEQ ID NO:273 or SEQ ID NO:274 (i.e. the wild-type human amino acid sequence). In some embodiments, the DPP4 receptor ectodomain polypeptide comprises one or more mutations (i.e., relative to the wild-type human sequence). In some embodiments, the one or more mutations are able to increase binding affinity of the DPP4 receptor ectodomain polypeptide for the RBD of a coronavirus spike protein. In some embodiments, the DPP4 receptor ectodomain polypeptide comprises one or more of the mutations described in Li, Y., et al., iScience 23.101160 (2020), Song, W., et al., Virology 471-473:49-53 (2014), or Wang, N., et al., Cell Research 23:986-993 (2013).

In some embodiments of the fusion proteins and modified proteins provided herein, the neutralizing polypeptide is a neutralizing antibody. In some embodiments, the neutralizing antibody binds to the RBD of a coronavirus spike protein. The neutralizing antibody of fusion proteins and modified proteins comprising a neutralizing antibody may be engineered in any suitable configuration (e.g., as an intact immunoglobulin, an scFv, an antigen binding fragment, or as part of a CrossMAb), as discussed further below for antibodies that specifically bind an epitope in a conserved region of a coronavirus spike protein. As fusion proteins and modified proteins comprising a neutralizing antibody and an antibody that specifically binds an epitope in a conserved region of a coronavirus spike protein are, in some embodiments, bispecific antibodies, they may be also be generated using any method known in the art for generating bispecific antibodies (see, e.g., Brinkmann and Kontermann, 2017, MAbs 9(2): 182-212).

Numerous neutralizing antibodies are known in the art (see, e.g., Cameroni et al., 2021, bioRxiv preprint published Dec. 14, 2021, doi: 10.1101/2021.12.12.472269 and Cameroni et al., 2021, Nature preprint published Dec. 23, 2021, doi: 10.1038/s41586-021-04386-2). For example, the neutralizing antibody sotrovimab binds to a conserved site on the RBD (see PCT Publication No. WO2021252878 by Alexander et al). Any antibody that is able to bind to the RBD and promote viral neutralization may be useful in the fusion proteins and modified proteins of the present disclosure. Additional exemplary neutralizing antibodies (along with clinical names where applicable) are listed in Table 1 below (neutralizing antibodies indicated in bold have been shown to neutralize the Omicron SARS-COV-2 variant effectively), and larger lists are available in the Coronavirus Antibody Database (Raybould et al., 2021, Bioinformatics 37(5). 734-735). In some embodiments, the fusion proteins and modified proteins of the present disclosure comprise any of the neutralizing antibodies discussed herein. Heavy chain (HC) and light chain (LC) sequences of particular neutralizing antibodies are set forth herein as SEQ ID NOs: 328-336. In some embodiments, the neutralizing antibody comprises a heavy chain variable region comprising an amino acid sequence that has at least 90% identity (e.g., at least 91%, at least 92%, at least 93%, at least 94%, at least 95%, at least 96%, at least 97%, at least 98%, or at least 99%) to any one of SEQ ID NOs: 328, 330, 331, 333, and 335. In some embodiments, the neutralizing antibody comprises a light chain variable region comprising an amino acid sequence that has at least 90% identity to any one of SEQ ID NOs: 329, 332, 334, and 336. In some embodiments, the neutralizing antibody comprises a heavy chain variable region comprising an amino acid sequence that has at least 90% identity to any one of any one of SEQ ID NOs: 328, 330, 331, 333, and 335 and a light chain variable region comprising an amino acid sequence that has at least 90% identity to any one of SEQ ID NOs: 329, 332, 334, and 336.

TABLE 1
Exemplary neutralizing antibodies.

| | |
|---|---|
| sotrovimab | S2H14 |
| CT-P59 (Regdanvimab) | S2D106 |
| COV2-2130 (Cilgavimab) | S2D8 |
| COV2-2196 (Tixagevimab) | S2D97 |
| REGN10933 (Casirivimab) | S2H7 |
| REGN10987 (Imdevimab) | S2H19 |
| LY-CoV555 (Bamlanivimab) | S2H70 |
| LY-CoV016 (also known as CB6; Etesevimab) | S2M11 |
| VIR-7832 | S2N12 |
| S2X303 | S2X128 |
| S2X333 | S2X192 |
| S2L50 | S2X58 |
| S2X28 | S2X259 |
| S2X324 | S2X219 |
| S2K146 | ADI-58125 |
| S2N28 | S2X35 |
| S2N22 | S2H90 |
| S2E12 | S2L37 |
| S2H71 | S2A4 |
| S2X30 | S304 |
| S2X16 | S2K63 |
| S2H58 | S2H97 |
| S2H13 | S309 |

In some embodiments, the antibody that specifically binds an epitope in a conserved region (discussed below) of a coronavirus spike protein (e.g., outside of the RBD) facilitates improved binding and neutralization of the neutralizing antibody (e.g., sotrovimab) against coronaviruses. For example, a fusion protein or modified protein as described herein comprising sotrovimab as the neutralizing polypeptide may improve binding to and neutralization of SARS-CoV-2 strains that are more difficult for sotrovimab alone to neutralize. Such strains include those comprising mutations at positions P337 or E340, which have been shown to be escape mutations for sotrovimab (Starr et al., 2021, Nature 597:97-102).

In some embodiments, neutralizing antibodies of the present disclosure target the RBD of a SARS-COV-2 spike protein and prevent the virus from binding ACE2. In some embodiments, neutralizing antibodies do not block ACE2 binding. For example, sotrovimab does not compete with ACE2 for binding in vitro (i.e., SARS-COV-2 virus can bind ACE2 in the presence of the neutralizing antibody in vitro). It is possible that such antibodies still neutralize because, on the surface of the virus, they are sterically occluding ACE2 from binding.

### C. Antibodies Specifically Binding an Epitope in a Conserved Region

The fusion proteins and modified proteins provided herein comprise an antibody that specifically binds an epitope in a conserved region of a coronavirus protein. In some embodiments, the coronavirus protein is a spike protein. In some embodiments, the antibody that specifically binds an epitope in a conserved region of a coronavirus spike protein is a non-neutralizing antibody, also referred to herein as a non-neutralizing coronavirus antibody. In some embodiments, a non-neutralizing antibody for a coronavirus can specifically bind to a coronavirus protein other than the spike protein or can specifically bind to a spike protein at a conserved epitope while having little to no ability of inhibiting viral infection (i.e., the level of inhibition of viral infection induced by a non-neutralizing antibody is 20% or less (e.g., 19%, 18%, 17%, 15%, 14%, 13%, 12%, 11%, 10%, 9%, 8%, 7%, 6%, 5%, 4%, 3%, 2%, 1% or 0%) as compared to the level of inhibition of viral infection observed in the absence of the non-neutralizing antibody). In some embodiments, a non-neutralizing antibody for a coronavirus can specifically bind to a coronavirus protein other than the spike protein or can specifically bind to a spike protein at a conserved epitope while not interfering with the interaction between the spike protein RBD and the coronavirus receptor protein (e.g., ACE2 or DPP4)). A non-neutralizing antibody with litAs such, the non-neutralizing antibody does not prevent coronavirus infection. Because such an antibody does not inhibit coronavirus propagation, there is little to no evolutionary pressure for coronaviruses to evolve to prevent non-neutralizing antibody binding, leading to high conservation of non-neutralizing antibody epitopes among different coronaviruses and variants of coronaviruses. Additionally, some non-neutralizing antibody epitopes occur in viral protein regions that are essential for viral function and are thus highly conserved across different viruses. Because the epitopes are highly conserved, non-neutralizing coronavirus antibodies are able to bind to different coronaviruses (e.g., SARS-COV-2, SARS-CoV, MERS-COV) and variants thereof, allowing fusion proteins and modified proteins comprising a non-neutralizing antibody described herein to specifically bind to and facilitate neutralization of various coronaviruses with high affinity.

As used herein, the term “conserved” in relation to a viral polypeptide sequence (e.g., a “conserved” sequence or a “conserved” region) indicates that the sequence is identical or highly similar across viral species or strains. In general, conservation of a sequence indicates that the sequence has been maintained by natural selection and suggests that the sequence has some functional importance. Amino acid sequences can be conserved to maintain the structure and/or function of a polypeptide or domain. Conserved polypeptide sequences undergo fewer amino acid replacements, or are more likely to substitute amino acids with similar biochemical properties (known as conservative substitutions, as discussed below). As such, within a polypeptide sequence, amino acids that are important for folding, structural stability, or that form a binding site may be more highly conserved than other amino acids. Various methods for identifying conserved sequences are known in the art, and generally involve bioinformatic approaches based on sequence alignment. Approaches include homology searches (e.g., BLAST, HMMER, OrthologR, and Infernal, with acceptable conservative substitution identified using substitution matrices such as PAM and BLOSUM), multiple sequence alignments (e.g., CLUSTuL format), whole genome alignments, and scoring systems (e.g., Genomic Evolutionary Rate Profiling (GERP), Local Identity and Shared Taxa (LIST), Aminode, PhyloP and PhyloHHM). One such method used to identify conserved regions in SARS-COV-2 spike protein is described in Example 7 herein.

Conserved regions may also be estimated by measuring sequence identity for a region of a protein (e.g., the coronavirus spike protein) across a group of related coronaviruses. In some embodiments, related coronaviruses are coronaviruses in which at least one protein in each coronavirus proteome comprises substantial amino acid sequence identity to a selected reference protein amino acid sequence. In some embodiments, related coronaviruses are coronaviruses in which at least one protein in each coronavirus proteome comprises a higher level of sequence identity to a selected reference protein amino acid sequence as compared to an unrelated coronavirus. In some embodiments, the reference spike protein is SARS-COV-2 spike protein (e.g., having the amino acid sequence set forth in SEQ ID NO:337), and related coronaviruses are those comprising spike proteins with amino acid sequences having 30% or greater (e.g., 35% or greater, 40% or greater, 45% or greater, 50% or greater, 55% or greater, 60% or greater, 65% or greater, 70% or greater, 75% or greater, 80% or greater, 85% or greater, or 90% or greater) amino acid sequence identity to SARS-COV-2 spike protein (e.g., as set forth in SEQ ID NO:337). A conserved region has substantial sequence identity across related coronaviruses. In some embodiments, the conserved region (e.g., in a coronavirus spike protein) comprises 75% or greater (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to a reference protein (e.g., SARS-COV-2 spike protein (SEQ ID NO:33)) across related coronaviruses, wherein the related coronaviruses are those comprising spike proteins with amino acid sequences having 30% or greater (e.g., 35% or greater, 40% or greater, 45% or greater, 50% or greater, 55% or greater, 60% or greater, 65% or greater, 70% or greater, 75% or greater, 80% or greater, 85% or greater, or 90% or greater) amino acid sequence identity to SARS-COV-2 spike protein (SEQ ID NO:337) In some embodiments, the conserved region has substantial sequence identity across all known human coronaviruses Exemplary conserved regions within the coronavirus spike protein are shown in bold in Table 12 below and include amino acid residues 740-746, 815-837, 855-866, 894-905, 910-931, 965-1034, 1039-1054, 1076-1082, and 1198-1206 of SEQ ID NO:337. Residues in SEQ ID NO:337 that have 100% identity across a set of spike protein sequences from 40 related coronaviruses are shown in Table 13, below. Additional information regarding sequence conservation among coronavirus spike proteins can be found, e.g., in Jungreis et al., 2021, Nat. Comm. 12.2642; Gupta et al., 2021, Cell. and Mol. Life Sci. 78:7967-7989; and Kumavath et al., 2021, Front. Immunol. 12:663912.

In some embodiments of the fusion proteins and modified proteins provided herein, the antibody that specifically binds an epitope in a conserved region of a coronavirus spike protein is an intact antibody (e.g., an intact immunoglobulin). In some instances, the antibody that specifically binds an epitope in a conserved region of a coronavirus spike protein is an antigen binding fragment comprising at least one antigen binding domain. In some instances, the antibody or antigen binding fragment comprises at least one of a heavy chain sequence or a light chain sequence. In some instances, the antibody or antigen binding fragment comprises an Fc domain. In some instances, the antibody comprises a single chain variable fragment (scFv). In some embodiments, the scFv can comprise amino acid sequences encoded by any of the nucleic acid sequences in Table 6 herein. In some embodiments, the antibody comprises a nanobody. In some instances, the antibody or antigen binding fragment comprises at least one CDR sequence of an antibody heavy chain sequence or CDR sequence of an antibody light chain sequence.

Heavy chain and light chain variable region sequences of antibodies that specifically bind epitopes in conserved regions of a coronavirus spike protein encompassed by this disclosure are set forth in Table 2 and Table 3. Heavy chain and light chain CDR sequences of antibodies that specifically bind epitopes in conserved regions of a coronavirus spike protein encompassed by this disclosure are set forth in Table 4 and Table 5, respectively.

In some embodiments, the antibody that specifically binds an epitope in a conserved region of a coronavirus spike protein comprises a heavy chain variable region comprising an amino acid sequence that has at least 90% identity (e.g., at least 91%, at least 92%, at least 93%, at least 94%, at least 95%, at least 96%, at least 97%, at least 98%, or at least 99%) to any one of SEQ ID NOs: 1-7, 11-15, 17-23, 26, 29-32, 34, 35, 37, and 38. In some embodiments, the antibody that specifically binds an epitope in a conserved region of a coronavirus spike protein comprises a light chain variable region comprising an amino acid sequence that has at least 90% identity (e.g., at least 91%, at least 92%, at least 93%, at least 94%, at least 95%, at least 96%, at least 97%, at least 98%, or at least 99%) to any one of SEQ ID NOs: 77-83, 87-91, 93-99, 102, 105-108, 110, 111, 113, and 114. In some embodiments, the antibody that specifically binds an epitope in a conserved region of a coronavirus spike protein comprises a heavy chain variable region comprising an amino acid sequence that has at least 90% identity to any one of SEQ ID NOs: 1-7, 11-15, 17-23, 26, 29-32, 34, 35, 37, and 38 and a light chain variable region comprising an amino acid sequence that has at least 90% identity to any one of SEQ ID NOs: 77-83, 87-91, 93-99, 102, 105-108, 110, 111, 113, and 114. In some embodiments, the antibody that specifically binds an epitope in a conserved region of a coronavirus spike protein comprises a heavy chain variable region having at least 90% identity to an amino acid sequence set forth in Table 2 and a corresponding light chain variable region having at least 90% identity to an amino acid sequence set forth in Table 3, wherein the corresponding heavy chain and light chain variable sequences are identified by the same antibody name in the “Antibody” columns of Table 2 and Table 3.

In some embodiments, the antibody that specifically binds an epitope in a conserved region of a coronavirus spike protein comprises a heavy chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:1. In some embodiments, the antibody that specifically binds an epitope in a conserved region of a coronavirus spike protein comprises a light chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:77. In some embodiments, the antibody that specifically binds an epitope in a conserved region of a coronavirus spike protein comprises a heavy chain variable region comprising an amino acid sequence that has at least 90% identity to SEQ ID NO: 1 and a light chain variable region comprising an amino acid sequence that has at least 90% identity to SEQ ID NO:77 An exemplary antibody is the antibody identified herein as CV27.

In some embodiments, the antibody that specifically binds an epitope in a conserved region of a coronavirus spike protein comprises a heavy chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:3. In some embodiments, the antibody that specifically binds an epitope in a conserved region of a coronavirus spike protein comprises a light chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:79. In some embodiments, the antibody that specifically binds an epitope in a conserved region of a coronavirus spike protein comprises a heavy chain variable region comprising an amino acid sequence that has at least 90% identity to SEQ ID NO:3 and a light chain variable region comprising an amino acid sequence that has at least 90% identity to SEQ ID NO:79. An exemplary antibody is the antibody identified herein as CV10.

In some embodiments, the antibody that specifically binds an epitope in a conserved region of a coronavirus spike protein comprises a heavy chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:4. In some embodiments, the antibody that specifically binds an epitope in a conserved region of a coronavirus spike protein comprises a light chain variable region comprising an amino acid sequence that has at least 90% identity (e.g., at least 91%, at least 92%, at least 93%, at least 94%, at least 95%, at least 96%, at least 97%, at least 98%, or at least 99%) to SEQ ID NO:80. In some embodiments, the antibody that specifically binds an epitope in a conserved region of a coronavirus spike protein comprises a heavy chain variable region comprising an amino acid sequence that has at least 90% identity to SEQ ID NO:4 and a light chain variable region comprising an amino acid sequence that has at least 90% identity to SEQ ID NO:80. An exemplary antibody is the antibody identified herein as COVA2-14.

In some embodiments, the antibody that specifically binds an epitope in a conserved region of a coronavirus spike protein comprises a heavy chain variable region that includes (i) a CDRH1 comprising any of SEQ ID NOs: 153-170; (ii) a CDRH2 comprising any of SEQ ID NOs: 171-188; and (iii) a CDRH3 comprising any of SEQ ID NOs: 189-214; and a light chain variable region that includes (i) a CDRL1 comprising any of SEQ ID NOs: 215-232; (ii) a CDRL2 comprising any of SEQ ID NOs: 233-241; and (iii) a CDRL3 comprising any of SEQ ID NOs: 242-268. In some embodiments, the antibody that specifically binds an epitope in a conserved region of a coronavirus spike protein comprises a heavy chain variable region that includes a CDRH1, a CDRH2, and a CDRH3 selected from the same row in Table 4. In some embodiments, the antibody that specifically binds an epitope in a conserved region of a coronavirus spike protein comprises a light chain variable region that includes a CDRL1, a CDRL2, and a CDRL3 selected from the same row in Table 5. In some embodiments, the antibody that specifically binds an epitope in a conserved region of a coronavirus spike protein comprises a heavy chain variable region that includes a CDRH1, a CDRH2, and a CDRH3 selected from a row in Table 4 and a CDRL1, a CDRL2, and a CDRL3 selected from a corresponding row in Table 5, wherein the corresponding row is identified by the same antibody name in the “Antibody” columns of Table 4 and Table 5.

In some embodiments, the antibody that specifically binds an epitope in a conserved region of a coronavirus spike protein comprises a heavy chain variable region that includes (i) a CDRH1 comprising SEQ ID NO:153; (ii) a CDRH2 comprising SEQ ID NO:171; and (iii) a CDRH3 comprising SEQ ID NO:189, and a light chain variable region that includes (i) a CDRL1 comprising SEQ ID NO:215; (ii) a CDRL2 comprising SEQ ID NO:233; and (iii) a CDRL3 comprising SEQ ID NO:242. In some embodiments, the antibody that specifically binds an epitope in a conserved region of a coronavirus spike protein comprises a heavy chain variable region that includes (i) a CDRH1 comprising SEQ ID NO:153; (ii) a CDRH2 comprising SEQ ID NO:171; and (iii) a CDRH3 comprising SEQ ID NO:189. In some embodiments, the antibody that specifically binds an epitope in a conserved region of a coronavirus spike protein comprises a light chain variable region that includes (i) a CDRL1 comprising SEQ ID NO:215; (ii) a CDRL2 comprising SEQ ID NO:233; and (iii) a CDRL3 comprising SEQ ID NO:242. An exemplary antibody is the antibody identified herein as CV27.

In some embodiments, the antibody that specifically binds an epitope in a conserved region of a coronavirus spike protein comprises a heavy chain variable region that includes (i) a CDRH1 comprising SEQ ID NO: 157; (ii) a CDRH2 comprising SEQ ID NO: 174; and (iii) a CDRH3 comprising SEQ ID NO: 194; and a light chain variable region that includes (i) a CDRL1 comprising SEQ ID NO:222; (ii) a CDRL2 comprising SEQ ID NO:238; and (iii) a CDRL3 comprising SEQ ID NO:248. In some embodiments, the antibody that specifically binds an epitope in a conserved region of a coronavirus spike protein comprises a heavy chain variable region that includes (i) a CDRH1 comprising SEQ ID NO: 157; (ii) a CDRH2 comprising SEQ ID NO:174; and (iii) a CDRH3 comprising SEQ ID NO:194. In some embodiments, the antibody that specifically binds an epitope in a conserved region of a coronavirus spike protein comprises a light chain variable region that includes (i) a CDRL1 comprising SEQ ID NO:222; (ii) a CDRL2 comprising SEQ ID NO:238; and (iii) a CDRL3 comprising SEQ ID NO:248. An exemplary antibody is the antibody identified herein as CV10.

In some embodiments, the antibody that specifically binds an epitope in a conserved region of a coronavirus spike protein comprises a heavy chain variable region that includes (i) a CDRH1 comprising SEQ ID NO: 162; (ii) a CDRH2 comprising SEQ ID NO:178; and (iii) a CDRH3 comprising SEQ ID NO:199, and a light chain variable region that includes (i) a CDRL1 comprising SEQ ID NO:224; (ii) a CDRL2 comprising SEQ ID NO:239; and (ii) a CDRL3 comprising SEQ ID NO:253. In some embodiments, the antibody that specifically binds an epitope in a conserved region of a coronavirus spike protein comprises a heavy chain variable region that includes (i) a CDRH1 comprising SEQ ID NO:162; (ii) a CDRH2 comprising SEQ ID NO: 178; and (iii) a CDRH3 comprising SEQ ID NO: 199. In some embodiments, the antibody that specifically binds an epitope in a conserved region of a coronavirus spike protein comprises a light chain variable region that includes (i) a CDRL1 comprising SEQ ID NO:224; (ii) a CDRL2 comprising SEQ ID NO:239; and (iii) a CDRL3 comprising SEQ ID NO:253. An exemplary antibody is the antibody identified herein as COVA2-14.

In another aspect, provided herein is an antibody comprising any of the sequences described above. The antibody may also be any of the formats described above (e.g., intact antibody or an antigen-binding fragment such as, e.g., Fv, Fab, scFv, nanobody, etc.). In some embodiments, the antibody is not part of a fusion protein or a modified protein.

TABLE 2
Heavy chains full amino acid and DNA sequences.

| Anti- | | |
|---|---|---|
| body | VDJ amino acid | VDJ nucleotides |
| CV27 | SEQ ID NO: 1 | SEQ ID NO: 39 |
| | QVQLVESGGGVVQPGRSLRLSCAA | CAGGTCCAACTCGTTGAAAGCGGGGGAGGTGTAGTCCAGCCAGGGCGGAGCCTCCGACTC |
| | SGFTFSSYAMHWVRQAPGKGLEW | AGTTGCGCTGCAAGTGGATTCACTTTCAGTTCCTACGCGATGCACTGGGTACGGCAGGCTC |
| | VALISYDGSNKYYADSVKGRFTISR | CGGGGAAAGGGTTGGAATGGGTAGCCTTGATCTCATATGACGGAAGTAATAAATATTACG |
| | DNSKNTLYLQMNSLRAEDTAVYY | CAGATAGCGTTAAGGGACGGTTCACAATATCTCGCGACAATTCTAAGAATACGCTGTACC |
| | CARSFGGSYYYGMDVWGQGTTVT | TTCAAATGAATTCTTTGCGCGCAGAGGATACGGCGGTATATTATTGCGCAAGAAGCTTCG |
| | VSS | GCGGATCTTACTATTATGGAATGGATGTGTGGGGTCAAGGAACAACGGTCACCGTGAGTT |
| | | CA |
| COV2- | SEQ ID NO: 2 | SEQ ID NO: 40 |
| 2147 | QVQLAESGGGVVQPGRSLRLSCAA | CAGGTGCAGCTGGCGGAGTCTGGGGGAGGCGTGGTCCAGCCTGGGAGGTCCCTGAGACTC |
| COV2- | SGFTFSSYAMHWVRQAPGKGLEW | TCCTGTGCAGCCTCTGGATTCACCTTCAGTAGCTATGCTATGCACTGGGTCCGCCAGGCTC |
| 2341 | VAVISYDGSNKYYADSVKGRFTIS | CAGGCAAGGGGCTGGAATGGGTGGCAGTTATATCATATGATGGAAGCAATAAATACTAC |
| COV2- | RDNSKNTLYLQMNSLRAEDTAVY | GCAGACTCCGTGAAGGGCCGATTCACCATCTCCAGAGACAATTCCAAGAACACGCTGTAT |
| 2160 | YCARSTSGSYYYGMDVWGQGTTV | CTGCAAATGAACAGCCTGAGAGCTGAGGACACGGCTGTGTATTACTGTGCGAGAAGCACG |
| COV2- | TVSS | AGTGGGAGCTACTACTACGGTATGGACGTCTGGGGCCAAGGGACCACGGTCACCGTCTCC |
| 2159 | | TCA |
| CV10 | SEQ ID NO: 3 | SEQ ID NO: 41 |
| | QVQLQESGPGLVKPSETLSLTCNVS | CAGGTTCAGCTCCAGGAAAGTGGGCCGGGTCTGGTTAAACCAAGCGAGACGTTGTCCCTT |
| | GGSISSYYWSWIRQPPGKGLEWIG | ACGTGTAATGTCTCTGGAGGGTCTATAAGTTCTTATTACTGGTCTTGGATTCGCCAGCCCC |
| | YIYYSGSTNYNPSLKSRVTISVDTS | CAGGGAAAGGGCTCGAGTGGATCGGCTATATTTATTACTCAGGTTCTACGAACTATAATC |
| | KNQFSLKLSSVTAADTAVYYCARG | CTTCCTTGAAATCCCGGGTGACCATCTCAGTTGACACAAGCAAGAACCAATTCAGTCTCA |
| | FDYWGQGTLVTVSS | AACTCTCTAGCGTCACTGCTGCGGATACGGCTGTGTACTATTGTGCTCGAGGTTTCGACTA |
| | | CTGGGGCCAGGGCACATTGGTCACAGTATCATCA |
| COVA2- | SEQ ID NO: 4 | SEQ ID NO: 42 |
| 14 | QVQLVQSGAEVKKPGSSVKVSCK | CAGGTCCAGCTGGTACAGTCTGGGGCTGAGGTGAAGAAGCCTGGGTCCTCGGTGAAGGTC |
| | ASGGTFSSYAIIWVRQAPGQGLEW | TCCTGCAAGGCTTCTGGAGGCACCTTCAGCAGCTATGCTATCATCTGGGTGCGACAGGCC |
| | MGGIIPIFGTANYAQKFQGRVTITT | CCTGGACAAGGGCTTGAGTGGATGGGAGGGATCATCCCTATCTTTGGTACAGCAAACTAC |
| | DESTSTAYMELSSLRSEDTAVYYC | GCACAGAAGTTCCAGGGCAGAGTCACGATTACCACGGACGAATCCACGAGCACAGCCTA |
| | ARVRYYDSSGYYEDYWGQGTLVT | CATGGAGCTGAGCAGCCTGAGATCTGAGGACACGGCCGTGTATTACTGTGCGAGAGTAAG |
| | VSS | ATACTATGATAGTAGTGGTTATTATGAGGACTACTGGGGCCAGGGAACGCTGGTCACCGT |
| | | CTCCTCA |
| COVA2- | SEQ ID NO: 5 | SEQ ID NO: 43 |
| 18 | EVQLVQSGAEVKKPGSSVKVSCKA | GAGGTGCAGCTGGTGCAGTCTGGGGCTGAGGTGAAGAAGCCTGGGTCCTCGGTGAAGGTC |
| | SGGTFSSYAISWVRQAPGQGLEWM | TCCTGCAAGGCTTCTGGAGGCACCTTCAGCAGCTATGCTATCAGCTGGGTGCGACAGGCC |
| | GGIIPIFGTTNYAQKFQGRVTITTDE | CCTGGACAAGGGCTTGAGTGGATGGGAGGGATCATCCCTATCTTTGGTACAACAAACTAC |
| | STSTAYMELSSLRSEDTAVYYCAR | GCACAGAAGTTCCAGGGCAGAGTCACGATTACCACGGACGAATCCACGAGCACAGCCTA |
| | VYSYDSSGYYLEYWGQGTRVTVS | CATGGAGCTGAGCAGCCTGAGATCTGAGGACACGGCCGTGTATTACTGTGCGAGAGTCTA |
| | S | TTCCTATGATAGTAGTGGTTATTACTTAGAGTACTGGGGCCAGGGAACGCGGGTCACCGT |
| | | CTCTTCA |
| COV2- | SEQ ID NO: 6 | SEQ ID NO: 44 |
| 2449 | QVQLVESGGGVVQPGRSLRLSCAT | CAGGTGCAGCTGGTGGAGTCTGGGGGAGGCGTGGTCCAGCCTGGGAGGTCCCTGAGACTC |
| | SGFTFSSFALHWVRQAPGKGLEWV | TCCTGTGCAACCTCTGGATTCACGTTCAGTAGTTTTGCTTTGCACTGGGTCCGCCAGGCTC |
| | TVISDDGNNKYYVDSVKGRFTISR | CAGGCAAGGGGCTGGAGTGGGTGACAGTTATATCAGATGATGGAAATAATAAATACTAC |
| | DNSKNTLFLQMNSLRVEDTAIYYC | GTCGACTCCGTGAAGGGCCGATTCACCATCTCCAGGGACAATTCCAAGAACACGCTGTTT |
| | ARASYNSNWSIGEYFRDWGQGTL | CTGCAAATGAACAGCCTGAGAGTTGAGGACACGGCTATCTATTACTGTGCGAGAGCCTCG |
| | VTVSS | TATAATAGCAATTGGTCTATTGGTGAATACTTCCGAGACTGGGGCCAGGGCACCCTGGTC |
| | | ACCGTCTCCTCA |
| COV2- | SEQ ID NO: 7 | SEQ ID NO: 45 |
| 2143 | EVQLVESGGGLVQPGGSLRLSCAA | GAGGTGCAGCTGGTGGAGTCTGGGGGAGGCTTGGTCCAGCCTGGGGGGTCCCTGAGACTC |
| | SGFTVSSNYMSWVRQAPGKGLEW | TCCTGTGCAGCCTCTGGATTCACCGTCAGTAGCAACTACATGAGCTGGGTCCGCCAGGCTC |
| | VSVIYSAGSTYYADSVKGRFSISRD | CAGGGAAGGGGCTGGAGTGGGTCTCAGTTATTTATAGCGCTGGTAGCACATACTACGCAG |
| | KSKNTLYLQMNSLRAEDTAVYYC | ACTCCGTGAAGGGCAGATTCAGCATCTCCAGAGACAAGTCCAAGAACACGCTGTATCTTC |
| | AKEGGSGSLRYYYYGMDVWGQG | AAATGAACAGCCTGAGAGCCGAGGACACGGCTGTATATTACTGTGCGAAAGAAGGTGGA |
| | TTVTVSS | TCGGGGAGCCTCCGCTACTACTACTACGGTATGGACGTCTGGGGCCAAGGGACCACGGTC |
| | | ACCGTCTCCTCA |
| COV2- | SEQ ID NO: 11 | SEQ ID NO: 49 |
| 2844 | QVELVESGGGVVQPGRSLRLSCAA | CAGGTGGAGCTGGTGGAGTCTGGGGGAGGCGTGGTCCAGCCTGGGAGGTCCCTGAGACTC |
| | SGFIFSSYAMHWVRQAPGKGLEW | TCCTGTGCAGCCTCTGGATTCATCTTCAGTAGCTATGCTATGCACTGGGTCCGCCAGGCTC |
| | VAVISYDGGNKYYADSVKGRFTIS | CAGGCAAGGGGCTGGAGTGGGTGGCAGTTATATCGTATGATGGAGGCAATAAATACTAC |
| | RDNSKNTLYLQMNSLRAEDTAVY | GCAGACTCCGTGAAGGGCCGATTCACCATCTCCAGAGACAATTCCAAGAACACGCTGTAT |
| | YCARAQGGNYYYGMDVWGQGTT | CTGCAAATGAACAGCCTGAGAGCTGAGGACACGGCTGTGTATTACTGTGCGAGAGCCCAG |
| | VTVSS | GGGGGGAACTACTACTACGGTATGGACGTCTGGGGCCAAGGGACCACGGTCACCGTCTCC |
| | | TCA |
| CV34 | SEQ ID NO: 12 | SEQ ID NO: 50 |
| | QVQLVESGGGVVQPGRSLRLSCAA | CAGGTGCAGTTGGTTGAATCCGGTGGTGGCGTAGTGCAACCGGGCCGGAGCCTTAGGCTG |
| | SGFTFSSYAMHWVRQAPGKGLEW | TCCTGTGCAGCTTCAGGGTTCACTTTCAGCTCTTACGCCATGCACTGGGTACGACAAGCAC |
| | VAVISYDGSNKYYADSVKGRFTIS | CGGGGAAAGGTCTGGAGTGGGTTGCGGTTATTTCCTACGACGGCAGCAACAAATATTACG |
| | RDNSKNTLYLQMNSLRAEDTAVY | CTGATTCAGTTAAAGGACGATTCACGATTTCTAGAGACAATAGCAAAAATACTCTGTACC |
| | YCARSYGGSYYYGMDVWGQGTT | TTCAAATGAATTCTCTGAGAGCCGAGGATACCGCCGTGTATTACTGCGCGAGGTCCTATG |
| | VTVSS | GAGGAAGTTACTACTACGGCATGGATGTTTGGGGTCAAGGGACCACTGTAACCGTCTCTT |
| | | CA |
| COV2- | SEQ ID NO: 13 | SEQ ID NO: 51 |
| 2564 | QVQLVESGGGVVQPGRSLRLSCAA | CAGGTGCAGCTGGTGGAGTCTGGGGGAGGCGTGGTCCAGCCTGGGAGGTCCCTGAGACTC |
| | SGFTFSSYAMHWVRQAPGKGLEW | TCCTGTGCAGCCTCTGGATTCACCTTCAGTAGTTACGCTATGCACTGGGTCCGCCAGGCTC |
| | VAVISYDGYNKYYADSVKGRFTIS | CAGGCAAGGGGCTGGAGTGGGTGGCAGTTATATCATATGATGGATACAATAAATACTACG |
| | RDNSKNTLYLQMNSLRAEDTAVY | CAGACTCCGTGAAGGGCCGATTCACCATCTCCAGAGACAATTCCAAGAACACGCTGTATC |
| | YCARAQGGNYYYGMDVWGQGTT | TGCAAATGAACAGCCTGAGAGCTGAGGACACGGCTGTGTATTACTGTGCGAGAGCCCAGG |
| | VTVSS | GGGGGAACTACTACTACGGTATGGACGTCTGGGGCCAAGGGACCACGGTCACCGTCTCCT |
| | | CA |
| COV2- | SEQ ID NO: 14 | SEQ ID NO: 52 |
| 2643 | QVQLVESGGGVVQPGRSLRLSCAA | CAGGTGCAGCTGGTGGAGTCTGGGGGAGGCGTGGTCCAGCCTGGGAGGTCCCTGAGACTC |
| | SGFTFSSYGMHWVRQAPGKGLEW | TCCTGTGCAGCCTCTGGATTCACCTTCAGTAGCTATGGCATGCACTGGGTCCGCCAGGCTC |
| | VAVISYDGSNKYYADSVKGRFTIS | CAGGCAAGGGGCTGGAGTGGGTGGCAGTTATATCATATGATGGAAGTAATAAATACTATG |
| | RDNAKNSLYLQMNSLRAEDTAVY | CAGACTCCGTGAAGGGCCGATTCACCATCTCCAGAGACAACGCCAAGAACTCACTGTATC |
| | YCARGSAGNYYYGMDVWGQGTT | TGCAAATGAACAGCCTGAGAGCTGAGGACACGGCTGTCTATTACTGTGCGAGAGGGTCAG |
| | VTVSS | CTGGAAACTACTACTACGGTATGGACGTCTGGGGCCAAGGGACCACGGTCACCGTCTCCT |
| | | CA |
| COV2- | SEQ ID NO: 15 | SEQ ID NO: 53 |
| 2203 | QVQLVESGGGVVQPGRSLRLSCAA | CAGGTGCAGCTGGTGGAGTCTGGGGGAGGCGTGGTCCAGCCTGGGAGGTCCCTGAGACTC |
| COV2- | SGFTFSTYAMHWVRQAPGKGLAW | TCCTGTGCAGCCTCTGGATTCACCTTCAGTACCTATGCTATGCACTGGGTCCGCCAGGCTC |
| 2250 | VALISYDGYNKYYADSVRGRFTIS | CAGGCAAGGGGCTGGCGTGGGTGGCACTTATATCATATGATGGATATAATAAATACTACG |
| | RINSKNTLSLQMNSLRAEDTAVYY | CAGACTCCGTGAGGGGCCGATTCACCATCTCCAGAATCAATTCCAAGAACACGCTGTCTC |
| | CARGSAGNYYYGMDVWGQGTTV | TGCAGATGAACAGCCTGAGAGCTGAGGACACGGCTGTCTATTACTGTGCGAGAGGGTCAG |
| | TVSS | CTGGAAACTACTACTACGGTATGGACGTCTGGGGCCAGGGGACCACGGTCACCGTCTCCT |
| | | CA |
| COV2- | SEQ ID NO: 17 | SEQ ID NO: 55 |
| 2656 | EVQLVQSGAEVKKPGESLKISCKG | GAGGTGCAGCTGGTGCAGTCTGGAGCAGAGGTGAAAAAGCCCGGGGAGTCTCTGAAGAT |
| | SGYSFSDYWIGWVRQMPGKGLEW | CTCCTGTAAAGGGTCTGGATACAGCTTTAGCGACTACTGGATCGGCTGGGTGCGCCAGAT |
| | MGIIYPGDSDTRYSPSFQGQVTISA | GCCCGGGAAAGGCCTGGAGTGGATGGGGATCATCTATCCTGGTGACTCTGATACCAGATA |
| | DKSISTAYLQWSSLKASDTAMYYC | CAGCCCGTCCTTCCAAGGCCAGGTCACCATCTCAGCCGACAAGTCCATCAGCACCGCCTA |
| | ARLTFGGSGSYYFYYNGMDVWGQ | CCTGCAGTGGAGCAGCCTGAAGGCCTCGGACACCGCCATGTATTACTGTGCGAGACTGAC |
| | GTTVTVSS | TTTTGGTGGTTCGGGGAGTTATTATTTCTACTACAACGGTATGGACGTCTGGGGCCAAGGG |
| | | ACCACGGTCACCGTCTCCTCA |
| CV8 | SEQ ID NO: 18 | SEQ ID NO: 56 |
| | QVQLVQSGAEVKKPGASVKVSCK | CAGGTACAGCTTGTGCAGTCCGGCGCTGAGGTCAAAAAGCCGGGAGCCTCTGTAAAAGTT |
| | ASGYTFTSYGISWVRQAPGQGLEW | TCTTGCAAAGCGAGCGGTTATACATTCACCTCTTACGGAATCTCTTGGGTGCGACAGGCTC |
| | MGWISAYNGNTNYAQKLQGRVT | CTGGACAAGGGCTTGAGTGGATGGGATGGATCAGCGCCTACAATGGGAACACAAATTAC |
| | MTTDTSTSTAYMELRSLRSDDTAV | GCGCAGAAACTCCAAGGAAGAGTGACAATGACCACTGATACCAGCACCTCCACGGCCTAT |
| | YYCARLVPTWASYYDFWSGYPGG | ATGGAACTTAGGAGCCTCCGATCCGACGACACGGCGGTGTATTATTGCGCAAGGCTTGTT |
| | YGMDVWGQGTTVTVSS | CCGACGTGGGCCAGCTACTACGATTTCTGGAGCGGATACCCGGGAGGATACGGGATGGAC |
| | | GTCTGGGGTCAGGGAACAACTGTAACTGTATCTTCA |
| COV2- | SEQ ID NO: 19 | SEQ ID NO: 57 |
| 2006 | QVQLVESGGGVVQPGRSLRLSCAA | CAGGTGCAGCTGGTGGAGTCTGGGGGAGGCGTGGTCCAGCCTGGGAGGTCCCTGAGACTC |
| | SGFTFSYYAILWFRQAPGKGLEWV | TCCTGTGCAGCCTCTGGATTCACCTTCAGTTACTATGCTATCCTCTGGTTCCGCCAGGCTC |
| | AIISYDGSNKYYADSVKGRFTISRD | CAGGCAAGGGGCTGGAGTGGGTGGCAATTATATCATATGATGGAAGCAATAAATACTACG |
| | NSKNTLYLQMNSLRPEDTAVYYC | CAGACTCCGTGAAGGGCCGATTCACCATCTCCAGAGACAATTCCAAGAACACGCTGTATC |
| | ARPQSGGYYAPLDYWGQGTLVTV | TGCAAATGAACAGCCTGAGACCGGAGGACACGGCTGTGTATTACTGTGCGAGACCACAA |
| | SS | AGTGGGGGCTACTATGCTCCCCTTGACTACTGGGGCCAGGGAACCCTGGTCACCGTCTCCT |
| | | CA |
| C205 | SEQ ID NO: 20 | SEQ ID NO: 58 |
| | QVQLVQSGAEVKKPGASVKVSCK | CAGGTGCAGCTGGTGCAGTCTGGGGCTGAGGTGAAGAAGCCTGGGGCCTCAGTGAAGGTT |
| | ASGHTFTSYYMHWVRQAPGQGLE | TCCTGCAAGGCATCTGGACACACCTTCACCAGCTACTATATGCACTGGGTGCGACAGGCC |
| | WMGIINPSGGSTSYAQKFQGRVTM | CCTGGACAAGGGCTTGAGTGGATGGGAATCATCAACCCTAGTGGTGGTAGCACAAGCTAC |
| | TRDTSTSTVYMELSSLRSEDTAVY | GCACAGAAGTTCCAGGGCAGAGTCACCATGACCAGGGACACGTCCACGAGCACAGTCTA |
| | YCARGPERGIVGATDYFDYWGQG | CATGGAGCTGAGCAGCCTGAGATCTGAGGACACGGCTGTGTATTACTGTGCTAGGGGGCC |
| | TLVTVSS | GGAACGGGGTATAGTGGGAGCTACTGACTACTTTGACTACTGGGGCCAGGGAACCCTGGT |
| | | CACCGTCTCCTCA |
| COV2- | SEQ ID NO: 21 | SEQ ID NO: 59 |
| 2270 | QVQLVQSGAEVKKPGSSVKVSCK | CAGGTGCAGCTGGTGCAGTCTGGGGCTGAGGTGAAGAAGCCTGGGTCCTCGGTGAAGGTC |
| | ASGGTFSSYAISWVRQAPGQGLEW | TCCTGCAAGGCTTCTGGAGGCACCTTCAGCAGCTATGCTATCAGCTGGGTGCGACAGGCC |
| | MGGIIPIFGTANYAQKFQGRVTITA | CCTGGACAAGGGCTTGAGTGGATGGGAGGGATCATCCCTATCTTTGGTACAGCAAACTAC |
| | DESTSTAYMELSSLRSEDTAVYYC | GCACAGAAGTTCCAGGGCAGAGTCACGATTACCGCGGACGAATCCACGAGCACAGCCTA |
| | AITYYYDSSGYWWDDWGQGTLVT | CATGGAGCTGAGCAGCCTGAGATCTGAGGACACGGCCGTGTATTACTGTGCGATTACGTA |
| | VSS | TTACTATGATAGTAGTGGTTATTGGTGGGACGACTGGGGCCAGGGAACCCTGGTCACCGT |
| | | CTCCTCA |
| COV2- | SEQ ID NO: 22 | SEQ ID NO: 60 |
| 2430 | QLQLQESGPGLVKPSETLSLTCTVS | CAGCTGCAGCTGCAGGAGTCGGGCCCAGGATTGGTGAAGCCTTCGGAGACCCTGTCCCTC |
| | GGSISSSSYYWGWIRQPPGKGLEWI | ACCTGCACTGTCTCTGGTGGCTCCATCAGCAGTAGTAGTTACTACTGGGGCTGGATCCGCC |
| | GSVYYIGSTYYNPSLKSRVTMSVD | AGCCCCCAGGGAAGGGGCTGGAGTGGATTGGGAGTGTCTATTATATTGGGAGCACCTACT |
| | TSKNQFSLKLSSVTAADTAVYYCA | ACAACCCGTCCCTCAAGAGTCGAGTCACCATGTCCGTAGACACGTCCAAGAACCAGTTCT |
| | RAPFQLLDKYYFFYYMDVWGKGT | CCCTGAAGCTGAGCTCTGTGACCGCCGCAGACACGGCTGTGTATTACTGTGCGAGGGCCC |
| | TVTVSS | CGTTCCAGCTGCTAGACAAATACTACTTCTTCTACTACATGGACGTCTGGGGCAAAGGGA |
| | | CCACGGTCACCGTCTCCTCA |
| COV2- | SEQ ID NO: 23 | SEQ ID NO: 61 |
| 2441 | QVQLVQSGAEVKKPGSSVKVSCK | CAGGTGCAGCTGGTGCAGTCTGGGGCTGAGGTGAAGAAGCCTGGGTCCTCGGTGAAGGTC |
| COV2- | ASGGTFSSYAIIWVRQAPGQGLEW | TCCTGCAAGGCTTCTGGAGGCACCTTCAGCAGCTATGCTATCATCTGGGTGCGACAGGCC |
| 2166 | MGGIIPIFGTTNYAQKFQGRVTITA | CCTGGACAAGGGCTTGAGTGGATGGGAGGGATCATCCCTATCTTTGGTACAACAAACTAC |
| COV2- | DESTSTAYVELSSLRSEDTAVYYC | GCACAGAAGTTCCAGGGCAGAGTCACGATTACCGCGGACGAATCCACGAGCACAGCCTA |
| 2214 | ARIGHFDSSGYYLDYWGQGTLVTV | CGTGGAACTGAGCAGCCTGAGATCTGAGGACACGGCCGTGTATTATTGTGCGAGAATAGG |
| | SS | CCATTTTGATAGTAGTGGTTATTACTTAGACTACTGGGGCCAGGGAACCCTGGTCACCGTC |
| | | TCCTCA |
| COV2- | SEQ ID NO: 26 | SEQ ID NO: 64 |
| 2367 | QVQLVQSGAEVKKPGSSVKVSCK | CAGGTGCAGCTGGTGCAGTCTGGGGCTGAGGTGAAGAAGCCTGGGTCCTCGGTGAAGGTC |
| COV2- | ASGGTFSSYAISWVRQAPGQGLEW | TCCTGCAAGGCTTCTGGAGGCACCTTCAGCAGCTATGCTATCAGCTGGGTGCGACAGGCC |
| 2216 | MGGIIPIFGAANYAQNFQGRVTITA | CCTGGACAAGGGCTTGAGTGGATGGGAGGGATCATCCCTATCTTTGGTGCAGCAAACTAC |
| COV2- | DESTSTGYMQLSSLRFEDTAVYYC | GCACAGAACTTCCAGGGCAGAGTCACGATTACCGCGGACGAATCCACGAGCACAGGCTA |
| 2169 | ARTSHYDSSGSYFEYWGQGTLVTV | CATGCAACTGAGCAGCCTGAGATTTGAGGACACGGCCGTGTATTACTGTGCGAGAACGTC |
| | SS | TCACTATGATAGTAGTGGTTCCTATTTTGAATACTGGGGCCAGGGAACCCTGGTCACCGTC |
| | | TCCTCA |
| COVA1- | SEQ ID NO: 29 | SEQ ID NO: 67 |
| 07 | QVQLVESGAEVKKPGSSVKVSCKA | CAGGTGCAGCTGGTGGAGTCTGGGGCTGAGGTGAAGAAGCCTGGGTCCTCGGTGAAGGTC |
| | SGGTLSSYAITWVRQAPGQGLEWV | TCTTGCAAGGCTTCTGGAGGCACCCTCAGCAGCTATGCTATCACCTGGGTGCGACAGGCC |
| | GGIIPIFGTANYAQKFQGRVTITAD | CCTGGACAAGGGCTTGAGTGGGTGGGAGGGATCATCCCTATCTTTGGTACAGCAAACTAC |
| | ESTSTAYMELSSLRSEDTAVYYCA | GCACAGAAGTTCCAGGGCAGAGTCACGATTACCGCGGACGAATCCACGAGCACAGCCTA |
| | RVGAYDSSGYSNDYWGQGTLVTV | CATGGAGCTGAGCAGCCTGAGATCTGAGGACACGGCCGTGTATTACTGTGCGAGAGTAGG |
| | SS | GGCCTATGATAGTAGTGGTTATTCCAATGACTACTGGGGCCAGGGAACCCTGGTCACCGT |
| | | CTCCTCA |
| Chi2M- | SEQ ID NO: 30 | SEQ ID NO: 68 |
| 8E7 | EVQLVESGAEVKKPGSSVKVSCKA | GAAGTGCAGCTGGTGGAGTCTGGGGCTGAGGTGAAGAAGCCTGGGTCCTCGGTGAAGGT |
| | SGGTFSSYAISWVRQAPGQGLEWM | CTCCTGCAAGGCTTCTGGAGGCACCTTCAGCAGCTATGCTATCAGCTGGGTGCGACAGGC |
| | GGIIPIFGTANYAQKFQGRVTVTAD | CCCTGGACAAGGGCTTGAGTGGATGGGAGGGATCATCCCTATCTTTGGTACAGCAAACTA |
| | ESTSTAYMELSSLRSEDTAVYYCA | CGCACAGAAGTTCCAGGGCAGAGTCACGGTTACCGCGGACGAATCCACGAGCACAGCCT |
| | RTYSFDSSGYYYDYWGQGTMVTV | ACATGGAGCTAAGCAGCCTGAGATCTGAGGACACGGCCGTGTATTACTGCGCGAGAACGT |
| | SS | ATTCCTTTGATAGTAGTGGATATTACTACGACTACTGGGGCCAGGGAACCATGGTCACCG |
| | | TCTCTTCA |
| COV2- | SEQ ID NO: 31 | SEQ ID NO: 69 |
| 2621 | QVQLVESGGGVVQPGRSLRLSCAA | CAGGTGCAGCTGGTGGAGTCTGGGGGAGGCGTGGTCCAGCCTGGGAGGTCCCTGAGACTC |
| | SGFTFSSYLMHWVRQAPGKGLEW | TCCTGTGCAGCGTCTGGATTCACCTTTAGTAGCTATCTCATGCACTGGGTCCGCCAGGCTC |
| | VAVIWANGNRYYADSVKGRFTISR | CAGGCAAGGGGCTGGAGTGGGTGGCAGTTATATGGGCTAATGGAAATAGATATTATGCA |
| | DISKNTLYLQMNSLRAEDTAMYYC | GACTCCGTGAAGGGCCGATTCACCATCTCCAGAGACATTTCCAAGAACACGCTGTATCTG |
| | ARDYCNGVTCNSNYWGQGTLVTV | CAGATGAATAGCCTGAGAGCCGAGGACACGGCCATGTATTACTGTGCGAGAGACTATTGT |
| | SS | AATGGTGTTACCTGCAACTCGAACTACTGGGGCCAGGGAACCCTGGTCACCGTCTCCTCA |
| COV2- | SEQ ID NO: 32 | SEQ ID NO: 70 |
| 2883 | QVQLVESGGGVVQPGRSLRLSCAA | CAGGTGCAGCTGGTGGAGTCCGGGGGAGGCGTGGTCCAGCCTGGGAGGTCCCTGAGACTC |
| COV2- | SGFTFSTYGMHWVRQAPGKGLEW | TCCTGTGCAGCCTCTGGATTCACCTTCAGTACCTATGGCATGCACTGGGTCCGCCAGGCTC |
| 2224 | VAVISYDGSNKYYADSVKGRFTIS | CAGGCAAGGGGCTGGAGTGGGTGGCAGTTATATCATATGATGGAAGTAATAAATACTATG |
| | RDNSKNTLYLQMNSLRAEDTAMY | CAGACTCCGTGAAGGGCCGATTCACCATCTCCAGAGACAATTCCAAGAACACACTGTATC |
| | YCAKDGSIAAADYWGQGTLVTVS | TGCAAATGAACAGCCTGAGAGCTGAGGACACGGCTATGTATTACTGTGCGAAAGATGGG |
| | S | AGTATAGCAGCAGCTGACTACTGGGGCCAGGGAACCCTGGTCACCGTCTCCTCG |
| Chi2M- | SEQ ID NO: 34 | SEQ ID NO: 72 |
| 8H10 | QVQLVESGGGVVLPGRSLRLSCAA | CAGGTTCAGCTGGTGGAGTCTGGGGGAGGCGTGGTCCTGCCTGGGAGGTCCCTGAGACTC |
| | SGFTFSTFAMHWVRQAPGKGLEW | TCCTGTGCAGCCTCTGGATTCACCTTCAGTACCTTCGCTATGCACTGGGTCCGCCAGGCTC |
| | VAVISDEGSNKYYADSVKGRFTISR | CAGGCAAGGGGCTGGAGTGGGTGGCAGTTATATCAGATGAAGGAAGTAATAAATACTAC |
| | DNSRNTLYLQMNSLRAEDTAVYY | GCAGACTCCGTGAAGGGCCGATTCACCATCTCCAGAGACAATTCCAGGAACACCCTTTAT |
| | CARAFYDSNWSVGSYFDSWGQGT | CTGCAAATGAACAGCCTGAGAGCTGAAGACACGGCTGTGTATTATTGTGCGAGAGCTTTT |
| | PVTVSS | TATGATAGTAACTGGTCCGTCGGATCCTACTTTGACTCCTGGGGCCAGGGAACCCCGGTC |
| | | ACCGTCTCCTCA |
| COV2- | SEQ ID NO: 35 | SEQ ID NO: 73 |
| 2401 | QVQLVESGGGVVQPGRSLRLSCAA | CAGGTGCAGCTGGTGGAGTCTGGGGGAGGCGTGGTCCAGCCTGGGAGGTCCCTGAGACTC |
| COV2- | SGFTFSSYALFWVRQAPGKGLEWV | TCCTGTGCAGCCTCTGGATTCACCTTCAGTAGTTATGCTCTGTTCTGGGTCCGCCAGGCTC |
| 2218 | AVISYDGNNKYYADSVRGRFTISR | CAGGCAAGGGGCTGGAGTGGGTGGCAGTTATTTCATATGATGGAAATAATAAATACTACG |
| | DNSKNTLYLQMNSLRPEDTAVYY | CAGACTCCGTGAGGGGCCGATTCACCATCTCCAGAGACAATTCCAAGAACACGCTGTATC |
| | CARPYTGSYKSYMDVWGKGTTVT | TGCAAATGAACAGCCTGAGACCTGAGGACACGGCTGTGTATTACTGTGCGAGACCATATA |
| | VSS | CTGGGAGCTACAAGAGCTACATGGACGTCTGGGGCAAAGGGACCACGGTCACCGTCTCCT |
| | | CA |
| Chi0304- | SEQ ID NO: 37 | SEQ ID NO: 75 |
| 4A2 | EVQLVESGPGLVKPSETLSLTCAVS | GAGGTGCAGCTGGTGGAGTCGGGCCCAGGACTGGTGAAGCCTTCGGAGACCCTGTCCCTC |
| | GDSTSSSSSYWDWIRQPPGKGLEW | ACCTGCGCTGTCTCTGGTGACTCCACCAGCAGTAGTAGTTCCTACTGGGACTGGATCCGCC |
| | IGNIYYTGTTYYNPSLKSRVTISVD | AGCCCCCAGGGAAGGGGCTGGAATGGATTGGGAATATCTATTATACTGGGACCACCTACT |
| | TSKDQFSLKLSSVTAADTAVYYCA | ACAACCCGTCCCTCAAGAGTCGAGTCACCATATCAGTAGACACGTCCAAGGACCAGTTCT |
| | RELFTAVAGKGGIDYWGQGTLVT | CCCTGAAACTGAGCTCTGTGACCGCCGCGGACACGGCCGTGTATTACTGTGCGAGAGAAC |
| | VSS | TATTTACGGCAGTGGCTGGCAAGGGGGGTATTGACTACTGGGGCCAGGGAACCCTGGTCA |
| | | CCGTCTCCTCA |
| COV2- | SEQ ID NO: 38 | SEQ ID NO: 76 |
| 2422 | QVQLVESGGGVVQPGRSLRLSCAA | CAGGTGCAGCTGGTGGAGTCTGGGGGAGGCGTGGTCCAGCCTGGGAGGTCCCTGAGACTC |
| | SGFTFSSYAMYWVRQAPGKGLEW | TCCTGTGCAGCCTCTGGATTCACCTTCAGTAGCTATGCTATGTACTGGGTCCGCCAGGCTC |
| | VAVISYDGINKYYADSVKGRFTISR | CAGGCAAGGGGCTGGAGTGGGTGGCAGTTATATCATATGATGGAATTAATAAATACTACG |
| | DNSKNTLYLQMNSLRAEDTAVYY | CAGACTCCGTGAAGGGCCGATTCACCATCTCCAGAGACAATTCCAAGAACACGCTGTATC |
| | CARVNSGSYYSYFDYWGQGTLVT | TGCAAATGAACAGCCTGAGAGCTGAGGACACGGCTGTGTATTACTGTGCGAGAGTGAACA |
| | VSS | GTGGGAGCTACTATTCCTACTTTGACTACTGGGGCCAGGGAACCCTGGTCACCGTCTCCTC |
| | | A |

TABLE 3
Light chain full amino acid and DNA sequences.

| Anti- | | |
|---|---|---|
| body | VJ amino acid | VJ nucleotides |
| CV27 | SEQ ID NO: 77 | SEQ ID NO: 115 |
| | QSALTQPASVSGSPGQSITISCT | CAGTCTGCTCTGACTCAACCCGCATCAGTTTCTGGTTCTCCGGGTCAGTCTATCACCAT |
| | GTSSDVGGYNYVSWYQQHPGKAP | ATCATGCACCGGCACTAGCTCTGACGTAGGAGGATATAACTACGTTAGTTGGTACCAAC |
| | KLMIYDVSNRPSGVSNRFSGSKS | AACACCCTGGAAAAGCACCGAAATTGATGATATATGATGTCAGTAACCGACCTAGCGGA |
| | GNTASLTISGLQAEDEADYYCSS | GTTTCCAATCGATTCTCCGGTTCAAAGAGCGGCAATACGGCTTCCTTGACAATCTCCGG |
| | YTSSSTPYVFGTGTKVTVL | GCTTCAAGCGGAAGATGAAGCTGACTACTATTGTTCTTCTTACACATCTAGCTCCACGC |
| | | CATATGTGTTCGGAACAGGCACTAAAGTGACAGTACTA |
| COV2- | SEQ ID NO: 78 | SEQ ID NO: 116 |
| 2147 | QSALTQPASVSGSPGQSITISCT | CAGTCTGCCCTGACTCAGCCTGCCTCCGTGTCTGGGTCTCCTGGACAGTCGATCACCAT |
| COV2- | GTSSDVGDYNYVSWYQQHPGKAP | CTCCTGCACTGGAACCAGCAGTGACGTTGGTGATTATAACTATGTCTCCTGGTACCAAC |
| 2341 | KLMIYDVSNRPSGVSNRFSGSKS | AACACCCAGGCAAAGCCCCCAAACTCATGATTTATGATGTCAGTAATCGGCCCTCAGGG |
| COV2- | GNTASLTISGLQAEDEAEYYCSS | GTTTCTAATCGCTTCTCTGGCTCCAAGTCTGGCAACACGGCCTCCCTGACCATCTCTGG |
| 2160 | YTSSSTLLYVFGTGTKVTVL | GCTCCAGGCTGAGGACGAGGCTGAATATTACTGCAGCTCATATACAAGCAGCAGCACTC |
| COV2- | | TACTTTATGTCTTCGGAACTGGGACCAAGGTCACCGTCCTA |
| 2159 | | |
| CV10 | SEQ ID NO: 79 | SEQ ID NO: 117 |
| | EIVLTQSPGTLSLSPGERATLSC | GAAATTGTGCTTACGCAGTCACCCGGAACTCTCAGTCTGTCCCCCGGTGAAAGGGCCAC |
| | RASQSVSSIYLAWYQQKPGQAPR | TCTCTCCTGTAGGGCATCCCAAAGCGTTTCTAGCATATATCTCGCTTGGTACCAACAGA |
| | LLIYGASSRATGIPDRFSGSGSG | AGCCGGGTCAAGCTCCGCGGCTGCTGATTTATGGCGCCTCTAGTCGGGCAACTGGTATC |
| | TDFTLTISRLEPEDFAVYYCQQY | CCTGATCGGTTTAGTGGGTCAGGAAGTGGTACAGATTTCACCCTTACGATTTCTCGGCT |
| | AGSPWTFGQGTKVEIK | CGAGCCCGAGGATTTTGCCGTATACTATTGTCAACAGTACGCAGGGTCTCCTTGGACGT |
| | | TTGGCCAGGGCACAAAGGTCGAGATCAAA |
| COVA2- | SEQ ID NO: 80 | SEQ ID NO: 118 |
| 14 | EIVLTQSPATLSLSPGERATLSC | GAAATTGTGTTGACACAGTCTCCAGCCACCCTGTCTTTGTCTCCAGGGGAAAGAGCCAC |
| | RASQSVSSYLAWYQQEPGQAPRL | CCTCTCCTGCAGGGCCAGTCAGAGTGTTAGCAGCTACTTAGCCTGGTACCAACAGGAAC |
| | LIYDASNRATGIPARFSGSGSGT | CTGGCCAGGCTCCCAGGCTCCTCATTTATGATGCATCCAACAGGGCCACTGGCATCCCA |
| | DFTLTISSLEPEDFAVYYCQQRS | GCCAGGTTCAGTGGCAGTGGGTCTGGGACAGACTTCACTCTCACCATCAGCAGCCTAGA |
| | NWPPMYTFGQGTKVEIK | GCCTGAAGATTTTGCAGTTTATTACTGTCAGCAGCGTAGCAACTGGCCTCCTATGTACA |
| | | CTTTTGGCCAGGGGACCAAGGTGGAGATCAAAC |
| COVA2- | SEQ ID NO: 81 | SEQ ID NO: 119 |
| 18 | EIVLTQSPATLSLSPGERATLSC | GAAATTGTGTTGACACAGTCTCCAGCCACCCTGTCTTTGTCTCCAGGGGAAAGAGCCAC |
| | RASQSVSSYLAWYQQKPGQAPRL | CCTCTCCTGCAGGGCCAGTCAGAGTGTTAGCAGCTACTTAGCCTGGTACCAACAGAAAC |
| | LIYDASNRATGIPARFSGSGSGT | CTGGCCAGGCTCCCAGGCTCCTCATCTATGATGCATCCAACAGGGCCACTGGCATCCCA |
| | DFTLTISSLEPEDFAVYYCQQRS | GCCAGGTTCAGTGGCAGTGGGTCTGGGACAGACTTCACTCTCACCATCAGCAGCCTAGA |
| | NWPPSITFGQGTRLEIK | GCCTGAAGATTTTGCAGTTTATTACTGTCAGCAGCGTAGCAACTGGCCTCCCTCGATCA |
| | | CCTTCGGCCAAGGGACACGACTGGAGATTAAAC |
| COV2- | SEQ ID NO: 82 | SEQ ID NO: 120 |
| 2449 | DIVMTQSPDSLAVSLGERATINC | GACATCGTGATGACCCAGTCTCCAGACTCCCTGGCTGTGTCTCTGGGCGAGAGGGCCAC |
| | KSSQSLLYTSNNKNYLAWYQQKP | CATCAACTGCAAGTCCAGCCAGAGTCTTTTATACACCTCCAACAATAAGAACTACTTAG |
| | GQPPKLLIYWASTRESGVPDRFS | CTTGGTACCAGCAGAAACCAGGACAGCCTCCTAAGCTGCTCATTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYSPPWTFGQGTKVEIK | CACCATCAGCAGCCTGCAGGCTGAAGATGTGGCAGTTTATTACTGTCAGCAATATTATA |
| | | GTCCTCCGTGGACGTTCGGCCAAGGGACCAAGGTGGAAATCAAA |
| COV2- | SEQ ID NO: 83 | SEQ ID NO: 121 |
| 2143 | QSVVTQPPSASGTPGQRVTISCS | CAGTCTGTGGTGACTCAGCCACCCTCAGCGTCTGGGACCCCCGGGCAGAGGGTCACCAT |
| | GSSSNIGYNIVNWYQQLPGTAPK | CTCTTGTTCTGGAAGCAGCTCCAACATCGGATATAATATTGTAAACTGGTACCAGCAGC |
| | LLIYSNNQRPSGVPDRFSGSKSG | TCCCAGGAACGGCCCCCAAACTCCTCATCTATAGTAATAATCAGAGGCCCTCAGGGGTC |
| | TSASLSISGLQSEDEADYYCAAW | CCTGACCGATTCTCTGGCTCCAAGTCTGGCACCTCAGCCTCCCTGTCCATCAGTGGGCT |
| | DDSLNGYVFGTGTKVTVL | CCAGTCTGAGGATGAGGCTGATTATTACTGTGCAGCATGGGATGACAGCCTGAATGGTT |
| | | ATGTCTTCGGAACTGGGACCAAGGTCACCGTCCTA |
| COV2- | SEQ ID NO: 87 | SEQ ID NO: 125 |
| 2844 | SYVLTQPPSVSVAPGKTARITCG | TCCTATGTGCTGACTCAGCCACCCTCGGTGTCAGTGGCCCCAGGAAAGACGGCCAGGAT |
| | GNNIGSKNVHWYQQKPGQAPVKV | TACCTGTGGGGGAAACAACATTGGAAGTAAAAATGTGCACTGGTACCAGCAGAAGCCA |
| | VYHDGDRPSGIPERFSGSNSGNT | GGCCAGGCCCCTGTGAAGGTCGTCTATCATGATGGCGACCGGCCCTCAGGGATCCCTGA |
| | ATLTINRVEAGDEADYSCQVWDS | GCGATTCTCTGGCTCCAACTCTGGGAACACGGCCACCCTGACCATCAACAGGGTCGAAG |
| | SSDHHVVFGGGTKLTVL | CCGGGGATGAGGCCGACTATTCCTGTCAGGTGTGGGATAGTAGTAGTGATCATCATGTG |
| | | GTTTTCGGCGGAGGGACCAAGCTGACCGTCCTA |
| CV34 | SEQ ID NO: 88 | SEQ ID NO: 126 |
| | SYELTQPHSVSVATAQMARITCG | TCCTATGAGTTGACTCAGCCTCATAGCGTATCAGTCGCAACTGCACAGATGGCGCGCAT |
| | GNNIGSKAVHWYQQKPGQDPVLV | CACATGCGGTGGAAACAATATAGGCAGTAAGGCGGTACATTGGTATCAGCAAAAACCT |
| | IYSDSNRPSGIPERFSGSNPGNT | GGTCAAGACCCCGTGTTGGTAATCTACTCCGATTCAAATCGACCCTCAGGGATTCCAGA |
| | ATLTISRIEAGDEADYYCQVWDS | ACGCTTCTCCGGGTCAAATCCGGGGAACACGGCTACACTCACTATAAGCAGAATTGAAG |
| | DSSHVVFGGGTKLTVL | CGGGAGATGAGGCGGACTATTACTGTCAAGTATGGGATTCCAGCTCAGATCATGTTGTC |
| | | TTCGGGGGGGGCACAAAACTCACAGTCCTA |
| COV2- | SEQ ID NO: 89 | SEQ ID NO: 127 |
| 2564 | SYVLTQPPSVSVAPGKTARITCG | TCCTATGTGCTGACTCAGCCACCCTCGGTGTCAGTGGCCCCAGGAAAGACGGCCAGGAT |
| | GNNIGTKGVHWYQQKPGQAPVLV | TACCTGTGGGGGAAACAACATTGGAACTAAAGGTGTGCACTGGTACCAGCAGAAGCCA |
| | VYDDSDRPSGIPGRFSGSNSGNT | GGCCAGGCCCCTGTGCTGGTCGTCTATGATGATAGCGACCGGCCCTCAGGGATCCCTGG |
| | ATLTISRVEAGDEADYFCQVWDS | GCGATTCTCTGGCTCCAACTCTGGGAACACGGCCACCCTGACCATCAGCAGGGTCGAAG |
| | SSDHHVVFGGGTKLTVL | CCGGGGATGAGGCCGACTATTTCTGTCAGGTGTGGGATAGTAGTAGTGATCATCATGTG |
| | | GTATTCGGCGGAGGGACCAAGCTGACCGTCCTA |
| COV2- | SEQ ID NO: 90 | SEQ ID NO: 128 |
| 2643 | DIQMTQSPSSLSASVGDRVTITC | GACATCCAGATGACCCAGTCTCCATCCTCCCTGTCTGCATCTGTAGGAGACAGAGTCAC |
| | RASQSISSYLNWYQQKPGKAPKL | CATCACTTGCCGGGCAAGTCAGAGCATTAGCAGCTATTTAAATTGGTATCAGCAGAAAC |
| | LIYAASSLQSGVPSRFSGSGSGT | CAGGGAAAGCCCCTAAGCTCCTGATCTATGCTGCATCCAGTTTGCAAAGTGGGGTCCCA |
| | DFTLTISSLQPEDYATYYCQQSY | TCAAGGTTCAGTGGCAGTGGATCTGGGACAGATTTCACTCTCACCATCAGCAGTCTGCA |
| | STPGTFGQGTRLEIK | ACCTGAAGATTATGCAACTTACTACTGTCAACAGAGTTACAGTACCCCTGGCACCTTCG |
| | | GCCAAGGGACACGACTGGAGATTAAA |
| COV2- | SEQ ID NO: 91 | SEQ ID NO: 129 |
| 2203 | DIQMTQSPSSLSASVGDRVTITC | GACATCCAGATGACCCAGTCTCCATCCTCCCTGTCTGCATCTGTAGGAGACAGAGTCAC |
| COV2- | RASQTITNYLNWYQLKSGRAPKL | CATCACTTGCCGGGCAAGTCAGACCATTACCAACTATTTAAATTGGTATCAGCTGAAAT |
| 2250 | LIVAASSLQSGVPSRFSGSGSGT | CAGGGAGAGCCCCCAAGCTCCTGATCTATGCTGCATCCAGTTTGCAAAGTGGGGTCCCA |
| | DFTLTISSLQPEDFATYYCQQS | TCAAGGTTCAGTGGCAGTGGATCTGGGACAGATTTCACTCTCACCATCAGCAGTCTGCA |
| | YSTPYTFGQGTKLEIK | ACCTGAAGATTTTGCAACTTACTACTGTCAACAGAGTTACAGTACGCCGTACACTTTTG |
| | | GCCAGGGGACCAAGCTGGAGATCAAA |
| COV2- | SEQ ID NO: 93 | SEQ ID NO: 131 |
| 2656 | EIVLTQSPGTLSLSPGERATLSC | GAAATTGTGTTGACGCAGTCTCCAGGCACCCTGTCTTTGTCTCCAGGGGAAAGAGCCAC |
| | RASQSVSSSYLAWYQQKPGQAPR | CCTCTCCTGCAGGGCCAGTCAGAGTGTTAGCAGCAGCTACTTAGCCTGGTACCAGCAAA |
| | LLIYGASSRATGIPDRFSGSGSG | AACCTGGCCAGGCTCCCAGGCTCCTCATCTATGGTGCATCCAGCAGGGCCACCGGCATC |
| | TDFTLTISRLEPEDFAVYYCQQY | CCAGACAGGTTCAGTGGCAGTGGGTCTGGGACAGACTTCACTCTCACCATCAGCAGACT |
| | GRSSGTFGQGTKVEIK | GGAGCCTGAAGATTTTGCAGTGTATTACTGTCAGCAGTATGGTAGATCATCAGGGACGT |
| | | TCGGCCAAGGGACCAAGGTGGAAATCAAA |
| CV8 | SEQ ID NO: 94 | SEQ ID NO: 132 |
| | EIVLTQSPGTLSLSPGERATLSC | GAAATTGTCTTGACGCAGAGTCCTGGTACGCTGAGCCTGAGCCCAGGCGAACGAGCTAC |
| | RASQSVSSSYLAWYQQKPGQAPR | CCTTAGCTGTAGGGCTTCTCAAAGTGTGTCTAGTAGCTATCTGGCGTGGTACCAACAGA |
| | LLIVGASSRATGIPDRFSGSGSG | AACCAGGCCAGGCCCCTAGACTTTTGATCTACGGAGCGAGTAGCCGCGCGACTGGCATC |
| | TDFTLTISRLEPEDFAVYYCQQY | CCTGACCGATTCTCCGGCAGTGGCAGTGGAACAGATTTTACTCTTACAATAAGTCGCCT |
| | GSSPGTFGQGTRLEIK | TGAGCCAGAGGATTTTGCTGTGTACTATTGTCAACAATACGGTAGTAGCCCGGGGACGT |
| | | TCGGGCAAGGCACCAGACTCGAGATCAAA |
| COV2- | SEQ ID NO: 95 | SEQ ID NO: 133 |
| 2006 | EIVLTQSPGTLSLSPGERATLSC | GAAATTGTGTTGACGCAGTCTCCAGGCACCCTGTCTTTGTCTCCAGGGGAAAGAGCCAC |
| | RASQSVSSSYLAWYQQKPGQAPR | CCTCTCCTGCAGGGCCAGTCAGAGTGTTAGCAGCAGCTACTTAGCCTGGTACCAGCAGA |
| | LLIYGASSRATGIPDRFSGSGSG | AACCTGGCCAGGCTCCCAGGCTCCTCATCTATGGTGCATCCAGCAGGGCCACIGGCATC |
| | TDFTLTISRLEPEDFAVYYCQQY | CCAGACAGGTTCAGTGGCAGTGGGTCTGGGACAGACTTCACTCTCACCATCAGCAGACT |
| | GSSPWTFGQGTKVEIK | GGAGCCTGAAGATTTTGCAGTGTATTACTGTCAGCAGTATGGTAGCTCACCCTGGACGT |
| | | TCGGCCAAGGGACCAAGGTGGAAATCAAA |
| C205 | SEQ ID NO: 96 | SEQ ID NO: 134 |
| | EIVLTQSPGTLSLSPGERATLSC | GAAATTGTGTTGACGCAGTCTCCAGGCACCCTGTCTTTGTCTCCAGGGGAAAGAGCCAC |
| | RASQSVSSSYLAWYQQKPGQAPR | CCTCTCCTGCAGGGCCAGTCAGAGTGTTAGCAGCAGCTACTTAGCCTGGTACCAGCAGA |
| | LLIYGASSRATGIPDRFSGSGSG | AACCTGGCCAGGCTCCCAGGCTCCTCATCTATGGTGCATCCAGCAGGGCCACTGGCATC |
| | TDFTLTISRLEPEDFAVYYCQQY | CCAGACAGGTTCAGTGGCAGTGGGTCTGGGACAGACTTCACTCTCACCATCAGCAGACT |
| | VSSPWTFGQGTKVEIK | GGAGCCTGAAGATTTTGCAGTGTATTACTGTCAGCAGTATGTTAGCTCACCGTGGACGT |
| | | TCGGCCAAGGGACCAAGGTGGAAATCAAA |
| COV2- | SEQ ID NO: 97 | SEQ ID NO: 135 |
| 2270 | EIVLTQSPATLSLSPGERATLSC | GAAATTGTGTTGACACAGTCTCCAGCCACCCTGTCTTTGTCTCCAGGGGAAAGAGCCAC |
| | RASQSVSSFLAWYQQKPGQAPRL | CCTCTCCTGCAGGGCCAGTCAGAGTGTTAGCAGCTTCTTAGCCTGGTACCAACAGAAAC |
| | LIYDASNRATGIPARFSGSGSGT | CTGGCCAGGCTCCCAGGCTCCTCATCTATGATGCATCCAACAGGGCCACTGGCATCCCA |
| | DFTLTISSLEPEDFAVYYCQQRP | GCCAGGTTCAGTGGCAGTGGGTCTGGGACAGACTTCACTCTCACCATCAGCAGCCTAGA |
| | SNWPSYTFGQGTKLEIK | GCCTGAAGATTTTGCAGTTTATTACTGTCAGCAGCGTAGCAACTGGCCTCCTTCGTACA |
| | | CTTTTGGCCAGGGGACCAAGCTGGAGATCAAA |
| COV2- | SEQ ID NO: 98 | SEQ ID NO: 136 |
| 2430 | EIVLTQSPATLSLSPGERATLSC | GAAATTGTGTTGACACAGTCTCCAGCCACCCTGTCTTTGTCTCCAGGGGAAAGAGCCAC |
| | RASQSVSSYLAWYQQKPGQAPRL | CCTCTCCTGCAGGGCCAGTCAGAGTGTTAGCAGCTACTTAGCCTGGTACCAACAGAAAC |
| | LIYDASNRATGIPARFSGSGSGT | CTGGCCAGGCTCCCAGGCTCCTCATCTATGATGCATCCAACAGGGCCACTGGCATCCCA |
| | DFTLTISSLEPEDFAVYYCQQRS | GCCAGGTTCAGTGGCAGTGGGTCTGGGACAGACTTCACTCTCACCATCAGCAGCCTAGA |
| | NWPPGVTFGQGTRLEIK | GCCTGAAGATTTTGCAGTTTATTACTGTCAGCAGCGTAGCAACTGGCCTCCGGGCGTCA |
| | | CCTTCGGCCAAGGGACACGACTGGAGATTAAA |
| COV2- | SEQ ID NO: 99 | SEQ ID NO: 137 |
| 2441 | EIVLTQSPATLSLSPGERATLSC | GAAATTGTGTTGACACAGTCTCCAGCCACCCTGTCTTTGTCTCCAGGGGAAAGAGCCAC |
| COV2- | RASQSVSSFLAWYQQKPGQAPRL | CCTCTCCTGCAGGGCCAGTCAGAGTGTTAGCAGCTTCTTAGCCTGGTACCAACAGAAAC |
| 2166 | LIYDASNRPTGIPARFTGSGSGT | CTGGCCAGGCTCCCAGGCTCCTCATCTATGATGCATCCAACAGGCCCACTGGCATCCCA |
| COV2- | DFTLTISSLEPEDFAVYYCQHR | GCCAGGTTCACTGGCAGTGGGTCTGGGACAGACTTCACTCTCACCATCAGCAGCCTAGA |
| 2214 | TNWPPLFTFGPGTKVDIK | GCCTGAAGATTTTGCAGTTTATTACTGTCAGCACCGTACCAACTGGCCTCCCTTATTCA |
| | | CTTTCGGCCCTGGGACCAAAGTGGATATCAAA |
| COV2- | SEQ ID NO: 102 | SEQ ID NO: 140 |
| 2367 | EIVLTQSPATLSLSPGERATLSC | GAAATTGTGTTGACACAGTCTCCAGCCACCCTGTCTTTGTCTCCAGGGGAAAGAGCCAC |
| COV2- | RASQSVSSYLAWYQQKPGQAPRL | CCTCTCCTGCAGGGCCAGTCAGAGTGTTAGCAGCTACTTAGCCTGGTACCAACAGAAAC |
| 2216 | LIYDASNRATGIPARFSGSGSGT | CTGGCCAGGCTCCCAGGCTCCTCATCTATGATGCATCCAACAGGGCCACTGGCATCCCA |
| COV2- | DFTLTISSLDPEDFAVYYCHKRS | GCCAGGTTCAGTGGCAGTGGGTCTGGGACAGACTTCACTCTCACCATCAGCAGCCTAGA |
| 2169 | NWPPSLTFGGGTKVEIK | CCCTGAAGATTTTGCAGTTTATTACTGTCACAAGCGTAGCAACTGGCCTCCTTCGCTCA |
| | | CTTTCGGCGGAGGGACCAAGGTGGAGATCAAG |
| COVA1- | SEQ ID NO: 105 | SEQ ID NO: 143 |
| 07 | DIQLTQSPATLSLSPGERATLSC | GACATCCAGTTGACCCAGTCTCCAGCCACCCTGTCTTTGTCTCCAGGGGAAAGAGCCAC |
| | RASQSVSSYLAWYQQKPGQAPRL | CCTCTCCTGCAGGGCCAGTCAGAGTGTTAGCAGCTACTTAGCCTGGTACCAACAGAAAC |
| | LIYDASNRATGIPARFSGSGSGT | CTGGCCAGGCTCCCAGGCTCCTCATCTATGATGCATCCAACAGGGCCACTGGCATCCCA |
| | DFTLTISSLEPEDFAVYYCQQRS | GCCAGGTTCAGTGGCAGTGGGTCTGGGACAGACTTCACTCTCACCATCAGCAGCCTAGA |
| | NWPPRVTFGGGTKVEIK | GCCTGAAGATTTTGCAGTTTATTACTGTCAGCAGCGTAGCAATTGGCCTCCGAGGGTCA |
| | | CTTTCGGCGGAGGGACCAAGGTGGAGATCAAA |
| Chi2M- | SEQ ID NO: 106 | SEQ ID NO: 144 |
| 8E7 | EIVLTQSPATLSLSPGERATLSC | GAAATTGTGTTGACACAGTCTCCAGCCACCCTGTCTTTGTCTCCAGGGGAAAGAGCCAC |
| | RASQSVSSYLAWYQQKPGQAPRL | CCTCTCCTGCAGGGCCAGTCAGAGTGTTAGCAGCTACTTAGCCTGGTACCAACAGAAAC |
| | LIYDASNRATGIPARFSGSGSGT | CTGGCCAGGCTCCCAGGCTCCTCATCTATGATGCATCCAACAGGGCCACTGGCATCCCA |
| | DFTLTISSLEPEDFAVYYCQQRS | GCCAGGTTCAGTGGCAGTGGGTCTGGGACAGACTTCACTCTCACCATCAGCAGCCTAGA |
| | NWPPKITFGQGTRLEIK | GCCTGAAGATTTTGCAGTTTATTACTGTCAGCAGCGTAGCAACTGGCCTCCGAAGATCA |
| | | CCTTCGGCCAAGGGACACGACTGGAGATTAAA |
| COV2- | SEQ ID NO: 107 | SEQ ID NO: 145 |
| 2621 | DFVMTQSPGSLAVSLGERATINC | GACTTCGTGATGACCCAGTCTCCAGGCTCCCTGGCTGTGTCTCTGGGCGAGAGGGCCAC |
| | RSSQSVLDNSSNKNHLAWHQQKP | CATCAATTGCAGGTCCAGTCAGAGTGTTTTAGACAACTCCAGCAATAAGAACCACTTAG |
| | GQPPKLLIYWASTRESGVPDRFS | CTTGGCACCAGCAGAAACCAGGACAGCCTCCTAAACTGCTCATTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYSSHWTFGQGTKVEIK | CACCATCAGCAGCCTGCAGGCTGAAGATGTGGCAGTTTATTACTGTCAGCAATATTATA |
| | | GTAGTCATTGGACGTTCGGCCAAGGGACCAAGGTGGAAATCAAA |
| COV2- | SEQ ID NO: 108 | SEQ ID NO: 146 |
| 2883 | DIVMTQSPDSLAVSLGERATINC | GACATCGTGATGACCCAGTCTCCAGACTCCCTGGCTGTGTCTCTGGGCGAGAGGGCCAC |
| COV2- | KSSQSVLHSSNNKDSLVWYQQKP | CATCAACTGCAAGTCCAGCCAGAGTGTCTTACACAGCTCCAACAACAAGGACTCCTTAG |
| 2224 | GQPPKLLIYWASSRESGVPDRFS | TTTGGTACCAGCAGAAACCAGGACAGCCTCCTAAGCTGCTCATTTACTGGGCATCTAGC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYSTPWTFGQGTKVEIK | CACCATCAGCAGCCTGCAGGCTGAAGATGTGGCAGTTTATTACTGTCAGCAATATTATA |
| | | GTACTCCTTGGACGTTCGGCCAAGGGACCAAGGTGGAAATCAAA |
| Chi2M- | SEQ ID NO: 110 | SEQ ID NO: 148 |
| 8H10 | DIQMTQSPDSLAVSLGERATIKC | GACATCCAGATGACCCAGTCTCCAGACTCCCTGGCTGTGTCTCTGGGCGAGAGGGCCAC |
| | KSSQSVLHSSNNKNYLAWYQQKA | CATCAAGTGCAAGTCCAGCCAGAGTGTTTTACACAGCTCCAACAATAAGAACTACTTAG |
| | GQPPSLLLYWASTRESGVPDRFS | CTTGGTACCAGCAAAAAGCGGGACAGCCTCCTAGCCTACTCCTTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYNNQWTFGQGTKVDIK | CACCATCAGCAGCCTGCAGGCTGAAGATGTGGCAGTTTATTACTGTCAGCAATATTATA |
| | | ATAATCAGTGGACGTTCGGCCAAGGGACCAAAGTGGATATCAAA |
| COV2- | SEQ ID NO: 111 | SEQ ID NO: 149 |
| 2401 | DIVMTQSPDSLAVSLGERATINC | GACATCGTGATGACCCAGTCTCCAGACTCCCTGGCTGTGTCTCTGGGCGAGAGGGCCAC |
| COV2- | KSSQSVLYSSNNKNSLAWYQQKP | CATCAACTGCAAGTCCAGCCAGAGTGTTTTATACAGCTCCAACAATAAGAACTCCTTAG |
| 2218 | GQPPKLLIYWASTRESGVPDRFS | CTTGGTACCAGCAGAAACCAGGACAGCCTCCTAAGCTGCTCATTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYSISWTFGQGTKVEIK | CACCATCAGCAGCCTGCAGGCTGAAGATGTGGCAGTATATTACTGTCAGCAATATTATA |
| | | GTATTTCTTGGACGTTCGGCCAAGGGACCAAGGTGGAAATCAAA |
| Chi0304- | SEQ ID NO: 113 | SEQ ID NO: 151 |
| 4A2 | EIVLTQSPDSLAVSLGERATINC | GAAATTGTGTTGACGCAGTCTCCAGACTCCCTGGCTGTGTCTCTGGGCGAGAGGGCCAC |
| | KSSQSVLYSSNNKNYLAWYQQKP | CATCAACTGCAAGTCCAGCCAGAGTGTTTTATACAGCTCCAACAATAAGAACTACCTAG |
| | GQPPKLLIYWASTRESGVPDRFS | CTTGGTACCAGCAGAAACCAGGACAGCCTCCTAAGCTGCTCATTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCHQYYNTPRTFGQGTKVEIK | CACCATCAGCAGCCTGCAGGCTGAAGATGTGGCAGTTTATTACTGTCACCAATATTATA |
| | | ATACTCCTCGAACGTTCGGCCAAGGGACCAAGGTGGAAATCAAA |
| COV2- | SEQ ID NO: 114 | SEQ ID NO: 152 |
| 2422 | DIVMTQSPDSLAVSLGERATINC | GACATCGTGATGACCCAGTCTCCAGACTCCCTGGCTGTGTCTCTGGGCGAGAGGGCCAC |
| | KSSQSVLYSSNNKNYLAWYQQKP | CATCAACTGCAAGTCCAGCCAGAGTGTTTTATACAGCTCCAACAATAAGAACTACTTAG |
| | GQPPKLLIYWASTRESGVPDRFS | CTTGGTACCAGCAGAAACCAGGACAGCCTCCTAAGCTGCTCATTTACTGGGCATCTACC |
| | GSGSGTDFTLTISSLQAEDVAVY | CGGGAATCCGGGGTCCCTGACCGATTCAGTGGCAGCGGGTCTGGGACAGATTTCACTCT |
| | YCQQYYSTPLTFGQGTKVEIK | CACCATCAGCAGCCTGCAGGCTGAAGATGTGGCAGTTTATTACTGTCAGCAATATTATA |
| | | GTACTCCTTTAACGTTCGGCCAAGGGACCAAGGTGGAAATCAAA |

TABLE 4
Heavy chain CDR amino acid sequences and
antibody lineages.

| Anti- | Line- | Iso- | | | | | |
|---|---|---|---|---|---|---|---|
| body | age | type | V | J | CDRH1 | CDRH2 | CDRH3 |
| CV27 | 185 | IGH | IGHV3- | IGHJ6 | SEQ | SEQ ID | SEQ ID |
| | | | 30- | | ID | NO: 171 | NO: 189 |
| | | | 3*01 | | NO: | | |
| | | | | | 153 | | |
| | | | | | GFTFS | ISYDGSN | ARSFGGS |
| | | | | | SYA | K | YYYGMDV |
| COV2- | 185 | IGH | IGHV3- | IGHJ6 | SEQ | SEQ ID | SEQ ID |
| 2147 | | | 30- | | ID | NO: 171 | NO: 190 |
| COV2- | | | 3*01 | | NO: | ISYDGSN | ARSTSGS |
| 2341 | | | | | 153 | K | YYYGMDV |
| COV2- | | | | | GFTFS | | |
| 2160 | | | | | SYA | | |
| COV2- | | | | | | | |
| 2159 | | | | | | | |
| COV2- | 185 | IGH | IGHV3- | IGHJ6 | SEQ | SEQ ID | SEQ ID |
| 2844 | | | 30- | | ID | NO: 172 | NO: 191 |
| | | | 3*01 | | NO: | ISYDGGN | ARAQGGN |
| | | | | | 154 | K | YYYGMDV |
| | | | | | GFIFS | | |
| | | | | | SYA | | |
| CV34 | 185 | IGH | IGHV3- | IGHJ6 | SEQ | SEQ ID | SEQ ID |
| | | | 30- | | ID | NO: 171 | NO: 192 |
| | | | 3*01 | | NO: | ISYDGSN | ARSYGGS |
| | | | | | 153 | K | YYYGMDV |
| | | | | | GFTFS | | |
| | | | | | SYA | | |
| COV2- | 185 | IGH | IGHV3- | IGHJ6 | SEQ | SEQ ID | SEQ ID |
| 2564 | | | 30- | | ID | NO: 173 | NO: 191 |
| | | | 3*01 | | NO: | ISYDGYN | ARAQGGN |
| | | | | | 153 | K | YYYGMDV |
| | | | | | GFTFS | | |
| | | | | | SYA | | |
| COV2- | 185 | IGH | IGHV3- | IGHJ6 | SEQ | SEQ ID | SEQ ID |
| 2643 | | | 30- | | ID | NO: 171 | NO: 193 |
| | | | 3*01 | | NO: | ISYDGSN | ARGSAGN |
| | | | | | 155 | K | YYYGMDV |
| | | | | | GFTFS | | |
| | | | | | SYG | | |
| COV2- | 185 | IGH | IGHV3- | IGHJ6 | SEQ | SEQ ID | SEQ ID |
| 2203 | | | 30- | | ID | NO: 173 | NO: 193 |
| COV2- | | | 3*01 | | NO: | ISYDGYN | ARGSAGN |
| 2250 | | | | | 156 | K | YYYGMDV |
| | | | | | GFTFS | | |
| | | | | | TYA | | |
| CV10 | 627 | IGH | IGHV4- | IGHJ4 | SEQ | SEQ ID | SEQ ID |
| | | | 59*03 | | ID | NO: 174 | NO: 194 |
| | | | | | NO: | IYYSGST | ARGFDY |
| | | | | | 157 | | |
| | | | | | GGSIS | | |
| | | | | | SYY | | |
| COV2- | 0 | IGH | IGHV5- | IGHJ6 | SEQ | SEQ ID | SEQ ID |
| 2656 | | | 51*01 | | ID | NO: 175 | NO: 195 |
| | | | | | NO: | IYPGDSD | ARLTFGG |
| | | | | | 158 | T | SGSYYFY |
| | | | | | GYSFS | | YNGMDV |
| | | | | | DYW | | |
| CV8 | 0 | IGH | IGHV1- | IGHJ6 | SEQ | SEQ ID | SEQ ID |
| | | | 18*04 | | ID | NO: 176 | NO: 196 |
| | | | | | NO: | ISAYNGN | ARLVPTW |
| | | | | | 159 | T | ASYYDFW |
| | | | | | GYTFT | | SGYPGGY |
| | | | | | SYG | | GMDV |
| COV2- | 0 | IGH | IGHV3- | IGHJ4 | SEQ | SEQ ID | SEQ ID |
| 2006 | | | 30- | | ID | NO: 171 | NO: 197 |
| | | | 3*01 | | NO: | ISYDGSN | ARPQSGG |
| | | | | | 160 | K | YYAPLDY |
| | | | | | GFTFS | | |
| | | | | | YYA | | |
| C205 | 0 | IGH | IGHV1- | IGHJ4 | SEQ | SEQ ID | SEQ ID |
| | | | 46*01 | | ID | NO: 177 | NO: 198 |
| | | | | | NO: | INPSGGS | ARGPERG |
| | | | | | 161 | T | IVGATDY |
| | | | | | GHTFT | | FDY |
| | | | | | SYY | | |
| COVA2- | 468 | IGH | IGHV1- | IGHJ4 | SEQ | SEQ ID | SEQ ID |
| 14 | | | 69*01 | | ID | NO: 178 | NO: 199 |
| | | | | | NO: | IIPIFGT | ARVRYYD |
| | | | | | 162 | A | SSGYYED |
| | | | | | GGTFS | | Y |
| | | | | | SYA | | |
| COV2- | 468 | IGH | IGHV1- | IGHJ4 | SEQ | SEQ ID | SEQ ID |
| 2270 | | | 69*01 | | ID | NO: 178 | NO: 200 |
| | | | | | NO: | IIPIFGT | AITYYYD |
| | | | | | 162 | A | SSGYWWD |
| | | | | | GGTFS | | D |
| | | | | | SYA | | |
| COV2- | 0 | IGH | IGHV4- | IGHJ6 | SEQ | SEQ ID | SEQ ID |
| 2430 | | | 39*01 | | ID | NO: 179 | NO: 201 |
| | | | | | NO: | VYYIGST | ARAPFQL |
| | | | | | 163 | | LDKYYFF |
| | | | | | GGSIS | | YYMDV |
| | | | | | SSSYY | | |
| COV2- | 468 | IGH | IGHV1- | IGHJ4 | SEQ | SEQ ID | SEQ ID |
| 2441 | | | 69*01 | | ID | NO: 180 | NO: 202 |
| COV2- | | | | | NO: | IIPIFGT | ARIGHFD |
| 2166 | | | | | 162 | T | SSGYYLD |
| COV2- | | | | | GGTFS | | Y |
| 2214 | | | | | SYA | | |
| COV2- | 468 | IGH | IGHV1- | IGHJ4 | SEQ | SEQ ID | SEQ ID |
| 2367 | | | 69*01 | | ID | NO: 181 | NO: 203 |
| COV2- | | | | | NO: | IIPIFGA | ARTSHYD |
| 2216 | | | | | 162 | A | SSGSYFE |
| COV2- | | | | | GGTFS | | Y |
| 2169 | | | | | SYA | | |
| COVA1- | 468 | IGH | IGHV1- | IGHJ4 | SEQ | SEQ ID | SEQ ID |
| 07 | | | 69*01 | | ID | NO: 178 | NO: 204 |
| | | | | | NO: | IIPIFGT | ARVGAYD |
| | | | | | 164 | A | SSGYSND |
| | | | | | GGTLS | | Y |
| | | | | | SYA | | |
| COVA2- | 468 | IGH | IGHV1- | IGHJ4 | SEQ | SEQ ID | SEQ ID |
| 18 | | | 69*01 | | ID | NO: 180 | NO: 205 |
| | | | | | NO: | IIPIFGT | ARVYSYD |
| | | | | | 162 | T | SSGYYLE |
| | | | | | GGTFS | | Y |
| | | | | | SYA | | |
| Chi2M- | 468 | IGH | IGHV1- | IGHJ4 | SEQ | SEQ ID | SEQ ID |
| 8E7 | | | 69*01 | | ID | NO: 178 | NO: 206 |
| | | | | | NO: | IIPIFGT | ARTYSFD |
| | | | | | 162 | A | SSGYYYD |
| | | | | | GGTFS | | Y |
| | | | | | SYA | | |
| COV2- | 401 | IGH | IGHV3- | IGHJI | SEQ | SEQ ID | SEQ ID |
| 2449 | | | 30*04 | | ID | NO: 182 | NO: 207 |
| | | | | | NO: | ISDDGNN | ARASYNS |
| | | | | | 165 | K | NWSIGEY |
| | | | | | GFTFS | | FRD |
| | | | | | SFA | | |
| COV2- | 0 | IGH | IGHV3- | IGHJ4 | SEQ | SEQ ID | SEQ ID |
| 2621 | | | 33*01 | | ID | NO: 183 | NO: 208 |
| | | | | | NO: | IWANGNR | ARDYCNG |
| | | | | | 166 | | VTCNSNY |
| | | | | | GFTFS | | |
| | | | | | SYL | | |
| COV2- | 0 | IGH | IGHV3- | IGHJ4 | SEQ | SEQ ID | SEQ ID |
| 2883 | | | 30*03 | | ID | NO: 171 | NO: 209 |
| COV2- | | | | | NO: | ISYDGSN | AKDGSIA |
| 2224 | | | | | 167 | K | AADY |
| | | | | | GFTFS | | |
| | | | | | TYG | | |
| Chi2M- | 0 | IGH | IGHV3- | IGHJ4 | SEQ | SEQ ID | SEQ ID |
| 8H10 | | | 30*04 | | ID | NO: 184 | NO: 210 |
| | | | | | NO: | ISDEGSN | ARAFYDS |
| | | | | | 168 | K | NWSVGSY |
| | | | | | GFTFS | | FDS |
| | | | | | TFA | | |
| COV2- | 0 | IGH | IGHV3- | IGHJ6 | SEQ | SEQ ID | SEQ ID |
| 2401 | | | 30*04 | | ID | NO: 185 | NO: 211 |
| COV2- | | | | | NO: | ISYDGNN | ARPYTGS |
| 2218 | | | | | 153 | K | YKSYMDV |
| | | | | | GFTFS | | |
| | | | | | SYA | | |
| Chi0304- | 0 | IGH | IGHV4- | IGHJ4 | SEQ | SEQ ID | SEQ ID |
| 4A2 | | | 39*07 | | ID | NO: 186 | NO: 212 |
| | | | | | NO: | IYYTGTT | ARELFTA |
| | | | | | 169 | | VAGKGGI |
| | | | | | GDSTS | | DY |
| | | | | | SSSSY | | |
| COV2- | 0 | IGH | IGHV3- | IGHJ4 | SEQ | SEQ ID | SEQ ID |
| 2422 | | | 30*04 | | ID | NO: 187 | NO: 213 |
| | | | | | NO: | ISYDGIN | ARVNSGS |
| | | | | | 153 | K | YYSYFDY |
| | | | | | GFTFS | | |
| | | | | | SYA | | |
| COV2- | 64 | IGH | IGHV3- | IGHJ6 | SEQ | SEQ ID | SEQ ID |
| 2143 | | | 66*01 | | ID | NO: 188 | NO: 214 |
| | | | | | NO: | IYSAGST | AKEGGSG |
| | | | | | 170 | | SLRYYYY |
| | | | | | GFTVS | | GMDV |
| | | | | | SNY | | |

TABLE 5
Light chain CDR amino acid sequences and
antibody lineages.

| Anti- | Line- | Iso- | | | | | |
|---|---|---|---|---|---|---|---|
| body | age | type | V | J | CDRL1 | CDRL2 | CDRL3 |
| CV27 | 1207 | IGL | IGLV2- | IGLJ6 | SEQ | SEQ | SEQ ID |
| | | | 14*01 | | ID | ID | NO: 242 |
| | | | | | NO: | NO: | SSYTSSS |
| | | | | | 215 | 233 | TPYV |
| | | | | | SSDVG | DVS | |
| | | | | | GYNY | | |
| COV2- | 1238 | IGL | IGLV2- | IGLJ1 | SEQ | SEQ | SEQ ID |
| 2147 | | | 14*01 | | ID | ID | NO: 243 |
| COV2- | | | | | NO: | NO: | SSYTSSS |
| 2341 | | | | | 216 | 233 | TLLYV |
| COV2- | | | | | SSDVG | DVS | |
| 2160 | | | | | DYNY | | |
| COV2- | | | | | | | |
| 2159 | | | | | | | |
| COV2- | 0 | IGL | IGLV3- | IGLJ2 | SEQ | SEQ | SEQ ID |
| 2844 | | | 21*03 | | ID | ID | NO: 244 |
| | | | | | NO: | NO: | QVWDSSS |
| | | | | | 217 | 234 | DHHVV |
| | | | | | NIGSK | HDG | |
| | | | | | N | | |
| CV34 | 0 | IGL | IGLV3 | IGLJ2 | SEQ | SEQ | SEQ ID |
| | | | 12*02 | | ID | ID | NO: 245 |
| | | | | | NO: | NO: | QVWDSSS |
| | | | | | 218 | 235 | DHVV |
| | | | | | NIGSK | SDS | |
| | | | | | A | | |
| COV2- | 0 | IGL | IGLV3 | IGLJ2 | SEQ | SEQ | SEQ ID |
| 2564 | | | 21*03 | | ID | ID | NO: 244 |
| | | | | | NO: | NO: | QVWDSSS |
| | | | | | 219 | 236 | DHHVV |
| | | | | | NIGTK | DDS | |
| | | | | | G | | |
| COV2- | 0 | IGK | IGKV1- | IGKJ5 | SEQ | SEQ | SEQ ID |
| 2643 | | | 39*01 | | ID | ID | NO: 246 |
| | | | | | NO: | NO: | QQSYSTP |
| | | | | | 220 | 237 | GT |
| | | | | | QSISS | AAS | |
| | | | | | Y | | |
| COV2- | 0 | IGK | IGKV1- | IGKJ2 | SEQ | SEQ | SEQ ID |
| 2203 | | | 39*01 | | ID | ID | NO: 247 |
| COV2- | | | | | NO: | NO: | QQSYSTP |
| 2250 | | | | | 221 | 237 | YT |
| | | | | | QTITN | AAS | |
| | | | | | Y | | |
| CV10 | 1068 | IGK | IGKV3- | IGKJ1 | SEQ | SEQ | SEQ ID |
| | | | 20*01 | | ID | ID | NO: 248 |
| | | | | | NO: | NO: | QQYAGSP |
| | | | | | 222 | 238 | WT |
| | | | | | QSVSS | GAS | |
| | | | | | IY | | |
| COV2- | 1068 | IGK | IGKV3- | IGKJ1 | SEQ | SEQ | SEQ ID |
| 2656 | | | 20*01 | | ID | ID | NO: 249 |
| | | | | | NO: | NO: | QQYGRSS |
| | | | | | 223 | 238 | GT |
| | | | | | QSVSS | GAS | |
| | | | | | SY | | |
| CV8 | 1068 | IGK | IGKV3- | IGKJ1 | SEQ | SEQ | SEQ ID |
| | | | 20*01 | | ID | ID | NO: 250 |
| | | | | | NO: | NO: | QQYGSSP |
| | | | | | 223 | 238 | GT |
| | | | | | QSVSS | GAS | |
| | | | | | SY | | |
| COV2- | 1068 | IGK | IGKV3- | IGKJ1 | SEQ | SEQ | SEQ ID |
| 2006 | | | 20*01 | | ID | ID | NO: 251 |
| | | | | | NO: | NO: | QQYGSSP |
| | | | | | 223 | 238 | WT |
| | | | | | QSVSS | GAS | |
| | | | | | SY | | |
| C205 | 1068 | IGK | IGKV3- | IGKJ1 | SEQ | SEQ | SEQ ID |
| | | | 20*01 | | ID | ID | NO: 252 |
| | | | | | NO: | NO: | QQYVSSP |
| | | | | | 223 | 238 | WT |
| | | | | | QSVSS | GAS | |
| | | | | | SY | | |
| COVA2- | 1186 | IGK | IGKV3- | IGKJ2 | SEQ | SEQ | SEQ ID |
| 14 | | | 11*01 | | ID | ID | NO: 253 |
| | | | | | NO: | NO: | QQRSNWP |
| | | | | | 224 | 239 | PMYT |
| | | | | | QSVSS | DAS | |
| | | | | | Y | | |
| COV2- | 1186 | IGK | IGKV3- | IGKJ2 | SEQ | SEQ | SEQ ID |
| 2270 | | | 11*01 | | ID | ID | NO: 254 |
| | | | | | NO: | NO: | QQRSNWP |
| | | | | | 225 | 239 | PSYT |
| | | | | | QSVSS | DAS | |
| | | | | | F | | |
| COV2- | 1202 | IGK | IGKV3- | IGKJ5 | SEQ | SEQ | SEQ ID |
| 2430 | | | 11*01 | | ID | ID | NO: 255 |
| | | | | | NO: | NO: | QQRSNWP |
| | | | | | 224 | 239 | PGVT |
| | | | | | QSVSS | DAS | |
| | | | | | Y | | |
| COV2- | 0 | IGK | IGKV3- | IGKJ3 | SEQ | SEQ | SEQ ID |
| 2441 | | | 11*01 | | ID | ID | NO: 256 |
| COV2- | | | | | NO: | NO: | QHRTNWP |
| 2166 | | | | | 225 | 239 | PLFT |
| COV2- | | | | | QSVSS | DAS | |
| 2214 | | | | | F | | |
| COV2- | 0 | IGK | IGKV3- | IGKJ4 | SEQ | SEQ | SEQ ID |
| 2367 | | | 11*01 | | ID | ID | NO: 257 |
| COV2- | | | | | NO: | NO: | HKRSNWP |
| 2216 | | | | | 224 | 239 | PSLT |
| COV2- | | | | | QSVSS | DAS | |
| 2169 | | | | | Y | | |
| COVA1- | 0 | IGK | IGKV3- | IGKJ4 | SEQ | SEQ | SEQ ID |
| 07 | | | 11*01 | | ID | ID | NO: 258 |
| | | | | | NO: | NO: | QQRSNWP |
| | | | | | 224 | 239 | PRVT |
| | | | | | QSVSS | DAS | |
| | | | | | Y | | |
| COVA2- | 1202 | IGK | IGKV3- | IGKJ5 | SEQ | SEQ | SEQ ID |
| 18 | | | 11*01 | | ID | ID | NO: 259 |
| | | | | | NO: | NO: | QQRSNWP |
| | | | | | 224 | 239 | PSIT |
| | | | | | QSVSS | DAS | |
| | | | | | Y | | |
| Chi2M- | 1202 | IGK | IGKV3- | IGKJ5 | SEQ | SEQ | SEQ ID |
| 8E7 | | | 11*01 | | ID | ID | NO: 260 |
| | | | | | NO: | NO: | QQRSNWP |
| | | | | | 224 | 239 | PKIT |
| | | | | | QSVSS | DAS | |
| | | | | | Y | | |
| COV2- | 1059 | IGK | IGKV4- | IGKJ1 | SEQ | SEQ | SEQ ID |
| 2449 | | | 1*01 | | ID | ID | NO: 261 |
| | | | | | NO: | NO: | QQYYSPP |
| | | | | | 226 | 240 | WT |
| | | | | | QSLLY | WAS | |
| | | | | | TSNNK | | |
| | | | | | NY | | |
| COV2- | 1059 | IGK | IGKV4- | IGKJ1 | SEQ | SEQ | SEQ ID |
| 2621 | | | 1*01 | | ID | ID | NO: 262 |
| | | | | | NO: | NO: | QQYYSSH |
| | | | | | 227 | 240 | WT |
| | | | | | QSVLD | WAS | |
| | | | | | NSSNK | | |
| | | | | | NH | | |
| COV2- | 1059 | IGK | IGKV4- | IGKJ1 | SEQ | SEQ | SEQ ID |
| 2883 | | | 1*01 | | ID | ID | NO: 263 |
| COV2- | | | | | NO: | NO: | QQYYSTP |
| 2224 | | | | | 228 | 240 | WT |
| | | | | | QSVLH | WAS | |
| | | | | | SSNNK | | |
| | | | | | DS | | |
| Chi2M- | 1059 | IGK | IGKV4- | IGKJ1 | SEQ | SEQ | SEQ ID |
| 8H10 | | | 1*01 | | ID | ID | NO: 264 |
| | | | | | NO: | NO: | QQYYNNQ |
| | | | | | 229 | 240 | WT |
| | | | | | QSVLH | WAS | |
| | | | | | SSNNK | | |
| | | | | | NY | | |
| COV2- | 1059 | IGK | IGKV4- | IGKJ1 | SEQ | SEQ | SEQ ID |
| 2401 | | | 1*01 | | ID | ID | NO: 265 |
| COV2- | | | | | NO: | NO: | QQYYSIS |
| 2218 | | | | | 230 | 240 | WT |
| | | | | | QSVLY | WAS | |
| | | | | | SSNNK | | |
| | | | | | NS | | |
| Chi0304- | 1059 | IGK | IGKV4- | IGKJ1 | SEQ | SEQ | SEQ ID |
| 4A2 | | | 1*01 | | ID | ID | NO: 266 |
| | | | | | NO: | NO: | HQYYNTP |
| | | | | | 231 | 240 | RT |
| | | | | | QSVLY | WAS | |
| | | | | | SSNNK | | |
| | | | | | NY | | |
| COV2- | 1059 | IGK | IGKV4- | IGKJ1 | SEQ | SEQ | SEQ ID |
| 2422 | | | 1*01 | | ID | ID | NO: 267 |
| | | | | | NO: | NO: | QQYYSTP |
| | | | | | 231 | 240 | LT |
| | | | | | QSVLY | WAS | |
| | | | | | SSNNK | | |
| | | | | | NY | | |
| COV2- | 1191 | IGL | IGLV1- | IGLJ1 | SEQ | SEQ | SEQ ID |
| 2143 | | | 44*01 | | ID | ID | NO: 268 |
| | | | | | NO: | NO: | AAWDDSL |
| | | | | | 232 | 241 | NGYV |
| | | | | | SSNIG | SNN | |
| | | | | | YNI | | |

### D. Linkers

The fusion proteins and modified proteins provided herein comprise at least one linker. Linkers, also referred to as spacers, as used herein are flexible molecules or a flexible stretch of molecules that joins or connects two portions (e.g., domains) of a fusion protein or a modified protein as provided herein. The linker may increase the range of orientations that may be adopted by the domains of the fusion protein or modified protein. The linker may be optimized to produce desired effects in the fusion protein or modified protein. Aspects of linker design and considerations are described, for example, in Chen, X, et al., Adv Drug Deliv Rev. 2013 Oct. 15; 65(10): 1357-1369, and Klein, J. S, et al. 2014 Protein Eng. Des. Sel. 27(10):325-330. In some embodiments, the proteins provided herein comprise a peptide linker (e.g., in the fusion proteins provided herein). In some embodiments, the proteins provided herein comprise a non-peptide linker (e.g., in the modified proteins provided herein). In some embodiments, the proteins provided herein comprise a peptide linker and a non-peptide linker. The proteins provided herein may also comprise a plurality of linkers, including at least one peptide linker, at least one non-peptide linker, or at least one peptide linker and at least one non-peptide linker.

In some embodiments, the length of a linker may affect the ability of the fusion protein or the modified protein to bind to and/or neutralize a coronavirus virion by facilitating the binding of both the neutralizing polypeptide and the antibody that specifically binds an epitope in a conserved region of a coronavirus spike protein to their respective viral protein binding sites. For example, a longer linker may be desirable when the RBD of a coronavirus spike protein (i.e., to which the neutralizing polypeptide of the fusion protein or modified protein binds) and the epitope of the coronavirus spike protein (i.e., to which the antibody that specifically binds an epitope in a conserved region of a coronavirus spike protein binds) are far away from each other. Conversely, a shorter linker may be desirable when the receptor binding domain and epitope are close to each other. The length of the linker may also be selected based on the binding orientation of the antibody of the fusion protein or modified protein that specifically binds an epitope in a conserved region of a coronavirus spike protein. If the antibody binds the epitope in the conserved region of the coronavirus spike protein in such a way that the neutralizing polypeptide of the fusion protein or modified protein is brought into close proximity of the coronavirus spike protein RBD, a shorter linker may be desirable. Various factors may influence the binding orientation of the antibody that specifically binds an epitope in a conserved region of a coronavirus spike protein (and, thus, the fusion protein or modified protein), including, but not limited to, the order of the domains in the fusion protein or modified protein and the antibody format used.

In some embodiments, the linkers of the fusion proteins or modified proteins provided herein are about 100 angstroms (Å) to about 450 Å in length (e.g., about 100 Å to about 300 Å, about 150 Å to about 400 Å, about 150 Å to about 350 Å, about 200 Å to about 300 Å, about 200 Å to about 400 Å, about 150 Å to about 45 Å, about 100 Å, about 150 Å, about 200 Å, about 250 Å, about 300 Å, about 350 Å, about 400 Å, or about 450 Å). Selection of linkers to achieve the desired length is within the ability of one skilled in the art. The average diameter of an amino acid is approximately 4 Å. Thus, a peptide linker may be, for example, 25 to 100 or more amino acids in length (e.g., 25 aa, 30 aa, 35 aa, 40 aa, 45 aa, 50 aa, 55 aa, 60 aa, 65 aa, 70 aa, 75 aa, 80 aa, 85 aa, 90 aa, 95 aa, or 100 aa). Non-peptide linkers may similarly be selected based on the size of the repeating molecule(s) which they are made. For example, a polyethylene glycol (PEG) monomer is also approximately 4 Å. Thus, a polyethylene glycol (PEG) linker may be, for example, 25 to 100 PEG linkages in length (e.g., 25 PEG linkages, 30 PEG linkages, 35 PEG linkages, 40 PEG linkages, 45 PEG linkages, 50 PEG linkages, 55 PEG linkages, 60 PEG linkages, 65 PEG linkages, 70 PEG linkages, 75 PEG linkages, 80 PEG linkages, 85 PEG linkages, 90 PEG linkages, 95 PEG linkages, or 100 PEG linkages). Peptide linkers and PEG linkers are described below.

Depending on length, linker sequence may have various conformations in secondary structure, such as helical, β-strand, coil/bend, and turns. In some instances, a linker sequence may have an extended conformation and function as an independent domain that does not interact with the adjacent protein domains. Linker sequences may be flexible or rigid. Flexible linkers provide a certain degree of movement or interaction between the polypeptide domains and are generally rich in small or polar amino acids such as Gly and Ser (e.g., at least 90%, at least 95%, at least 98%, at least 99%, or all of the amino acid residues of the linker are either Gly or Ser). A rigid linker can be used to keep a fixed distance between the domains and to help maintain their independent functions. Linker attachment can be through an amide linkage (e.g., a peptide bond) or other functionalities as discussed further below.

In some embodiments, a peptide linker described herein comprises an amino acid sequence with at least 90% sequence identity to any one of SEQ ID NOs: 294-299. In some embodiments, the linker comprises one or more repeats of GGGGS (SEQ ID NO:305) and/or one or more repeats of GSSGSS (SEQ ID NO:306). Additional exemplary peptide linkers include, but are not limited to, peptide linkers comprising SGSETPGTSESATPE (SEQ ID NO:307), SGSETPGTSESATPES (SEQ ID NO:308), (GGGGS); (SEQ ID NO:309), (GGGGS); (SEQ ID NO:310), (GGGGS) 10 (SEQ ID NO:311), GGGGGGGG (SEQ ID NO:312), GSAGSAAGSGEF (SEQ ID NO:313), A(EAAAK); A (SEQ ID NO:314), or A(EAAAK)₁₀A (SEQ ID NO:315). Additional non-limiting exemplary linkers that can be used include those disclosed in Chen et al., Adv. Drug. Deliv. Rev. 65 (10): 1357-1369 (2014) and Rosemalen et al., Biochemistry 2017, 56, 50, 6565-6574, the entire contents of both of which are herein incorporated by reference.

In some embodiments (e.g., for research purposes), the peptide linker comprises a protease recognition site, e.g., a Tobacco Etch Virus (TEV) protease cut site (ENLYFQG (SEQ ID NO:316)). Such protease recognition sites may be useful for testing binding of the fusion proteins compared to the individual domains, as in the Examples herein. In some embodiments, the peptide linker does not comprise a protease recognition site.

In some embodiments, a non-peptide linker can comprise any of a number of known chemical linkers. Exemplary chemical linkers can include one or more units of beta-alanine, 4-aminobutyric acid (GABA), (2-aminoethoxy) acetic acid (AEA), 5-aminobexanoic acid (Ahx), PEG multimers, and trioxatricdeacan-succinamic acid (Ttds). In some embodiments, the non-peptide linker comprises one or more units of polyethylene glycol (PEG), which is commonly used as a linker for conjugation of polypeptide domains due to its water solubility, lack of toxicity, low immunogenicity, and well-defined chain lengths. See, e.g., Ramirez-Paz, J., et al., PLoS One 13(7):e0197643 (2018). As described above, the number of PEG linkage units may be selected based on the desired length of the linker.

Modified proteins comprising a linker can be produced in a variety of ways. For example, a neutralizing polypeptide and an antibody that specifically binds an epitope in a conserved region of a coronavirus spike protein may be produced separately (e.g., in vitro or by expression in and purification from host cells) and chemically linked in vitro. In some embodiments, a neutralizing polypeptide, an antibody that specifically binds an epitope in a conserved region of a coronavirus spike protein, and a linker can each be produced separately and chemically linked in vitro. In some embodiments, provided herein is a partial modified protein comprising a neutralizing polypeptide with or without a linker. In some embodiments, provided herein is a partial modified protein comprising an antibody that specifically binds an epitope in a conserved region of a coronavirus spike protein as described above with or without a linker. Various chemical linkers may be used to cross link two amino acid residues, as described further in Section II below.

### E. Additional Domains and Sequences

Fusion proteins and modified proteins described in the present disclosure can comprise a domain or sequence useful for protein isolation. In some embodiments, the fusion proteins and modified proteins can comprise an affinity tag, for example an AviTag™, a Myc tag, a polyhistidine tag (such as 8 XHis tag), an albumin-binding protein, an alkaline phosphatase, an AUI epitope, an AUS epitope, a biotin-carboxy carrier protein (BCCP), or a FLAG epitope, to name a few. In some embodiments, the affinity tags are useful for protein isolation. See, for example, Kimple et al., 2013. In some embodiments, the fusion proteins and modified proteins comprise a signal sequence useful for protein isolation, for example a mutated Interleukin-2 signal peptide sequence, which promotes secretion and facilitates protein isolation. See, for example, Low et al., 2013. In some embodiments, a fusion protein or modified protein comprises a protease recognition site, for example, TEV protease cut site, which may be useful for, among other things, removal of a signal peptide or affinity purification tag following isolation of the fusion protein or modified protein.

In some embodiments, the fusion proteins or modified proteins provided herein comprise amino acid substitutions that improve binding or other properties. For example, one or more cysteine substitutions, or substitutions with noncanonical amino acids containing long side-chain thiols, may be introduced into the polypeptides that can form disulfide bonds between two polypeptides that have interacted to form a dimer. In some embodiments, the substitutions improve polypeptide stability. In some instances, amino acids found to not contribute to the binding specificity and/or affinity of a fusion protein or modified protein can be deleted without a loss in the respective activity. Insertions, deletions, substitutions, or other selected mutations of particular regions or specific amino acids residues, provided the activity of the fragment is not significantly altered or impaired compared to the non-mutated fusion protein, modified protein, or components thereof can be made. Such methods are readily apparent to a skilled practitioner in the art and can include site specific mutagenesis of the nucleic acid encoding the fusion protein, modified protein, or fragment thereof. (Zoller et al., Nucl. Acids Res. 10:6487-500 (1982)).

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

In any of the polypeptides described herein, where a specific amino acid sequence is recited, embodiments comprising a sequence having at least 90% (e.g. 90%, 91%, 92%, 93%, 94%, 95%, 96%, 97%, 98%, or 99%) identity to the recited sequence are also provided.

The terms “identity,” “substantial identity,” “similarity,” “substantial similarity,” “homology” and the related terms and expressions used in the context of describing nucleic acid or amino acid sequences refer to a sequence that has at least 60% sequence identity to a reference sequence. Percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity are the BLAST and BLAST 2.0 algorithms, which are described in Altschul et al. (1990). J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

Sequence identity can also be determined by inspection. For example, the sequence identity between sequence A and sequence B, aligned using the software above or manually (to maximize alignment), can be determined by dividing the length of sequence A, minus the number of gap residues in sequence A, minus the number of gap residues in sequence B, by the sum of the residue matches between sequence A and sequence B, times one hundred.

Any of the fusion proteins and modified proteins described herein can be further modified. The modifications can be covalent or non-covalent modifications. Such modifications can be introduced into the fusion protein or modified protein by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the fusion proteins or modified proteins. In some instances, the fusion proteins or modified proteins may be labeled by a variety of means for use in diagnostic and/or pharmaceutical applications.

In some embodiments, the fusion proteins or modified proteins can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK) (SEQ ID NO:317), polyhistidine (e.g., 6 X-His; HHHHHH (SEQ ID NO:318)), hemagglutinin (HA; YPYDVPDYA (SEQ ID NO:319)), glutathione-S-transferase (GST), or maltose-binding protein (MBP)) for use in purifying the fusion proteins or modified proteins. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase. Another labeling technique which may result in greater sensitivity consists of coupling the fusion proteins or modified proteins to low molecular weight haptens. These haptens can then be specifically altered by means of a second reaction. For example, it is common to use haptens such as biotin, which reacts with avidin, or dinitrophenol, pyridoxal, or fluorescein, which can react with specific antihapten antibodies.

Two proteins (e.g., an antibody and a heterologous moiety) can be cross-linked using any of a number of known chemical cross linkers. Examples of such cross linkers are those that link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-a-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the fusion protein or modified protein. Alternatively, the radioactive label can be included as part of a larger molecule (e.g., 125I in meta-[¹²⁵I]iodophenyl-N-hydroxysuccinimide ([¹²⁵I]mIPNHS), which binds to free amino groups to form meta-jodophenyl (mIP) derivatives of relevant proteins (see, e.g., Rogers et al. (1997) J Nucl Med 38:1221-1229) or chelate (e.g., to DOTA or DTPA), which is in turn bound to the protein backbone. Methods of conjugating the radioactive labels or larger molecules/chelates containing them to the fusion proteins or modified proteins described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a fluorophore) to a protein (e.g., a fusion protein or a modified protein) are known in the art of protein chemistry. For example, fluorophores can be conjugated to free amino groups (e.g., of lysines) or sulfhydryl groups (e.g., cysteines) of proteins using succinimidyl (NHS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein or fragment thereof with the fluorophore under conditions that facilitate binding of the fluorophore to the protein. See, e.g., Welch and Redvanly (2003) Handbook of Radiopharmaceuticals: Radiochemistry and Applications, John Wiley and Sons.

In some embodiments, the fusion protein or modified protein can be modified, e.g., with a moiety that improves the stabilization and/or retention of the fusion protein or modified protein in circulation, e.g., in blood, serum, or other tissues. For example, the fusion protein or modified protein can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476, or HESylated (Fresenius Kabi, Germany) (see, e.g., Pavisić et al. (2010) Int J Pharm 387(1-2): 110-119). The stabilization moiety can improve the stability, or retention of, the fusion protein or modified protein (or fragment) by at least 1.5 (e.g., at least 2, 5, 10, 15, 20, 25, 30, 40, or 50 or more) fold.

In some embodiments, the fusion protein or modified protein described herein can be glycosylated. In some embodiments, a fusion protein or modified protein described herein can be subjected to enzymatic or chemical treatment, or produced from a cell, such that the fusion protein or modified protein has reduced or absent glycosylation. Methods for producing fusion proteins or modified proteins with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### F. Viral Protein Binding and Neutralization

The fusion proteins and modified proteins provided herein specifically bind to one or more coronavirus spike proteins. As described below, the neutralizing polypeptide of a fusion protein or a modified protein described herein binds to a first coronavirus spike protein (e.g., through binding of the RBD of a first coronavirus spike protein). In some embodiments, the first coronavirus spike protein is a SARS-COV-1 spike protein, a SARS-COV-2 spike protein, and/or a MERS-COV spike protein. As described above, the antibody of a fusion protein or a modified protein described herein that specifically binds an epitope in a conserved region of a coronavirus spike protein specifically binds an epitope in a conserved region of a second coronavirus spike protein. In some embodiments, the antibody that specifically binds an epitope in a conserved region of a coronavirus spike protein specifically binds an epitope in a conserved region of SARS-COV-1 spike protein, SARS-COV-2 spike protein, and/or MERS-COV spike protein. In some embodiments, the region is conserved in at least two of the group consisting of SARS-CoV-1, SARS-COV-2, and MERS-COV. In some embodiments, the second coronavirus spike protein is a SARS-COV-1 spike protein, a SARS-COV-2 spike protein, and/or a MERS-COV spike protein. In some embodiments, the first coronavirus spike protein and the second coronavirus spike protein are both a SARS-COV-1 spike protein, both a SARS-COV-2 spike protein, and/or both a MERS-COV spike protein. In some embodiments, the first coronavirus spike protein and the second coronavirus spike protein are the same protein (i.e., a single coronavirus spike protein). In some embodiments, the neutralizing polypeptide and the antibody that specifically binds an epitope in a conserved region of a coronavirus spike protein do not bind competitively to their respective binding sites. For example, the neutralizing polypeptide binds to the spike protein RBD while the antibody that specifically binds an epitope in a conserved region of a coronavirus spike protein binds to another region on the spike protein or to the spike protein RBD but without interfering with binding of the neutralizing polypeptide to the spike protein RBD. In some embodiments, the first coronavirus spike protein and the second coronavirus spike protein are different proteins, e.g., co-monomers of a spike protein homotrimer. In some embodiments, the first coronavirus spike protein and the second coronavirus spike protein are different coronavirus spike proteins selected from the group consisting of a SARS-COV-1 spike protein, a SARS-COV-2 spike protein, and a MERS-COV spike protein.

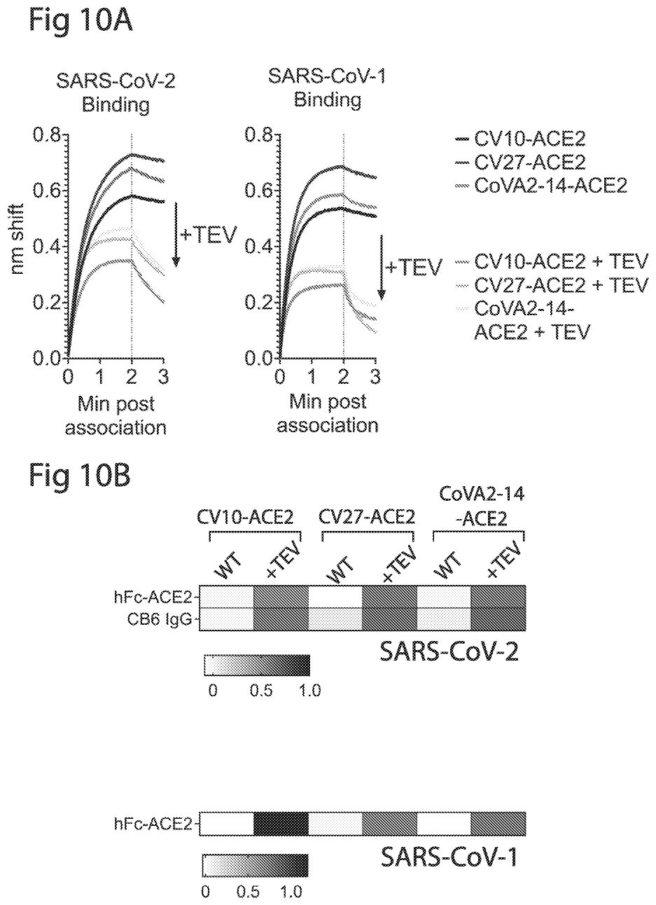

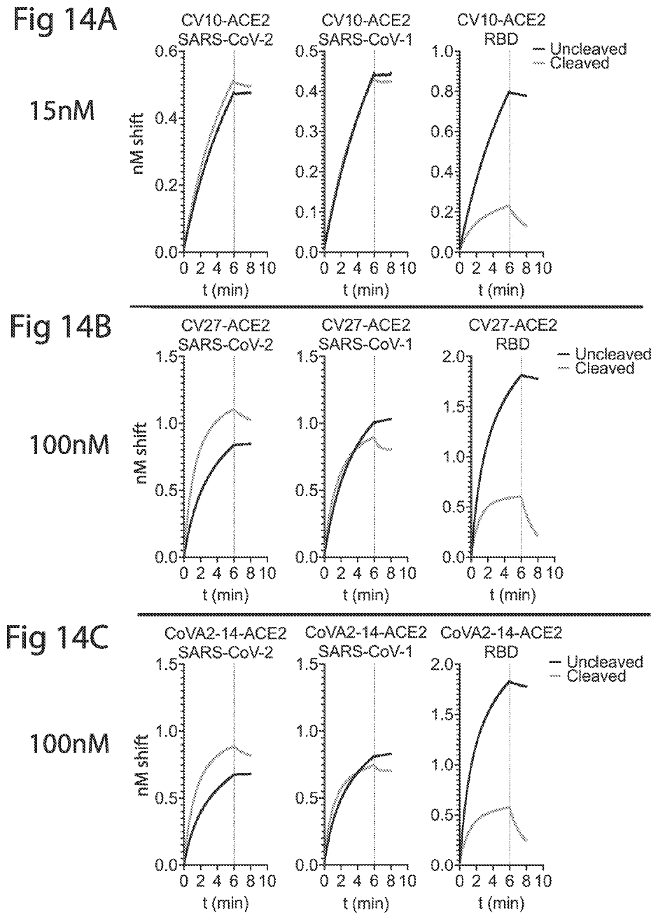

In some instances, the fusion proteins and modified proteins provided herein have increased binding affinity for a coronavirus spike protein relative to the binding affinity of the individual domains (e.g., the neutralizing polypeptide and the antibody that specifically binds an epitope in a conserved region of a coronavirus spike protein). See, e.g., Examples 3 and 4, FIG. 10, and FIG. 14 herein, which show a decrease in binding of SARS-COV-2 and SARS-COV-1 spike proteins by the fusion protein domains following cleavage of the fusion protein. As used herein, binding affinity of the provided fusion proteins or modified proteins may be measured as the dissociation constant “K_{D}” or apparent K_{D}. Binding affinity can be determined by a variety of methods known in the art. In some instances, bio-layer inferometry can be used to measure binding affinity. For example, as described in the Examples herein, the binding of fusion proteins or modified proteins to coronavirus spike protein can be measured by bio-layer interferometry. Bio-layer interferometry (“BLI”) is an optical technique for measuring macromolecular interactions by analyzing interference patterns of white light reflected from the surface of a biosensor tip coated with an immobilized protein, with any change in the number of molecules bound to the biosensor tip (i.e. protein-protein interactions) causing a shift in the interference pattern. Other methods of measuring binding affinity include yeast surface display binding assays, ELISA, surface plasmon resonance, or kinetic exclusion assays (Kinexa®). The K_{D} range in which measurements are accurate for different analytical methods may vary. One of skill in the art will appreciate that, within the accurate range, these methods will result in similar binding affinity measurements or similar trends in relative binding affinities for the various fusion proteins or modified proteins described herein as compared to their respective individual domains.

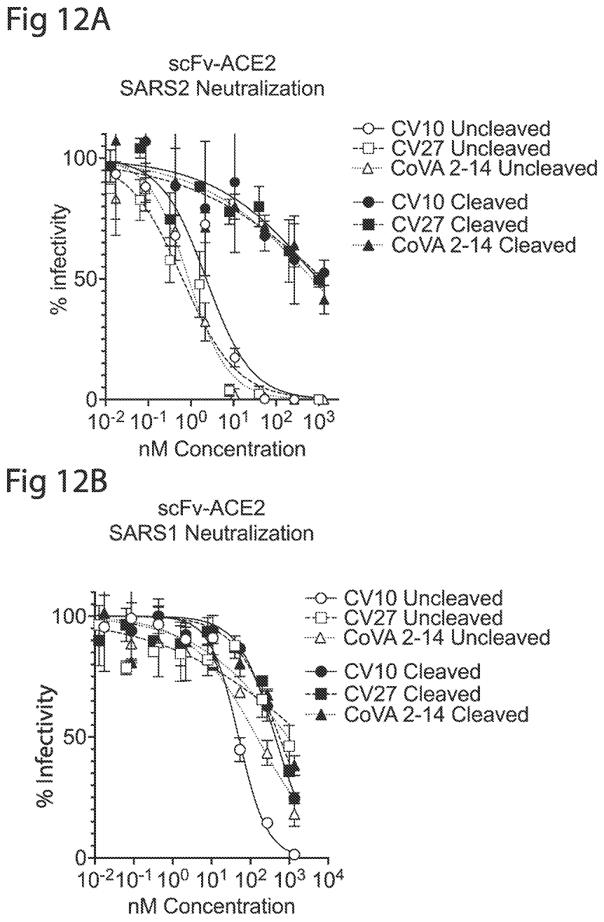

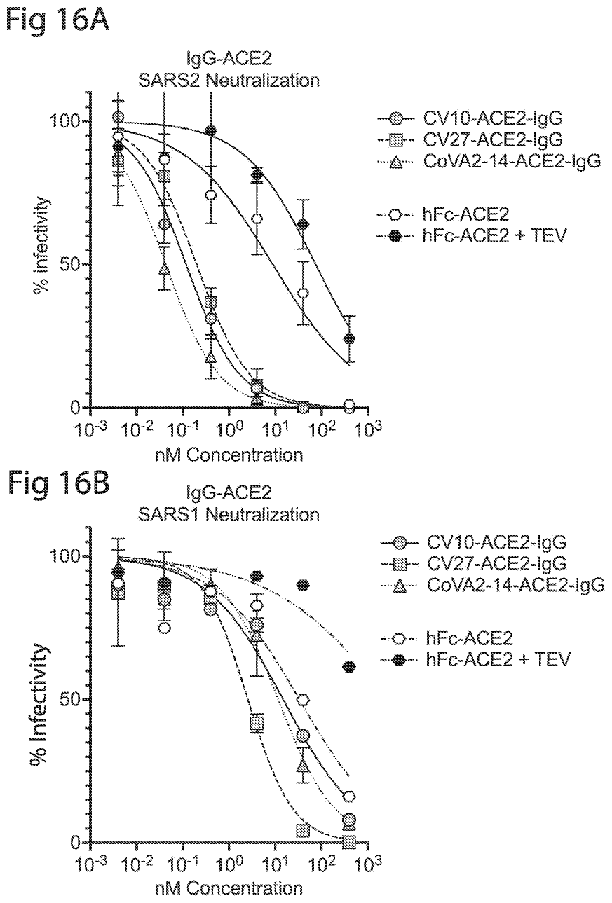

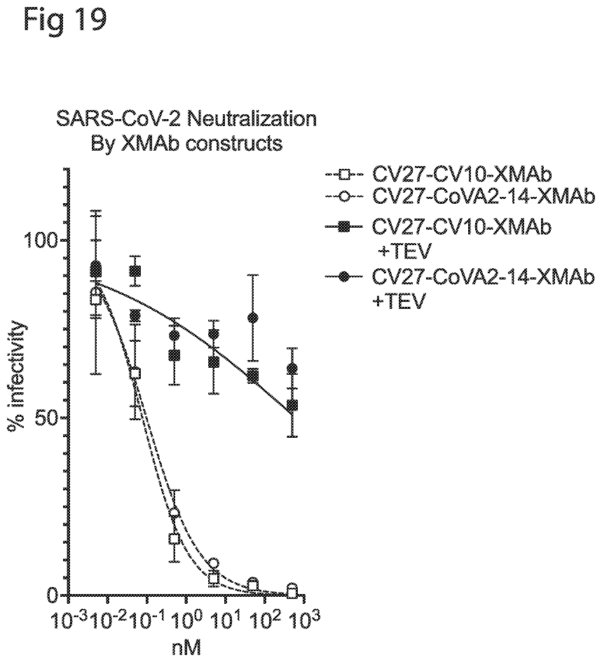

In some instances, the fusion proteins and modified proteins provided herein have increased efficacy in neutralizing coronaviruses compared to the individual domains (e.g., the neutralizing polypeptide and the antibody that specifically binds an epitope in a conserved region of a coronavirus spike protein). See, e.g., Examples 3 and 4, FIG. 12, FIG. 16, and FIG. 19 herein, which show a decrease in neutralization of SARS-COV-2 and SARS-COV-1 pseudotyped lentiviruses by the fusion protein domains following cleavage of the fusion protein. In some embodiments, fusion proteins provided herein have up to about 1000-fold increased potency for neutralization of SARS-COV-2 compared to the cleaved individual domains (see Example 3).

In some instances, as shown in FIG. 16 and described in Example 4 herein, the fusion proteins and modified proteins have increased efficacy in neutralizing SARS-COV-2 and SARS-CoV-1 coronaviruses (the assays described herein use SARS-COV-2 and SARS-COV-1 pseudotyped lentiviruses) relative to bivalent ACE2 (e.g., ACE2 polypeptides present as a dimer through fusion to an Fc dimerization domain) and monovalent ACE2 (e.g., ACE2 polypeptides cleaved from the Fc dimerization domains using TEV protease). In some embodiments, fusion proteins provided herein have up to about 44-fold increased potency for neutralization of SARS-CoV-2 and 13-fold increased potency for neutralization of SARS-COV-1 compared to bivalent ACE2 and up to about 376-fold increased potency for neutralization of SARS-COV-2 and 1162-fold increased potency for neutralization of SARS-COV-1 compared to monovalent ACE2. See Example 4 and Table 10 herein.

### II. Nucleic Acids, Vectors, Host Cells, and Related Methods

Any of the fusion proteins or modified proteins described herein can be purified or isolated from a host cell or population of host cells. For example, a recombinant nucleic acid encoding any of the proteins described herein can be introduced into a host cell under conditions that allow expression of the protein. In some embodiments, the recombinant nucleic acid is codon-optimized for expression. After expression in the host cell, the recombinant protein can be isolated or purified using purification methods known in the art. In some embodiments, a recombinant nucleic acid encoding a fusion protein, modified protein, or one or more domains thereof as described herein can be introduced into a host cell under conditions that allow expression thereof. In some embodiments, the expressed polypeptide forms a protein dimer. In some instances, the protein dimer comprises an antibody. In some embodiments, a plurality of recombinant nucleic acids each encoding a different fusion protein or modified protein as described herein can be introduced into a host cell under conditions that allow expression of the fusion proteins or modified proteins, with the expressed polypeptides forming a multimeric protein complex. In some instances, the multimeric protein complex comprises an antibody. After expression in the host cell, the protein dimer or multimeric protein complex can be isolated or purified using purification methods known in the art. In some embodiments, the fusion protein or modified protein is isolated as a monomer and allowed to dimerize or multimerize in vitro. In some instances, one or more of the domains of the fusion protein or modified protein can be expressed and/or isolated individually and then assembled with the remaining domains to form the fusion protein or modified protein.

Recombinant nucleic acids encoding any of the polypeptides or proteins described herein are provided. As used throughout, the terms “nucleic acid,” “nucleic acid sequence,” “nucleotide sequence,” “oligonucleotide,” “polynucleotide” and the related terms and expressions refer to deoxyribonucleic acids (DNA) or ribonucleic acids (RNA) and their polymers. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

Nucleic acid sequences encoding the heavy chain and light chain variable region sequences of the antibodies that specifically bind epitopes in conserved regions of a coronavirus spike protein encompassed by this disclosure are set forth in Table 2 and Table 3. In some embodiments, the nucleic acid sequence encodes an antibody comprising a heavy chain variable region, the nucleic acid sequence having at least 90% identity (e.g., at least 91%, at least 92%, at least 93%, at least 94%, at least 95%, at least 96%, at least 97%, at least 98%, or at least 99%) to any of SEQ ID NOs: 39-45, 49-53, 55-61, 64, 67-70, 72, 73, 75, and 76. In some embodiments, the nucleic acid sequence encodes an antibody comprising a light chain variable region, the nucleic acid sequence having at least 90% identity (e.g., at least 91%, at least 92%, at least 93%, at least 94%, at least 95%, at least 96%, at least 97%, at least 98%, or at least 99%) to any of SEQ ID NOs: 115-121, 125-129, 131-137, 140, 143-146, 148, 149, 151, and 152. In some embodiments, the nucleic acid sequence encodes an antibody comprising a heavy chain variable region and a light chain variable region, wherein the nucleic acid sequence encoding the heavy chain variable region has at least 90% identity to any of SEQ ID NOs: 39-45, 49-53, 55-61, 64, 67-70, 72, 73, 75, and 76 and wherein the nucleic acid sequence encoding the light chain variable region has at least 90% identity to any of SEQ ID NOs: 115-121, 125-129, 131-137, 140, 143-146, 148, 149, 151, and 152. In some embodiments, provided herein are nucleic acid sequences encoding an antibody comprising a heavy chain variable region having at least 90% identity (e.g., at least 91%, at least 92%, at least 93%, at least 94%, at least 95%, at least 96%, at least 97%, at least 98%, or at least 99%) to any of SEQ ID NOs: 1-7, 11-15, 17-23, 26, 29-32, 34, 35, 37, 38, 328, 330, 331, 333, and 335. In some embodiments, provided herein are nucleic acid sequences encoding an antibody comprising a light chain variable region having at least 90% identity (e.g., at least 91%, at least 92%, at least 93%, at least 94%, at least 95%, at least 96%, at least 97%, at least 98%, or at least 99%) to any of SEQ ID NOs: 77-83, 87-91, 93-99, 102, 105-108, 110, 111, 113, 114, 329, 332, 334, and 336. In some embodiments, provided herein are nucleic acid sequences encoding an antibody comprising a heavy chain variable region having at least 90% identity to any of SEQ ID NOs: 1-7, 11-15, 17-23, 26, 29-32, 34, 35, 37, 38, 328, 330, 331, 333, and 335 and a light chain variable region having at least 90% identity to any of SEQ ID NOs: 77-83, 87-91, 93-99, 102, 105-108, 110, 111, 113, 114, 329, 332, 334, and 336.

Also provided are nucleic acid sequences encoding the coronavirus receptor polypeptides described above. In some embodiments, the nucleic acid sequence encodes an ACE2 receptor ectodomain polypeptide having at least 80% identity (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to the amino acid sequence of SEQ ID NO:270 or SEQ ID NO:271. In some embodiments, the nucleic acid sequence encodes an ACE2 receptor ectodomain polypeptide comprising one or more mutations (i.e., relative to SEQ ID NO:269). In some embodiments, the one or more mutations are able to increase binding affinity of the ACE2 receptor ectodomain polypeptide for the RBD of a coronavirus spike protein. In some embodiments, the nucleic acid sequence encodes an ACE2 receptor ectodomain polypeptide comprising amino acid substitions at one or more of the following residues (the positions listed are relative to SEQ ID NO:269): the arginine at position 273 (R273), the histidine at position 378 (H378), the glutamate at position 402 (E402), the histidine at position 374 (H374), and the histidine at position 345 (H345). In some embodiments, the nucleic acid sequence encodes an ACE2 receptor ectodomain polypeptide comprising one or more of the following amino acid substitions: R273A (i.e., the arginine residue at position 273 (relative to SEQ ID NO:269) is substituted for an alanine residue), H378A, E402A, H374N, and H345L.

In some embodiments, the nucleic acid sequence encodes a DPP4 receptor ectodomain polypeptide having at least 80% identity (e.g., at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%) to the amino acid sequence of SEQ ID NO:273 or SEQ ID NO:274. In some embodiments, the nucleic acid sequence encodes a DPP4 receptor ectodomain polypeptide comprising one or more mutations (i.e., relative to SEQ ID NO:272). In some embodiments, the one or more mutations are able to increase binding affinity of the DPP4 receptor ectodomain polypeptide for the RBD of a coronavirus spike protein.

Also provided is a DNA construct comprising a promoter operably linked to a recombinant nucleic acid described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

The recombinant nucleic acids provided herein can be included in expression cassettes for expression in a host cell or an organism of interest. The cassette will include 5′ and 3′ regulatory sequences operably linked to a recombinant nucleic acid provided herein that allows for expression of the modified polypeptide. The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette will include in the 5′ to 3′ direction of transcription, a transcriptional and translational initiation region (i.e., a promoter), a polynucleotide disclosed herein, and a transcriptional and translational termination region (i.e., termination region) functional in the cell or organism of interest. The promoters described herein are capable of directing or driving expression of a coding sequence in a host cell. The regulatory regions (i.e., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.); Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring bygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4th ed., Cold Spring Harbor Laboratory Press, Cold Spring Harbor, 2012). The vector, for example, can be a plasmid.

There are numerous E, coli expression vectors known to one of ordinary skill in the art, which are useful for the expression of a nucleic acid. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used. Provided herein is a nucleic acid encoding a polypeptide of the present invention, wherein the nucleic acid can be expressed by a yeast cell. More specifically, the nucleic acid can be expressed by Pichia pastoris or S, cerevisiae.

Mammalian cells also permit the expression of proteins in an environment that favors important post-translational modifications such as folding and cysteine pairing, addition of complex carbohydrate structures, and secretion of active protein. Vectors useful for the expression of active proteins in mammalian cells are known in the art and can contain genes conferring hygromycin resistance, geneticin or G418 resistance, or other genes or phenotypes suitable for use as selectable markers, or methotrexate resistance for gene amplification. A number of suitable host cell lines capable of secreting intact human proteins have been developed in the art, and include CHO cells, HEK293 cells, HeLa cells, COS-7 cells, myeloma cell lines, Jurkat cells, derivatives of any of the above (e.g., Expi-HEK cells, Expi-CHO cells), etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E, coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors described herein can also include the nucleic acids as described herein under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

The DNA constructs and vectors provided herein may comprise the nucleic acids described above in a variety of configurations to allow expression and purification of the fusion proteins and/or modified proteins described herein. As described above, the domains of the fusion proteins and/or modified proteins provided herein may be present in a variety of configurations (e.g., one or more neutralizing polypeptide domains, one or more antibodies, peptide and/or non-peptide linkers, various orientations and orders for domain arrangement, etc.). Provided herein are DNA constructs and vectors that are able to express the fusion proteins, modified proteins, or domains thereof in any desired configuration. For example, a DNA construct or vector may comprise nucleic acids encoding an antibody sequence (e.g., a heavy chain sequence, a light chain sequence, or an antibody fragment sequence such as an scFv fragment) linked (e.g., via a peptide linker) to a neutralizing polypeptide (e.g., an ACE2 ectodomain polypeptide, a DPP4 ectodomain polypeptide, or a neutralizing antibody).

As described further below, various domains (e.g., antibody sequences and coronavirus receptor polypeptides) or domain components (e.g., antibody heavy chain and antibody light chain) may be expressed from the same DNA construct or vector or from different DNA constructs or vectors (i.e., they are expressed separately and linked or associated in vivo or in vitro to produce a fusion protein, modified protein, or domain thereof as described herein). As an example, one DNA construct or vector may comprise nucleic acids encoding an antibody heavy chain sequence linked to a coronavirus receptor polypeptide and another DNA construct or vector may comprise nucleic acids encoding a corresponding antibody light chain sequence. These two DNA constructs or vectors could be introduced into a host cell, as described below, and the expressed polypeptides allowed to associate to form a dimer (e.g., the antibody heavy chain and light chain may associate) prior to purification. As another example, two or more modified protein domains may be produced from separate DNA constructs or vectors. The DNA constructs or vectors may then be introduced into a host cell, as described below, and the expressed polypeptides (e.g., an scFv antibody fragment and a coronavirus receptor polypeptide) may be chemically linked with a non-peptide linker to produce a modified protein.

A host cell comprising a nucleic acid, a DNA construct, or a vector described herein is also provided. The host cell can be an in vitro, ex vivo, or in vivo host cell. Populations of any of the host cells described herein are also provided. A cell culture comprising one or more host cells described herein is also provided. Appropriate host cells for the expression of antibodies or antigen binding fragments thereof include yeast, bacteria, insect, plant, and mammalian cells.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell, or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host. Insect cells also permit the expression of the polypeptides. Recombinant proteins produced in insect cells such as Sf9 with baculovirus vectors undergo post-translational modifications similar to that of wild-type mammalian proteins.

The fusion proteins or modified proteins disclosed herein may be produced by recombinant expression in a human or non-human cell. In some instances, the cell is a synthetic antibody-producing cell, such as non-human cells expressing heavy chains, light chains, or both heavy and light chains; human cells that are not immune cells that express heavy chains, light chains, or both heavy and light chains; and human B cells that produce heavy chains or light chains, but not both heavy and light chains. The fusion proteins or modified proteins of this disclosure may be heterologously expressed, in vitro or in vivo, in cells other than human B cells, such as non-human cells and human cells other than B cells, optionally other than immune cells, and optionally in cells other than cells in a B cell lineage.

The fusion proteins and modified proteins provided herein can be produced from the cells by culturing a host cell containing the nucleic acid encoding the fusion protein or modified protein, under conditions and for an amount of time sufficient to allow expression of the proteins. Such conditions for protein expression vary with the choice of the expression vector and the host cell. Methods for the culture and production of many cells are available in the art. See e.g., Sambrook, Ausubel, and Berger (all supra), as well as Freshney (1994) Culture of Animal Cells, a Manual of Basic Technique, 3^{rd} Ed., Wiley-Liss, New York and the references cited therein, Doyle and Griffiths (1997) Mammalian Cell Culture: Essential Techniques John Wiley and Sons, NY; Humason (1979) Animal Tissue Techniques, 4^{th} Ed. W. H. Freeman and Company; and Ricciardelli, et al., (1989) In vitro Cell Dev. Biol. 25:1016-1024.

The nucleic acids, DNA constructs, and expression vectors can be introduced into cells in a manner suitable for subsequent expression of the nucleic acid. As used herein, the phrase “introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection. Various methods of translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTuL (MT) (Li et al. Signal Transduction and Targeted Therapy 5, Article No. 1 (2020)) can also be used to introduce a nucleic acid, for example, a nucleic acid encoding a recombinant protein described herein, into a host cell.

In some embodiments, a fusion protein or modified protein can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999). J Immunol Methods 231(1-2): 147-157.

Following expression, the fusion proteins or modified proteins can be isolated. A fusion protein or modified protein can be isolated or purified in a variety of ways known in the art depending on what other components are present in the sample Standard purification methods include electrophoretic, molecular, immunological, and chromatographic techniques, including ion exchange, hydrophobic, affinity, and reverse-phase HPLC chromatography. For example, a fusion protein or modified protein comprising an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

In vitro methods are also suitable for preparing the fusion proteins and modified proteins described herein. For example, digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment that has two antigen combining sites and is still capable of cross-linking antigen. The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing fusion proteins is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). A fusion protein provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N. Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form a fusion protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al, J. Biol. Chem. 269:16075 (1994); Clark et al., Biochemistry 30:3128 (1991); Rajarathnam et al., Biochemistry 33:6623-30 (1994)).

In some embodiments, production of the modified proteins described herein comprises chemical linkage of unprotected peptide segments where the bond formed between the peptide segments as a result of the chemical ligation is an unnatural (non-peptide) bond (Schnolzer et al, Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)). Various chemical linkers may be used to link two amino acid residues. For example, two amino acid residues may be cross linked via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., m-maleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate). In some embodiments, a neutralizing polypeptide or an antibody described herein, one of which is attached to a linker having a chemical functional group at the free/unattached end is produced and joined to another neutralizing polypeptide domain or antibody comprising a complementary reactive chemical functional group (e.g., at an end or internally). In some embodiments, two domains of the modified protein having a full or partial linker sequence with a chemical functional group at the end are produced and chemically linked in vitro via the free/unattached ends of the full or partial linker.

As described above, additional non-peptide linkers may be used to join domains of a modified protein described herein. In some embodiments, non-peptide linkers comprise functional groups on at least one terminus to allow attachment to a polypeptide domain. For example, PEG linkers can be designed with N-hydroxy-succinimide (NHS) esters at one end or both ends that react specifically and efficiently with lysine and N-terminal amino groups to form amide bonds. In another example, linkers can also be designed with sulfhydryl-reactive crosslinkers at one end or both ends that react with reduced sulfhydryls to form stable thioether bonds.

Methods for determining the yield or purity of a purified fusion protein or modified protein are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

An “isolated” or “purified” polypeptide or protein (e.g., fusion protein or modified protein) is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals.

### III. Pharmaceutical Compositions and Formulations

Compositions comprising a fusion protein or modified protein of the present disclosure and a pharmaceutically acceptable carrier are also provided. The compositions may further comprise a diluent, solubilizer, emulsifier, preservative, and/or adjuvant to be used with the methods disclosed herein. Such compositions can be used in a subject infected with a coronavirus that would benefit from the activity of any of the fusion proteins or modified proteins described herein.

In certain embodiments, acceptable formulation materials preferably are nontoxic to recipients at the dosages and concentrations employed. In certain embodiments, the formulation material(s) are for subcutaneous and/or intravenous administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers, monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Allen (2012) Remington—The Science and Practice of Pharmacy, 22d Edition, Lloyd V, Allen, ed., The Pharmaceutical Press). In certain embodiments, the optimal pharmaceutical composition is determined by one skilled in the art depending upon, for example, the intended route of administration, delivery format and desired dosage. See, for example, Allen (2012) Remington—The Science and Practice of Pharmacy, 22d Edition, Lloyd V, Allen, ed., The Pharmaceutical Press. In certain embodiments, such compositions may influence the physical state, stability, rate of in vivo release and/or rate of in vivo clearance of the fusion protein or modified protein.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising a fusion protein or modified protein disclosed herein can be prepared for storage by mixing the selected composition having the desired degree of purity with optional formulation agents (see Allen (2012) Remington—The Science and Practice of Pharmacy, 22d Edition, Lloyd V, Allen, ed., The Pharmaceutical Press) in the form of a lyophilized cake or an aqueous solution. Further, in certain embodiments, a composition comprising a fusion protein or modified protein disclosed herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5.

In certain embodiments when parenteral administration is contemplated, a therapeutic composition can be in the form of a pyrogen-free, parenterally acceptable aqueous solution comprising a fusion protein or modified protein in a pharmaceutically acceptable vehicle. In certain embodiments, a vehicle for parenteral injection is sterile distilled water in which a fusion protein or modified protein is formulated as a sterile, isotonic solution and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, a fusion protein or modified protein can be formulated as a dry powder for inhalation. In certain embodiments, an inhalation solution comprising a fusion protein or modified protein can be formulated with a propellant for aerosol delivery. In certain embodiments, solutions can be nebulized. Pulmonary administration is further described in International Application Publication No. WO/1994/020069, which describes pulmonary delivery of chemically modified proteins.

In certain embodiments, it is contemplated that formulations can be administered orally. In certain embodiments, a fusion protein or modified protein that is administered in this fashion can be formulated with or without carriers customarily used in compounding solid dosage forms, such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of a fusion protein or modified protein. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a fusion protein or modified protein in a mixture with non-toxic excipients suitable for the manufacture of tablets. In certain embodiments, by dissolving the tablets in sterile water or other appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions can be selected by one skilled in the art, including formulations involving a fusion protein or modified protein in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, International Application Publication No. WO/1993/015722, which describes the controlled release of porous polymeric microparticles for the delivery of pharmaceutical compositions. In certain embodiments, sustained-release preparations can include semipermeable polymer matrices in the form of shaped articles, e.g., films, or microcapsules. Sustained release matrices can include polyesters, hydrogels, polylactides (see, e.g., U.S. Pat. Nos. 3,773,919; 5,594,091; 8,383,153; 4,767,628; International Application Publication No. WO1998043615, Calo, E, et al. (2015) Eur. Polymer J 65:252-267 and European Patent No. EP 058,481), including, for example, chemically synthesized polymers, starch based polymers, and poly hydroxyalkanoates (PHAs), copolymers of L-glutamic acid and gamma ethyl-L-glutamate (Sidman et al. (1993) Biopolymers 22:547-556), poly(2-hydroxyethyl-methacrylate) (Langer et al. (1981) J Biomed Mater Res. 15: 167-277; and Langer (1982) Chem Tech 12:98-105), ethylene vinyl acetate (Hsu and Langer (1985) J Biomed Materials Res 19(4): 445-460) or poly-D(-)-3-hydroxybutyric acid (European Patent No EP0133988). In certain embodiments, sustained release compositions can also include liposomes, which can be prepared by any of several methods known in the art. (See, e.g., Eppstein et al. (1985) Proc. Natl. Acad. Sci. USA 82:3688-3692; European Patent No. EP 036,676; and U.S. Pat. Nos. 4,619,794 and 4,615,885).

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes are included.

In certain embodiments, the effective amount of a pharmaceutical composition comprising a fusion protein or modified protein as described herein to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a fusion protein or modified protein is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the fusion protein or modified protein in the formulation used. In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data. Dosing considerations and administration are discussed further below.

### IV. Methods of Treatment

As described herein, the present disclosure provides a method of treating a subject infected with a coronavirus infection, comprising administering to the subject a therapeutically effective amount of a fusion protein or modified protein as described in the present disclosure. In some embodiments, the subject has or is determined to have a coronavirus infection. In the discussion below, reference to a fusion protein, modified protein, fusion protein composition, or modified protein composition encompasses pharmaceutical compositions as discussed above.

The provided fusion proteins and modified proteins can also be used as a prophylactic therapy for coronavirus infection. The provided fusion proteins and modified proteins may be used either in prophylactic and therapeutic administration as well as by passive immunization with substantially purified polypeptide products or gene therapy by transfer of polynucleotide sequences encoding the fusion protein, modified protein, or part thereof. Thus, the provided fusion proteins and modified proteins can be administered to high-risk subjects in order to lessen the likelihood and/or severity of a coronavirus infection or administered to subjects already evidencing active coronavirus infection.

The compositions described herein are useful in, inter alia, methods for treating a coronavirus infection in a subject. As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have a coronavirus infection. In some embodiments, the subject is diagnosed with a coronavirus infection. In some embodiments, the subject is a human that is suspected of having a coronavirus infection. In some embodiments, the subject has or is suspected to have a SARS-COV-2 virus infection. In some embodiments, the subject has symptoms indicative of a SARS-COV-2 infection. In some embodiments, the subject is diagnosed as having a SARS-COV-2 virus infection. In some embodiments, the subject has been diagnosed with COVID-19.

In some embodiments, the subject may be asymptomatic or symptomatic. The subject may be male or female and may be a juvenile or an adult (e.g., at least 30 years old, at least 40 years old, or at least 50 years old). In some embodiments, the subject is displaying one or more symptoms indicative of SARS-COV-2 (or SARS-COV-2 variant) infection (i.e. of COVID-19). Such symptoms include, but are not limited to, any of a new loss of taste or smell, myalgia, fatigue, shortness of breath or difficulty breathing, fever, and/or cough. Symptoms may also include pharyngitis, headache, productive cough (i.e. a cough that produces mucus or phlegm), gastrointestinal symptoms (e.g., diarrhea, nausea, vomiting, or abdominal pain), hemoptysis, chest pressure or pain, confusion, cyanosis, and/or chills. In some embodiments, the patient has at least two symptoms selected from the group consisting of a new loss of taste or smell, shortness of breath or difficulty breathing, fever, cough, chills, or muscle aches. In some embodiments, the patient may have a blood oxygen level reading of 94 or less, e.g., as determined by an oximeter. In some embodiments, the subject may have radiographic evidence of pulmonary infiltrates. In some embodiments, the subject may have been receiving standard support care, e.g., such as being administered oxygen, fluids, and/or other therapeutic procedures or agents.

In some embodiments, the subject may not manifest any symptoms that are typically associated with a coronavirus infection (e.g., a SARS-COV-2 infection). In some cases the subject is known or believed to have been exposed to a coronavirus, suspected of having exposure to a coronavirus or believed not to have had exposure to a coronavirus. In some cases, the subject may have recovered from a prior coronavirus infection. In some cases, the subject has received a SARS-COV-2 vaccine. The SARS-COV-2 vaccine can be any of the DNA, RNA, or protein, or inactive SARS-COV-2 virus that is capable of inducing immune response in a patient to generate anti SARS-COV-2 antibodies. In some cases, the subject has been free of symptoms suggestive of a coronavirus infection for at least 14 days. In some cases, the subject may have one or more of other conditions of hypertension, coronary artery disease, diabetes, chronic obstructive pulmonary disease.

A coronavirus infection (e.g., a SARS-COV-2 infection) in a subject can be detected by various assays performed on a biological sample from the subject. The biological sample may be from a throat swab, a nasopharyngeal swab, sputum or tracheal aspirate, urine, feces, or blood. In some instances, nucleic acids are isolated from the biological sample and tested for the presences of viral genomic sequences. In some embodiments, PCR is performed to detect coronavirus nucleic acids from the biological sample. In some embodiments, a subject may have antibodies that selectively bind to coronavirus proteins, e.g., coronavirus spike protein. Antibodies can be detected in a blood sample from the subject by immunoassay (e.g., lateral flow assay or ELISA). In some embodiments, coronavirus infection can be detected using a proximity-based binding assay for detection of virus and/or anti-virus antibodies, as described in Lui, I., et al., “Trimeric SARS-COV-2 Spike interacts with dimeric ACE2 with limited intra-Spike avidity,” bioRxiv, doi.org/10.1101/2020.05.21.109157, published May 21, 2020 and Elledge et al., 2021, “Engineering luminescent biosensors for point-of-care SARS-COV-2 antibody detection,” Nat. Biotech., doi:10.1038/s41587-021-00878-8.

As used herein, “treating” or “treatment” of any disease or disorder refers to preventing or ameliorating a disease or disorder in a subject or a symptom thereof. The term ameliorating refers to any therapeutically beneficial result in the treatment of a disease state, e.g., a coronavirus infection, such as a SARS-COV-2 virus infection and/or COVID-19, lessening in the severity or progression, or curing thereof. Treating or treatment also encompass prophylactic treatments that reduce the incidence of a disease or disorder in a subject and/or reduce the incidence or reduce severity of a symptom thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying, preventing increases in, or decreasing viral load. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a coronavirus infection in a subject by administering a fusion protein or modified protein as described in this disclosure is considered to be a treatment if there is a 10% reduction in one or more symptoms of the coronavirus infection in a subject as compared to a control. Thus the reduction can be a 10%, 20%, 30%, 40%, 50%, 60%, 70%, 80%, 90%, 100%, or any percent reduction in between 10% and 100% as compared to native or control levels. In some embodiments, formulations comprising a fusion protein or modified protein as described herein are administered to the subject until the subject exhibits amelioration of at least one symptom of a coronavirus infection and/or is demonstrated to have a sustained decrease in viral load, e.g., as measured by immunoassay and/or quantitative amplification method, including PCR or sequencing. In some instances, the formulation is administered to the subject until viral load is undetectable, i.e. below the level of detection, such that no coronavirus RNA copies can be detected by the assay methodology employed. In some instances, the subject exhibits undetectable viral load 1-4 weeks, 2-4 weeks, 2-12 weeks, 4-12 weeks, or 12-24 weeks after last administration of the formulation. It is understood that treatment does not necessarily refer to a cure or complete ablation of the disease, condition, or symptoms of the disease or condition.

In some embodiments, the subject is administered a fusion protein or modified protein (e.g., fusion protein composition or modified protein composition) as described herein within 1, 2, 3, 4, or 5 days from the onset of symptoms or within 1, 2, 3, 4, or 5 days from testing positive for a coronavirus infection (e.g., SARS-COV-2 infection). In some embodiments, the subject is administered a fusion protein or modified protein as described herein within 1 or 2 days of hospitalization with one or more symptoms indicative of a coronavirus infection (e.g., SARS-CoV-2 infection) . . .

In some embodiments, the fusion protein or modified protein (e.g., fusion protein composition or modified protein composition) is administered to the subject at least once a day, at least twice a day, or at least three times a day. In some embodiments, the fusion protein or modified protein is administered on consecutive days or on non-consecutive days. In some instances, the fusion protein or modified protein is administered to the subject for at least 1 day, at least 2 days, at least 4 days, at least 5 days, at least 6 days, at least 1 week, at least 2 weeks, at least 3 weeks, at least 1 month, at least 2 months, or at least 3 months. In some embodiments, the fusion protein or modified protein is administered to the subject for 2 to 5 or more days after the viral load is undetectable in order avoid “rebound” of virus replication.

Passive immunization with the fusion proteins or modified proteins provided herein is an option for prevention and treatment of coronavirus infection. The fusion proteins and modified proteins described herein can also be used as a prophylactic therapy for a coronavirus infection (e.g., SARS-COV-2 virus infection), such that the provided fusion proteins or modified proteins are administered to high-risk subjects in a therapeutically effective amount in order to lessen the likelihood and/or severity of a coronavirus infection. When administered prophylactically, the proteins, complexes, and compositions are administered prior to the onset of symptoms of an infection. Prophylactic administration may prevent exposure to a coronavirus from progressing to a coronavirus infection or prevent a coronavirus infection from progressing to symptomatic disease (e.g., COVID-19 for SARS-COV-2 infection). In some embodiments, the subject has been exposed to a coronavirus. In some embodiments, the subject is at risk of exposure to a coronavirus. For example, the subject may be a healthcare worker who has been exposed to a human patient with a suspected or confirmed coronavirus infection. As another example, the subject may be identified through contact tracing efforts as having come into physical contact with a human having a confirmed coronavirus infection. In some embodiments, the subject is administered a fusion protein or modified protein as described herein within 1, 2, 3, 4, or 5 days from exposure or suspected exposure to a coronavirus. In some embodiments, the subject is administered a fusion protein or modified protein as described herein with 1, 2, 3, 4, or 5 days from identification of the subject as having a high risk of coronavirus infection.

A pharmaceutical preparation as described herein can comprise an effective amount of a fusion protein or modified protein (e.g., fusion protein composition or modified protein composition) described herein. Such effective amounts can be readily determined by one of ordinary skill in the art as described below. Considerations include the effect of the administered fusion protein or modified protein, or the combinatorial effect of the fusion protein or modified protein with one or more additional active agents, if more than one agent is used in or with the pharmaceutical composition.

The terms “administering” or “administration,” when using in the context of administration of a composition described in the present disclosure to a subject (and the related terms and expression), refer to the act of physically delivering a substance as it exists outside the body (for example, an immunogenic composition described in the present disclosure) into a subject. Administration can be by mucosal, intradermal, intravenous, intramuscular, subcutaneous delivery and/or by any other known methods of physical delivery. When a disease, or a symptom thereof, is being treated, administration of the substance typically occurs after the onset of the disease or symptoms thereof. When a disease, or symptoms thereof, are being prevented, delayed, or reduced in severity, administration of the substance typically occurs before the onset of the disease or symptoms thereof. Administration encompasses direct administration, such as administration to a subject by a medical professional or self-administration, or indirect administration, which may be the act of prescribing a composition described in the present disclosure.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing polypeptides are well known to those of skill in the art. Generally, such systems should utilize components that will not significantly impair the biological properties of the polypeptides, such as the capacity to bind the spike RBD (see, for example, Remington's Pharmaceutical Sciences, 18th edition, 1990, Mack Publishing). Those of skill in the art can readily determine the various parameters and conditions for producing polypeptide injectates or infusates without resorting to undue experimentation. Administration can be achieved by, e.g., topical administration, local administration, injection, by means of an implant.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and the like.

As used herein, the term “therapeutically effective amount” refers to an amount of fusion protein or modified protein (e.g., a fusion protein composition or a modified protein composition) as described herein that, when administered to a subject, is effective to achieve an intended purpose, e.g., to reduce viral load or prevent viral load from increasing, to reduce or ameliorate at least one symptom of a coronavirus infection (e.g., a SARS-COV-2 infection), and/or otherwise reduce the length of time that a patient experiences a symptom of a coronavirus infection, or extend the length of time before a symptom may recur.

As used herein, a “prophylactically effective amount” of a fusion protein or modified protein (e.g., a fusion protein composition or a modified protein composition) as described herein is a dosage large enough to produce the desired effect in the protection of individuals against a coronavirus infection for a reasonable period of time, such as one to two months or longer following administration.

The terms therapeutically effective amount and prophylactically effective amount may each be referred to herein as effective amounts, with the context depending on the subject who is receiving treatment (i.e. having an infection or not). An effective amount is also one in which any toxic or detrimental effects of the composition are outweighed by the therapeutically beneficial effects. In some instances, an effective amount is not a dosage so large as to cause adverse side effects, such as hyperviscosity syndromes, pulmonary edema, congestive heart failure, and the like. An effective amount may vary with the subject's age, condition, and sex, the extent of the disease in the subject, frequency of treatment, the nature of concurrent therapy (if any), the method of administration, and the nature and scope of the desired effect(s) (Nies et ah, Chapter 3 In: Goodman & Gilman's The Pharmacological Basis of Therapeutics, 9th Ed., Hardman et ah, eds., McGraw-Hill, New York, NY, 1996), and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics or treatments that are administered to the subject. Although individual needs may vary, determination of optimal ranges for effective amounts of formulations is within the skill of the art. It should also be understood that a specific dosage and treatment regimen for any particular subject also depends upon the judgment of the treating medical practitioner (e.g., doctor or nurse). The dosage of the effective amount may be adjusted by the individual physician or veterinarian in the event of any complication.

In some instances, a therapeutically effective amount may vary from about 0.01 mg/kg to about 50 mg/kg, preferably from about 0.1 mg/kg to about 20 mg/kg, most preferably from about 0.2 mg/kg to about 2 mg/kg, in one or more dose administrations daily, for one or several days. In some instances, a prophylactically effective amount may vary from about 0.01 mg/kg to about 50 mg/kg, preferably from about 0.1 mg/kg to about 20 mg/kg, most preferably from about 0.2 mg/kg to about 2 mg/kg, in one or more administrations (priming and boosting).

The data obtained from the cell culture assays and animal studies can be used in formulating a range of dosage for use in humans. The dosage of a fusion protein, modified protein, fusion protein composition, or modified protein composition lies generally within a range of circulating concentrations of the fusion protein, modified protein, fusion protein composition, or modified protein composition that includes the ED₅₀ with little or no toxicity. The dosage may vary within this range depending upon the dosage form employed and the route of administration utilized. For fusion proteins and modified proteins described herein, the therapeutically effective dose can be estimated initially from cell culture assays. A dose can be formulated in animal models to achieve a circulating plasma concentration range that includes the EC₅₀ (i.e., the concentration of the construct—e.g., polypeptide—that achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

Suitable human doses of any of the fusion proteins or modified proteins described herein can further be evaluated in, e.g., Phase I dose escalation studies. See, e.g., van Gurp et al. (2008) Am J Transplantation 8(8): 1711-1718; Hanouska et al. (2007) Clin Cancer Res 13(2, part 1): 523-531; and Hetherington et al. (2006) Antimicrobial Agents and Chemotherapy 50(10): 3499-3500.

Toxicity and therapeutic efficacy of the fusion proteins, modified proteins, fusion protein compositions, or modified protein compositions described herein can be determined by known pharmaceutical procedures in cell cultures or experimental animals (e.g., animal models of any of the disease states described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A fusion protein, modified protein, fusion protein composition, or modified protein composition that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects. Wild-type (WT) human recombinant ACE2 (hrACE2/APN01) was previously found to be safe in humans for the treatment of hypertension and acute respiratory distress syndrome (see Haschke et al., 2013, “Pharmacokinetics and pharmacodynamics of recombinant human angiotensin-converting enzyme 2 in healthy human subjects,” Clin Pharmacokinet 52:783-792 and Khan et al., 2017, “A pilot clinical trial of recombinant human angiotensin-converting enzyme 2 in acute respiratory distress syndrome,” Crit Care Lond Engl 21:234). The fusion proteins and modified proteins of this disclosure are expected to be similarly safe for therapeutic use.

In some embodiments, a fusion protein or modified protein (e.g., fusion protein composition or modified protein composition) described herein can be administered to a subject as a monotherapy. Alternatively, the fusion protein or modified protein can be administered in conjunction with other therapies for viral infection (combination therapy). For example, the fusion protein or modified protein can be administered to a subject at the same time, prior to, or after, a second therapy. In some embodiments, the fusion protein or modified protein and the one or more additional active agents are administered at the same time. Optionally, the fusion protein or modified protein can be administered first in time and the one or more additional active agents are administered second in time. In some embodiments, the one or more additional active agents are administered first in time and the fusion protein or modified protein is administered second in time. Optionally, the fusion protein or modified protein and the one or more additional agents can be administered simultaneously in the same or different routes. For example, a composition comprising the fusion protein or modified protein optionally contains one or more additional agents.

In certain embodiments, the other therapies may include administration of, for example, remdesivir, chloroquine, tenofovir, entecavir, and/or protease inhibitors (lopinavir/ritonavir). In certain embodiments, the other therapies may include administration of annexin-5, anti-PS monoclonal or polyclonal antibodies, bavituximab, and/or bind to viral glucocorticoid response elements (GREs), retinazone and RU486 or derivatives, cell entry inhibitors, uncoating inhibitors, reverse transcriptase inhibitors, integrase inhibitors, transcription inhibitors, antisense translation inhibitors, ribozyme translation inhibitors, prein processing and targeting inhibitors, protease inhibitors, assembly inhibitors, release phase inhibitors, immunosystem modulators and vaccines, including, but not limited to Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Synergistic enhancer, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, and combinations thereof.

A fusion protein or modified protein described herein can replace or augment a previously or currently administered therapy. For example, upon treating with a fusion protein or modified protein, administration of the one or more additional active agents can cease or diminish, e.g., be administered at lower levels or dosages. In some embodiments, administration of the previous therapy can be maintained. In some embodiments, a previous therapy is maintained until the level of the fusion protein or modified protein reaches a level sufficient to provide a therapeutic effect.

Monitoring a subject (e.g., a human patient) for an improvement of a coronavirus infection (e.g., a SARS-COV-2 infection) refers to evaluating the subject for a change in a disease parameter, e.g., a reduction in one or more symptoms of a coronavirus infection exhibited by the subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment, or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a viral infection described herein.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties. The following description provides further non-limiting examples of the disclosed compositions and methods.

### Examples

### EXAMPLE 1. Materials and Methods

The materials and methods described in this example are used in the examples that follow.

A yeast library, developed from a small but diverse pool of non-RBD-directed scFvs, was used to isolate unique scFvs that bind to both SARS-COV-1 and SARS-COV-2. These broadly-reactive scFvs were linked to ACE2 ectodmain through a TEV cleavable linker to form scFv-based non-neutralizing broadly neutralizing antibodies (nn-bnAb). The linkage between the scFv and ACE2 was shown to be a primary determinant of action for neutralization against both SARS-COV-2 and SARS-COV-1 by these nn-bnAb. IgG-based nn-bnAbs fusion proteins were then developed that are sub-nanomolar inhibitors against SARS-COV-2 and low nanomolar inhibitors against SARS-COV-1. Cleavage of the linker joining the IgG and ACE2 ectodomain coverts these compositions into micromolar inhibitors. The diverse set of antibodies isolated from the yeast was used in a sort to profile the cross-reactive, non-neutralizing antibody landscape of SARS-based coronaviruses. Three unique epitope-targets of these cross-reactive antibodies were identified.

### A. Yeast Display

Library Design. A library of antibodies directed against SARS-COV-2 Spike (S) protein was developed using paired antibody sequences, meaning antibody sequences for which the heavy and light chain are both known, from the Coronavirus Antibody Database (opig.stats.ox.ac.uk/webapps/covabdab/; Matthew I. J. Raybould, Aleksandr Kovaltsuk, Claire Marks, Charlotte M. Deane 2020 CoV-AbDab: the Coronavirus Antibody Database. Bioinformatics. doi=10.1093/bioinformatics/btaa739). Antibody sequences were inserted into a table, categorized by their binding to the CoV2 RBD portion of the spike protein or to a non-RBD portion of CoV2 spike. Following this analysis, antibodies which were cataloged for non-RBD binding were preferentially identified, resulting in a total of 385 paired antibody sequences. For these non-RBD binding antibodies, the amino acid sequences of the corresponding heavy chain and light chain genes, already compiled from the Coronavirus Antibody Database, were imported into Geneious Prime v2021.1.1 (a bioinformatics software; geneious.com). Using Geneious Prime, the heavy chain sequences and light chain sequences were separately analyzed to produce phylogenetic trees. For these phylogenetic trees, RBD binding antibodies were also included to ensure selection of antibody sequences that were both non-RBD binding and clearly distinct from RBD binding sequences. An additional 370 RBD binding antibody amino acid sequences of the corresponding heavy chain and light chain genes were imported, for a total of 755 heavy chain and light chain sequences. The sequences were first aligned using the MUSCLE algorithm (Muscle 3.8.425; Edgar, R. C. 2004 MUSCLE: multiple sequence alignment with high accuracy and high throughput Nucleic Acids Res. 32:1792-1797. DOI: 10.1093/nar/gkh340), and then two phylogenetic trees were made, both using PhyML 3.3.20180621 (Guindon et al 2010 Syst Biol 59:307-321. DOI:10.1093/sysbio/syq010). The sequence similarities used to produce phylogenetic trees account for antibody germlines, CDR lengths, and amount of somatic hypermutation. After producing phylogenetic trees based on the heavy chain and light chain sequences, a total of 48 sequences were identified based on their location in the phylogeny. Distinct clusters, composed of only non-RBD sequences, on the heavy chain phylogenetic trees were noted, and a single representative sequence was selected from each, chosen to also include distinct light chain sequences whenever possible. The sequences of these 48 antibodies were then converted into scFv sequences by linking the HC variable region to the LC variable region with a G4S-3 linker (GGGGSGGGGSGGGGS (SEQ ID NO:309)). All scFvs were designed in the order: signal sequence-HC-G4-S-3-LC. This vector also contained the HVM06_Mouse Ig heavy chain V region 102 signal peptide (MGWSCIILFLVATATGVHS (SEQ ID NO:320)) to allow for protein secretion and purification from the supernatant. Following construct design, the plasmids were ordered with the sequences inserted at the XhoI and NheI sites in the pTwist CMV BetaGlobin vector (Twist Biosciences).

Library Production. 4 μg of pPNL6 vector in Cut Smart buffer was digested using 1 μl of NheI HF and BamHI HF (NEB Biolabs) at 37° C. for 1 h. Digested plasmid was then gel extracted using Thermofisher Scientific Gel Extraction Kit. Equimolar aliquots of each scFv plasmid were pooled and the resultant pool was amplified using primers which annealed to the hexa-his Tag (reverse primer) or signal peptide (forward primer) and had a 50 bp overlap with the pPNL6 vector digested with NheI and BamHI. The pooled amplification was gel extracted to ensure it was the correct size. Yeast were prepared by first streaking a YPAD plate and incubating for 2-3 days until a single colonies were identifiable. A single colony was inoculated in 5 mL of YPAD overnight shaking at 30° C. Cultures were harvested into 6 tubes and pelleted. Yeast were resuspended in electroporation buffer (10 mM Tris Base, 250 mM sucrose, 2 mM MgCl) containing the gel extracted library amplification and digested pPNL6 vector. This mixture was then pulsed and the electroporated yeast were recovered in SD-CAA media overnight (30° C. shaking). These yeast were then induced by a 1:10 dilution into SG-CAA media and grown at 20° C. shaking for 2-3 days.

Yeast Binding Experiments-Binding to individual antigens. Following induction in SG-CAA shaking for 2-3 days at 20° C., the yeast library, expressing surface exposed scFvs, was incubated for 15 mins with a dilution of preformed baits. Baits were formed by mixing biotinylated baits and streptavidin 647 (Jackson Immunoresearch) at a 4:1 ratio. For example, 12.5 nM bait would be produced by incubation of 50 nM biotinylated antigens and 12.5 nM streptavidin 647. Yeast were flowed with two colors of “bait,” the first (FITC) stains for a c-myc tag. The c-myc tag is a surrogate for expression as the scFv constructs contain an in-frame C-terminal c-myc tag, so any yeast which are c-myc positive are displaying full-length antibodies. The second color bait (Alexa Flour 647-APC channel) stains for the antigen-target of the scFv. To make the stain, streptavidin with an Alexa Flour 647 tag is incubated with biotinylated bait protein. This complex is then used to stain the yeast. Any yeast which are positive for Alexa Flour 647, are then binding to the protein antigen. Yeast were spun down and resuspended in in 50 μl PBSM containing the respective concentration of tetrameric bait. After 15 mins cells were then washed 1× with PBSM and then resuspended in 50 μL PBSM containing 1 μl of anti-c-myc FITC (Miltenyi) for 15 mins. Samples were then washed 2× with PBSM and then resuspended in 50 μL of PBSM. These samples were flowed (Accuri C6 flow cytometer) and the percent antigen positive was determined as the ratio of antigen positive cells divided by all cells expressing scFv (c-myc positive) multiplied by 100. Therefore, if all yeast expressing scFv proteins were doubly positive the equation would result in the number 100. Numbers closer to 100 suggest that the yeast library is binding better to that specific antigen. Gates were set such that ˜. 5% of yeast were antigen positive in the streptavidin alone control.

Yeast Binding Experiments-Sorts. The yeast library was incubated with 125 nM of tetrameric SARS-COV-1 and 1 μl of anti-c-myc FITC (Miltenyi) for 1 hour. Samples were then washed 2× with PBSM and then resuspended in 50 μl PBSM. These libraries were then sorted on an FACSAria IIu using the Stanford FACS Facility (Stanford CA). The samples were gated such that all antigen positive cells were collected (gates set such that ˜0% anti-cmyc FITC alone controls fell within the gate). Two populations were sorted, a hi-gate, consisting of the highest intensity binders (3.8% of all cells), and a low-gate, consisting of all other antigen positive cell (3.7% of all cells). Cells were sorted directly into tubes containing 4 mL of SD-CAA media. These sorted libraries were grown for 1 day at 30° C. shaking in SD-CAA media and then 300 μl of the cultures were miniprepped (Zymo Research) following the manufacturer's protocol. Miniprepped DNA was transformed into STELLAR Competent Cells (Clontech) and plated on carbenicillin LB agar plates (as per pPNL6's resistance marker). E, coli cells that grow should, theoretically, contain only a single sequence from each of the yeast that were sorted above. 10 E. coli colonies from the hi-gate and 20 E, coli colonies from the low-gate sort were sent for sequencing (Sequetech, Mountain View CA). The sequences were then analyzed by sequence alignment using SnapGene software.

### B. Constructs

scFv-ACE2 fusion proteins. scFvs identified from the above-described SARS-COV-1 sort were cloned into the same pTwist CMV BetaGlobin vector such that they contained a linker (GGSGSHHHHHHASTGGGSGGPSGQAGAAASEENLYFQGSLFVSNHAYGGSGGEARV (SEQ ID NO:294)) followed by the ectodomain of human ACE2 (SEQ ID NO:271).

Light chain (LC)-ACE2 fusion proteins. Antibody sequences were cloned into the CMV/R plasmid backbone for expression under a CMV promoter. The antibodies variable LC were cloned between the CMV promoter and the bGH poly(A) signal sequence of the CMV/R plasmid to facilitate improved protein expression. The variable region was cloned into the human IgG1 backbone with a kappa LC. This vector also contained the HVM06_Mouse (P01750) Ig heavy chain V region 102 signal peptide to allow for protein secretion and purification from the supernatant. The light chains from the scFvs from the above-described SARS-COV-1 sort were cloned into the CMV/R vector with a C terminal linker (GGSGSHHHHHHASTGGGSGGPSGQAGAAASEENLYFQGSLFVSNHAYGGSGGEARV (SEQ ID NO:294)) followed by the ectodomain of human ACE2 (SEQ ID NO:271).

Heavy Chain (HC) IgG plasmids. Antibody sequences were cloned into the CMV/R plasmid backbone for expression under a CMV promoter. The antibodies variable HC were cloned between the CMV promoter and the bGH poly(A) signal sequence of the CMV/R plasmid to facilitate improved protein expression. The variable region was cloned into the human IgG1 backbone with a kappa LC. This vector also contained the HVM06_Mouse (P01750) Ig heavy chain V region 102 signal peptide to allow for protein secretion and purification from the supernatant. The heavy chains from the scFvs from the above-described SARS-COV-1 sort were cloned into the CMV/R vector.

hCoV spike proteins. Full-length spike proteins from hCovs were cloned into a pADD2 vector between the rBeta-globin intron β-globin poly(A). The constructs were followed by the sequence: GGGGSRMKQIEDKIEEILSKQYHIENEIARIKKLIGERGGSGGGLNDIFEAQKIEWHEGHH HHHH (SEQ ID NO:321), containing a GCN4 sequence (RMKQIEDKIEEILSKQYHIENEIARIKKLIGER (SEQ ID NO:322)), an Avi tag (GLNDIFEAQKIEWHE (SEQ ID NO:323)), and a 6 X-His tag. Sequences contained their native signal peptide. Furin cleavage sites were mutated, and the 2 proline mutations were installed for improved stability and expression, as previously described.

Lentivirus plasmids. Plasmids encoding the full length spike proteins with native signal peptides were cloned into the background of the HDM-SARS2-Spike-delta21 plasmid (Addgene Plasmid #155130). This construct contains a 21 amino acid c-terminal deletion to promote viral expression. The SARS-COV-1 spike was used as the full length construct without a C-terminal deletion. The other viral plasmids that were used were previously described (doi: 10.3390/v12050513). They are: pHAGE-Luc2-IRS-ZsGreen (NR-52516), HDM-Hgpm2 (NR-52517), pRC-CMV-Rev1b (NR-52519), and HDM-tat1b (NR-52518).

### C. Protein Production

Protein Expression. All proteins were expressed in Expi293F cells. Expi293F cells were cultured in media containing 66% Freestyle/33% Expi media (ThermoFisher) and grown in TriForest polycarbonate shaking flasks at 37° C. in 8% CO₂. The day before transfection cells were spun down and resuspended to a density of 3×10⁶ cells/mL in fresh media. The following day cells were diluted and transfected at a density of approximately 3-4×10⁶ cells/mL. Transfection mixtures were made by adding the following components: mirA-prepped or maxi-prepped DNA, culture media, and FectoPro (Polyplus) would be added to cells to a ratio of 0.5-0.8 μg: 100 μL:1.3 μL:900 μL. For example, for a 100 mL transfection, 50-80 μg of DNA would be added to 10 mL of culture media and then 130 μL of FectoPro would be added to this. Following mixing and a 10 min incubation, the resultant transfection cocktail would be added to 90 mL of cells. The cells were harvested 3-5 days post-transfection by spinning the cultures at >7,000×g for 15 minutes. Supernatants were filtered using a 0.22 μm filter. To determine scFv binding and expression spun-down Expi293F supernatant was used without further purification. For proteins containing a biotinylation tag (Avi-Tag) Expi293F cells containing a stable BirA enzyme insertion were used, resulting in spontaneous biotinylation during protein expression.

Protein purification-Fc Tag containing proteins. All proteins containing an Fc tag (for example, IgGs, IgG-Ace2 fusions, hFc-ACE2) were purified using a 5 mL MAb Select Sure PRISMIM column on the AKTA pure FPLC. (Cytiva). Filtered cell supernatants were diluted with 1/10^{th} volume 10× Phosphate Buffered Saline (PBS). The AKTA system was equilibrated with, A1-1×PBS, A2-100 mM Glycine pH 2.8, B1-0.5M NaOH, Buffer line-1×PBS, Sample lines-H₂O. The protocol washes the column with A1, followed by loading of the sample in Sample line 1 until air is detected in the air sensor of the sample pumps, followed by 5 column volume washes with A1, elution of the sample by flowing of 20 mL of A2 (directly into a 50 ml conical containing 2 mL of IM Tris pH 8.0) followed by 5 column volumes A1, B1, A1. The resultant Fc-containing samples were concentrated using 50 or 100 kDa cutoff centrifugal concentrators. Proteins were buffer exchanged using a PD-10 column (SEPHADEX) which had been preequilibrated into 20 mM HEPSE, 150 mM NaCl. IgGs used for competition, binding, and neutralization experiments were not further purified. IgG-ACE2 fusions were then further purified using the S6 column on the AKTA as above.

Protein purification-His-tagged proteins. All proteins not containing an Fc tag (for example, scFvs and scFv fusions, receptor binding domain (RBD), and FL Spike trimers from hCoVs polypeptide antigens) were purified using HisPur™ Ni-NTA resin (ThermoFisher). Cell supernatants were diluted with ⅓rd volume wash buffer (20 mM imidazole, 20 mM HEPES pH 7.4, 150 mM NaCl) and the Ni-NTA resin was added to diluted cell supernatants. For all mixtures not containing SARS-COV-2 spike protein, the samples were then incubated at 4° C. while stirring overnight SARS-COV-2 spike proteins were incubated at room temperature. Resin/supernatant mixtures were added to chromatography columns for gravity flow purification. The resin in the column was washed with wash buffer (20 mM imidazole, 20 mM HEPES pH 7.4, 150 mM NaCl) and the proteins were eluted with 250 mM imidazole, 20 mM HEPES pH 7.4, 105 mM NaCl. Column elutions were concentrated using centrifugal concentrators (10 kDa cutoff for RBD, 50 kDa cutoff for scFv-ACE2-fusions, and 100 kDa cutoff for trimer constructs), followed by size-exclusion chromatography on a AKTA Pure system (Cytiva). AKTA pure FPLC with a Superdex 6 Increase gel filtration column (S6) was used for purification. 1 mL of sample was injected using a 2 mL loop and run over the S6 which had been preequilibrated in degassed 20 mM HEPES, 150 mM NaCl prior to use. Biotinylated antigens were not purified using the AKTA pure.

### D. TEV Digestion

TEV digestion of scFv-ACE2 fusions. 1 μL of TEV protease (New England BioLabs) was added to 200 μL of scFv-ACE2 fusions at ˜4 μM in 20 mM HEPES, 150 mM NaCl. The reaction was left to incubate overnight at room temperature. Extent of cleavage was determined by and SDS-PAGE analysis on 4-20% Mini-PROTEAN® TGX™ protein gels stained with GelCode™ Blue Stain Reagent (ThermoFisher).

TEV digestion of IgG-ACE2 fusions. 3 μL of TEV protease (New England BioLabs) was added to 200 μL of scFv-ACE2 fusions at ˜2 μM in 20 mM HEPES, 150 mM NaCl. The reaction was left to incubate overnight at 37° C. Extent of cleavage was determined by and SDS-PAGE analysis on 4-20% Mini-PROTEAN® TGX™ protein gels stained with GelCode™ Blue Stain Reagent (ThermoFisher).

### E. Biolayer Interferometry Binding

Biolayer interferometry (Octet) Binding Experiments-scFv binding and expression. All reactions were run on an Octet Red 96 and samples were run in PBS with 0.1% BSA and 0.05% Tween 20 (octet buffer). scFvs from the above sort were assessed for binding using streptavidin (SA) biosensors (Sartorius/ForteBio) loaded for 2 mins with 50-100 nM biotinylated antigens (SARS-COV-2 or SARS-COV-1 spike proteins). SA bionsensor tips are coated with streptavidin and designed to bind biotinylated antigens. Following loading, tips were then washed and base-lined in wells containing only octet buffer. Samples were then associated in wells containing 150 μL octet buffer and 50 μL of spun-down Expi-cell supernatant. A control well containing 150 μL octet buffer and 50 μL of mock-transfected media was used as a baseline subtraction for data analysis. To determine expression the same plate was used but anti-Penta His (His1K) tips were used. These tips are designed to bind specifically to a penta-His tag on proteins. For this experiment, no SARS-COV-2 or SARS-COV-1 antigen was loaded, tips were just baselined in a blank well and then associated in the wells containing 50 μL of scFv expression media. Response values (I.E. peak reached after 5 mins of association) was determined using the Octet data analysis software. Final data analysis was done in Prism.

Biolayer interferometry (Octet) Binding Experiments IgG binding. All reactions were run on an Octet Red 96 and samples were run in PBS with 0.1% BSA and 0.05% Tween 20 (octet buffer). IgGs produced from the scfvs from the above sort were assessed for binding using streptavidin (SA) biosensors (Sartorius/ForteBio) loaded to a threshold of 0.8 nm of SARS-COV-2, SARS-COV-1, and MERS biotinylated spike proteins. Tips were then washed and base-lined in wells containing only octet buffer. Samples were then associated in wells containing 100 nM IgG. A control well which loaded antigen but associated in a well containing only 200 μL octet buffer was used as a baseline subtraction for data analysis.

Biolayer interferometry (Octet) Binding Experiments IoG competition. All reactions were run on an Octet Red 96 and samples were run in PBS with 0.1% BSA and 0.05% Tween 20 (octet buffer). IgGs produced from the scFvs from the above sort were assessed for their competition of binding with one another using anti-Penta HIS (His1K) biosensors (Sartorius/ForteBio). His1K tips were pre-quenched with buffer containing >10 nM biotin. Tips were then loaded with 100 nM protein for 2 mins (SARS-COV-2 spike) or 4 mins (SARS-COV-1 spike). These tips were then associated with one of seven antibodies (either CV27, COV2-2147, CV10, COVA2-14, COVA2-18, COV2-2449, COV2-2143) at 100 nM for 5 mins to reach saturation. Tips were then baselined and associated with either 1 of the 7 antibodies. For this step all 8 tips went into the same antibody at 100 nM. Response values (I.E. peak reached after 2 mins of association) was determined using the Octet data analysis software. Values were normalized to the tip loaded with either SARS-COV-2 or SARS-COV-1 spike but without a competing antibody. These values were set as a value of 1 for each antibody. This is simply the antibody binding to the protein. Additionally, the antibody competing with itself was set to a value of zero. Final data analysis was done in Prism.

Biolayer interferometry (Octet) Binding Experiments-scFv-ACE2-Fusion and IgG-ACE2-Fusion binding. All reactions were run on an Octet Red 96 and samples were run in PBS with 0.1% BSA and 0.05% Tween 20 (octet buffer). Streptavidin (SA) biosensors (Sartorius/ForteBio) were loaded for 2 mins with 100 nM biotinylated antigens (SARS-COV-2 or SARS-COV-1 spike proteins or RBD (IgG binding only)). Samples were then washed and baselined in wells containing octet buffer. Association occurred in samples containing ACE2-fusion proteins either without or with TEV protease (NEB) treatment. scFv-ACE2 fusions were tested at 200 nM while IgG-ACE2 fusions were tested at 100 nM (CV27 and COVA2-14) or 15 nM (CV10). Association was conducted for 2 min (scFv) or 6 min (IgG) and dissociation was conducted for 1 min (scFv) or 2 min (IgG).

Biolayer interferometry (Octet) Binding Experiments-scFv-ACE2-fusion and IgG-ACE2-fusion competition with hFc-ACE2 and CB6. All reactions were run on an Octet Red 96 and samples were run in PBS with 0.1% BSA and 0.05% Tween 20 (octet buffer). Streptavidin (SA) biosensors (Sartorius/ForteBio) were loaded for 2 mins with 100 nM biotinylated antigens (SARS-COV-2 or SARS-COV-1 spike). Samples were then washed and baselined in wells containing octet buffer. scFv-ACE2-fusions were then associated for 5 mins. Samples were baselined and then associated with either hFc-ACE2 (SARS-COV-2 and SARS-COV-1) or CB6 (SARS-COV-2) for 2 mins. Response values (I.E. peak reached after 2 mins of association) was determined using the Octet data analysis software. Samples which loaded SARS-COV-2 or SARS-COV-1 but did not associate with any hFc-ACE2 or CB6 were used as a baseline subtraction. Values were normalized to the binding of hFc-ACE2 or CB6 without a competitor.

### F. Lentivirus Production

SARS-COV-2 Spike pseudotyped lentiviral particles were produced. Viral transfections were done in HEK293T cells using calcium phosphate transfection reagent. Six million cells were seeded in D10 media (DMEM+additives: 10% FBS, L-glutamate, penicillin, streptomycin, and 10 mM HEPES) in 10 cm plates one day prior to transfection. A five-plasmid system (plasmids described above) was used for viral production, as described in Crawford et al., 2020. The Spike vector contained the 21 amino acid truncated form of the SARS-COV-2 Spike sequence from the Wuhan-Hu-1 strain of SARS-COV-2. The plasmids were added to filter-sterilized water in the following ratios: 10 μg pHAGE-Luc2-IRS-ZsGreen, 3.4 μg FL Spike, 2.2 ng HDM-Hgpm2, 2.2 μg HDM-Tat1b, 2.2 μg pRC-CMV-Revlb in a final volume of 500 μL. HEPES Buffered Saline (2 X, pH 7.0) was added dropwise to this mixture to a final volume of 1 mL. To form transfection complexes, 100 L 2.5 M CaCl2 was added dropwise while gently agitating the solution. Transfection reactions were incubated for 20 min at RT, and then slowly added dropwise to plated cells. Culture medium was removed 24 hours post-transfection and replaced with fresh D10 medium. Viral supernatants were harvested 72 hours post-transfection by spinning at 300×g for 5 min followed by filtering through a 0.45 μm filter. Viral stocks were aliquoted and stored at −80° C. until further use.

### G. Neutralization

The target cells used for infection in viral neutralization assays were from a HeLa cell line stably overexpressing the SARS-COV-2 receptor, ACE2, as well as the protease known to process SARS-COV-2, TMPRSS2. Production of this cell line is described in detail in Rogers et al., 2020, with the addition of stable TMPRSS2 incorporation. ACE2/TMPRSS2/HeLa cells were plated one day prior to infection at 5,000 cells per well or 2 days prior to infection at 2,500 cells per well. 96 well white walled, clear bottom plates were used for the assay (Thermo Fisher Scientific). On the day of the assay, purified IgG- or scFv-ACE2 fusions in HEPES (20 mM), NaCl (150 mM), which either had or had not been treated with TEV protease, were sterile filtered using a 0.22 μm filter. Dilutions of this filtered stock were made into sterile 1×DPBS (Thermo Fisher Scientific). Each dilution well contained 30 μL of IgG- or scFv-ACE2 fusions. Samples were run in technical duplicate in each experiment. All other wells contained only 30 μL 1×DPBS

A virus mixture was made containing the virus of interest (for example SARS-COV-2 with a 21 amino acid deletion on the C terminus), D10 media (DMEM+additives: 10% FBS, L-glutamate, penicillin, streptomycin, and 10 mM HEPES), and polybrene (such that the final concentration of 5 μg/mL in inhibitor/virus dilutions). Virus dilutions into media were selected such that a suitable signal would be obtained in the virus only wells. A suitable signal was selected such that the virus only wells would achieve a luminescence of at least >1,000 RLU. 90 μL of this virus mixture was added to each of the inhibitor dilutions to make a final volume of 120 μL in each well. Virus only wells were made which contained 30 μL 1×DPBS and 90 μL virus mixture. Cells only wells were made which contained 30 μL 1×DPBS and 90 μL D10 media.

The inhibitor/virus mixture was left to incubate for 1 hour at 37° C. Following incubation, the medium was removed from the cells on the plates made either 1 day or 2 days prior. This was replaced with 100 μL of inhibitor/virus dilutions and incubated at 37° C. for approximately 24 hours. At 24 hours post infection the media was exchanged for fresh media in all samples containing a TEV cleavable linker with our without cleavage; media was not exchanged on samples which did not have a TEV cleavable linker (for example WT IgGs). Infectivity readout was performed by measuring luciferase levels. 48 hours post infection 50 μL of medium was removed from all cells and cells were lysed by the addition of 50 μL BriteLite™ assay readout solution (Perkin Elmer) into each well. Alternatively, all the medium was removed and a 1:1 dilution of BriteLite™ was used. Luminescence values were measured using a BioTek Synergy™ HT Microplate Reader (BioTek) plate reader. Each plate was normalized by averaging cells only (0% infectivity) and virus only (100% infectivity) wells. Cells only and virus only wells were averaged. Normalized values were fit with a three parameter or four parameter non-linear regression inhibitor curve in Prism to obtain IC50 values. Where possible, the average NT₅₀ of two independent experiments are shown.

### H. ELISA

IgG ELISAs against hCoV strains were performed. Streptavidin solution (5 μg/mL) was plated in 50 μL in each well on a MaxiSorp (Thermo Fisher Scientific) microtiter plate in 50 mM sodium bicarbonate pH 8.75. This was left to incubate for 1 hour at room temperature. These were washed 3× with 300 μL of ddH₂O using an ELx 405 Bio-Tex plate washer and blocked with 150 μL Chonblock (Chondrex) for at least 1 hour at room temperature. Biotinylated hCoV spike proteins were added to each well at a concentration of 1 μg/mL and left to incubate overnight at 4° C. Plates were washed 3× with 300 μL of 1×PBST and serial dilution of monoclonal antibodies (described above) were added, starting at 1 μM and undergoing 10-fold serial dilutions. These were left to incubate for 1 hour at room temperature and then washed 3× with PBST. Goat anti-human HRP (abcam ab7153) was added at a 1:5,000 dilution in PBST. This was left to incubate at room temperature for 1 hour and then washed 6× with PBST. Finally, the plate was developed using 50 μL of 1-Step™ Turbo-TMB-ELISA Substrate Solution (ThermoFisher) per well and the plates were quenched with 50 μL of 2M H₂SO₄ to each well. Plates were read at 450 nm and normalized for path length using a BioTek Synergy™ HT Microplate Reader. Anti-His ELISA signal was determined by incubation of the coated plates with a 1:500 dilution of mouse anti-his IgG1 (SigmaAldrich) for 1 hour, washed, and then secondary of anti-mouse IgG1-HRP (abcam ab97240). These wells were developed in the same way as above.

### I. Western Blot Analysis

Western blots were performed to ensure that the fusion proteins were not degraded (e.g., by reagents in the culture medium) during the SARS-COV-2 and SARS-COV-1 neutralization assays described above. Supernatants of viral neutralization cell plates were collected 1 day post-infection. 2 μL of each sample at the highest concentration (total of 4 μL) were diluted into 4 μL of Laemmli SDS-loading buffer (Bio-Rad) and boiled at 98° C. for at least 10 mins. Samples were then run on a 4-20% Mini-PROTEAN® TGX protein gel (Bio-Rad) at 250V for 25 mins. Proteins were transferred to nitrocellulose membranes using a Trans-Blot® Turbo™ transfer system (Bio-Rad). Blots were blocked in 10 mL of 5% milk in PBST. Following blocking anti-his IgG1 (SigmaAldrich) antibody was added at a 1:5000 dilution (scFv) or Goat anti-human HRP (abcam ab7153) was added. Following at least 1 hour incubation at room temperature the blots were washed 12× with ˜10 mL of 1×PBST and directly developed (IgG) or a secondary anti-mouse IgG1-HRP (abcam ab97240) was added at a 1:10,000 dilution in 5% milkin PBST. scFv blots were washed again 12× with ˜10 mL of 1×PBST and the blots were then developed using Pierce™ ECL Western blotting substrate (ThermoFisher). Developed blots were imaged using an GE A1600 RGB Gel Imaging System (GE Healthcare Life Sciences).

### EXAMPLE 2. Identification of Cross-Reactive Non-neutralizing SARS-COV-2 Antibodies

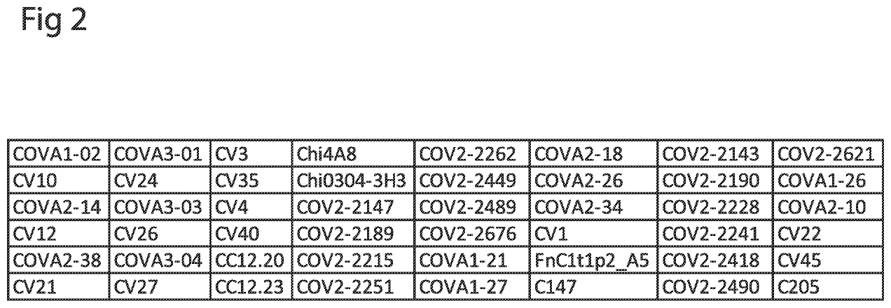

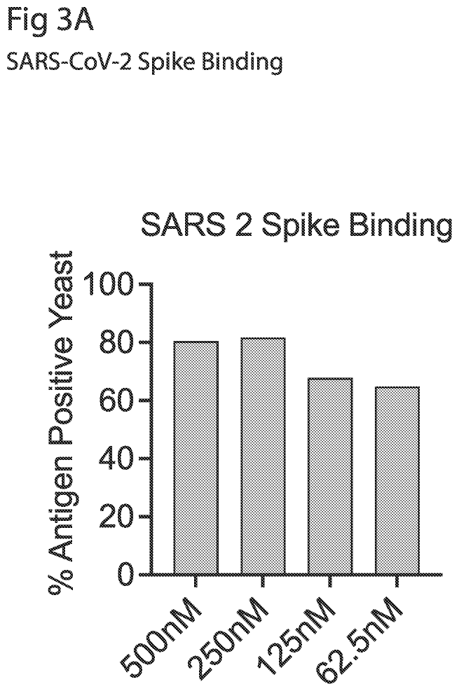

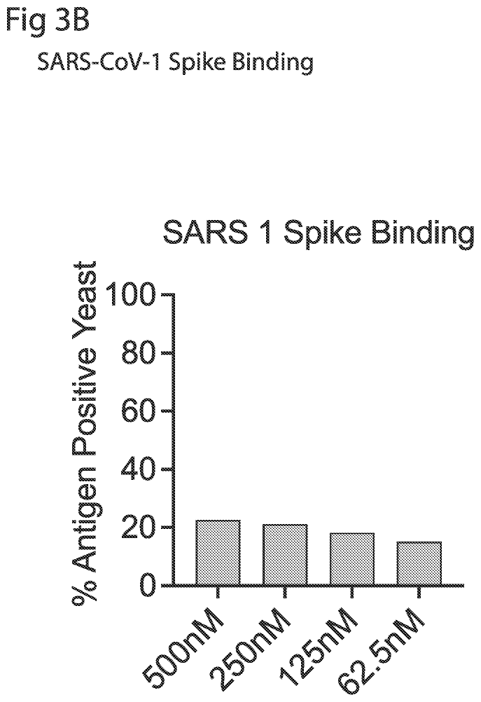

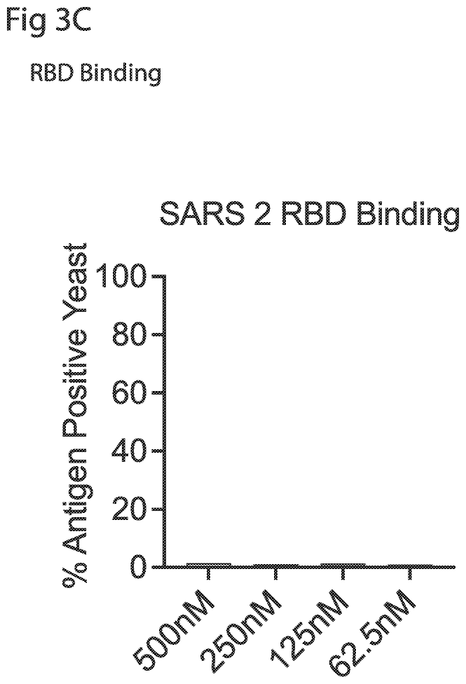

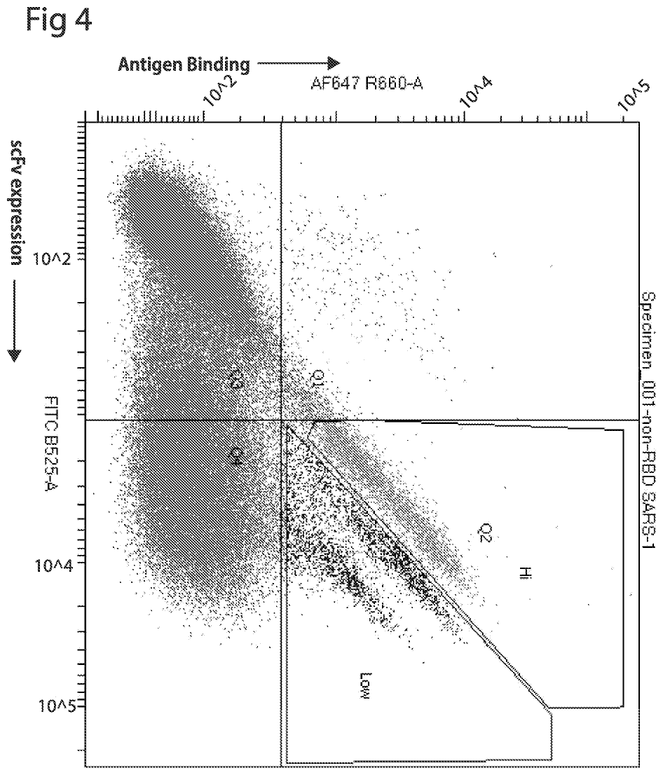

To identify cross-reactive non-neutralizing antibodies a yeast library was produced from a diverse set of non-RBD targeting mAbs. This was done by clustering sequences of known non-RBD and RBD binding antibodies against SARS-COV-2 S protein and then looking at the resultant trees to identify a large set of unique antibodies, as described in Example 1. The 48 identified sequences were used to produce scFvs (see FIG. 2 and Table 6). Plasmids containing the scFvs were ordered (Twist Bioscience) and developed into a yeast library. The yeast library was profiled by binding to dilutions of tetrameric SARS-COV-2, SARS-COV-1, and the RBD of SARS-COV-2 (FIGS. 3A-3C). The yeast bound well to SARS-COV-2 S protein, SARS-COV-1 S protein, but did not bind significantly to the SARS-COV-2 RBD, consistent with the library design targeting specifically non-RBD sequences. Next yeast were sorted using the Stanford Shared FACS Facility to select for those that bound specifically to SARS-COV-1 (FIG. 4). It was expected that these would similarly bind to SARS-COV-2 since they were derived from an anti-SARS-COV-2 S library. Two gates were used, one which selected for “high” binders, meaning the highest affinity clones, and one which was for “low” binders, meaning all SARS-COV-1 positive clones that were not in the “high” gate.

TABLE 6
scFv construct insert sequences.

| # | Construct name | Insert sequence |
|---|---|---|
| 1 | COVA1-02 | SEQ ID NO: 338 |
| 2 | CV10 | SEQ ID NO: 339 |
| 3 | COVA2-14 | SEQ ID NO: 340 |
| 4 | CV12 | SEQ ID NO: 341 |
| 5 | COVA2-38 | SEQ ID NO: 342 |
| 6 | CV21 | SEQ ID NO: 343 |
| 7 | COVA3-01 | SEQ ID NO: 344 |
| 8 | CV24 | SEQ ID NO: 345 |
| 9 | COVA3-03 | SEQ ID NO: 346 |
| 10 | CV26 | SEQ ID NO: 347 |
| 11 | COVA3-04 | SEQ ID NO: 348 |
| 12 | CV27 | SEQ ID NO: 349 |
| 13 | CV3 | SEQ ID NO: 350 |
| 14 | CV35 | SEQ ID NO: 351 |
| 15 | CV4 | SEQ ID NO: 352 |
| 16 | CV40 | SEQ ID NO: 353 |
| 17 | CC12.20 | SEQ ID NO: 354 |
| 18 | CC12.23 | SEQ ID NO: 355 |
| 19 | Chi4A8 | SEQ ID NO: 356 |
| 20 | Chi0304-3H3 | SEQ ID NO: 357 |
| 21 | COV2-2147 | SEQ ID NO: 358 |
| 22 | COV2-2189 | SEQ ID NO: 359 |
| 23 | COV2-2215 | SEQ ID NO: 360 |
| 24 | COV2-2251 | SEQ ID NO: 361 |
| 25 | COV2-2262 | SEQ ID NO: 362 |
| 26 | COV2-2449 | SEQ ID NO: 363 |
| 27 | COV2-2489 | SEQ ID NO: 364 |
| 28 | COV2-2676 | SEQ ID NO: 365 |
| 29 | COVA1-21 | SEQ ID NO: 366 |
| 30 | COVA1-27 | SEQ ID NO: 367 |
| 31 | COVA2-18 | SEQ ID NO: 368 |
| 32 | COVA2-26 | SEQ ID NO: 369 |
| 33 | COVA2-34 | SEQ ID NO: 370 |
| 34 | CV1 | SEQ ID NO: 371 |
| 35 | FnC1t1p2_A5 | SEQ ID NO: 372 |
| 36 | C147 | SEQ ID NO: 373 |
| 37 | COV2-2143 | SEQ ID NO: 374 |
| 38 | COV2-2190 | SEQ ID NO: 375 |
| 39 | COV2-2228 | SEQ ID NO: 376 |
| 40 | COV2-2241 | SEQ ID NO: 377 |
| 41 | COV2-2418 | SEQ ID NO: 378 |
| 42 | COV2-2490 | SEQ ID NO: 379 |
| 43 | COV2-2621 | SEQ ID NO: 380 |
| 44 | COVA1-26 | SEQ ID NO: 381 |
| 45 | COVA2-10 | SEQ ID NO: 382 |
| 46 | CV22 | SEQ ID NO: 383 |
| 47 | CV45 | SEQ ID NO: 384 |
| 48 | C205 | SEQ ID NO: 385 |

The resultant yeast were sequenced to identify the specific scFy that was encoded. Two clones appeared in the “high” gate and 9 in the “low” gate (Table 7).

TABLE 7
Antibodies identified in SARS-CoV-1 binding sort.

| | | Number of | True SARS- |
|---|---|---|---|
| Sequence | Gate | sequences | CoV-1 Binder |
| COV2-2449 | Hi | 5 | Yes |
| COVA2-18 | Hi | 5 | Yes |
| CV10 | Low | 1 | Yes |
| COVA2-14 | Low | 2 | Yes |
| CV21 | Low | 1 | No |
| CV27 | Low | 7 | Yes |
| COVA1-21 | Low | 1 | No |
| COV2-2147 | Low | 4 | Yes |
| COV2-2251 | Low | 1 | No |
| COV2-2143 | Low | 2 | Yes |
| COV2-2490 | Low | 1 | No |

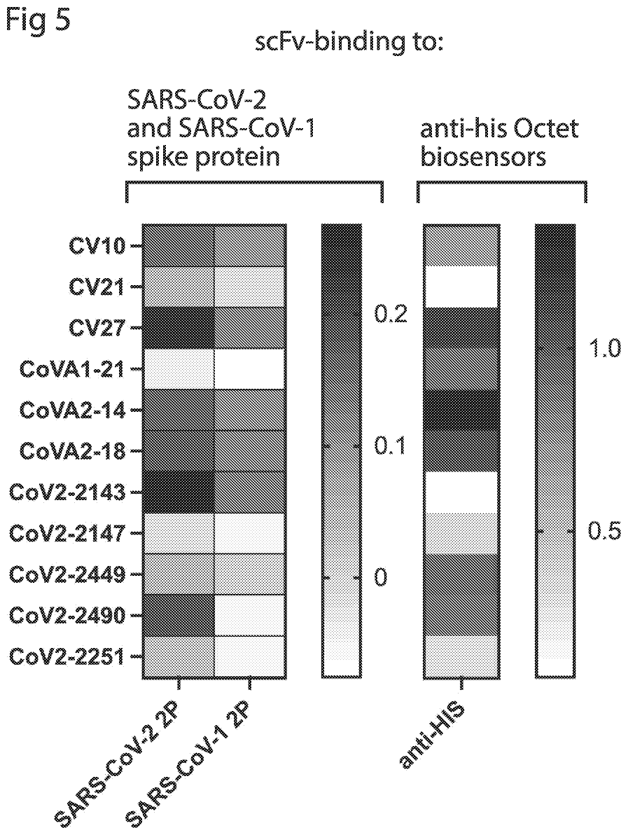

To confirm binding of these scFvs to both SARS-COV-2 and SARS-COV-1 spike proteins, the scFvs were transiently expressed in Expi293F cells and the cell supernatant was used to probe binding and expression (FIG. 5). Supernatant of transfected cells was tested in binding to biotinylated SARS-COV-2 and SARS-COV-1 by biolayer interferometry (BLI). Because the scFvs contain an N-terminal signal sequence that results in protein secretion from cells, the cell supernatant of the transfected cells is expected to contain the transfected scFv. The scFvs were designed to contain a hexa-his tag, therefore binding to biolayer interferometry biosensor tips that target his tags can be used as a surrogate for expression, where higher binding means better expression. Additionally, testing the supernatants for binding to SARS-COV-2 and SARS-COV-1 spike proteins enables an understanding of the level of cross-reactivity of the scFvs. It is expected that there would be a similar level binding to SARS-COV-2 spike and SARS-COV-1 spike if the clones were cross-reactive and bound with high affinity to both proteins. Therefore, clones which have a more similar intensity on the heat map for binding to both SARS-COV-2 and SARS-COV-1 spike are more cross-reactive. From this analysis it was determined that 4 of these identified clones were not true SARS-1 binders (Table 7) but likely fell into the positive gate through stochastic noise in the sort.

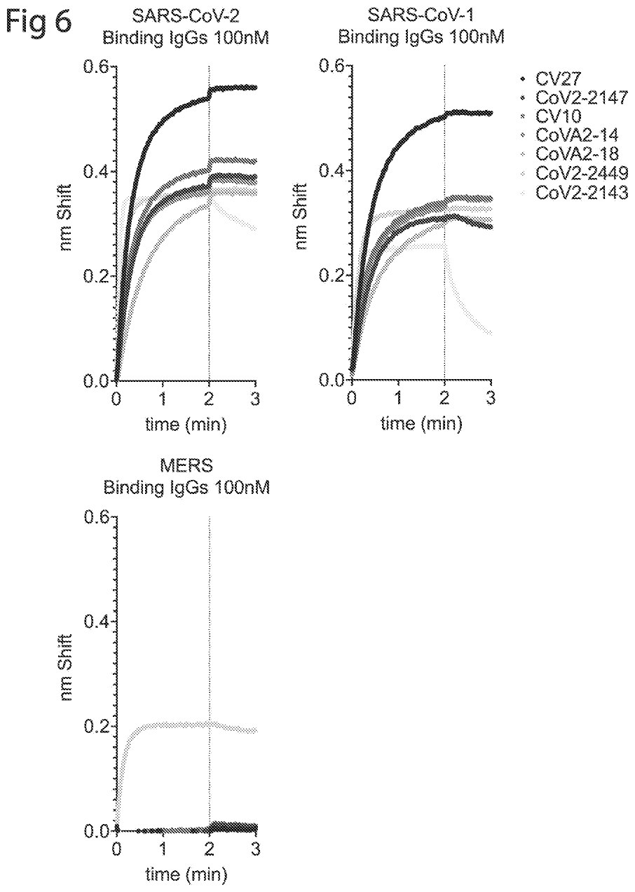

To confirm that these antibodies were true SARS-COV-2 and SARS-COV-1 cross reactive binders they were cloned into IgG expression plasmids encoding the full-length human kappa IgG1, expressed in Expi293F cells, and purified using protein A. The resultant antibodies were then tested for binding to biotinylated SARS-COV-2, SARS-COV-1, and MERS-COV spike proteins using BLI. All identified antibodies bound to both SARS spike proteins and the antibody 2449 additionally bound to MERS spike protein (FIG. 6).

Following the identification of the seven cross-reactive antibodies, the 385 non-RBD binding antibodies were reanalyzed for clonal similarity to the seven selected antibodies. For these non-RBD binding antibodies, the nucleic acid sequences of the corresponding heavy chain and light chain genes were acquired using the NCBI database. The Immcantation Pipeline (immcantation.readthedocs.io/en/latest/index.html; Gupta NT*, Vander Heiden J A*, Uduman M, Gadala-Maria D, Yaari G, Kleinstein S H. Change-O: a toolkit for analyzing large-scale B cell immunoglobulin repertoire sequencing data. Bioinformatics 31:3356-8 2015. doi:10.1093/bioinformatics/btv359) was used to cluster both the heavy chain and light chain nucleic acid sequences. The heavy chain and light chain sequences were separately clustered using the following criteria. First, sequences were grouped by germline V gene assignment, plus germline J gene assignment, plus CDR3 amino acid length. Next, the sequences grouped by these criteria were examined, and all sequences that share a greater than 75% amino acid identity in their CDR3 sequence were considered as a single heavy chain or light chain cluster. This analysis allowed for the identification of 26 heavy chain antibody sequences which clustered with at least 1 of the 7 antibodies and 25 light chain antibody sequences which clustered with at least 1 of the 7 antibodies. Heavy chain sequence clustered antibodies are shown in Table 8 and light chain sequence clustered antibodies are shown in Table 9. Antibodies in the same column in a table clustered together. The 7 previously identified antibodies are shown in bold. The antibodies listed in Table 8 and Table 9 were combined to make a complete degenerate set of 38 paired antibodies. The V-gene sequences of the heavy chains and light chains for these antibodies are shown in Table 2 and Table 3, and their CDRs are shown in Table 4 and Table 5.

TABLE 8
Antibody clusters identified using heavy chain sequences.

| | | | | |
|---|---|---|---|---|
| CV27 | CV10 | COVA2-14 | COV2-2449 | COV2-2143 |
| COV2-2147 | | COVA2-18 | | |
| COV2-2341 | | COV2-2270 | | |
| COV2-2160 | | COV2-2430 | | |
| COV2-2159 | | COV2-2441 | | |
| COV2-2844 | | COV2-2166 | | |
| CV34 | | COV2-2214 | | |
| COV2-2564 | | COV2-2367 | | |
| COV2-2643 | | COV2-2216 | | |
| COV2-2203 | | COV2-2169 | | |
| COV2-2250 | | COVA1-07 | | |
| | | Chi2M-8E7 | | |

TABLE 9
Antibody clusters identified using light chain sequences.

| CV27 | COV2-2147 | CV10 | COVA2−14 | COVA2−18 | COV2-2449 | COV2-2143 |
|---|---|---|---|---|---|---|
| | COV2-2341 | COV2-2656 | COV2-2270 | COV2-2430 | COV2-2621 | |
| | COV2-2160 | CV8 | | Chi2M-8E7 | COV2-2883 | |
| | COV2-2159 | COV2-2006 | | | COV2-2224 | |
| | | C205 | | | Chi2M-8H10 | |
| | | | | | COV2-2401 | |
| | | | | | COV2-2218 | |
| | | | | | Chi0304-4A2 | |
| | | | | | COV2-2422 | |

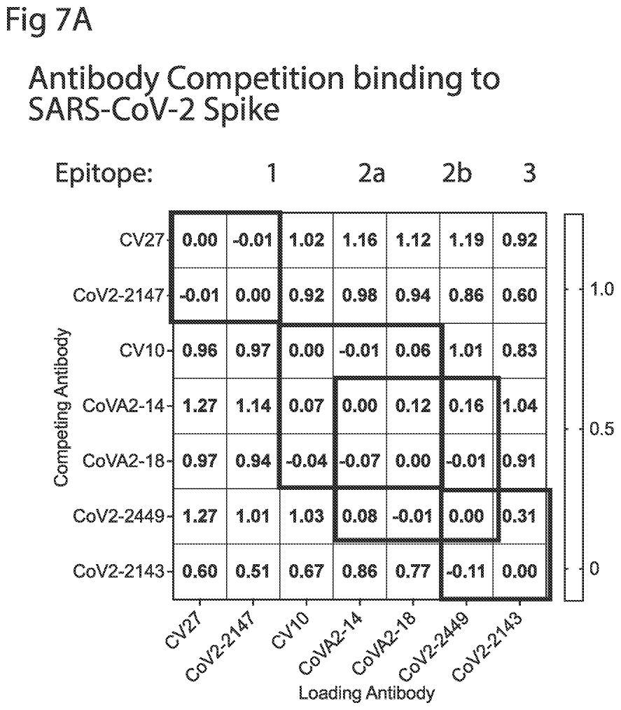

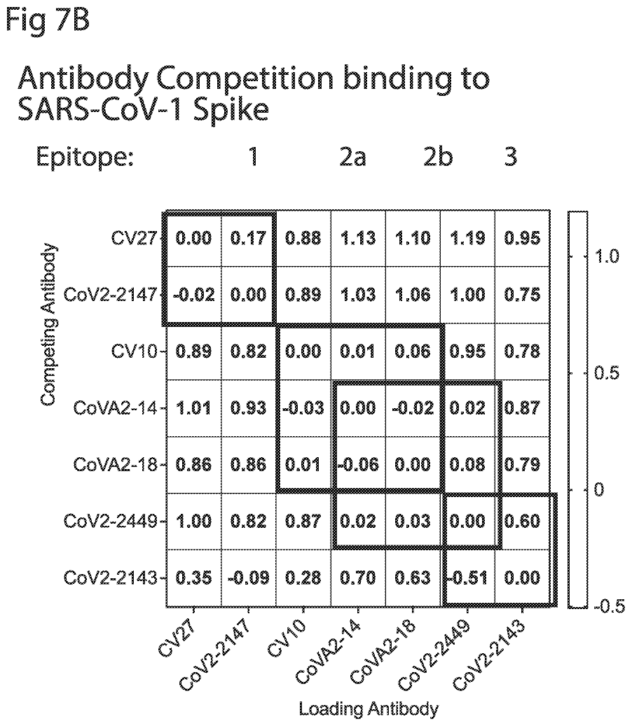

To next determine the number of epitopes contained within the pool of 7 cross-reactive antibodies described above, a competition experiment was set up. This was conducted by loading BLI sensor tips with either SARS-COV-2 or SARS-COV-1 spike proteins and then associating 1 of the 7 antibodies to saturation. This complex was then added to wells containing either the same or 1 of the other 6 antibodies. If the antibodies competed, there wouldn't be any association. If they came from distinct epitopes the second antibody could also associate. This assay reveled 3-4 potential epitopes within these 7 antibodies (FIG. 7A). These competition groups were consistent between SARS-COV-2 and SARS-COV-1 (FIG. 7B).

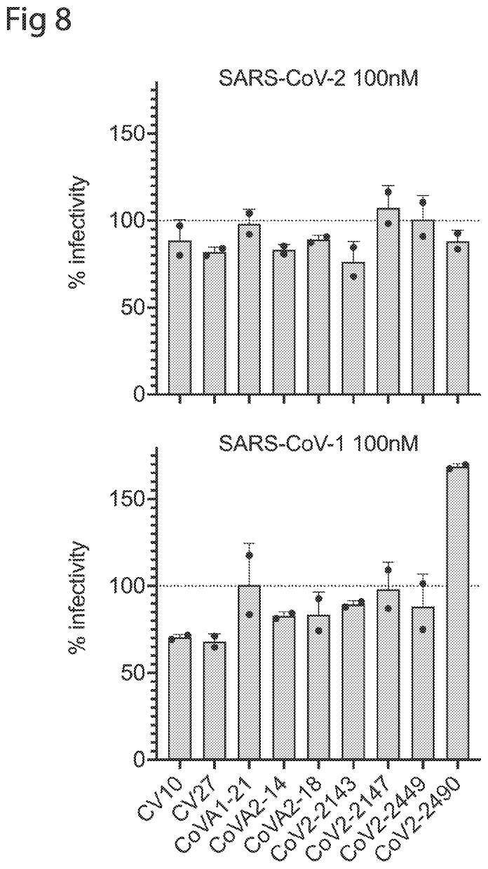

Having identified a series of competition groups, 3 antibodies were selected to move forward from groups 1, 2a, 2a/b. These antibodies were CV27, CV10, and COVA2-14, respectively. COV2-2143, despite existing in a more unique competition group, was not selected because of its lower affinity (notable off-rate in FIG. 6). As a final check, it was confirmed that these antibodies were in fact “non-neutralizing” by doing a single point neutralization at 100 nM of antibody. All of the antibodies at 100 nM had no appreciable neutralization (FIG. 8).

### EXAMPLE 3. Development and Characterization of scFv-ACE2 Fusion Proteins

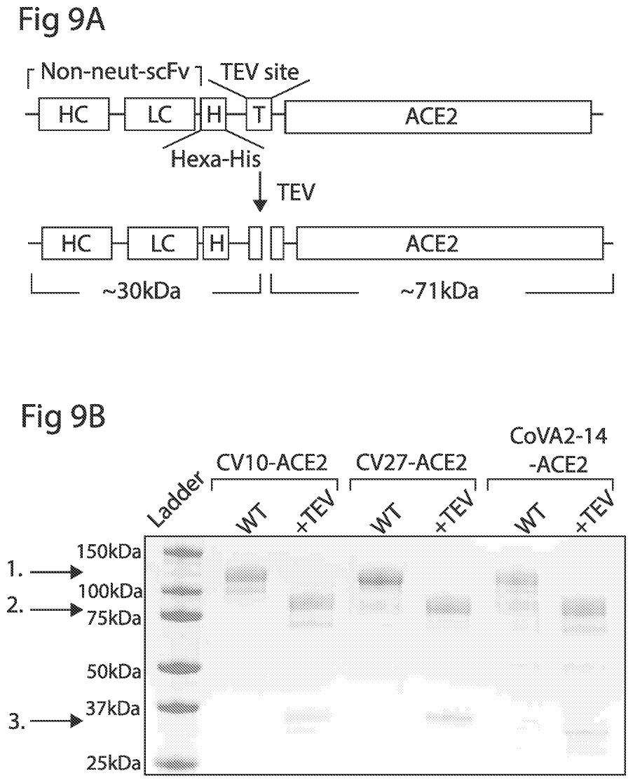

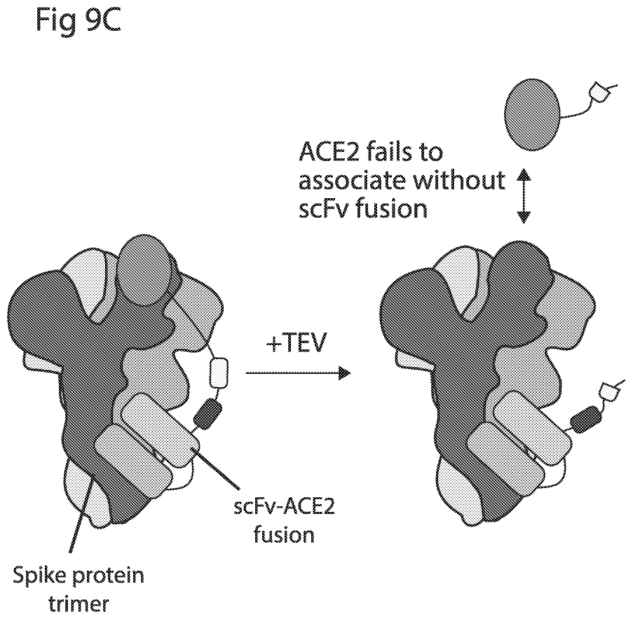

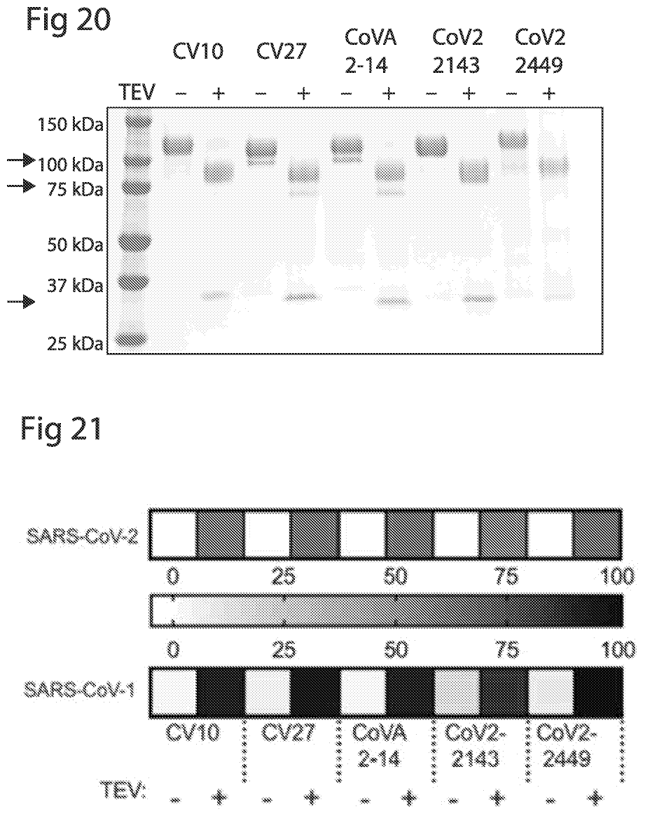

To test the efficacy of the 3 antibodies (CV10, CV27, COVA2-14) as potential non-neutralizing broadly neutralizing antibodies (nn-bnAbs) their scFv forms were then converted into scFv—ACE2 fusions. The hypothesis was that by linking the receptor of SARS-COV-2 and SARS-COV-1 onto scFvs that targeted non-neutralizing but conserved epitopes, it would convert these non-neutralizing scFvs into broadly neutralizing scFvs. The scFv component allows for high affinity binding, while the ACE2 binding blocks cell-surface ACE2 interactions. The constructs were designed as in FIG. 9A. The scFv-ACE2 fusions of CV10, CV27, and COVA2-14 scFvs were expressed and purified using Ni-NTA resin and size exclusion, producing proteins of the expected molecular weight (˜100 kDa, lanes 2, 4, and 6 respectively, FIG. 9B). These proteins were designed to contain a TEV cleavage site in the linker between the scFv and ACE2. Treatment with TEV protease separates the two domains into an ACE2 (˜71 kDa) and an scFv (˜30 kDa) (FIG. 9A). TEV digestion of the proteins in lane 2, 4, and 6 yielded the proteins in lanes 3, 5, and 7 demonstrating clear cleavage into the two constituent components (FIG. 9B). Similar results were obtained when the cleavage experiment was repeated with scFv-ACE2 fusions of CV10, CV27, and COVA2-14 along with COV2-2449 and COV2-2143 (FIG. 20).

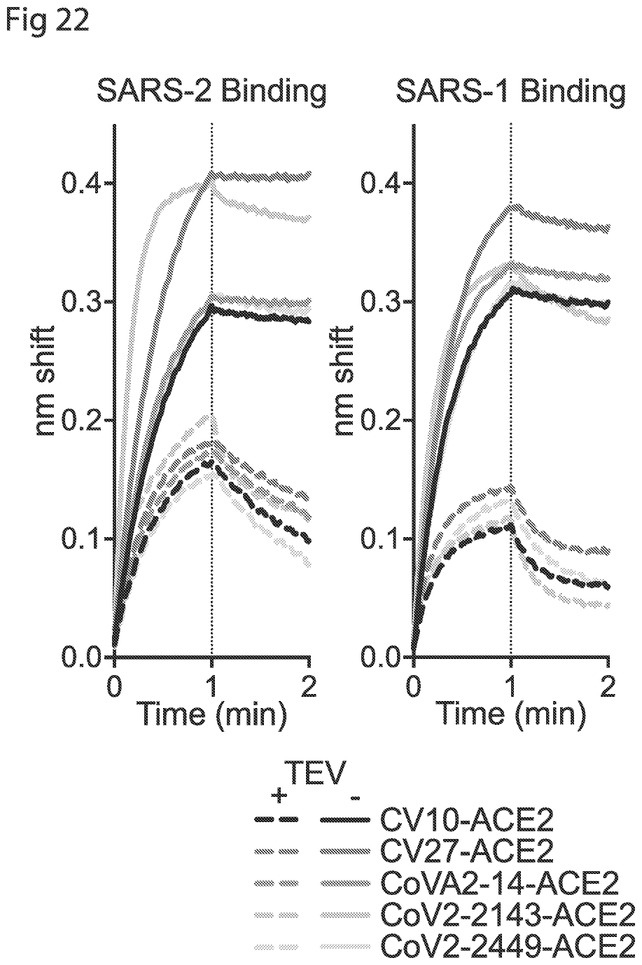

These proteins were expressed and purified for CV10, CV27, COVA2-14, COV2-2449, and COV2-2143 scFv-ACE2 fusions and were shown to be cleavable by TEV protease to separate the scFv and ACE2 components (FIG. 9B and FIG. 20). This is envisioned to work as shown in FIG. 9C. When fused, the scFv-ACE2 complex is able to bind at both the epitope of the scFv, as well as the ACE2 binding site. However, after TEV-clevage the scFv will still bind, but the ACE2 component is unable to bind with high affinity and therefore will significantly decrease the neutralization of the complex. BLI was used to determine how TEV cleavage impacted binding to SARS-COV-2 and SARS-COV-1 spike proteins. Proteins at 200 nM were tested pre and post TEV cleavage for binding. Clearly, TEV cleavage greatly decreased the affinity of these complexes (FIG. 10A and FIG. 22). The TEV digested complex curves are a combination of two binding curves: one due to the scFv and the other due to ACE2.

To further confirm that the scFv-ACE2 fusion was working as expected a competition experiment was set up to test how well the uncleaved scFv-ACE2 fusion competed with either hFc-ACE2 or the mAb CB6 (which is known to target the ACE2 binding site on the RBD). The uncleaved scFv-ACE2 fusion competes very significantly with hFc-ACE2 or CB6 when bound to either SARS-COV-2 or SARS-COV-1 spike proteins (FIG. 10B). Conversely, the cleaved proteins compete significantly less with either ACE2 or CB6 (FIG. 10B). Similar results were obtained when the competition experiment was repeated (with only hFc-ACE2 competition) with scFv-ACE2 fusions of CV10, CV27, and COVA2-14 along with COV2-2449 and COV2-2143 (FIG. 21). This demonstrates that non-neutralizing scFvs, when fused with ACE2, can block ACE2 binding, lending to a potential mechanism of action for neutralization.

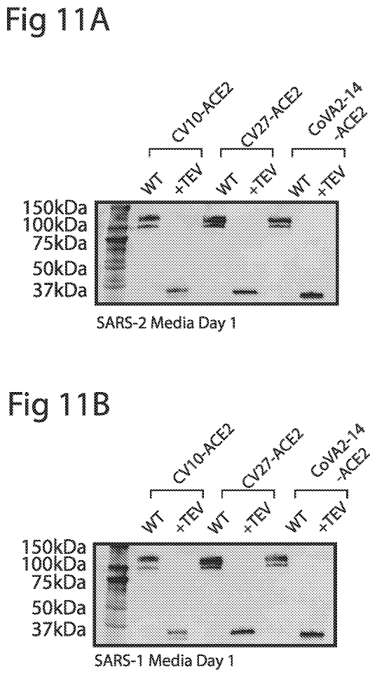

Neutralization of these scFv-ACE2 fusion complexes was then tested against both SARS-COV-2 and SARS-COV-1. This was tested with and without TEV cleavage to determine if the fusion of these two domains (scFv and ACE2) was essential for function. Because of the protease susceptibility of the linker between the scFv and ACE2 the media was changed 1 day post-infection and western blotted to ensure that the constructs had not been cleaved. The western blot determined that the uncleaved constructs remained uncleaved over the course of the 1 day infection in both SARS-COV-2 neutralization (FIG. 11A) and SARS-COV-1 neutralization (FIG. 11B). One day after the media was collected and exchanged the neutralization assay was read out using luminescence as a reporter for infection. This revealed that fusion of ACE2 to these non-neutralizing scFvs increased the potency (compared to the TEV cleaved versions) by ˜1000 fold (˜1 nM IC50 in the fusion compared to ˜1 μM IC50 in the TEV cleaved form) against SARS-COV-2 (FIG. 12A). Interestingly, the same extent of increase was not seen against SARS-CoV-1 virus (FIG. 12B). Against SARS-COV-1 the CV27 scFv fusion yielded no benefit compared to the TEV-digested protein. COVA2-14 yielded ˜5 fold improvement (195 nM IC50 uncleaved compared to 916 nM IC50 cleaved). Interestingly, CV10 scFv fusion was more potent than the other two, the fusion had an IC50 of 50 nM.

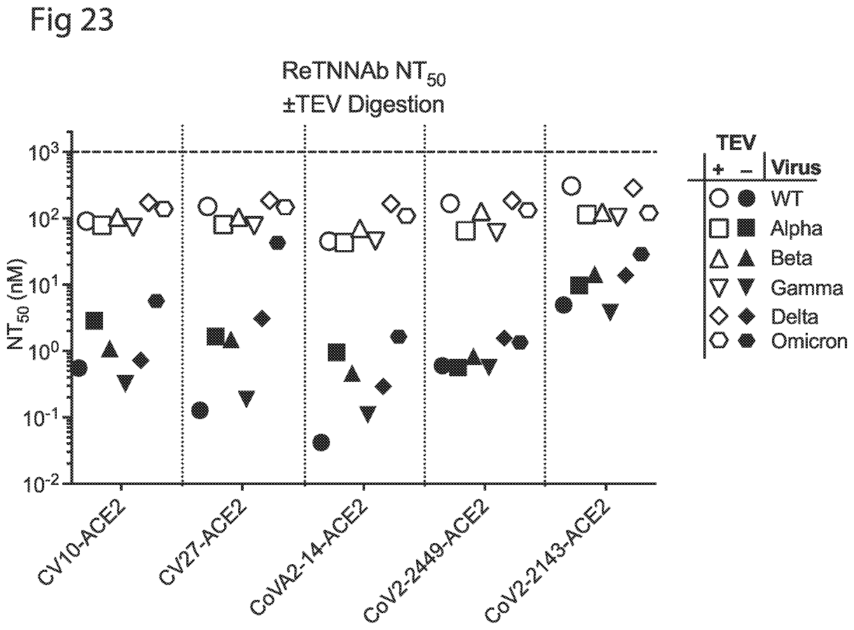

Neutralization of the scFv-ACE2 fusions was also tested against a panel of SARS-COV-2 variants of concern (VOCs; FIG. 23). Intact scFv-ACE2 fusions (FIG. 23, filled in symbols) show broad spectrum neutralization of SARS-COV-2 VOCs. Inhibition is markedly reduced upon TEV cleavage of scFv-ACE2 fusions (FIG. 23, open symbols). Pseudoviral 50% inhibitory concentration (NT50) for ReconnAbs (bottom) against a range of SARS-COV-2 VOCs with and without TEV cleavage. NT50 values shown are the average of two independent experiments.

Strikingly, these results indicate that ACE2 fusion was able to potently convert these non-neutralizing scFvs into broadly neutralizing scFvs (able to effectively neutralize both SARS-CoV-2 and SARS-COV-1, as well as several SARS-COV-2 variants of concern). Additionally, it is important to note that CV10 and COVA2-14 both showed increased potency against SARS-CoV-1 compared to their TEV cleaved counterparts. However, CV27 did not show the same potentiation. This result is consistent with their initial competition groups CV27 fell into a competition group unique from the other two (competition group 1, FIG. 7A), whereas CV10 and COVA2-14 both fell into a competition group 2a (FIG. 7B).

### EXAMPLE 4. Development and Characterization of IgG-ACE2 Fusion Proteins

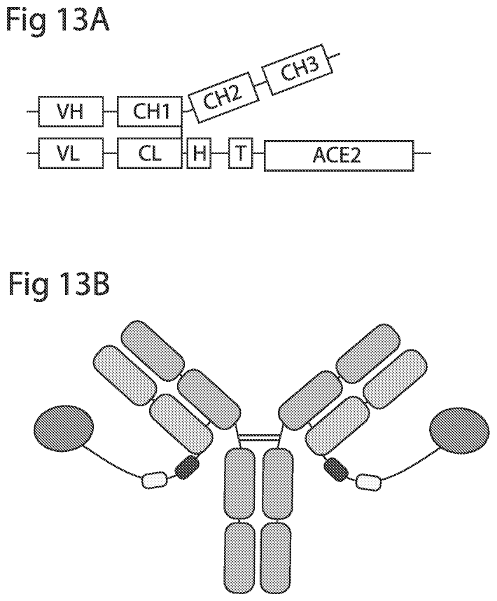

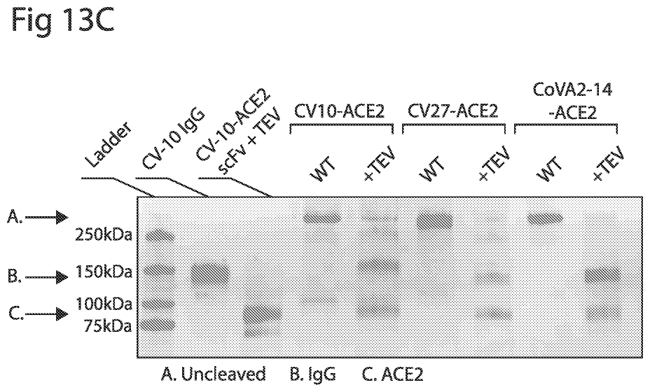

Given that ACE2 fusion to the scFv lead to significantly improved potency and was able to neutralize both SARS-COV-2 and SARS-COV-1 it was expected that fusion of ACE2 to an IgG might further increase this potency. To do this constructs were designed where ACE2 was linked to the end of the LC of a human IgG1 through the same linker as above (FIG. 13A). It is expected that this would self-assemble into a full-length IgG1 construct with two ACE2 fusion proteins per IgG (FIG. 13B). These proteins were designed to contain a TEV cleavage site in the linker between the LC and ACE2, and thus were able to be cleaved by TEV protease again to yield full length IgG and monovalent ACE2 (FIG. 13C) The IgG-ACE2 fusions of CV10, CV27, and COVA2-14 scFvs were expressed and purified by protein A resin and size exclusion, producing proteins of the expected molecular weight (˜300 kDa, lanes 4, 6, and 8 respectively in FIG. 13C). Treatment with TEV protease separates the IgG (˜150 kDa) from the ACE2 (˜71 kDa). TEV digestion of the proteins in lane 4, 6, and 8 yielded the proteins in lanes 5, 7, and 9 demonstrating clear cleavage into the two constituent components (FIG. 13C). CV10 IgG alone was run in lane 2 to depict a standard IgG size, and TEV digested CV10 scFv-ACE2 fusion was run in lane 3 to depict a standard ACE2 size (FIG. 13C).

These constructs were again tested for their affinity by BLI against either SARS-COV-2 S, SARS-COV-1 S, and the RBD of SARS-COV-2. There was less of a change in affinity in this experiment following TEV cleavage (FIGS. 14A-C), compared to FIG. 10A. This is consistent with the bivalent IgG still having a high affinity even after cleavage of the ACE2 component. Strikingly, however, there was a significant change in affinity to the RBD of SARS-COV-2. This is consistent with changing of the ACE2 component from bivalent to monovalent (FIG. 14A-C, right column).

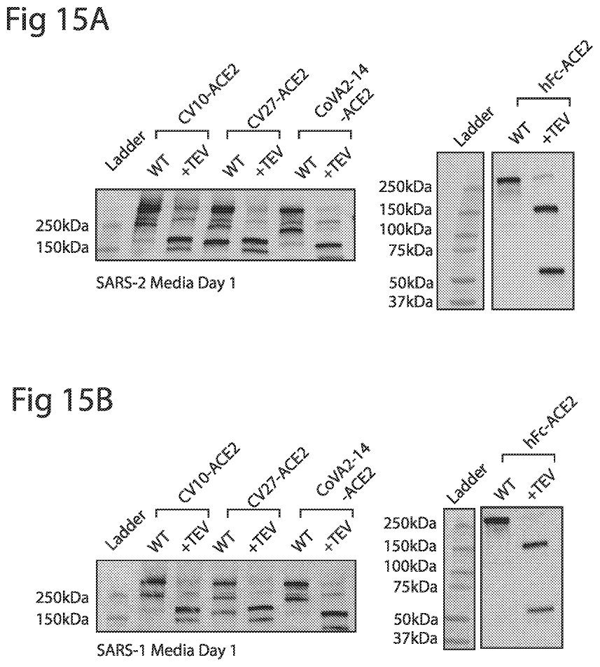

A similar neutralization experiment was conducted using the uncleaved and cleaved IgG-ACE2 fusions as that for the scFv-ACE2 fusions. However, in the assay an additional control was carried, hFc-ACE2. hFc-ACE2 was an important control to carry given that it is important to ensure the neutralization of these constructs is not simply due to the multivalency of the ACE2 component. Again, at day 1 post infection the supernatants were examined to ensure that off-target TEV cleavage was not a significant factor. The western blots of the supernatant from day 1 generally show the expected result that the majority of the uncleaved protein is in the uncleaved state. The western blots also reveal that there was still a minor amount of uncleaved IgG-ACE2 fusions in the TEV cleaved samples and a minor amount of cleaved sample (detectable only in the CV27-IgG-ACE2 fusion protein sample), suggesting that off-target cleavage likely did not play a large role (FIG. 15A-B). These western blots were also used to investigate hFc-ACE2 with and without TEV cleavage. There was little to no residual uncleaved hFc-ACE2 in the cleaved sample, while the uncleaved sample showed no real signs of cleavage in either SARS-COV-2 and SARS-COV-1 (FIG. 15A-B (right)). The western blots reveal that the cleavage is not complete (showing some amount of monomeric ACE2-hFc). This incomplete cleavage is not concerning, however, because even this partially cleaved form of ACE2-hFc is still a monovalent neutralizer, since little to no bivalent ACE2-hFc remained.

Reading out this neutralization assay, it became clear that the IgG-ACE2 fusions significantly potentiated the neutralization of ACE2 compared to either bivalent hFc-ACE2 or monovalent, TEV-cleaved ACE2. This result was true for both SARS-COV-2 and SARS-COV-1 (FIG. 16A-B). As with the scFv-ACE2 constructs the neutralization was better against SARS-CoV-2 (subnanomolar IC50 s for all IgG-ACE2 constructs). However, in marked contrast to the scFv-ACE2 constructs, the CV27-IgG-ACE2 fusion protein was significantly more potent than CV10- or COVA2-14-IgG-ACE2 fusions against SARS-COV-1. The IC50 neutralization potency of the CV27-IgG-ACE2 fusion compared to uncleaved or cleaved hFc-ACE2 is shown in Table 10.

TABLE 10
Neutralization potency of CV27-IgG-ACE2 fusion
compared to uncleaved or cleaved hFc-ACE2.

| | | | | | | | | Fold |
|---|---|---|---|---|---|---|---|---|
| | IC50 | | | | Fold | | | enhancement |
| | CV27- | | IC50 | | enhancement | IC50 | | compared to |
| | IgG- | | uncleaved | | compared to | cleaved | | cleaved |
| Virus | ACE2 | | hFc-ACE2 | | hFc-ACE2 | hFc-ACE2 | | hFc-ACE2 |
| SARS- | .199 | nM | 8.84 | nM | 44.4 fold | 74.9 | nM | 376 fold |
| CoV-2 | | | | | | | | |
| SARS- | 2.58 | nM | 33.4 | nM | 12.9 fold | 2999 | nM | 1162 fold |
| CoV-1 | | | | | | | | |

Collectively, this work represents the first example of converting a non-neutralizing antibody, which targets a broadly reactive epitope, into a broadly neutralizing antibody through linkage with the cellular receptor. The antibody components of these constructs target epitopes which are unlikely to evolve because the antibodies are not neutralizing and are, therefore, not applying a selective pressure to the virus. And, despite undergoing significant mutation in the RBD between SARS-COV-2 and SARS-COV-1, the viruses still utilize the same receptor. Suggesting that antibodies against this region would be ineffective, but the receptor would remain effective. The advantage of utilizing a cross-reactive epitope that is unlikely to undergo antigenic drift, and a receptor which the viruses need to function, makes these nn-bnAbs an extremely attractive platform to treat future pandemics.

### EXAMPLE 5. Development and Characterization of CrossMAb-ACE2 Fusion Proteins

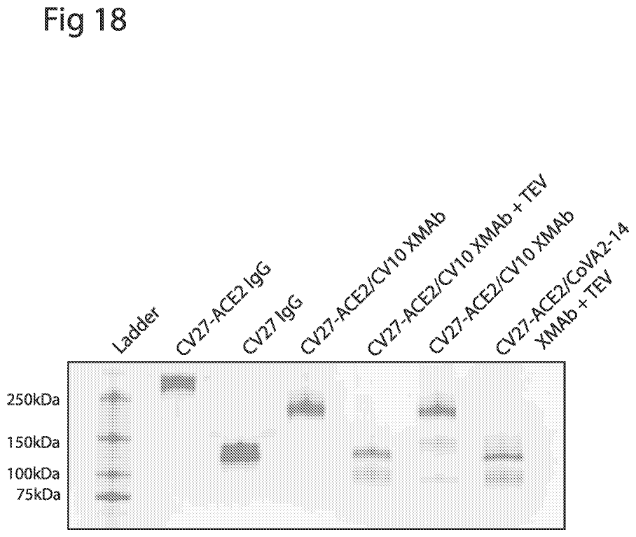

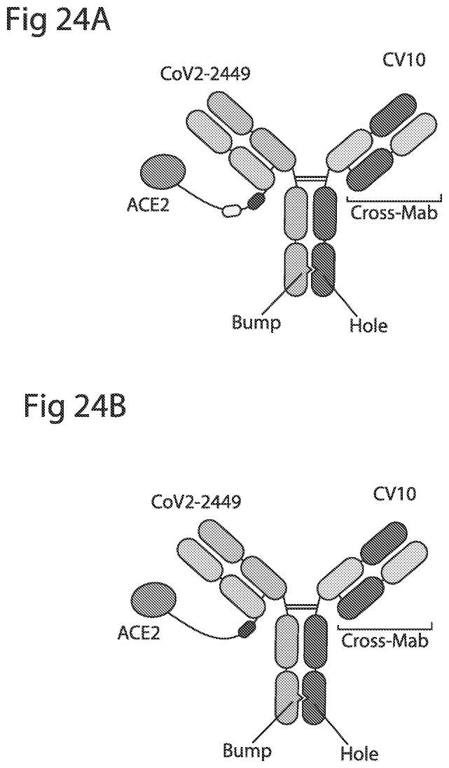

In order to test efficacy of constructs in which ACE2 was fused to an IgG where each of the two binding ends of the IgG had unique epitope targets, CrossMAb-ACE2 fusion proteins were produced. These constructs were designed with two unique binding arms (FIGS. 17A and 17B), one of CV27 linked to ACE2 through a 63 amino acid linker, and the other of either CV10 or COVA2-14 not linked to ACE2. Additional constructs were designed with one arm of COV2-2449 linked to ACE2, and the other of CV10 not linked to ACE2 (FIGS. 24A and 24B). In order to produce these constructs, CV27 or COV2-2449 heavy chain was designed with a “knob” mutation (also referred to as a “bump” mutation), T366W, as well as a disulfide partner mutation, S354C. The heavy chain of CV10 or COVA2-14 was designed to contain “hole” mutations T366S, L368A, and Y407V, as well as a disulfide partner mutation, Y349C. These mutations ensure correct pairing of the heavy chain domains (i.e., a heavy chain with a knob mutation can only pair with a heavy chain with a hole mutation). In order to ensure correct light chain pairing, CrossMAbs were produced. The CV27/COV2-2449 light chain was not modified in any way, while the light chain constant domain of CV10 or COVA2-14 was swapped with the CH1 domain of the heavy chain (shown in FIG. 17 and FIG. 24 and described in Schaefer et al., 2011, Proc. Natl. Acad. Sci. 108(27): 11187-11192). After these unique plasmids were cloned, the antibodies were expressed using Expi 293F cells and purified using protein A and size exclusion chromatography. The resulting proteins were of the expected MW (˜225 kDa; FIG. 18). These constructs also contain a TEV cleavage site, as in other constructs described above, which enables the testing of uncleaved and cleaved constructs Since there is only a single ACE2 molecule in a single CrossMAb, the cleaved control has the same number and valency of ACE2. When comparing the neutralizing potency of uncleaved CrossMAb constructs against SARS-CoV-2, both CV27/CV10 or CV27/COVA2-14 CrossMAbs showed potent neutralization (˜60 pM; FIG. 19). When cleaved using TEV, the neutralization was markedly decreased, not even reaching 50% inhibition at the highest dilution tested (500 nM; FIG. 19). These CrossMAb constructs clearly demonstrate the efficacy of ACE2 fusion proteins against SARS-COV-2, as, although the valency of ACE2 is monomeric in the CrossMAbs, the uncleaved CrossMAb-ACE2 fusion protein neutralizing potency is approximately 4 orders of magnitude improved relative to cleaved CrossMAb-ACE2.

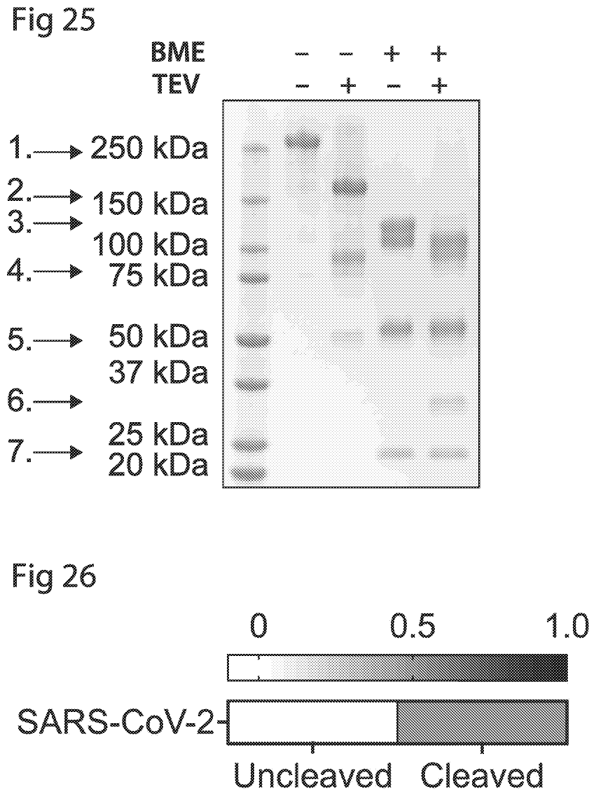

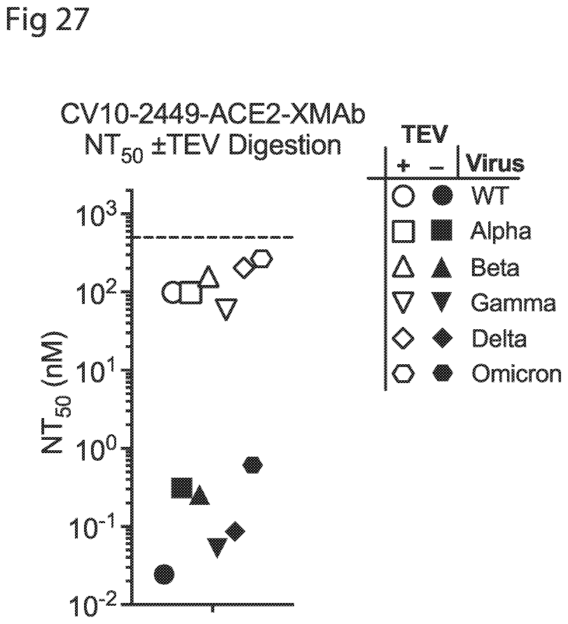

The CV10/COV2-2449 CrossMAb shows the correct molecular weight by SDS-Page gel and the TEV cleavage results in the correct separation of ACE2 from the LC of COV2-2449 (FIG. 25). This is further confirmed by reducing the complexes using 2-mercapto-ethanol: all bands of the expected MW bands are visible (FIG. 25). The uncleaved CV10/COV2-2449 complex is able to potently block hFc-ACE2 from interacting with SARS-COV-2 spike (FIG. 26), consistent with facilitating binding to both the non-neutralizing cross-reactive site and the RBD. However, the TEV cleaved form does not show similar efficacy in blocking hFc-ACE2 binding (FIG. 26). The uncleaved CrossMAb of CV10/COV2-2449 shows potent viral inhibition, able to neutralize all SARS-COV-2 variants of concern at sub-nanomolar concentrations (FIG. 27). The cleaved form, however, does not show the same level of inhibition (FIG. 27). These results demonstrate that a CrossMAb containing CV10 and COV2-2449 linked to ACE2 can act as a potent inhibitor of SARS-COV-2 VOCs.

### EXAMPLE 6. Development of Fusion Proteins of Non-Neutralizing, Non-RBD Binding Antibodies and Neutralizing, RBD-Binding Antibodies

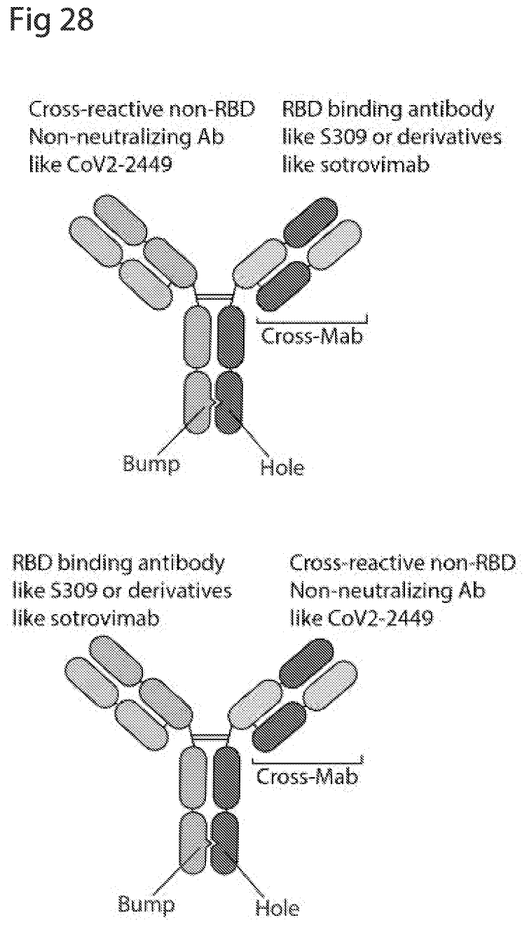

A fusion protein or modified protein can be made as a bispecific protein (e.g., a bispecific antibody) comprising a non-neutralizing antibody that targets a highly conserved region outside the RBD and an RBD-directed neutralizing antibody. This configuration would decrease the likelihood of escape from the RBD-directed neutralizing antibody. Such protein molecules would be capable of binding to a large range of SARS-COV-2 viruses (through the non-neutralizing antibody component) and then neutralizing them (through the RBD-directed neutralizing antibody component). Even with mutations in the RBD, which may decrease the ability for the RBD-directed antibody to neutralize, the non-neutralizing antibody would facilitate binding and increase the effective concentration of the RBD-directed antibody and, therefore, promote neutralization of the mutant virus. This configuration is predicted to overcome SARS-COV-2 evolution, for example as seen in the Omicron variant, to at least some extent. Such bispecific proteins could be made in a number of ways. One such way is a CrossMAb where an antibody is engineered such that each FAb domain contains a different binding specificity (FIG. 28). CrossMAbs of non-neutralizing, conserved epitope binding, non-RBD binding antibodies and neutralizing RBD-directed antibodies could play a significant role in pandemic mitigation.

One exemplary CrossMAb can include the variable domain of S309 or S2×259 as one of the arms of the CrossMAb and the other arm can be either CV10 or COV2-2449 (FIG. 28). The resultant IgG will have 2 unique binding moieties, one that binds to the RBD (through either S309 or S2 X259) and one that binds to a conserved, non-neutralizing, non-RBD epitope (through CV10 or COV2-2449). Either arm of the CrossMAb can be either the RBD-directed neutralizing antibody or the cross-reactive non-neutralizing antibody. The binding arms can be swapped, such that the Fab arm on the “knob” side could be moved to the “hole” side, and that of the “hole” side moved to the “knob” side.

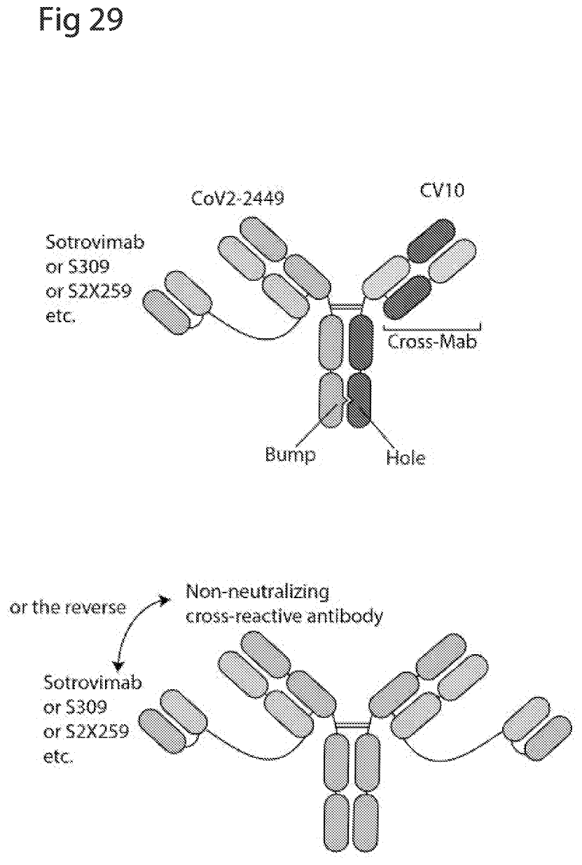

In the same way that AwCE2 polypeptides were fused to non-neutralizing antibodies that bind to conserved sites in SARS-COV-2 spike protein in the Examples above to produce potently neutralizing fusion proteins, the variable domains from neutralizing antibodies (for example, S309 or S2 X259) can be used to produce fusion proteins comprising neutralizing antibodies and non-neutralizing antibodies. A fusion protein will also be produced where the CrossMAb of CV10 and COV2-2449 is utilized in combination with the scFv domain of S309 (FIG. 29, top panel). This will produce a trispecific antibody, one arm will have CV10, one will have COV2-2449, and the scFv of the S309 will be tethered to the light chain of COV2-2449. This trispecific antibody will work in the same way as the CrossMAb with an ACE2 fusion, except that the neutralization will be due to S309 and not ACE2. A similar construct could be produced without the CrossMAb, which would be a bispecific antibody fusion protein where S309, or another RBD directed antibody, is one arm, and CV10, or another non-neutralizing highly conserved antibody, is the other arm (FIG. 29, bottom panel).

### EXAMPLE 7. Identification of Conserved Regions of SARS COV-2 Spike Protein

A multiple sequence alignment (MSA) was generated using. The Consurf Server (ConSurf 2016: an improved methodology to estimate and visualize evolutionary conservation in macromolecules; DOI: 10.1093/nar/gkw408) using the PDB ID:6VXX as the template search structure-residue numbers correspond to the SARS-COV-2 spike protein sequence deposited in 6VXX (and included herein as SEQ ID NO:337). The following parameters were utilized during the search: Search algorithm-HMMER, Number of iterations-1, E-value cutoff-0.0001, Protein Data Base-Uniref-90, 150 sequences, Maximal % ID between sequences-90%, Minimal % ID for homologs-30%, alignment method-MAFFT-L-INS-i. This is to say that the primary selection criteria was proteins, which have between 30% and 90% sequence conservation with the SARS-COV-2 spike protein, regardless of their species of origin. The resultant output sequences from the MSA were curated to remove all sequences with poor sequencing result, i.e., all sequences containing “X”s in the sequencing result. “X”s are shown in sequencing results when there is insufficient sequencing data to identify a residue at that position. Such “X”s can convolute MSA generation because they can be read as mutations when they may just indicate lacking information. The sequences used in the MSA are listed in Table 11. Indicated residue numbers are those which were utilized in the sequence alignments, as they had the most sequence identity.

TABLE 11
MSA sequences.

| Sequence identifier | Residues included in MSA |
|---|---|
| AAT84362.1 | All residues |
| UniRef90_L7UP84 | 299-1269 |
| UniRef90_A0A5Q0TVR4 | 279-1268 |
| UniRef90_U5LMM7 | 298-1272 |
| UniRef90_U5NJG5 | 296-1285 |
| UniRef90_K9N5Q8 | 310-1294 |
| YP_9047204.1 | All residues |
| UniRef90_S4X276 | 304-1301 |
| UniRef90_A0A2R4KP93 | 314-1292 |
| UniRef90_A0A2R4KP86 | 314-1297 |
| UniRef90_A0A2I6PIW5 | 316-1286 |
| UniRef90_A0A678TRJ7 | 311-1288 |
| UniRef90_A0A5H2WTJ3 | 315-1288 |
| UniRef90_A0A023Y9K3 | 285-1263 |
| UniRef90_A0A2Z4EVN2 | 217-1209 |
| UniRef90_A0A2Z4EVN5 | 51-1211 |
| UniRef90_A0A2Z4EVK1 | 219-1204 |
| UniRef90_A3EXG6 | 194-1210 |
| UniRef90_F1DAZ9 | 220-1214 |
| UniRef90_E0ZN60 | 210-1218 |
| UniRef90_E0ZN36 | 209-1207 |
| UniRef90_A0A4Y6GL90 | 213-1215 |
| UniRef90_E0XIZ3 | 8-1198 |
| UniRef90_A0A3Q8AKM0 | 15-1197 |
| UniRef90_A0A7R6WCE7 | 14-1174 |
| UniRef90_A0A2R3SUW7 | 11-1185 |
| UniRef90_A0A6M3G9R1 | 12-1204 |
| QHR63300.2 | All residues |
| UniRef90_A0A7U3W1C7 | 5-1166 |
| UniRef90_USWHZ7 | 15-1195 |
| AGZ48828.1 | All residues |
| UniRef90_A0A0U1UYX4 | 33-1175 |
| UniRef90_A0A0U1WJY8 | 14-1172 |
| UniRef90_A0A166ZND9 | 4-851 |
| UniRef90_A0A0U1WHI2 | 5-1180 |
| UniRef90_A0A7G6UAJ9 | 11-1198 |
| UniRef90_A0A0K1Z054 | 12-1180 |
| UniRef90_F1BYL9 | 42-1239 |
| UniRef90_A0A088DJY6 | 57-1260 |

The resultant curated 40 sequences (i.e., the sequences listed in Table 11 along with SEQ ID NO:337) were used as a MSA to generate conservation scores using. The Consurf Server. The “Amino Acid Conservation Score” is a normalized conservation score where the average score for all residues is zero, and the standard deviation is one. So, a residue with a value of 1 is 1 standard deviation more likely to be mutated than a generic residue in the protein. These values are relative to one another and the sequences used in the MSA. The lowest score represents the most conserved amino acid in the protein.

The resulting conservation scores were analyzed, and all residues that had a conservation score greater than-0.5 (that is to say all residues that were more than half a standard deviation below average in likelihood to be mutated) were excluded. All residues before position 540 were also excluded. Table 12 displays residues, outside the RBD, which are generally conserved, as defined here. Table 13 lists all SARS-COV-2 residues that had 100% identity across all 40 sequences (i.e., the sequences listed in Table 11 along with SEQ ID NO:337).

Bolded residues indicate consecutive residues (at least 6 residues in length) that are conserved, as defined here. Bolded and italicized resides indicate a non-exhaustive list of exposed conserved residues in the structure (PDB ID: 6VXX), and demonstrate sites in which antibodies could potentially bind.

TABLE 12
Conserved residues.

| Residue Number | SEQ | SCORE |
|---|---|---|
| 541 | F | −0.979 |
| 544 | N | −0.547 |
| 545 | G | −1.07 |
| 548 | G | −1.07 |
| 550 | G | −1.07 |
| 551 | V | −0.956 |
| 552 | L | −0.547 |
| 555 | S | −0.659 |
| 563 | Q | −1.077 |
| 565 | F | −0.84 |
| 567 | R | −0.581 |
| 568 | D | −0.675 |
| 585 | L | −0.661 |
| 587 | I | −0.625 |
| 589 | P | −0.649 |
| 590 | G | −1.068 |
| 591 | S | −0.752 |
| 593 | G | −0.679 |
| 595 | V | −0.75 |
| 596 | S | −1.083 |
| 597 | V | −0.854 |
| 599 | T | −0.68 |
| 600 | P | −0.577 |
| 601 | G | −0.765 |
| 605 | S | −0.55 |
| 609 | A | −0.965 |
| 610 | V | −0.753 |
| 611 | L | −0.572 |
| 612 | Y | −0.708 |
| 614 | D | −0.641 |
| 615 | V | −0.697 |
| 617 | C | −1.068 |
| 628 | Q | −0.712 |
| 633 | W | −0.993 |
| 644 | Q | −0.943 |
| 645 | T | −1.059 |
| 647 | A | −0.756 |
| 648 | G | −1.07 |
| 649 | C | −1.068 |
| 650 | L | −0.566 |
| 652 | G | −0.871 |
| 655 | H | −0.51 |
| 659 | S | −0.844 |
| 662 | C | −1.068 |
| 664 | I | −0.785 |
| 667 | G | −1.07 |
| 668 | A | −0.613 |
| 669 | G | −0.846 |
| 671 | C | −1.068 |
| 672 | A | −0.583 |
| 698 | S | −0.752 |
| 707 | Y | −0.631 |
| 710 | N | −0.662 |
| 714 | I | −0.516 |
| 715 | P | −1.092 |
| 716 | T | −0.507 |
| 717 | N | −1.077 |
| 718 | F | −1.107 |
| 720 | I | −0.912 |
| 723 | T | −0.662 |
| 725 | E | −1.147 |
| 727 | L | −0.704 |
| 728 | P | −0.694 |
| 729 | V | −0.958 |
| 731 | M | −0.78 |
| 733 | K | −1.141 |
| 735 | S | −0.724 |
| 736 | V | −0.775 |
| 737 | D | −1.133 |
| 738 | C | −1.068 |
| 740 | M | −0.702 |
| 741 | Y | −0.98 |
| 742 | I | −0.936 |
| 743 | C | −1.068 |
| 744 | G | −0.987 |
| 745 | D | −0.862 |
| 746 | S | −0.769 |
| 749 | C | −1.068 |
| 752 | L | −0.74 |
| 753 | L | −1.108 |
| 755 | Q | −0.851 |
| 756 | Y | −1.109 |
| 757 | G | −1.07 |
| 758 | S | −0.815 |
| 759 | F | −1.107 |
| 760 | C | −1.068 |
| 762 | Q | −0.782 |
| 763 | L | −0.858 |
| 764 | N | −1.156 |
| 766 | A | −1.021 |
| 767 | L | −1.108 |
| 770 | I | −0.574 |
| 771 | A | −0.729 |
| 774 | Q | −0.895 |
| 775 | D | −1.133 |
| 777 | N | −0.69 |
| 781 | V | −0.614 |
| 784 | Q | −0.523 |
| 786 | K | −0.55 |
| 792 | P | −0.64 |
| 798 | G | −0.574 |
| 799 | G | −0.627 |
| 800 | F | −0.84 |
| 801 | N | −1.156 |
| 802 | F | −0.664 |
| 803 | S | −0.662 |
| 805 | I | −0.508 |
| 807 | P | −0.591 |
| 815 | R | −1.132 |
| 816 | S | −1.154 |
| 817 | F | −0.934 |
| 818 | I | −0.642 |
| 819 | E | −1.067 |
| 820 | D | −0.936 |
| 821 | L | −1.108 |
| 822 | L | −0.843 |
| 823 | F | −0.566 |
| 824 | N | −0.691 |
| 825 | K | −1.053 |
| 826 | V | −0.954 |
| 827 | T | −0.546 |
| 829 | A | −0.589 |
| 830 | D | −1.133 |
| 831 | A | −0.63 |
| 832 | G | −1.07 |
| 833 | F | −0.975 |
| 834 | I | −0.746 |
| 835 | K | −0.728 |
| 836 | Q | −0.617 |
| 837 | Y | −1.109 |
| 840 | C | −1.068 |
| 841 | L | −0.677 |
| 842 | G | −0.786 |
| 847 | R | −1.03 |
| 848 | D | −1.133 |
| 849 | L | −1.108 |
| 851 | C | −1.068 |
| 852 | A | −0.961 |
| 853 | Q | −1.153 |
| 855 | F | −0.685 |
| 856 | N | −0.826 |
| 857 | G | −1.07 |
| 858 | L | −0.881 |
| 859 | T | −0.66 |
| 860 | V | −1.139 |
| 861 | L | −1.108 |
| 862 | P | −1.092 |
| 863 | P | −1.092 |
| 864 | L | −0.902 |
| 865 | L | −0.607 |
| 866 | T | −0.884 |
| 869 | M | −0.988 |
| 870 | I | −0.656 |
| 871 | A | −0.678 |
| 873 | Y | −1.109 |
| 874 | T | −1.143 |
| 876 | A | −0.871 |
| 877 | L | −0.826 |
| 879 | A | −0.652 |
| 880 | G | −0.561 |
| 881 | T | −0.787 |
| 882 | I | −0.788 |
| 883 | T | −0.518 |
| 887 | T | −1.134 |
| 888 | F | −0.841 |
| 889 | G | −0.859 |
| 891 | G | −0.769 |
| 892 | A | −1.063 |
| 894 | L | −0.774 |
| 895 | Q | −0.562 |
| 896 | I | −0.714 |
| 897 | P | −1.09 |
| 898 | F | −0.973 |
| 899 | A | −0.932 |
| 900 | M | −0.817 |
| 901 | Q | −1 |
| 902 | M | −0.571 |
| 903 | A | −0.733 |
| 904 | Y | −0.843 |
| 905 | R | −1.131 |
| 907 | N | −1.155 |
| 908 | G | −1.068 |
| 910 | G | −0.907 |
| 911 | V | −1.049 |
| 912 | T | −1.142 |
| 913 | Q | −0.99 |
| 914 | N | −0.652 |
| 915 | V | −1.138 |
| 916 | L | −1.107 |
| 918 | E | −0.793 |
| 919 | N | −1.155 |
| 920 | Q | −1.152 |
| 921 | K | −0.772 |
| 923 | I | −1.146 |
| 924 | A | −1.14 |
| 925 | N | −1.086 |
| 926 | Q | −0.771 |
| 927 | F | −1.106 |
| 928 | N | −1.155 |
| 930 | A | −1.14 |
| 931 | I | −0.748 |
| 934 | I | −0.982 |
| 935 | Q | −1.152 |
| 937 | S | −0.959 |
| 938 | L | −0.973 |
| 941 | T | −1.142 |
| 942 | A | −0.705 |
| 944 | A | −1.14 |
| 945 | L | −0.807 |
| 947 | K | −1.053 |
| 948 | L | −0.792 |
| 949 | Q | −1.152 |
| 951 | V | −0.952 |
| 952 | V | −0.951 |
| 953 | N | −1.155 |
| 955 | N | −1.082 |
| 956 | A | −1.14 |
| 958 | A | −1.056 |
| 959 | L | −1.107 |
| 960 | N | −0.767 |
| 961 | T | −0.665 |
| 962 | L | −1.107 |
| 963 | V | −0.839 |
| 965 | Q | −1.076 |
| 966 | L | −1.107 |
| 967 | S | −0.772 |
| 968 | S | −1.085 |
| 969 | N | −0.871 |
| 970 | F | −1.106 |
| 971 | G | −1.068 |
| 972 | A | −1.14 |
| 973 | I | −1.146 |
| 974 | S | −1.153 |
| 975 | S | −0.7 |
| 976 | V | −0.905 |
| 977 | L | −0.844 |
| 978 | N | −0.817 |
| 979 | D | −0.946 |
| 980 | I | −1.146 |
| 981 | L | −0.555 |
| 982 | S | −0.722 |
| 983 | R | −1.131 |
| 984 | L | −1.107 |
| 985 | D | −1.034 |
| 987 | P | −0.752 |
| 988 | E | −1.065 |
| 989 | A | −1.058 |
| 990 | E | −0.597 |
| 991 | V | −0.668 |
| 992 | Q | −1.003 |
| 993 | I | −1.061 |
| 994 | D | −1.132 |
| 995 | R | −1.131 |
| 996 | L | −1.107 |
| 997 | I | −1.146 |
| 998 | T | −1.003 |
| 999 | G | −1.068 |
| 1000 | R | −1.131 |
| 1001 | L | −0.883 |
| 1002 | Q | −0.882 |
| 1003 | S | −0.644 |
| 1004 | L | −1.107 |
| 1005 | Q | −1.083 |
| 1006 | T | −1.048 |
| 1007 | Y | −0.811 |
| 1008 | V | −1.138 |
| 1009 | T | −0.835 |
| 1010 | Q | −1.152 |
| 1011 | Q | −1.066 |
| 1012 | L | −1.107 |
| 1013 | I | −0.747 |
| 1014 | R | −0.646 |
| 1015 | A | −0.881 |
| 1016 | A | −0.804 |
| 1017 | E | −0.544 |
| 1018 | I | −0.736 |
| 1019 | R | −0.728 |
| 1020 | A | −0.502 |
| 1021 | S | −1 |
| 1022 | A | −0.674 |
| 1023 | N | −0.503 |
| 1024 | L | −0.975 |
| 1025 | A | −1.052 |
| 1027 | T | −0.78 |
| 1028 | K | −1.14 |
| 1029 | M | −0.874 |
| 1030 | S | −1.079 |
| 1031 | E | −1.146 |
| 1032 | C | −1.067 |
| 1033 | V | −1.138 |
| 1034 | L | −0.904 |
| 1036 | Q | −1.152 |
| 1037 | S | −1.153 |
| 1039 | R | −1.131 |
| 1040 | V | −0.568 |
| 1041 | D | −0.889 |
| 1042 | F | −1.106 |
| 1043 | C | −1.067 |
| 1044 | G | −1.068 |
| 1045 | K | −0.65 |
| 1046 | G | −1.068 |
| 1048 | H | −1.132 |
| 1049 | L | −0.927 |
| 1050 | M | −0.548 |
| 1051 | S | −1.081 |
| 1052 | F | −0.616 |
| 1053 | P | −0.723 |
| 1054 | Q | −0.732 |
| 1056 | A | −1.14 |
| 1057 | P | −1.09 |
| 1058 | H | −0.714 |
| 1059 | G | −1.068 |
| 1061 | V | −0.615 |
| 1062 | F | −1.106 |
| 1064 | H | −1.132 |
| 1065 | V | −0.784 |
| 1066 | T | −0.549 |
| 1067 | Y | −0.978 |
| 1069 | P | −1.09 |
| 1070 | A | −0.591 |
| 1074 | N | −0.637 |
| 1076 | T | −0.719 |
| 1077 | T | −0.673 |
| 1078 | A | −0.547 |
| 1079 | P | −0.84 |
| 1080 | A | −0.97 |
| 1081 | I | −1.013 |
| 1082 | C | −1.067 |
| 1087 | A | −0.804 |
| 1088 | H | −0.555 |
| 1089 | F | −0.915 |
| 1090 | P | −0.944 |
| 1093 | G | −1.068 |
| 1094 | V | −0.658 |
| 1095 | F | −1.106 |
| 1096 | V | −0.759 |
| 1102 | W | −0.871 |
| 1105 | T | −1.142 |
| 1106 | Q | −0.665 |
| 1107 | R | −0.785 |
| 1108 | N | −0.677 |
| 1112 | P | −0.939 |
| 1113 | Q | −0.674 |
| 1115 | I | −0.972 |
| 1116 | T | −0.796 |
| 1119 | N | −1.155 |
| 1126 | C | −0.57 |
| 1128 | V | −0.95 |
| 1131 | G | −0.583 |
| 1134 | N | −0.645 |
| 1135 | N | −0.693 |
| 1137 | V | −0.568 |
| 1140 | P | −0.919 |
| 1148 | F | −1.106 |
| 1151 | E | −1.146 |
| 1152 | L | −0.974 |
| 1153 | D | −0.597 |
| 1154 | K | −0.878 |
| 1156 | F | −0.677 |
| 1157 | K | −1.14 |
| 1158 | N | −1.155 |
| 1160 | T | −0.841 |
| 1161 | S | −0.719 |
| 1162 | P | −0.586 |
| 1163 | D | −0.563 |
| 1166 | L | −0.637 |
| 1172 | I | −0.953 |
| 1173 | N | −1.155 |
| 1175 | S | −0.82 |
| 1177 | V | −0.896 |
| 1179 | I | −0.653 |
| 1182 | E | −0.988 |
| 1183 | I | −0.625 |
| 1186 | L | −0.976 |
| 1189 | V | −0.95 |
| 1190 | A | −0.86 |
| 1191 | K | −0.587 |
| 1193 | L | −1.107 |
| 1194 | N | −1.155 |
| 1196 | S | −1.153 |
| 1198 | I | −1.146 |
| 1199 | D | −0.631 |
| 1200 | L | −1.107 |
| 1201 | Q | −0.972 |
| 1202 | E | −0.69 |
| 1203 | L | −0.844 |
| 1204 | G | −0.937 |
| 1205 | K | −0.77 |
| 1206 | Y | −0.71 |
| 1208 | Q | −0.612 |
| 1209 | Y | −0.991 |
| 1211 | K | −1.14 |
| 1212 | G | −0.642 |
| 1215 | R | −0.564 |
| 1218 | L | −0.926 |
| 1226 | S | −0.689 |
| 1248 | L | −0.565 |
| 1252 | F | −0.53 |

TABLE 13
Spike residues with 100% identity across 40 related sequences.
Residue number (relative to SEQ ID NO: 337)

548
550
725
737
738
743
749
753
756
760
767
815
816
832
837
840
848
849
851
853
857
860
861
862
873
874
897
905
907
915
916
919
920
924
927
928
930
944
949
953
959
962
966
970
972
973
974
980
983
984
994
995
996
997
999
1000
1004
1008
1012
1028
1031
1032
1033
1036
1037
1039
1042
1043
1044
1046
1048
1056
1057
1059
1062
1064
1069
1082
1105
1173
1194
1200
1211
1218
1260

1. A fusion protein comprising a neutralizing polypeptide that binds to a receptor binding domain (RBD) of a first coronavirus spike protein, a peptide linker, and an antibody that specifically binds an epitope in a conserved region of a second coronavirus spike protein.

2. The fusion protein of claim 1, wherein the neutralizing polypeptide is a coronavirus receptor polypeptide.

3. The fusion protein of claim 2, wherein the coronavirus receptor polypeptide comprises an ACE2 receptor ectodomain polypeptide or a DPP4 receptor ectodomain polypeptide.

4. The fusion protein of claim 1, wherein the neutralizing polypeptide is a neutralizing antibody or a non-neutralizing antibody.

5. (canceled)

6. The fusion protein of claim 1, wherein the conserved region comprises 90% or greater conservation across related coronaviruses.

7. The fusion protein of claim 6, wherein the related coronaviruses comprise spike proteins with amino acid sequences having 40% or greater amino acid sequence identity to SEQ ID NO:337.

8-9. (canceled)

10. The fusion protein of claim 1, wherein the first coronavirus spike protein and the second coronavirus spike protein are the same protein or different coronavirus spike proteins.

11. The fusion protein of claim 1, wherein the neutralizing polypeptide and the antibody that specifically binds an epitope in a conserved region of the second coronavirus spike protein do not bind competitively to their respective binding sites.

12. (canceled)

13. The fusion protein of claim 3, wherein the ACE2 receptor ectodomain polypeptide comprises an amino acid sequence having at least 90% sequence identity to the amino acid sequence as set forth in SEQ ID NO:270 or SEQ ID NO:271, and/or wherein the DPP4 receptor ectodomain polypeptide comprises an amino acid sequence having at least 90% sequence identity to the amino acid sequence as set forth in SEQ ID NO:273 or SEQ ID NO:274.

14. (canceled)

15. The fusion protein of claim 1, wherein the antibody that specifically binds an epitope in a conserved region of the second coronavirus spike protein comprises:
(a) a heavy chain variable region comprising
(i) a CDRH1 comprising any of SEQ ID NOs: 153-170
(ii) a CDRH2 comprising any of SEQ ID NOs: 171-188
(iii) a CDRH3 comprising any of SEQ ID NOs: 189-214 and
(b) a light chain variable region comprising
(i) a CDRL1 comprising any of SEQ ID NOs: 215-232
(ii) a CDRL2 compring any of SEQ ID NOs: 233-241
(iii) a CDRL3 comprising any of SEQ ID NOs: 242-268.

16. The fusion protein of claim 1, wherein the antibody that specifically binds an epitope in a conserved region of the second coronavirus spike protein comprises a heavy chain variable region having at least 90% sequence identity to any of SEQ ID NOs: 1-7, 11-15, 17-23, 26, 29-32, 34, 35, 37, and 38 and a light chain variable region having at least 90% sequence identity to any of SEQ ID NOs: 77-83, 87-91, 93-99, 102, 105-108, 110, 111, 113, and 114, and wherein the conserved region is a region of a SARS-COV-2 spike protein.

17-18. (canceled)

19. The fusion protein of claim 1, wherein the fusion protein has at least one of: about 1000-fold increased neutralization potency for SARS-CoV-2 relative to the cleaved fusion protein domains, about 44-fold increased neutralization potency for SARS-COV-2, or about 13-fold increased neutralization potency for SARS-COV-1 relative to bivalent ACE2, about 376-fold increased neutralization potency for SARS-COV-2, or about 1162-fold increased neutralization potency for SARS-COV-1 relative to monovalent ACE2.

20-21. (canceled)

22. A recombinant nucleic acid encoding the fusion protein of claim 1.

23. A DNA construct comprising a promoter operably linked to the recombinant nucleic acid of claim 22.

24. A vector comprising the DNA construct of claim 23.

25. A host cell comprising the recombinant nucleic acid of claim 22.

26-28. (canceled)

29. A method of producing a fusion protein comprising culturing the host cell of claim 25, under conditions sufficient for the production of the fusion protein by the host cell.

30. A pharmaceutical preparation comprising:
(a) the fusion protein of claim 1; and
(b) a pharmaceutically acceptable carrier.

31. A method for treating a subject infected with a SARS-COV-2 virus or having symptoms suggestive of a SARS-COV-2 infection, the method comprising administering to the subject a therapeutically effective amount of the pharmaceutical preparation of claim 30.

32. (canceled)

33. A method for treating a subject exposed to a SARS-COV-2 virus or at risk of exposure to SARS-COV-2 virus, the method comprising administering to the subject a therapeutically effective amount of the pharmaceutical preparation of claim 30.

34-36. (canceled)


SEQUENCE LISTING (385 sequences)

SEQ ID NO: 1 - protein, 121 aa, Homo sapiens
QVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARSFGGSYYYGMDVWGQGTTVTVSS

SEQ ID NO: 2 - protein, 121 aa, Homo sapiens
QVQLAESGGGVVQPGRSLRLSCAASGFTFSSYAMHWVRQAPGKGLEWVAVISYDGSNKYYADSVKGRFTISRDNSKNTLYLQMNSLRAEDTAVYYCARSTSGSYYYGMDVWGQGTTVTVSS

SEQ ID NO: 3 - protein, 112 aa, Homo sapiens
QVQLQESGPGLVKPSETLSLTCNVSGGSISSYYWSWIRQPPGKGLEWIGYIYYSGSTNYNPSLKSRVTISVDTSKNQFSLKLSSVTAADTAVYYCARGFDYWGQGTLVTVSS

SEQ ID NO: 4 - protein, 122 aa, Homo sapiens
QVQLVQSGAEVKKPGSSVKVSCKASGGTFSSYAIIWVRQAPGQGLEWMGGIIPIFGTANYAQKFQGRVTITTDESTSTAYMELSSLRSEDTAVYYCARVRYYDSSGYYEDYWGQGTLVTVSS

SEQ ID NO: 5 - protein, 122 aa, Homo sapiens
EVQLVQSGAEVKKPGSSVKVSCKASGGTFSSYAISWVRQAPGQGLEWMGGIIPIFGTTNYAQKFQGRVTITTDESTSTAYMELSSLRSEDTAVYYCARVYSYDSSGYYLEYWGQGTRVTVSS

SEQ ID NO: 6 - protein, 124 aa, Homo sapiens
QVQLVESGGGVVQPGRSLRLSCATSGFTFSSFALHWVRQAPGKGLEWVTVISDDGNNKYYVDSVKGRFTISRDNSKNTLFLQMNSLRVEDTAIYYCARASYNSNWSIGEYFRDWGQGTLVTVSS

SEQ ID NO: 7 - protein, 124 aa, Homo sapiens
EVQLVESGGGLVQPGGSLRLSCAASGFTVSSNYMSWVRQAPGKGLEWVSVIYSAGSTYYADSVKGRFSISRDKSKNTLYLQMNSLRAEDTAVYYCAKEGGSGSLRYYYYGMDVWGQGTTVTVSS

SEQ ID NO: 8-10 - skipped (3 entries)

SEQ ID NO: 11 - protein, 121 aa, Homo sapiens
QVELVESGGGVVQPGRSLRLSCAASGFIFSSYAMHWVRQAPGKGLEWVAVISYDGGNKYYADSVKGRFTISRDNSKNTLYLQMNSLRAEDTAVYYCARAQGGNYYYGMDVWGQGTTVTVSS

SEQ ID NO: 12 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSYGGSYYYGMDVWGQGTTVTVSS

SEQ ID NO: 13 - protein, 121 aa, Homo sapiens
QVQLVESGGGVVQPGRSLRLSCAASGFTFSSYAMHWVRQAPGKGLEWVAVISYDGYNKYYADSVKGRFTISRDNSKNTLYLQMNSLRAEDTAVYYCARAQGGNYYYGMDVWGQGTTVTVSS

SEQ ID NO: 14 - protein, 121 aa, Homo sapiens
QVQLVESGGGVVQPGRSLRLSCAASGFTFSSYGMHWVRQAPGKGLEWVAVISYDGSNKYYADSVKGRFTISRDNAKNSLYLQMNSLRAEDTAVYYCARGSAGNYYYGMDVWGQGTTVTVSS

SEQ ID NO: 15 - protein, 121 aa, Homo sapiens
QVQLVESGGGVVQPGRSLRLSCAASGFTFSTYAMHWVRQAPGKGLAWVALISYDGYNKYYADSVRGRFTISRINSKNTLSLQMNSLRAEDTAVYYCARGSAGNYYYGMDVWGQGTTVTVSS

SEQ ID NO: 16 - skipped

SEQ ID NO: 17 - protein, 127 aa, Homo sapiens
EVQLVQSGAEVKKPGESLKISCKGSGYSFSDYWIGWVRQMPGKGLEWMGIIYPGDSDTRYSPSFQGQVTISADKSISTAYLQWSSLKASDTAMYYCARLTFGGSGSYYFYYNGMDVWGQGTTVTVSS

SEQ ID NO: 18 - protein, 132 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARLVPTWASYYDFWSGYPGGYGMDVWGQGTTVTVSS

SEQ ID NO: 19 - protein, 121 aa, Homo sapiens
QVQLVESGGGVVQPGRSLRLSCAASGFTFSYYAILWFRQAPGKGLEWVAIISYDGSNKYYADSVKGRFTISRDNSKNTLYLQMNSLRPEDTAVYYCARPQSGGYYAPLDYWGQGTLVTVSS

SEQ ID NO: 20 - protein, 124 aa, Homo sapiens
QVQLVQSGAEVKKPGASVKVSCKASGHTFTSYYMHWVRQAPGQGLEWMGIINPSGGSTSYAQKFQGRVTMTRDTSTSTVYMELSSLRSEDTAVYYCARGPERGIVGATDYFDYWGQGTLVTVSS

SEQ ID NO: 21 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAITYYYDSSGYWWDDWGQGTLVTVSS

SEQ ID NO: 22 - protein, 127 aa, Homo sapiens
QLQLQESGPGLVKPSETLSLTCTVSGGSISSSSYYWGWIRQPPGKGLEWIGSVYYIGSTYYNPSLKSRVTMSVDTSKNQFSLKLSSVTAADTAVYYCARAPFQLLDKYYFFYYMDVWGKGTTVTVSS

SEQ ID NO: 23 - protein, 122 aa, Homo sapiens
QVQLVQSGAEVKKPGSSVKVSCKASGGTFSSYAIIWVRQAPGQGLEWMGGIIPIFGTTNYAQKFQGRVTITADESTSTAYVELSSLRSEDTAVYYCARIGHFDSSGYYLDYWGQGTLVTVSS

SEQ ID NO: 24-25 - skipped (2 entries)

SEQ ID NO: 26 - protein, 122 aa, Homo sapiens
QVQLVQSGAEVKKPGSSVKVSCKASGGTFSSYAISWVRQAPGQGLEWMGGIIPIFGAANYAQNFQGRVTITADESTSTGYMQLSSLRFEDTAVYYCARTSHYDSSGSYFEYWGQGTLVTVSS

SEQ ID NO: 27-28 - skipped (2 entries)

SEQ ID NO: 29 - protein, 122 aa, Homo sapiens
QVQLVESGAEVKKPGSSVKVSCKASGGTLSSYAITWVRQAPGQGLEWVGGIIPIFGTANYAQKFQGRVTITADESTSTAYMELSSLRSEDTAVYYCARVGAYDSSGYSNDYWGQGTLVTVSS

SEQ ID NO: 30 - protein, 122 aa, Homo sapiens
EVQLVESGAEVKKPGSSVKVSCKASGGTFSSYAISWVRQAPGQGLEWMGGIIPIFGTANYAQKFQGRVTVTADESTSTAYMELSSLRSEDTAVYYCARTYSFDSSGYYYDYWGQGTMVTVSS

SEQ ID NO: 31 - protein, 120 aa, Homo sapiens
QVQLVESGGGVVQPGRSLRLSCAASGFTFSSYLMHWVRQAPGKGLEWVAVIWANGNRYYADSVKGRFTISRDISKNTLYLQMNSLRAEDTAMYYCARDYCNGVTCNSNYWGQGTLVTVSS

SEQ ID NO: 32 - protein, 118 aa, Homo sapiens
QVQLVESGGGVVQPGRSLRLSCAASGFTFSTYGMHWVRQAPGKGLEWVAVISYDGSNKYYADSVKGRFTISRDNSKNTLYLQMNSLRAEDTAMYYCAKDGSIAAADYWGQGTLVTVSS

SEQ ID NO: 33 - skipped

SEQ ID NO: 34 - protein, 124 aa, Homo sapiens
QVQLVESGGGVVLPGRSLRLSCAASGFTFSTFAMHWVRQAPGKGLEWVAVISDEGSNKYYADSVKGRFTISRDNSRNTLYLQMNSLRAEDTAVYYCARAFYDSNWSVGSYFDSWGQGTPVTVSS

SEQ ID NO: 35 - protein, 121 aa, Homo sapiens
QVQLVESGGGVVQPGRSLRLSCAASGFTFSSYALFWVRQAPGKGLEWVAVISYDGNNKYYADSVRGRFTISRDNSKNTLYLQMNSLRPEDTAVYYCARPYTGSYKSYMDVWGKGTTVTVSS

SEQ ID NO: 36 - skipped

SEQ ID NO: 37 - protein, 124 aa, Homo sapiens
EVQLVESGPGLVKPSETLSLTCAVSGDSTSSSSSYWDWIRQPPGKGLEWIGNIYYTGTTYYNPSLKSRVTISVDTSKDQFSLKLSSVTAADTAVYYCARELFTAVAGKGGIDYWGQGTLVTVSS

SEQ ID NO: 38 - protein, 121 aa, Homo sapiens
QVQLVESGGGVVQPGRSLRLSCAASGFTFSSYAMYWVRQAPGKGLEWVAVISYDGINKYYADSVKGRFTISRDNSKNTLYLQMNSLRAEDTAVYYCARVNSGSYYSYFDYWGQGTLVTVSS

SEQ ID NO: 39 - DNA, 363 bp, Homo sapiens
caggtccaactcgttgaaagcgggggaggtgtagtccagccagggcggagcctccgactcagttgcgctgcaagtggattcactttcagttcctacgcgatgcactgggtacggcaggctccggggaaagggttggaatgggtagccttgatctcatatgacggaagtaataaatattacgcagatagcgttaagggacggttcacaatatctcgcgacaattctaagaatacgctgtaccttcaaatgaattctttgcgcgcagaggatacggcggtatattattgcgcaagaagcttcggcggatcttactattatggaatggatgtgtggggtcaaggaacaacggtcaccgtgagttca

SEQ ID NO: 40 - DNA, 363 bp, Homo sapiens
caggtgcagctggcggagtctgggggaggcgtggtccagcctgggaggtccctgagactctcctgtgcagcctctggattcaccttcagtagctatgctatgcactgggtccgccaggctccaggcaaggggctggaatgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagaagcacgagtgggagctactactacggtatggacgtctggggccaagggaccacggtcaccgtctcctca

SEQ ID NO: 41 - DNA, 336 bp, Homo sapiens
caggttcagctccaggaaagtgggccgggtctggttaaaccaagcgagacgttgtcccttacgtgtaatgtctctggagggtctataagttcttattactggtcttggattcgccagcccccagggaaagggctcgagtggatcggctatatttattactcaggttctacgaactataatccttccttgaaatcccgggtgaccatctcagttgacacaagcaagaaccaattcagtctcaaactctctagcgtcactgctgcggatacggctgtgtactattgtgctcgaggtttcgactactggggccagggcacattggtcacagtatcatca

SEQ ID NO: 42 - DNA, 366 bp, Homo sapiens
caggtccagctggtacagtctggggctgaggtgaagaagcctgggtcctcggtgaaggtctcctgcaaggcttctggaggcaccttcagcagctatgctatcatctgggtgcgacaggcccctggacaagggcttgagtggatgggagggatcatccctatctttggtacagcaaactacgcacagaagttccagggcagagtcacgattaccacggacgaatccacgagcacagcctacatggagctgagcagcctgagatctgaggacacggccgtgtattactgtgcgagagtaagatactatgatagtagtggttattatgaggactactggggccagggaacgctggtcaccgtctcctca

SEQ ID NO: 43 - DNA, 366 bp, Homo sapiens
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacaacaaactacgcacagaagttccagggcagagtcacgattaccacggacgaatccacgagcacagcctacatggagctgagcagcctgagatctgaggacacggccgtgtattactgtgcgagagtctattcctatgatagtagtggttattacttagagtactggggccagggaacgcgggtcaccgtctcttca

SEQ ID NO: 44 - DNA, 372 bp, Homo sapiens
caggtgcagctggtggagtctgggggaggcgtggtccagcctgggaggtccctgagactctcctgtgcaacctctggattcacgttcagtagttttgctttgcactgggtccgccaggctccaggcaaggggctggagtgggtgacagttatatcagatgatggaaataataaatactacgtcgactccgtgaagggccgattcaccatctccagggacaattccaagaacacgctgtttctgcaaatgaacagcctgagagttgaggacacggctatctattactgtgcgagagcctcgtataatagcaattggtctattggtgaatacttccgagactggggccagggcaccctggtcaccgtctcctca

SEQ ID NO: 45 - DNA, 372 bp, Homo sapiens
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcgctggtagcacatactacgcagactccgtgaagggcagattcagcatctccagagacaagtccaagaacacgctgtatcttcaaatgaacagcctgagagccgaggacacggctgtatattactgtgcgaaagaaggtggatcggggagcctccgctactactactacggtatggacgtctggggccaagggaccacggtcaccgtctcctca

SEQ ID NO: 46-48 - skipped (3 entries)

SEQ ID NO: 49 - DNA, 363 bp, Homo sapiens
caggtggagctggtggagtctgggggaggcgtggtccagcctgggaggtccctgagactctcctgtgcagcctctggattcatcttcagtagctatgctatgcactgggtccgccaggctccaggcaaggggctggagtgggtggcagttatatcgtatgatggaggcaataaatactacgcagactccgtgaagggccgattcaccatctccagagacaattccaagaacacgctgtatctgcaaatgaacagcctgagagctgaggacacggctgtgtattactgtgcgagagcccagggggggaactactactacggtatggacgtctggggccaagggaccacggtcaccgtctcctca

SEQ ID NO: 50 - DNA, 363 bp, Homo sapiens
caggtgcagttggttgaatccggtggtggcgtagtgcaaccgggccggagccttaggctgtcctgtgcagcttcagggttcactttcagctcttacgccatgcactgggtacgacaagcaccggggaaaggtctggagtgggttgcggttatttcctacgacggcagcaacaaatattacgctgattcagttaaaggacgattcacgatttctagagacaatagcaaaaatactctgtaccttcaaatgaattctctgagagccgaggataccgccgtgtattactgcgcgaggtcctatggaggaagttactactacggcatggatgtttggggtcaagggaccactgtaaccgtctcttca

SEQ ID NO: 51 - DNA, 363 bp, Homo sapiens
caggtgcagctggtggagtctgggggaggcgtggtccagcctgggaggtccctgagactctcctgtgcagcctctggattcaccttcagtagttacgctatgcactgggtccgccaggctccaggcaaggggctggagtgggtggcagttatatcatatgatggatacaataaatactacgcagactccgtgaagggccgattcaccatctccagagacaattccaagaacacgctgtatctgcaaatgaacagcctgagagctgaggacacggctgtgtattactgtgcgagagcccagggggggaactactactacggtatggacgtctggggccaagggaccacggtcaccgtctcctca

SEQ ID NO: 52 - DNA, 363 bp, Homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaacgccaagaactcactgtatctgcaaatgaacagcctgagagctgaggacacggctgtctattactgtgcgagagggtcagctggaaactactactacggtatggacgtctggggccaagggaccacggtcaccgtctcctca

SEQ ID NO: 53 - DNA, 363 bp, Homo sapiens
caggtgcagctggtggagtctgggggaggcgtggtccagcctgggaggtccctgagactctcctgtgcagcctctggattcaccttcagtacctatgctatgcactgggtccgccaggctccaggcaaggggctggcgtgggtggcacttatatcatatgatggatataataaatactacgcagactccgtgaggggccgattcaccatctccagaatcaattccaagaacacgctgtctctgcagatgaacagcctgagagctgaggacacggctgtctattactgtgcgagagggtcagctggaaactactactacggtatggacgtctggggccaggggaccacggtcaccgtctcctca

SEQ ID NO: 54 - skipped

SEQ ID NO: 55 - DNA, 381 bp, Homo sapiens
gaggtgcagctggtgcagtctggagcagaggtgaaaaagcccggggagtctctgaagatctcctgtaaagggtctggatacagctttagcgactactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagactgacttttggtggttcggggagttattatttctactacaacggtatggacgtctggggccaagggaccacggtcaccgtctcctca

SEQ ID NO: 56 - DNA, 396 bp, Homo sapiens
caggtacagcttgtgcagtccggcgctgaggtcaaaaagccgggagcctctgtaaaagtttcttgcaaagcgagcggttatacattcacctcttacggaatctcttgggtgcgacaggctcctggacaagggcttgagtggatgggatggatcagcgcctacaatgggaacacaaattacgcgcagaaactccaaggaagagtgacaatgaccactgataccagcacctccacggcctatatggaacttaggagcctccgatccgacgacacggcggtgtattattgcgcaaggcttgttccgacgtgggccagctactacgatttctggagcggatacccgggaggatacgggatggacgtctggggtcagggaacaactgtaactgtatcttca

SEQ ID NO: 57 - DNA, 363 bp, Homo sapiens
caggtgcagctggtggagtctgggggaggcgtggtccagcctgggaggtccctgagactctcctgtgcagcctctggattcaccttcagttactatgctatcctctggttccgccaggctccaggcaaggggctggagtgggtggcaattatatcatatgatggaagcaataaatactacgcagactccgtgaagggccgattcaccatctccagagacaattccaagaacacgctgtatctgcaaatgaacagcctgagaccggaggacacggctgtgtattactgtgcgagaccacaaagtgggggctactatgctccccttgactactggggccagggaaccctggtcaccgtctcctca

SEQ ID NO: 58 - DNA, 372 bp, Homo sapiens
caggtgcagctggtgcagtctggggctgaggtgaagaagcctggggcctcagtgaaggtttcctgcaaggcatctggacacaccttcaccagctactatatgcactgggtgcgacaggcccctggacaagggcttgagtggatgggaatcatcaaccctagtggtggtagcacaagctacgcacagaagttccagggcagagtcaccatgaccagggacacgtccacgagcacagtctacatggagctgagcagcctgagatctgaggacacggctgtgtattactgtgctagggggccggaacggggtatagtgggagctactgactactttgactactggggccagggaaccctggtcaccgtctcctca

SEQ ID NO: 59 - DNA, 366 bp, Homo sapiens
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgattacgtattactatgatagtagtggttattggtgggacgactggggccagggaaccctggtcaccgtctcctca

SEQ ID NO: 60 - DNA, 381 bp, Homo sapiens
cagctgcagctgcaggagtcgggcccaggattggtgaagccttcggagaccctgtccctcacctgcactgtctctggtggctccatcagcagtagtagttactactggggctggatccgccagcccccagggaaggggctggagtggattgggagtgtctattatattgggagcacctactacaacccgtccctcaagagtcgagtcaccatgtccgtagacacgtccaagaaccagttctccctgaagctgagctctgtgaccgccgcagacacggctgtgtattactgtgcgagggccccgttccagctgctagacaaatactacttcttctactacatggacgtctggggcaaagggaccacggtcaccgtctcctca

SEQ ID NO: 61 - DNA, 366 bp, Homo sapiens
caggtgcagctggtgcagtctggggctgaggtgaagaagcctgggtcctcggtgaaggtctcctgcaaggcttctggaggcaccttcagcagctatgctatcatctgggtgcgacaggcccctggacaagggcttgagtggatgggagggatcatccctatctttggtacaacaaactacgcacagaagttccagggcagagtcacgattaccgcggacgaatccacgagcacagcctacgtggaactgagcagcctgagatctgaggacacggccgtgtattattgtgcgagaataggccattttgatagtagtggttattacttagactactggggccagggaaccctggtcaccgtctcctca

SEQ ID NO: 62-63 - skipped (2 entries)

SEQ ID NO: 64 - DNA, 366 bp, Homo sapiens
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtgcagcaaactacgcacagaacttccagggcagagtcacgattaccgcggacgaatccacgagcacaggctacatgcaactgagcagcctgagatttgaggacacggccgtgtattactgtgcgagaacgtctcactatgatagtagtggttcctattttgaatactggggccagggaaccctggtcaccgtctcctca

SEQ ID NO: 65-66 - skipped (2 entries)

SEQ ID NO: 67 - DNA, 366 bp, Homo sapiens
caggtgcagctggtggagtctggggctgaggtgaagaagcctgggtcctcggtgaaggtctcttgcaaggcttctggaggcaccctcagcagctatgctatcacctgggtgcgacaggcccctggacaagggcttgagtgggtgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtaggggcctatgatagtagtggttattccaatgactactggggccagggaaccctggtcaccgtctcctca

SEQ ID NO: 68 - DNA, 366 bp, Homo sapiens
gaagtgcagctggtggagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacggttaccgcggacgaatccacgagcacagcctacatggagctaagcagcctgagatctgaggacacggccgtgtattactgcgcgagaacgtattcctttgatagtagtggatattactacgactactggggccagggaaccatggtcaccgtctcttca

SEQ ID NO: 69 - DNA, 360 bp, Homo sapiens
caggtgcagctggtggagtctgggggaggcgtggtccagcctgggaggtccctgagactctcctgtgcagcgtctggattcacctttagtagctatctcatgcactgggtccgccaggctccaggcaaggggctggagtgggtggcagttatatgggctaatggaaatagatattatgcagactccgtgaagggccgattcaccatctccagagacatttccaagaacacgctgtatctgcagatgaatagcctgagagccgaggacacggccatgtattactgtgcgagagactattgtaatggtgttacctgcaactcgaactactggggccagggaaccctggtcaccgtctcctca

SEQ ID NO: 70 - DNA, 354 bp, Homo sapiens
caggtgcagctggtggagtccgggggaggcgtggtccagcctgggaggtccctgagactctcctgtgcagcctctggattcaccttcagtacctatggcatgcactgggtccgccaggctccaggcaaggggctggagtgggtggcagttatatcatatgatggaagtaataaatactatgcagactccgtgaagggccgattcaccatctccagagacaattccaagaacacactgtatctgcaaatgaacagcctgagagctgaggacacggctatgtattactgtgcgaaagatgggagtatagcagcagctgactactggggccagggaaccctggtcaccgtctcctcg

SEQ ID NO: 71 - skipped

SEQ ID NO: 72 - DNA, 372 bp, Homo sapiens
caggttcagctggtggagtctgggggaggcgtggtcctgcctgggaggtccctgagactctcctgtgcagcctctggattcaccttcagtaccttcgctatgcactgggtccgccaggctccaggcaaggggctggagtgggtggcagttatatcagatgaaggaagtaataaatactacgcagactccgtgaagggccgattcaccatctccagagacaattccaggaacaccctttatctgcaaatgaacagcctgagagctgaagacacggctgtgtattattgtgcgagagctttttatgatagtaactggtccgtcggatcctactttgactcctggggccagggaaccccggtcaccgtctcctca

SEQ ID NO: 73 - DNA, 363 bp, Homo sapiens
caggtgcagctggtggagtctgggggaggcgtggtccagcctgggaggtccctgagactctcctgtgcagcctctggattcaccttcagtagttatgctctgttctgggtccgccaggctccaggcaaggggctggagtgggtggcagttatttcatatgatggaaataataaatactacgcagactccgtgaggggccgattcaccatctccagagacaattccaagaacacgctgtatctgcaaatgaacagcctgagacctgaggacacggctgtgtattactgtgcgagaccatatactgggagctacaagagctacatggacgtctggggcaaagggaccacggtcaccgtctcctca

SEQ ID NO: 74 - skipped

SEQ ID NO: 75 - DNA, 372 bp, Homo sapiens
gaggtgcagctggtggagtcgggcccaggactggtgaagccttcggagaccctgtccctcacctgcgctgtctctggtgactccaccagcagtagtagttcctactgggactggatccgccagcccccagggaaggggctggaatggattgggaatatctattatactgggaccacctactacaacccgtccctcaagagtcgagtcaccatatcagtagacacgtccaaggaccagttctccctgaaactgagctctgtgaccgccgcggacacggccgtgtattactgtgcgagagaactatttacggcagtggctggcaaggggggtattgactactggggccagggaaccctggtcaccgtctcctca

SEQ ID NO: 76 - DNA, 363 bp, Homo sapiens
caggtgcagctggtggagtctgggggaggcgtggtccagcctgggaggtccctgagactctcctgtgcagcctctggattcaccttcagtagctatgctatgtactgggtccgccaggctccaggcaaggggctggagtgggtggcagttatatcatatgatggaattaataaatactacgcagactccgtgaagggccgattcaccatctccagagacaattccaagaacacgctgtatctgcaaatgaacagcctgagagctgaggacacggctgtgtattactgtgcgagagtgaacagtgggagctactattcctactttgactactggggccagggaaccctggtcaccgtctcctca

SEQ ID NO: 77 - protein, 111 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVTVL

SEQ ID NO: 78 - protein, 112 aa, Homo sapiens
QSALTQPASVSGSPGQSITISCTGTSSDVGDYNYVSWYQQHPGKAPKLMIYDVSNRPSGVSNRFSGSKSGNTASLTISGLQAEDEAEYYCSSYTSSSTLLYVFGTGTKVTVL

SEQ ID NO: 79 - protein, 108 aa, Homo sapiens
EIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIK

SEQ ID NO: 80 - protein, 109 aa, Homo sapiens
EIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIK

SEQ ID NO: 81 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPSITFGQGTRLEIK

SEQ ID NO: 82 - protein, 113 aa, Homo sapiens
DIVMTQSPDSLAVSLGERATINCKSSQSLLYTSNNKNYLAWYQQKPGQPPKLLIYWASTRESGVPDRFSGSGSGTDFTLTISSLQAEDVAVYYCQQYYSPPWTFGQGTKVEIK

SEQ ID NO: 83 - protein, 110 aa, Homo sapiens
QSVVTQPPSASGTPGQRVTISCSGSSSNIGYNIVNWYQQLPGTAPKLLIYSNNQRPSGVPDRFSGSKSGTSASLSISGLQSEDEADYYCAAWDDSLNGYVFGTGTKVTVL

SEQ ID NO: 84-86 - skipped (3 entries)

SEQ ID NO: 87 - protein, 109 aa, Homo sapiens
SYVLTQPPSVSVAPGKTARITCGGNNIGSKNVHWYQQKPGQAPVKVVYHDGDRPSGIPERFSGSNSGNTATLTINRVEAGDEADYSCQVWDSSSDHHVVFGGGTKLTVL

SEQ ID NO: 88 - protein, 108 aa, Homo sapiens
SYELTQPHSVSVATAQMARITCGGNNIGSKAVHWYQQKPGQDPVLVIYSDSNRPSGIPERFSGSNPGNTATLTISRIEAGDEADYYCQVWDSSSDHVVFGGGTKLTVL

SEQ ID NO: 89 - protein, 109 aa, Homo sapiens
SYVLTQPPSVSVAPGKTARITCGGNNIGTKGVHWYQQKPGQAPVLVVYDDSDRPSGIPGRFSGSNSGNTATLTISRVEAGDEADYFCQVWDSSSDHHVVFGGGTKLTVL

SEQ ID NO: 90 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDYATYYCQQSYSTPGTFGQGTRLEIK

SEQ ID NO: 91 - protein, 107 aa, Homo sapiens
DIQMTQSPSSLSASVGDRVTITCRASQTITNYLNWYQLKSGRAPKLLIYAASSLQSGVPSRFSGSGSGTDFTLTISSLQPEDFATYYCQQSYSTPYTFGQGTKLEIK

SEQ ID NO: 92 - skipped

SEQ ID NO: 93 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSSGTFGQGTKVEIK

SEQ ID NO: 94 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPGTFGQGTRLEIK

SEQ ID NO: 95 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 96 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYVSSPWTFGQGTKVEIK

SEQ ID NO: 97 - protein, 109 aa, Homo sapiens
EIVLTQSPATLSLSPGERATLSCRASQSVSSFLAWYQQKPGQAPRLLIYDASNRATGIPARFSGSGSGTDFTLTISSLEPEDFAVYYCQQRSNWPPSYTFGQGTKLEIK

SEQ ID NO: 98 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPGVTFGQGTRLEIK

SEQ ID NO: 99 - protein, 109 aa, Homo sapiens
EIVLTQSPATLSLSPGERATLSCRASQSVSSFLAWYQQKPGQAPRLLIYDASNRPTGIPARFTGSGSGTDFTLTISSLEPEDFAVYYCQHRTNWPPLFTFGPGTKVDIK

SEQ ID NO: 100-101 - skipped (2 entries)

SEQ ID NO: 102 - protein, 109 aa, Homo sapiens
EIVLTQSPATLSLSPGERATLSCRASQSVSSYLAWYQQKPGQAPRLLIYDASNRATGIPARFSGSGSGTDFTLTISSLDPEDFAVYYCHKRSNWPPSLTFGGGTKVEIK

SEQ ID NO: 103-104 - skipped (2 entries)

SEQ ID NO: 105 - protein, 109 aa, Homo sapiens
DIQLTQSPATLSLSPGERATLSCRASQSVSSYLAWYQQKPGQAPRLLIYDASNRATGIPARFSGSGSGTDFTLTISSLEPEDFAVYYCQQRSNWPPRVTFGGGTKVEIK

SEQ ID NO: 106 - protein, 109 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPPKITFGQGTRLEIK

SEQ ID NO: 107 - protein, 113 aa, Homo sapiens
DFVMTQSPGSLAVSLGERATINCRSSQSVLDNSSNKNHLAWHQQKPGQPPKLLIYWASTRESGVPDRFSGSGSGTDFTLTISSLQAEDVAVYYCQQYYSSHWTFGQGTKVEIK

SEQ ID NO: 108 - protein, 113 aa, Homo sapiens
DIVMTQSPDSLAVSLGERATINCKSSQSVLHSSNNKDSLVWYQQKPGQPPKLLIYWASSRESGVPDRFSGSGSGTDFTLTISSLQAEDVAVYYCQQYYSTPWTFGQGTKVEIK

SEQ ID NO: 109 - skipped

SEQ ID NO: 110 - protein, 113 aa, Homo sapiens
DIQMTQSPDSLAVSLGERATIKCKSSQSVLHSSNNKNYLAWYQQKAGQPPSLLLYWASTRESGVPDRFSGSGSGTDFTLTISSLQAEDVAVYYCQQYYNNQWTFGQGTKVDIK

SEQ ID NO: 111 - protein, 113 aa, Homo sapiens
DIVMTQSPDSLAVSLGERATINCKSSQSVLYSSNNKNSLAWYQQKPGQPPKLLIYWASTRESGVPDRFSGSGSGTDFTLTISSLQAEDVAVYYCQQYYSISWTFGQGTKVEIK

SEQ ID NO: 112 - skipped

SEQ ID NO: 113 - protein, 113 aa, Homo sapiens
EIVLTQSPDSLAVSLGERATINCKSSQSVLYSSNNKNYLAWYQQKPGQPPKLLIYWASTRESGVPDRFSGSGSGTDFTLTISSLQAEDVAVYYCHQYYNTPRTFGQGTKVEIK

SEQ ID NO: 114 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIK

SEQ ID NO: 115 - DNA, 333 bp, Homo sapiens
cagtctgctctgactcaacccgcatcagtttctggttctccgggtcagtctatcaccatatcatgcaccggcactagctctgacgtaggaggatataactacgttagttggtaccaacaacaccctggaaaagcaccgaaattgatgatatatgatgtcagtaaccgacctagcggagtttccaatcgattctccggttcaaagagcggcaatacggcttccttgacaatctccgggcttcaagcggaagatgaagctgactactattgttcttcttacacatctagctccacgccatatgtgttcggaacaggcactaaagtgacagtacta

SEQ ID NO: 116 - DNA, 336 bp, Homo sapiens
cagtctgccctgactcagcctgcctccgtgtctgggtctcctggacagtcgatcaccatctcctgcactggaaccagcagtgacgttggtgattataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgaatattactgcagctcatatacaagcagcagcactctactttatgtcttcggaactgggaccaaggtcaccgtccta

SEQ ID NO: 117 - DNA, 324 bp, Homo sapiens
gaaattgtgcttacgcagtcacccggaactctcagtctgtcccccggtgaaagggccactctctcctgtagggcatcccaaagcgtttctagcatatatctcgcttggtaccaacagaagccgggtcaagctccgcggctgctgatttatggcgcctctagtcgggcaactggtatccctgatcggtttagtgggtcaggaagtggtacagatttcacccttacgatttctcggctcgagcccgaggattttgccgtatactattgtcaacagtacgcagggtctccttggacgtttggccagggcacaaaggtcgagatcaaa

SEQ ID NO: 118 - DNA, 328 bp, Homo sapiens
gaaattgtgttgacacagtctccagccaccctgtctttgtctccaggggaaagagccaccctctcctgcagggccagtcagagtgttagcagctacttagcctggtaccaacaggaacctggccaggctcccaggctcctcatttatgatgcatccaacagggccactggcatcccagccaggttcagtggcagtgggtctgggacagacttcactctcaccatcagcagcctagagcctgaagattttgcagtttattactgtcagcagcgtagcaactggcctcctatgtacacttttggccaggggaccaaggtggagatcaaac

SEQ ID NO: 119 - DNA, 328 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccctcgatcaccttcggccaagggacacgactggagattaaac

SEQ ID NO: 120 - DNA, 339 bp, Homo sapiens
gacatcgtgatgacccagtctccagactccctggctgtgtctctgggcgagagggccaccatcaactgcaagtccagccagagtcttttatacacctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtcctccgtggacgttcggccaagggaccaaggtggaaatcaaa

SEQ ID NO: 121 - DNA, 330 bp, Homo sapiens
cagtctgtggtgactcagccaccctcagcgtctgggacccccgggcagagggtcaccatctcttgttctggaagcagctccaacatcggatataatattgtaaactggtaccagcagctcccaggaacggcccccaaactcctcatctatagtaataatcagaggccctcaggggtccctgaccgattctctggctccaagtctggcacctcagcctccctgtccatcagtgggctccagtctgaggatgaggctgattattactgtgcagcatgggatgacagcctgaatggttatgtcttcggaactgggaccaaggtcaccgtccta

SEQ ID NO: 122-124 - skipped (3 entries)

SEQ ID NO: 125 - DNA, 327 bp, Homo sapiens
tcctatgtgctgactcagccaccctcggtgtcagtggccccaggaaagacggccaggattacctgtgggggaaacaacattggaagtaaaaatgtgcactggtaccagcagaagccaggccaggcccctgtgaaggtcgtctatcatgatggcgaccggccctcagggatccctgagcgattctctggctccaactctgggaacacggccaccctgaccatcaacagggtcgaagccggggatgaggccgactattcctgtcaggtgtgggatagtagtagtgatcatcatgtggttttcggcggagggaccaagctgaccgtccta

SEQ ID NO: 126 - DNA, 324 bp, Homo sapiens
tcctatgagttgactcagcctcatagcgtatcagtcgcaactgcacagatggcgcgcatcacatgcggtggaaacaatataggcagtaaggcggtacattggtatcagcaaaaacctggtcaagaccccgtgttggtaatctactccgattcaaatcgaccctcagggattccagaacgcttctccgggtcaaatccggggaacacggctacactcactataagcagaattgaagcgggagatgaggcggactattactgtcaagtatgggattccagctcagatcatgttgtcttcgggggcggcacaaaactcacagtccta

SEQ ID NO: 127 - DNA, 327 bp, Homo sapiens
tcctatgtgctgactcagccaccctcggtgtcagtggccccaggaaagacggccaggattacctgtgggggaaacaacattggaactaaaggtgtgcactggtaccagcagaagccaggccaggcccctgtgctggtcgtctatgatgatagcgaccggccctcagggatccctgggcgattctctggctccaactctgggaacacggccaccctgaccatcagcagggtcgaagccggggatgaggccgactatttctgtcaggtgtgggatagtagtagtgatcatcatgtggtattcggcggagggaccaagctgaccgtccta

SEQ ID NO: 128 - DNA, 321 bp, Homo sapiens
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattatgcaacttactactgtcaacagagttacagtacccctggcaccttcggccaagggacacgactggagattaaa

SEQ ID NO: 129 - DNA, 321 bp, Homo sapiens
gacatccagatgacccagtctccatcctccctgtctgcatctgtaggagacagagtcaccatcacttgccgggcaagtcagaccattaccaactatttaaattggtatcagctgaaatcagggagagcccccaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacgccgtacacttttggccaggggaccaagctggagatcaaa

SEQ ID NO: 130 - skipped

SEQ ID NO: 131 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcaaaaacctggccaggctcccaggctcctcatctatggtgcatccagcagggccaccggcatcccagacaggttcagtggcagtgggtctgggacagacttcactctcaccatcagcagactggagcctgaagattttgcagtgtattactgtcagcagtatggtagatcatcagggacgttcggccaagggaccaaggtggaaatcaaa

SEQ ID NO: 132 - DNA, 324 bp, Homo sapiens
gaaattgtcttgacgcagagtcctggtacgctgagcctgagcccaggcgaacgagctacccttagctgtagggcttctcaaagtgtgtctagtagctatctggcgtggtaccaacagaaaccaggccaggcccctagacttttgatctacggagcgagtagccgcgcgactggcatccctgaccgattctccggcagtggcagtggaacagattttactcttacaataagtcgccttgagccagaggattttgctgtgtactattgtcaacaatacggtagtagcccggggacgttcgggcaaggcaccagactcgagatcaaa

SEQ ID NO: 133 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccctggacgttcggccaagggaccaaggtggaaatcaaa

SEQ ID NO: 134 - DNA, 324 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatgttagctcaccgtggacgttcggccaagggaccaaggtggaaatcaaa

SEQ ID NO: 135 - DNA, 327 bp, Homo sapiens
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccttcgtacacttttggccaggggaccaagctggagatcaaa

SEQ ID NO: 136 - DNA, 327 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgggcgtcaccttcggccaagggacacgactggagattaaa

SEQ ID NO: 137 - DNA, 327 bp, Homo sapiens
gaaattgtgttgacacagtctccagccaccctgtctttgtctccaggggaaagagccaccctctcctgcagggccagtcagagtgttagcagcttcttagcctggtaccaacagaaacctggccaggctcccaggctcctcatctatgatgcatccaacaggcccactggcatcccagccaggttcactggcagtgggtctgggacagacttcactctcaccatcagcagcctagagcctgaagattttgcagtttattactgtcagcaccgtaccaactggcctcccttattcactttcggccctgggaccaaagtggatatcaaa

SEQ ID NO: 138-139 - skipped (2 entries)

SEQ ID NO: 140 - DNA, 327 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagaccctgaagattttgcagtttattactgtcacaagcgtagcaactggcctccttcgctcactttcggcggagggaccaaggtggagatcaag

SEQ ID NO: 141-142 - skipped (2 entries)

SEQ ID NO: 143 - DNA, 327 bp, Homo sapiens
gacatccagttgacccagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaattggcctccgagggtcactttcggcggagggaccaaggtggagatcaaa

SEQ ID NO: 144 - DNA, 327 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgaagatcaccttcggccaagggacacgactggagattaaa

SEQ ID NO: 145 - DNA, 339 bp, Homo sapiens
gacttcgtgatgacccagtctccaggctccctggctgtgtctctgggcgagagggccaccatcaattgcaggtccagtcagagtgttttagacaactccagcaataagaaccacttagcttggcaccagcagaaaccaggacagcctcctaaactgctcatttactgggcatctacccgggaatccggggtccctgaccgattcagtggcagcgggtctgggacagatttcactctcaccatcagcagcctgcaggctgaagatgtggcagtttattactgtcagcaatattatagtagtcattggacgttcggccaagggaccaaggtggaaatcaaa

SEQ ID NO: 146 - DNA, 339 bp, Homo sapiens
gacatcgtgatgacccagtctccagactccctggctgtgtctctgggcgagagggccaccatcaactgcaagtccagccagagtgtcttacacagctccaacaacaaggactccttagtttggtaccagcagaaaccaggacagcctcctaagctgctcatttactgggcatctagccgggaatccggggtccctgaccgattcagtggcagcgggtctgggacagatttcactctcaccatcagcagcctgcaggctgaagatgtggcagtttattactgtcagcaatattatagtactccttggacgttcggccaagggaccaaggtggaaatcaaa

SEQ ID NO: 147 - skipped

SEQ ID NO: 148 - DNA, 339 bp, Homo sapiens
gacatccagatgacccagtctccagactccctggctgtgtctctgggcgagagggccaccatcaagtgcaagtccagccagagtgttttacacagctccaacaataagaactacttagcttggtaccagcaaaaagcgggacagcctcctagcctactcctttactgggcatctacccgggaatccggggtccctgaccgattcagtggcagcgggtctgggacagatttcactctcaccatcagcagcctgcaggctgaagatgtggcagtttattactgtcagcaatattataataatcagtggacgttcggccaagggaccaaagtggatatcaaa

SEQ ID NO: 149 - DNA, 339 bp, Homo sapiens
gacatcgtgatgacccagtctccagactccctggctgtgtctctgggcgagagggccaccatcaactgcaagtccagccagagtgttttatacagctccaacaataagaactccttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtatattactgtcagcaatattatagtatttcttggacgttcggccaagggaccaaggtggaaatcaaa

SEQ ID NO: 150 - skipped

SEQ ID NO: 151 - DNA, 339 bp, Homo sapiens
gaaattgtgttgacgcagtctccagactccctggctgtgtctctgggcgagagggccaccatcaactgcaagtccagccagagtgttttatacagctccaacaataagaactacctagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcaccaatattataatactcctcgaacgttcggccaagggaccaaggtggaaatcaaa

SEQ ID NO: 152 - DNA, 339 bp, Homo sapiens
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctttaacgttcggccaagggaccaaggtggaaatcaaa

SEQ ID NO: 153 - protein, 8 aa, Homo sapiens
GFTFSSYA

SEQ ID NO: 154 - protein, 8 aa, Homo sapiens
GFIFSSYA

SEQ ID NO: 155 - protein, 8 aa, Homo sapiens
GFTFSSYG

SEQ ID NO: 156 - protein, 8 aa, Homo sapiens
GFTFSTYA

SEQ ID NO: 157 - protein, 8 aa, Homo sapiens
GGSISSYY

SEQ ID NO: 158 - protein, 8 aa, Homo sapiens
GYSFSDYW

SEQ ID NO: 159 - protein, 8 aa, Homo sapiens
GYTFTSYG

SEQ ID NO: 160 - protein, 8 aa, Homo sapiens
GFTFSYYA

SEQ ID NO: 161 - protein, 8 aa, Homo sapiens
GHTFTSYY

SEQ ID NO: 162 - protein, 8 aa, Homo sapiens
GGTFSSYA

SEQ ID NO: 163 - protein, 10 aa, Homo sapiens
GGSISSSSYY

SEQ ID NO: 164 - protein, 8 aa, Homo sapiens
GGTLSSYA

SEQ ID NO: 165 - protein, 8 aa, Homo sapiens
GFTFSSFA

SEQ ID NO: 166 - protein, 8 aa, Homo sapiens
GFTFSSYL

SEQ ID NO: 167 - protein, 8 aa, Homo sapiens
GFTFSTYG

SEQ ID NO: 168 - protein, 8 aa, Homo sapiens
GFTFSTFA

SEQ ID NO: 169 - protein, 10 aa, Homo sapiens
GDSTSSSSSY

SEQ ID NO: 170 - protein, 8 aa, Homo sapiens
GFTVSSNY

SEQ ID NO: 171 - protein, 8 aa, Homo sapiens
ISYDGSNK

SEQ ID NO: 172 - protein, 8 aa, Homo sapiens
ISYDGGNK

SEQ ID NO: 173 - protein, 8 aa, Homo sapiens
ISYDGYNK

SEQ ID NO: 174 - protein, 7 aa, Homo sapiens
IYYSGST

SEQ ID NO: 175 - protein, 8 aa, Homo sapiens
IYPGDSDT

SEQ ID NO: 176 - protein, 8 aa, Homo sapiens
ISAYNGNT

SEQ ID NO: 177 - protein, 8 aa, Homo sapiens
INPSGGST

SEQ ID NO: 178 - protein, 8 aa, Homo sapiens
IIPIFGTA

SEQ ID NO: 179 - protein, 7 aa, Homo sapiens
VYYIGST

SEQ ID NO: 180 - protein, 8 aa, Homo sapiens
IIPIFGTT

SEQ ID NO: 181 - protein, 8 aa, Homo sapiens
IIPIFGAA

SEQ ID NO: 182 - protein, 8 aa, Homo sapiens
ISDDGNNK

SEQ ID NO: 183 - protein, 7 aa, Homo sapiens
IWANGNR

SEQ ID NO: 184 - protein, 8 aa, Homo sapiens
ISDEGSNK

SEQ ID NO: 185 - protein, 8 aa, Homo sapiens
ISYDGNNK

SEQ ID NO: 186 - protein, 7 aa, Homo sapiens
IYYTGTT

SEQ ID NO: 187 - protein, 8 aa, Homo sapiens
ISYDGINK

SEQ ID NO: 188 - protein, 7 aa, Homo sapiens
IYSAGST

SEQ ID NO: 189 - protein, 14 aa, Homo sapiens
ARSFGGSYYYGMDV

SEQ ID NO: 190 - protein, 14 aa, Homo sapiens
ARSTSGSYYYGMDV

SEQ ID NO: 191 - protein, 14 aa, Homo sapiens
ARAQGGNYYYGMDV

SEQ ID NO: 192 - protein, 14 aa, Homo sapiens
ARSYGGSYYYGMDV

SEQ ID NO: 193 - protein, 14 aa, Homo sapiens
ARGSAGNYYYGMDV

SEQ ID NO: 194 - protein, 6 aa, Homo sapiens
ARGFDY

SEQ ID NO: 195 - protein, 20 aa, Homo sapiens
ARLTFGGSGSYYFYYNGMDV

SEQ ID NO: 196 - protein, 25 aa, Homo sapiens
ARLVPTWASYYDFWSGYPGGYGMDV

SEQ ID NO: 197 - protein, 14 aa, Homo sapiens
ARPQSGGYYAPLDY

SEQ ID NO: 198 - protein, 17 aa, Homo sapiens
ARGPERGIVGATDYFDY

SEQ ID NO: 199 - protein, 15 aa, Homo sapiens
ARVRYYDSSGYYEDY

SEQ ID NO: 200 - protein, 15 aa, Homo sapiens
AITYYYDSSGYWWDD

SEQ ID NO: 201 - protein, 19 aa, Homo sapiens
ARAPFQLLDKYYFFYYMDV

SEQ ID NO: 202 - protein, 15 aa, Homo sapiens
ARIGHFDSSGYYLDY

SEQ ID NO: 203 - protein, 15 aa, Homo sapiens
ARTSHYDSSGSYFEY

SEQ ID NO: 204 - protein, 15 aa, Homo sapiens
ARVGAYDSSGYSNDY

SEQ ID NO: 205 - protein, 15 aa, Homo sapiens
ARVYSYDSSGYYLEY

SEQ ID NO: 206 - protein, 15 aa, Homo sapiens
ARTYSFDSSGYYYDY

SEQ ID NO: 207 - protein, 17 aa, Homo sapiens
ARASYNSNWSIGEYFRD

SEQ ID NO: 208 - protein, 14 aa, Homo sapiens
ARDYCNGVTCNSNY

SEQ ID NO: 209 - protein, 11 aa, Homo sapiens
AKDGSIAAADY

SEQ ID NO: 210 - protein, 17 aa, Homo sapiens
ARAFYDSNWSVGSYFDS

SEQ ID NO: 211 - protein, 14 aa, Homo sapiens
ARPYTGSYKSYMDV

SEQ ID NO: 212 - protein, 16 aa, Homo sapiens
ARELFTAVAGKGGIDY

SEQ ID NO: 213 - protein, 14 aa, Homo sapiens
ARVNSGSYYSYFDY

SEQ ID NO: 214 - protein, 18 aa, Homo sapiens
AKEGGSGSLRYYYYGMDV

SEQ ID NO: 215 - protein, 9 aa, Homo sapiens
SSDVGGYNY

SEQ ID NO: 216 - protein, 9 aa, Homo sapiens
SSDVGDYNY

SEQ ID NO: 217 - protein, 6 aa, Homo sapiens
NIGSKN

SEQ ID NO: 218 - protein, 6 aa, Homo sapiens
NIGSKA

SEQ ID NO: 219 - protein, 6 aa, Homo sapiens
NIGTKG

SEQ ID NO: 220 - protein, 6 aa, Homo sapiens
QSISSY

SEQ ID NO: 221 - protein, 6 aa, Homo sapiens
QTITNY

SEQ ID NO: 222 - protein, 7 aa, Homo sapiens
QSVSSIY

SEQ ID NO: 223 - protein, 7 aa, Homo sapiens
QSVSSSY

SEQ ID NO: 224 - protein, 6 aa, Homo sapiens
QSVSSY

SEQ ID NO: 225 - protein, 6 aa, Homo sapiens
QSVSSF

SEQ ID NO: 226 - protein, 12 aa, Homo sapiens
QSLLYTSNNKNY

SEQ ID NO: 227 - protein, 12 aa, Homo sapiens
QSVLDNSSNKNH

SEQ ID NO: 228 - protein, 12 aa, Homo sapiens
QSVLHSSNNKDS

SEQ ID NO: 229 - protein, 12 aa, Homo sapiens
QSVLHSSNNKNY

SEQ ID NO: 230 - protein, 12 aa, Homo sapiens
QSVLYSSNNKNS

SEQ ID NO: 231 - protein, 12 aa, Homo sapiens
QSVLYSSNNKNY

SEQ ID NO: 232 - protein, 8 aa, Homo sapiens
SSNIGYNI

SEQ ID NO: 233 - protein, 3 aa, Homo sapiens
DVS

SEQ ID NO: 234 - protein, 3 aa, Homo sapiens
HDG

SEQ ID NO: 235 - protein, 3 aa, Homo sapiens
SDS

SEQ ID NO: 236 - protein, 3 aa, Homo sapiens
DDS

SEQ ID NO: 237 - protein, 3 aa, Homo sapiens
AAS

SEQ ID NO: 238 - protein, 3 aa, Homo sapiens
GAS

SEQ ID NO: 239 - protein, 3 aa, Homo sapiens
DAS

SEQ ID NO: 240 - protein, 3 aa, Homo sapiens
WAS

SEQ ID NO: 241 - protein, 3 aa, Homo sapiens
SNN

SEQ ID NO: 242 - protein, 11 aa, Homo sapiens
SSYTSSSTPYV

SEQ ID NO: 243 - protein, 12 aa, Homo sapiens
SSYTSSSTLLYV

SEQ ID NO: 244 - protein, 12 aa, Homo sapiens
QVWDSSSDHHVV

SEQ ID NO: 245 - protein, 11 aa, Homo sapiens
QVWDSSSDHVV

SEQ ID NO: 246 - protein, 9 aa, Homo sapiens
QQSYSTPGT

SEQ ID NO: 247 - protein, 9 aa, Homo sapiens
QQSYSTPYT

SEQ ID NO: 248 - protein, 9 aa, Homo sapiens
QQYAGSPWT

SEQ ID NO: 249 - protein, 9 aa, Homo sapiens
QQYGRSSGT

SEQ ID NO: 250 - protein, 9 aa, Homo sapiens
QQYGSSPGT

SEQ ID NO: 251 - protein, 9 aa, Homo sapiens
QQYGSSPWT

SEQ ID NO: 252 - protein, 9 aa, Homo sapiens
QQYVSSPWT

SEQ ID NO: 253 - protein, 11 aa, Homo sapiens
QQRSNWPPMYT

SEQ ID NO: 254 - protein, 11 aa, Homo sapiens
QQRSNWPPSYT

SEQ ID NO: 255 - protein, 11 aa, Homo sapiens
QQRSNWPPGVT

SEQ ID NO: 256 - protein, 11 aa, Homo sapiens
QHRTNWPPLFT

SEQ ID NO: 257 - protein, 11 aa, Homo sapiens
HKRSNWPPSLT

SEQ ID NO: 258 - protein, 11 aa, Homo sapiens
QQRSNWPPRVT

SEQ ID NO: 259 - protein, 11 aa, Homo sapiens
QQRSNWPPSIT

SEQ ID NO: 260 - protein, 11 aa, Homo sapiens
QQRSNWPPKIT

SEQ ID NO: 261 - protein, 9 aa, Homo sapiens
QQYYSPPWT

SEQ ID NO: 262 - protein, 9 aa, Homo sapiens
QQYYSSHWT

SEQ ID NO: 263 - protein, 9 aa, Homo sapiens
QQYYSTPWT

SEQ ID NO: 264 - protein, 9 aa, Homo sapiens
QQYYNNQWT

SEQ ID NO: 265 - protein, 9 aa, Homo sapiens
QQYYSISWT

SEQ ID NO: 266 - protein, 9 aa, Homo sapiens
HQYYNTPRT

SEQ ID NO: 267 - protein, 9 aa, Homo sapiens
QQYYSTPLT

SEQ ID NO: 268 - protein, 11 aa, Homo sapiens
AAWDDSLNGYV

SEQ ID NO: 269 - protein, 805 aa, Homo sapiens [sequence omitted]

SEQ ID NO: 270 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 271 - protein, 597 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 272 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 273 - protein, 738 aa, Homo sapiens
NKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 274 - protein, 728 aa, Homo sapiens
SRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 275 - protein, 909 aa, Artificial Sequence
  Synthetic construct
MGWSCIILFLVATATGVHSQVQLQESGPGLVKPSETLSLTCNVSGGSISSYYWSWIRQPPGKGLEWIGYIYYSGSTNYNPSLKSRVTISVDTSKNQFSLKLSSVTAADTAVYYCARGFDYWGQGTLVTVSSASGGGGSGGGGSGGGGSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 276 - protein, 915 aa, Artificial Sequence
  Synthetic construct
MGWSCIILFLVATATGVHSQVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARSFGGSYYYGMDVWGQGTTVTASGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 277 - protein, 920 aa, Artificial Sequence
  Synthetic construct
MGWSCIILFLVATATGVHSQVQLVQSGAEVKKPGSSVKVSCKASGGTFSSYAIIWVRQAPGQGLEWMGGIIPIFGTANYAQKFQGRVTITTDESTSTAYMELSSLRSEDTAVYYCARVRYYDSSGYYEDYWGQGTLVTVSSASGGGGSGGGGSGGGGSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 278 - protein, 887 aa, Artificial Sequence
  Synthetic construct
MGWSCIILFLVATATGVHSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 279 - protein, 234 aa, Artificial Sequence
  Synthetic construct
MGWSCIILFLVATATGVHSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 461 aa, Artificial Sequence
  Synthetic construct
MGWSCIILFLVATATGVHSQVQLQESGPGLVKPSETLSLTCNVSGGSISSYYWSWIRQPPGKGLEWIGYIYYSGSTNYNPSLKSRVTISVDTSKNQFSLKLSSVTAADTAVYYCARGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 890 aa, Artificial Sequence
  Synthetic construct
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 282 - protein, 237 aa, Artificial Sequence
  Synthetic construct
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 470 aa, Artificial Sequence
  Synthetic construct
MGWSCIILFLVATATGVHSQVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARSFGGSYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 888 aa, Artificial Sequence
  Synthetic construct
MGWSCIILFLVATATGVHSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 285 - protein, 235 aa, Artificial Sequence
  Synthetic construct
MGWSCIILFLVATATGVHSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 471 aa, Artificial Sequence
  Synthetic construct
MGWSCIILFLVATATGVHSQVQLVQSGAEVKKPGSSVKVSCKASGGTFSSYAIIWVRQAPGQGLEWMGGIIPIFGTANYAQKFQGRVTITTDESTSTAYMELSSLRSEDTAVYYCARVRYYDSSGYYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 853 aa, Artificial Sequence
  Synthetic construct
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADENLYFQGGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 288 - protein, 894 aa, Artificial Sequence
  Synthetic construct
MGWSCIILFLVATATGVHSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 289 - protein, 911 aa, Artificial Sequence
  Synthetic construct
MGWSCIILFLVATATGVHSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLSGGASSGGSGGSSAGGGFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 290 - protein, 897 aa, Artificial Sequence
  Synthetic construct
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 291 - protein, 914 aa, Artificial Sequence
  Synthetic construct
MGWSCIILFLVATATGVHSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLSGGASSGGSGGSSAGGGFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 292 - protein, 895 aa, Artificial Sequence
  Synthetic construct
MGWSCIILFLVATATGVHSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 293 - protein, 912 aa, Artificial Sequence
  Synthetic construct
MGWSCIILFLVATATGVHSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLSGGASSGGSGGSSAGGGFVSNHSGQAGAAAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 294 - protein, 56 aa, Artificial Sequence
  Synthetic construct
GGSGSHHHHHHASTGGGSGGPSGQAGAAASEENLYFQGSLFVSNHAYGGSGGEARV

SEQ ID NO: 295 - protein, 63 aa, Artificial Sequence
  Synthetic construct
GGSGSHHHHHHASTGGGSGGPSGQAGAAASEENLYFQGSLFVSNHSGQAGAAAYGGSGGEARV

SEQ ID NO: 296 - protein, 80 aa, Artificial Sequence
  Synthetic construct
GGSGSHHHHHHASTGGGSGGPSGQAGAAASEENLYFQGSLSGGASSGGSGGSSAGGGFVSNHSGQAGAAAYGGSGGEARV

SEQ ID NO: 297 - protein, 90 aa, Artificial Sequence
  Synthetic construct
GGSGSHHHHHHASTGGGSGGPSGQAGAGSSGGSGGSSAASEENLYFQGSLSGGASSGGSGGSSAGGGFVSNHSGQAGAAAYGGSGGEARV

SEQ ID NO: 298 - protein, 101 aa, Artificial Sequence
  Synthetic construct
GGSGSHHHHHHASTGGGSGGPSGQAGAGSSGGSGGSSAASEENLYFQGSLSGGASSGGSGGSSAGGGFVSNHSGQAGAGGSSGGSGGSSAAYGGSGGEARV

SEQ ID NO: 299 - protein, 113 aa, Artificial Sequence
  Synthetic construct
GGSGSHHHHHHASTGGGSGGPSGQAGAGGSSGGSGGSSAASEENLYFQGSLSGGASSGGSGGSSAGGGFVSNHSGQAGAGGSSGGSGGSSAAYGGSGGEAGGSSGGSGGSSRV

SEQ ID NO: 300 - protein, 470 aa, Artificial Sequence
  Synthetic construct
MGWSCIILFLVATATGVHSQVQLVESGGGVVQPGRSLRLSCAASGFTFSSYAMHWVRQAPGKGLEWVALISYDGSNKYYADSVKGRFTISRDNSKNTLYLQMNSLRAEDTAVYYCARSFGGSYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 232 aa, Artificial Sequence
  Synthetic construct
MGWSCIILFLVATATGVHSEIVLTQSPGTLSLSPGERATLSCRASQSVSSIYLAWYQQKPGQAPRLLIYGASSRATGIPDRFSGSGSGTDFTLTISRLEPEDFAVYYCQQYAGSPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 302 - protein, 465 aa, Artificial Sequence
  Synthetic construct
MGWSCIILFLVATATGVHSQVQLQESGPGLVKPSETLSLTCNVSGGSISSYYWSWIRQPPGKGLEWIGYIYYSGSTNYNPSLKSRVTISVDTSKNQFSLKLSSVTAADTAVYYCARGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 303 - protein, 233 aa, Artificial Sequence
  Synthetic construct
MGWSCIILFLVATATGVHSEIVLTQSPATLSLSPGERATLSCRASQSVSSYLAWYQQEPGQAPRLLIYDASNRATGIPARFSGSGSGTDFTLTISSLEPEDFAVYYCQQRSNWPPMYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 304 - protein, 475 aa, Artificial Sequence
  Synthetic construct
MGWSCIILFLVATATGVHSQVQLVQSGAEVKKPGSSVKVSCKASGGTFSSYAIIWVRQAPGQGLEWMGGIIPIFGTANYAQKFQGRVTITTDESTSTAYMELSSLRSEDTAVYYCARVRYYDSSGYYEDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 305 - protein, 5 aa, Unknown
  Peptide linker
GGGGS

SEQ ID NO: 306 - protein, 6 aa, Unknown
  Peptide linker
GSSGSS

SEQ ID NO: 307 - protein, 15 aa, Unknown
  Peptide linker
SGSETPGTSESATPE

SEQ ID NO: 308 - protein, 16 aa, Unknown
  Peptide linker
SGSETPGTSESATPES

SEQ ID NO: 309 - protein, 15 aa, Unknown
  Peptide linker
GGGGSGGGGSGGGGS

SEQ ID NO: 310 - protein, 25 aa, Unknown
  Peptide linker
GGGGSGGGGSGGGGSGGGGSGGGGS

SEQ ID NO: 311 - protein, 50 aa, Unknown
  Peptide linker
GGGGSGGGGSGGGGSGGGGSGGGGSGGGGSGGGGSGGGGSGGGGSGGGGS

SEQ ID NO: 312 - protein, 8 aa, Unknown
  Peptide linker
GGGGGGGG

SEQ ID NO: 313 - protein, 12 aa, Unknown
  Peptide linker
GSAGSAAGSGEF

SEQ ID NO: 314 - protein, 17 aa, Unknown
  Peptide linker
AEAAAKEAAAKEAAAKA

SEQ ID NO: 315 - protein, 52 aa, Unknown
  Peptide linker
AEAAAKEAAAKEAAAKEAAAKEAAAKEAAAKEAAAKEAAAKEAAAKEAAAKA

SEQ ID NO: 316 - protein, 7 aa, Unknown
  TEV protease cleavage site
ENLYFQG

SEQ ID NO: 317 - protein, 8 aa, Unknown
  FLAG tag
DYKDDDDK

SEQ ID NO: 318 - protein, 6 aa, Unknown
  Hexa-HIS tag
HHHHHH

SEQ ID NO: 319 - protein, 9 aa, Unknown
  HA tag
YPYDVPDYA

SEQ ID NO: 320 - protein, 19 aa, Mus musculus
MGWSCIILFLVATATGVHS

SEQ ID NO: 321 - protein, 65 aa, Artificial Sequence
  Synthetic construct
GGGGSRMKQIEDKIEEILSKQYHIENEIARIKKLIGERGGSGGGLNDIFEAQKIEWHEGHHHHHH

SEQ ID NO: 322 - protein, 33 aa, Unknown
  GCN4 sequence
RMKQIEDKIEEILSKQYHIENEIARIKKLIGER

SEQ ID NO: 323 - protein, 15 aa, Unknown
  Avi tag
GLNDIFEAQKIEWHE

SEQ ID NO: 324 - protein, 926 aa, Artificial Sequence
  Synthetic construct
MGWSCIILFLVATATGVHSQVQLVESGGGVVQPGRSLRLSCATSGFTFSSFALHWVRQAPGKGLEWVTVISDDGNNKYYVDSVKGRFTISRDNSKNTLFLQMNSLRVEDTAIYYCARASYNSNWSIGEYFRDWGQGTLVTVSSASGGGGSGGGGSGGGGSDIVMTQSPDSLAVSLGERATINCKSSQSLLYTSNNKNYLAWYQQKPGQPPKLLIYWASTRESGVPDRFSGSGSGTDFTLTISSLQAEDVAVYYCQQYYSPPWTFGQGTKVEIKGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 325 - protein, 923 aa, Artificial Sequence
  Synthetic construct
MGWSCIILFLVATATGVHSEVQLVESGGGLVQPGGSLRLSCAASGFTVSSNYMSWVRQAPGKGLEWVSVIYSAGSTYYADSVKGRFSISRDKSKNTLYLQMNSLRAEDTAVYYCAKEGGSGSLRYYYYGMDVWGQGTTVTVSSASGGGGSGGGGSGGGGSQSVVTQPPSASGTPGQRVTISCSGSSSNIGYNIVNWYQQLPGTAPKLLIYSNNQRPSGVPDRFSGSKSGTSASLSISGLQSEDEADYYCAAWDDSLNGYVFGTGTKVTVLGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 326 - protein, 892 aa, Artificial Sequence
  Synthetic construct
MGWSCIILFLVATATGVHSDIVMTQSPDSLAVSLGERATINCKSSQSLLYTSNNKNYLAWYQQKPGQPPKLLIYWASTRESGVPDRFSGSGSGTDFTLTISSLQAEDVAVYYCQQYYSPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGSHHHHHHASTGGGSGGPSGQAGAAASEENLYFQGSLFVSNHAYGGSGGEARVSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 327 - protein, 473 aa, Artificial Sequence
  Synthetic construct
MGWSCIILFLVATATGVHSQVQLVESGGGVVQPGRSLRLSCATSGFTFSSFALHWVRQAPGKGLEWVTVISDDGNNKYYVDSVKGRFTISRDNSKNTLFLQMNSLRVEDTAIYYCARASYNSNWSIGEYFRDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 328 - protein, 129 aa, Homo sapiens
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSAS

SEQ ID NO: 329 - protein, 104 aa, Homo sapiens
IVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVE

SEQ ID NO: 330 - protein, 129 aa, Artificial Sequence
  Synthetic construct
QVQLVQSGAEVKKPGASVKVSCKASGYPFTSYGISWVRQAPGQGLEWMGWISTYQGNTNYAQKFQGRVTMTTDTSTTTGYMELRRLRSDDTAVYYCARDYTRGAWFGESLIGGFDNWGQGTLVTVSSAS

SEQ ID NO: 331 - protein, 126 aa, Homo sapiens
QVQLVQSGAEVKKPGSSVKVSCKASGGIFNTYTISWVRQAPGQGLEWMGRIILMSGMANYAQKIQGRVTITADKSTSTAYMELTSLRSDDTAVYYCARGFNGNYYGWGDDDAFDIWGQGTLVTVYS

SEQ ID NO: 332 - protein, 113 aa, Homo sapiens
QTVLTQPPSVSGAPGQRVTISCTGSNSNIGAGYDVHWYQQLPGTAPKLLICGNSNRPSGVPDRFSGSKSGTSASLAITGLQAEDEADYYCQSYDSSLSGPNWVFGGGTKLTVL

SEQ ID NO: 333 - protein, 122 aa, Homo sapiens
QVQLVESGGVVVQPGGSLRLSCAASGFTFHDHTMHWVRQAPGKGLEWVSLITWNGGTIHYSDSVKGRFTISRDNSKNSLYLQMNSLRTEDTALYYCAKDLGRGGWYLPSDAWGQGTLVTVSS

SEQ ID NO: 334 - protein, 109 aa, Homo sapiens
QSVLTQPPSASGTPGQRVTISCSGSSANIGSNTVNWYQHLPGTAPKLLIYSNNQRPSGVPDRFSGSKSGTSASLAISGLQSEDEADYYCAAWDDSLKGVFGGGTKLTVL

SEQ ID NO: 335 - protein, 120 aa, Homo sapiens
QVRLVQSGAEVKKSGESLKISCKGSGYSFTSYWIGWVRQMPGKGLEWMGIIYPGDSDTRYSPSFQGQVTISADKSISTVYLQWSSLKASDTAMYYCARQWSHYTYDYYYWGQGTLVTISS

SEQ ID NO: 336 - protein, 112 aa, Homo sapiens
QSVLTQPASVSGSPGQSITISCTGISSDVGGYNSVSWYQQHPGKAPKLMIYDVTNRPSGVSNRFSGSKSGNTASLTISGLQAEDEADYYCSSYTSSSTPPYVFGTGTKVSVL

SEQ ID NO: 337 - protein, 1281 aa, Severe acute respiratory syndrome coronavirus 2
MGILPSPGMPALLSLVSLLSVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKGSGRENLYFQGGGGSGYIPEAPRDGQAYVRKDGEWVLLSTFLGHHHHHHHH

SEQ ID NO: 338 - DNA, 840 bp, Artificial Sequence
  Synthetic construct
atgggatggtcatgtatcatcctttttctagtagcaactgcaaccggtgtacattcacaggtgcagctggtggagtctgggggaggcgtggtccagcctgggaggtccctgagactctcctgtgcagcctctggattcaccttcagtatctatgctatgcactgggtccgccaggctccaggcaaggggctggagtgggtggcagttatatcatatgatggaagcaatcaatactacgcagactccgtgaggggccgattcaccatctccagaggcaattccaagaacacgctgtatctgcaaatgaacagcctgagacctgaggacacggctgtgtactactgtgcgagagcgaggggtgggagctacaatgatgcttttgatatctggggccaagggacaatggtcaccgtctcttcagctagcggcggaggcggatcaggcggaggaggctctggcggaggcggatcgcagtctgccctgactcagcctgcctccgtgtctgggtctcctggacagtcgatcaccatctcctgcactggagccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcacccccgtggtattcggcggagggaccgaactgaccgtcctaggcggatctggatctcaccaccaccaccaccactga

SEQ ID NO: 339 - DNA, 804 bp, Artificial Sequence
  Synthetic construct
atgggatggtcatgtatcatcctttttctagtagcaactgcaaccggtgtacattcacaggttcagctccaggaaagtgggccgggtctggttaaaccaagcgagacgttgtcccttacgtgtaatgtctctggagggtctataagttcttattactggtcttggattcgccagcccccagggaaagggctcgagtggatcggctatatttattactcaggttctacgaactataatccttccttgaaatcccgggtgaccatctcagttgacacaagcaagaaccaattcagtctcaaactctctagcgtcactgctgcggatacggctgtgtactattgtgctcgaggtttcgactactggggccagggcacattggtcacagtatcatcagctagcggcggaggcggatcaggcggaggaggctctggcggaggcggatcggaaattgtgcttacgcagtcacccggaactctcagtctgtcccccggtgaaagggccactctctcctgtagggcatcccaaagcgtttctagcatatatctcgcttggtaccaacagaagccgggtcaagctccgcggctgctgatttatggcgcctctagtcgggcaactggtatccctgatcggtttagtgggtcaggaagtggtacagatttcacccttacgatttctcggctcgagcccgaggattttgccgtatactattgtcaacagtacgcagggtctccttggacgtttggccagggcacaaaggtcgagatcaaaggcggatctggatctcaccaccaccaccaccactga

SEQ ID NO: 340 - DNA, 837 bp, Artificial Sequence
  Synthetic construct
atgggatggtcatgtatcatcctttttctagtagcaactgcaaccggtgtacattcacaggtccagctggtacagtctggggctgaggtgaagaagcctgggtcctcggtgaaggtctcctgcaaggcttctggaggcaccttcagcagctatgctatcatctgggtgcgacaggcccctggacaagggcttgagtggatgggagggatcatccctatctttggtacagcaaactacgcacagaagttccagggcagagtcacgattaccacggacgaatccacgagcacagcctacatggagctgagcagcctgagatctgaggacacggccgtgtattactgtgcgagagtaagatactatgatagtagtggttattatgaggactactggggccagggaacgctggtcaccgtctcctcagctagcggcggaggcggatcaggcggaggaggctctggcggaggcggatcggaaattgtgttgacacagtctccagccaccctgtctttgtctccaggggaaagagccaccctctcctgcagggccagtcagagtgttagcagctacttagcctggtaccaacaggaacctggccaggctcccaggctcctcatttatgatgcatccaacagggccactggcatcccagccaggttcagtggcagtgggtctgggacagacttcactctcaccatcagcagcctagagcctgaagattttgcagtttattactgtcagcagcgtagcaactggcctcctatgtacacttttggccaggggaccaaggtggagatcaaaggcggatctggatctcaccaccaccaccaccactga

SEQ ID NO: 341 - DNA, 837 bp, Artificial Sequence
  Synthetic construct
atgggatggtcatgtatcatcctttttctagtagcaactgcaaccggtgtacattcacaggtgcagcttgtagaaagcggaggtggagttgtacaaccggggaggtcattgcggctgagttgtgccgcctcaggttttaatttgtctaattatgctatgcactgggtcagacaagcatcagggaaagggctcgaatgggtaagtctgatttcatacgacggcagcgtaaagtactataccgactcagtcaaagggaggtttactgttagtggagataattcaaagaacacattgtttcttcaaatgaatagtctgcggcccgacgacagcgccctttattactgcgtgaggggcggggtcagcggacctaacagctttgatatgtggggccaggggactaccgtcaccgtgtctgctagcggcggaggcggatcaggcggaggaggctctggcggaggcggatcggatgtagtgatgacacaatcacctttgtctctcccggtaacacttggacagccggcaactattagttgcagatcatcccagagtttggtatattctgacgggaacacctatgttaactggttccagcaacgcccaggtcaaagtcccaggaggcttatttatcaagtatccatacgcgcctcaggggtaccagatcgattttctggttcagggtcaggaacagatttcgcccttaagatctcaagggttgaggcagaagatgttggtgtctattattgtatgcaaggaactcattggccggtgaccttcgggcaaggaacaaaggtggaaataaaaggcggatctggatctcaccaccaccaccaccactga

SEQ ID NO: 342 - DNA, 834 bp, Artificial Sequence
  Synthetic construct
atgggatggtcatgtatcatcctttttctagtagcaactgcaaccggtgtacattcacaggtgcagctacagcagtcgggcccaggactggtgaagccttcggagaccctgcccctcacctgcactgtctctggtggctccatcagcagtagtagttactactggggctggatccgccagcccccagggaaggggctggagtggattgggagtgtcttttatagtgggagcacctactacaacccgtccctcaagagtcgagtcaccatatccgtagacacgtccaagaaccagttgtccctgaagctgagctctgtgaccgccgcagacacggctgtgtattactgtgcgagacaagtaaggcagtggctggaggatgatgcttttgatatctggggccaagggacaatggtcaccgtctcttcagctagcggcggaggcggatcaggcggaggaggctctggcggaggcggatcggacatccagttgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaagtggatatcaaaggcggatctggatctcaccaccaccaccaccactga

SEQ ID NO: 343 - DNA, 846 bp, Artificial Sequence
  Synthetic construct
atgggatggtcatgtatcatcctttttctagtagcaactgcaaccggtgtacattcagaggtacagctggttgaatcaggaggaggcttggtaaagccgggtgggtctcttaggctttcttgtgcagcaagcgggttcacgtttagtaatgcatggatgtcctgggtaaggcaagcgcccggaaaggggcttgaatgggtcggtcggataaagtccaaaactgatggaggaaccacggactatgcagcaccagtgaaaggccgcttcactatcagccgggatgactcaaagaatacactttaccttcagatgaactctttgaaaacggaggatacagcagtatactattgcacgacggaccgggtttatgattatatatggggttcttatcggtatttggactattggggacaaggcactctggttacggtgagctcagctagcggcggaggcggatcaggcggaggaggctctggcggaggcggatcggaaattgtgctgactcagtcccctgctacattgtcactttctccaggcgagcgggcaacactgtcatgtcgagctagccagtccgtatccagctacttggcatggtatcaacaaaagcccggacaggccccgcgattgctgatatacgacgccagtaatcgcgcgaccgggatcccggcacgattttctggcagcggaagcggcactgatttcacactgactatcagctcactggagccagaggattttgcagtgtactactgtcaacagagatctaactggcccctcacattcgggggaggaacgaaggtcgaaataaaaggcggatctggatctcaccaccaccaccaccactga

SEQ ID NO: 344 - DNA, 822 bp, Artificial Sequence
  Synthetic construct
atgggatggtcatgtatcatcctttttctagtagcaactgcaaccggtgtacattcacaggtgcagctggtggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgaggggccccgcggctacctactactactacatggacgtctggggcaaagggaccagggtcaccgtctcttcagctagcggcggaggcggatcaggcggaggaggctctggcggaggcggatcggacatccagatgacgcagtctccatcctccctgtctgcatctgtaggagacagagtcaccatcacttgccgggcaagtcagagcatgagcagctatttaaattggtatcagcagaaaccagggaaagcccctaagctcctgatctatgctgcatccagtttgcagagtggggtcccatcaaggttcagtggcagtggatctgggacagatttcactctcaccatcagcagtctgcaacctgaagattttgcaacttactactgtcaacagagttacagtactctcactttcggcggagggaccaaggtggagatcaaaggcggatctggatctcaccaccaccaccaccactga

SEQ ID NO: 345 - DNA, 837 bp, Artificial Sequence
  Synthetic construct
atgggatggtcatgtatcatcctttttctagtagcaactgcaaccggtgtacattcacaggtccagttggtacaaagtggagccgaagttaaaaagcctggagcatctgtgaaagtctcttgtaaagtgagcggatacacgcttacagaactttctatgcattgggtgaggcaggctccaggtaagggcctcgaatggatgggaggattcgaccccgaggatggagaaacaatttacgctcagaaatttcaggggcgggtcacgatgacagaggatacaagtactgatacagcatacatggagttgtctagcttgcgctccgaagacacggcggtttattactgtgccacagccccgccttacagtccaccgagctcctggttcgatccatgggggcaggggacgctcgtgactgtcgctagcggcggaggcggatcaggcggaggaggctctggcggaggcggatcgcagagtgtcctcacccaaccacctagcgtaagcgccgcgcctgggcagaaagtaacgataagttgcagcggaagctcctccaacattggaaataattacgtcagttggtaccagcaactgccaggcactgcaccaaagcttcttatctacgacaacaacaagcgcccgagtggcattccagaccggttttcaggctctaaatccgggactagtgcaaccttgggcataacaggattgcaaacaggagatgaagccgactactactgtggtacctgggatagttcactgagcgcgtcttacgttttcggaactgggacaaaggtaacggtactaggcggatctggatctcaccaccaccaccaccactga

SEQ ID NO: 346 - DNA, 825 bp, Artificial Sequence
  Synthetic construct
atgggatggtcatgtatcatcctttttctagtagcaactgcaaccggtgtacattcagaagtgcagctggtggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtaacacatactacgcagactccgtgaagggccggttcaccatctccagagacaattccaagaacacgctgtatctgcaaatgaacagcctgagagccgaggacacggccgtatattactgtgcgaaagagatagcagtggctggttgttttgactactggggccagggaaccctggtcaccgtctcctcagctagcggcggaggcggatcaggcggaggaggctctggcggaggcggatcgtcctatgtggtgacccagccaccctcggtgtcagtgtccccaggacagacggccaggatcacctgctctggagatgcattaccaaagcaatatgcttattggtaccagcagaagccaggccaggcccctgtgctggtgatatataaagacagtgagaggccctcagggatccctgagcgattctctggctccagctcagggacaacagtcacgttgaccatcagtggagtccaggcagaagacgaggctgactattactgtcaatcagcagacagcagtggtacttatagggtgttcggcggagggaccaagctgaccgtcctaggcggatctggatctcaccaccaccaccaccactga

SEQ ID NO: 347 - DNA, 867 bp, Artificial Sequence
  Synthetic construct
atgggatggtcatgtatcatcctttttctagtagcaactgcaaccggtgtacattcacaggttcagctggtggaaagtggtggaggcgttgttcagccgggccggagtcttcggctttcttgcgcggctagcgggtttacattttctagctatgctatgcattgggttcgccaagctccaggtaagggactcgaatgggtagctgttatatcctatgatggttccaataaatactatgcggatagcgtgaaaggtaggttcaccataagtagggataactccaaaaacactctttatctccaaatgaactctcttcgagcggaggacacagcagtttactactgcgcaagagacgaagcttactatgacatattgacagggtacatcaacgccccgaagaactattattactatggcatggatgtatggggacaggggactactgtcacggtgtcctcagctagcggcggaggcggatcaggcggaggaggctctggcggaggcggatcggacatccagatgactcagagtccaagtagtctttccgcgagcgtgggggatagagtaaccatcacttgtagagcaagccaagggattagaaatgacctgggatggtaccagcagaagcccgggaaggctccgaagcgacttatctatgcagccagtagcttgcaatccggagtaccctcacgcttcagtggtagtgggtccggaacggaattcacactgactattagctccctgcagccggaggatttcgcgacctactactgcttgcaacataacagctaccctttcaccttcggtcctgggacaaaggttgacattaaaggcggatctggatctcaccaccaccaccaccactga

SEQ ID NO: 348 - DNA, 855 bp, Artificial Sequence
  Synthetic construct
atgggatggtcatgtatcatcctttttctagtagcaactgcaaccggtgtacattcacaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagagttgggtccgtgaaatcaactgccggatacgatttttggagtggtgatccctttgactactggggccagggaaccctggtcaccgtctcctcagctagcggcggaggcggatcaggcggaggaggctctggcggaggcggatcgtcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattatgtcttcggaactgggaccaaggtcaccgtcctaggcggatctggatctcaccaccaccaccaccactga

SEQ ID NO: 349 - DNA, 822 bp, Artificial Sequence
  Synthetic construct
atgggatggtcatgtatcatcctttttctagtagcaactgcaaccggtgtacattcacaggtccaactcgttgaaagcgggggaggtgtagtccagccagggcggagcctccgactcagttgcgctgcaagtggattcactttcagttcctacgcgatgcactgggtacggcaggctccggggaaagggttggaatgggtagccttgatctcatatgacggaagtaataaatattacgcagatagcgttaagggacggttcacaatatctcgcgacaattctaagaatacgctgtaccttcaaatgaattctttgcgcgcagaggatacggcggtatattattgcgcaagaagcttcggcggatcttactattatggaatggatgtgtggggtcaaggaacaacggtcaccgctagcggcggaggcggatcaggcggaggaggctctggcggaggcggatcgcagtctgctctgactcaacccgcatcagtttctggttctccgggtcagtctatcaccatatcatgcaccggcactagctctgacgtaggaggatataactacgttagttggtaccaacaacaccctggaaaagcaccgaaattgatgatatatgatgtcagtaaccgacctagcggagtttccaatcgattctccggttcaaagagcggcaatacggcttccttgacaatctccgggcttcaagcggaagatgaagctgactactattgttcttcttacacatctagctccacgccatatgtgttcggaacaggcactaaagtgggcggatctggatctcaccaccaccaccaccactga

SEQ ID NO: 350 - DNA, 810 bp, Artificial Sequence
  Synthetic construct
atgggatggtcatgtatcatcctttttctagtagcaactgcaaccggtgtacattcacaggtacaacttgtgcaatcaggctccgagctcaaaaagcccggtgcatccgtcaagctgtcttgtcaggccagcggctatagtttcactaaccacgccatgaactgggtccgccaagctccgggtcaagggctggagtggatggggtggattaacacaaacaccggaaatccaacgtacgcacaaggctttacaggccgcttcgttttctccttggatacgagcgttagtacaacctatttgcatatcagcagcttgaaagccgaggacacggcggtatattattgtgcaagggcttcagcaagacctggagtagcgacgaatcttgacttctgggggcaaggggctagcggcggaggcggatcaggcggaggaggctctggcggaggcggatcggacatacaaatgacgcaatccccgtctagtttgtcagcctccgtcggtgatcgagtgacaatcacctgtagggcaagccagaacatagacaactacctcaactggtaccaacagaagccgggaaaagccccaaagctgctgatatatgccgcctcaagactgcacagcggcgttccgtcacgctttagcggaagtggatccggtacagacttcacgttgattataagcagtctgcagcctgaggaccttgcaacctactactgccaacaaagttattctaatcctttgacgtttggtccaggtacaaaggtcgatattagaggcggatctggatctcaccaccaccaccaccactga

SEQ ID NO: 351 - DNA, 846 bp, Artificial Sequence
  Synthetic construct
atgggatggtcatgtatcatcctttttctagtagcaactgcaaccggtgtacattcacaggtccagctccaagaatctggcccagggctcgtcaaaccttctgagactttgtcattgacctgcacagtgtctggttatagcatctcttccggctattattggggatggattcgacagcccccgggtaagggactggaatggattggcagtatataccactctggaagtacctattataaccctagtctcaagtcccgcgtcaccatcagtgtggatacatctaagaaccagttttccctgaagttgagctctgttaccgctgccgatactgccgtctactactgtgcccgcacgcccctctccctcaggctgcgctataattggtactttgatctctggggacgagggacgttggtaacagtctcctcagctagcggcggaggcggatcaggcggaggaggctctggcggaggcggatcgcagagtgttttgacccaacctccatcagcttctggaactcctgggcaacgagttacaatctcatgttctgggtcctcctctaatatcggaagtaacacagttaattggtatcaacaacttcctggtacggcgccaaagttgctgatttatagtaataatcagcgaccttccggcgtcccggatcgcttttctgggagcaagagtgggacttcagccagcctcgcgataagcgggcttcaatctgaagacgaggctgactactattgtgccgcatgggacgatagtttgaacggacctgttttcggtggagggaccaaattgactgttctaggcggatctggatctcaccaccaccaccaccactga

SEQ ID NO: 352 - DNA, 798 bp, Artificial Sequence
  Synthetic construct
atgggatggtcatgtatcatcctttttctagtagcaactgcaaccggtgtacattcacaggttcaattggttgaatctgggggaggggtagtccaacctggccgaagcctgcgattgtcctgtgctgcaagcgggtttacatttagtagctatgcaatgcattgggtccgacaagcccccgggaagggtttggagtgggttgccgtgattagttacgatgggagtaacaaatactatgcggacagtgtcaagggtcgctttacgatatctagggacaattctaaaaatactctttatcttcaaatgaattctctgcgagccgaagataccgcagtctactactgcgcgcgctctatatcaggcagttaccttggcgcattcgactattgggggcaagctagcggcggaggcggatcaggcggaggaggctctggcggaggcggatcggacatccagatgacccaatccccgtccactcttagcgccagtgttggcgatcgcgtaacgattacatgcagggcctcacagagcatcagttcttggctcgcgtggtatcagcagaagccgggtaaggcacccaagttgctcatctataaagcttctagcttggagagtggtgtgccatctaggttcagtggaagcggctcaggaactgagtttacattgacgatatcttcactgcagcctgacgacttcgctacttactattgtcaacagtataactcttatacttttggacagggcaccaaactggaaataaaaggcggatctggatctcaccaccaccaccaccactga

SEQ ID NO: 353 - DNA, 834 bp, Artificial Sequence
  Synthetic construct
atgggatggtcatgtatcatcctttttctagtagcaactgcaaccggtgtacattcacaggttcaacttgtgcaatctggtgccgaagtaaagaaacctggtgcctctgttaaggtatcttgcaaggcttcaggttacacgttttcatcttacggaattagttgggtacgccaagacccgggacagggcttggagtggatggggtggatctccgcctacaacggaaacacaaactacgcacaaaagctccaggggcgcgtaactatgaccactgatacaagcacgagcaccgcatacatggaactgcggtctctgagaagcgatgacaccgcagtttactactgcgctcgcgttggcttgtggtggctcggtcacccagatgccttcgacatctgggggcagggtactatggtaaccgtttcttcagctagcggcggaggcggatcaggcggaggaggctctggcggaggcggatcggacattcaaatgacgcagagtccaagtagcctttccgcatctgtaggtgaccgagtgaccattacgtgcagggctagtcaaggtatccgcaatgatttgggctggtaccaacaaaagccggggaaagcaccgaaacgactcatttacgcagcaagttccctccagtcaggagttccctcaagattctccggctccggctcaggaaccgagtttactttgacaatttcatccctgcaaccagaagactttgcaacgtattactgccttcaacataactcttatccattgactttcgggggaggcacgaaagttgaaatcaaaggcggatctggatctcaccaccaccaccaccactga

SEQ ID NO: 354 - DNA, 879 bp, Artificial Sequence
  Synthetic construct
atgggatggtcatgtatcatcctttttctagtagcaactgcaaccggtgtacattcacaggtacaactcgttgaatctggcggaggggtcgttcaacctggacgctccctcagactgagctgcgcggccagtggcttcacgtttagctcctacggaatgcactgggtcagacaggctccagggaagggactggagtgggtggccgtaatctcatacgacgggagcaataaatactacgcggattctgtcaaggggcgatttacaatctcccgagataacagtaagaataccctgtacttacagatgagcagtcttcggggggaggacactgccgtgtactactgtgctaaggaccaggcctactatgacatcttgacagggtatctcaaccccccgaagaactactactactacggtatggatgtgtggggtcaggggaccacagtgactgtctcatccgcatctggaggcggaggctccggggggggtggttctggaggggggggatctcagtccgtcctgacacagccccccagtgcatcctccacacctgggcagcgagtgaccattagttgtagcgggtccagctcaaacatcggaagtaactacgtgtattggtaccaacagctgcccgggaccgccccaaagctgctgatatatactaataatcagcgcccgtctggggtccccgatcgctttagtggtagcaaatccggcaccagtgcctccttagcaattagcggtctgcgcagtgaggacgaggccgactattattgtgcggcttgggacgatagcttgtccggacgcgtggtgtttggaggagggactaagcttactgtcttaggcggatctggatctcaccaccaccaccaccactga

SEQ ID NO: 355 - DNA, 840 bp, Artificial Sequence
  Synthetic construct
atgggatggtcatgtatcatcctttttctagtagcaactgcaaccggtgtacattcacaagtgcaactgcaggaaagcgggcccggtctggtcaagcccagtgaaaccttgtcactgacctgtactgtgagtggaggatcaatcagtagctccagttattactggggttggatcaggcagcccccaggtaaaggactggaatggatcggatccatctactattccggtagcacctattacaacccttcactcaaatctcgagttaccatatccgttgatactagtaagaaccagttttctctgaaactctcaagcgtaaccgctgccgacacagccgtgtattattgtgctaggcagggggactgcagcacaacgtcttgtgcctacgattactggggacaagggacattggtcaccgtcagcagcgcctccggcgggggcgggtctgggggaggggggtcaggtgggggcggctctagctatgagctcacacagcctccatctgtgtcagtgagccccggacaaacagctcgcattacctgcagcggcgatgccctgcccaaacagtatgcatactggtaccagcagaaacctggccaggcccctgtgctggttatttataaggactccgaacgtcctagtggaattcctgaacgtttttcagggagttcatccgggacaactgcaacactgaccatcagtggggtccaggctgaggacgaggccgactattactgccagtccgcagacagttccggcacgtacttggtggtctttggcgggggtaccaagctggccgtgctgggcggatctggatctcaccaccaccaccaccactga

SEQ ID NO: 356 - DNA, 864 bp, Artificial Sequence
  Synthetic construct
atgggatggtcatgtatcatcctttttctagtagcaactgcaaccggtgtacattcagaagtccagctggtcgaaagtggtgcagaagtcaagaaacctggtgcctctgtgaaagtatcatgcaaagtctctggctacaccctgaccgaactttctatgcactgggtgagacaagctcccggaaagggactggagtggatgggcgggtttgatccagaggacggtgagactatgtacgctcaaaagtttcaggggcgggtaactatgactgaagacacatcaacagataccgcttatatggagctgagctcactccggtcagaggatacagccgtctattactgcgctacttcaacggcagtagcggggactcccgacctgtttgattattattacggaatggacgtgtggggccagggaacaacagtgacagtgtcctctgcatcaggcggcggtggttctggtggtggcggctccggtggcggcggttctgaaatcgtgatgacacaaagcccgttgagtagccccgtcactctcgggcaacctgcatccatcagctgtcgatcttctcagtccctcgttcactcagacggcaatacatatctcagttggctgcaacagcgccccgggcagccacccaggctgcttatatataaaattagcaacagattttcaggagtgcctgaccggtttagtggctcaggtgctggaacagacttcaccctcaagatttctcgggtggaagctgaggatgttggtgtttattattgcacacaggcgacccagttcccatatacatttggccaagggactaaagttgacattaaaggcggatctggatctcaccaccaccaccaccactga

SEQ ID NO: 357 - DNA, 840 bp, Artificial Sequence
  Synthetic construct
atgggatggtcatgtatcatcctttttctagtagcaactgcaaccggtgtacattcagaagtccagctggtcgagagtggtcccggattggtgaagccatctgaaacactgtcactcacctgtacagcttccggtggatccatttccacatactactggagctggattcgccagccgcccgggaagggtctcgaatggataggctacatctattacagtgggtcaacgaactataatcccagtctcaagtcccgtgttaccatcagcgtcgataccagtaagaatcagttcagcctgaagctctcctccgttacagcggcagacacggccgtgtactattgcgccagagacagaattgctcctgtcggcaagttcttcgggtggtatttcgatttgtggggcagaggcactctcgttacagtgagtagcgcaagcgggggaggcggatccggcggggggggatcaggtggcggaggtagcgacatagtaatgacccagagccctgccaccttatcagttagtcccgaagagcgggccacattgagctgccgggcctcccaatccgtctcttcaaacctcgcctggtaccagcagaagccaggccaagctccgcggctcttgatatacggcgcctccactcgcgcaaccggtattcctgccagattttcaggtagcggcagcggtactgagttcactctgacaatcagcagccttcaatccgaggactttgccgtatactattgccaacagtacaataagtggccaccatggacttttggccagggcaccaaagtggatatcaaaggcggatctggatctcaccaccaccaccaccactga

SEQ ID NO: 358 - DNA, 843 bp, Artificial Sequence
  Synthetic construct
atgggatggtcatgtatcatcctttttctagtagcaactgcaaccggtgtacattcacaagtccagctcgcagaaagcggcggcggcgtggtgcaacccggccggtccctcagattatcctgcgcggcaagcgggttcaccttctcatcctacgccatgcactgggttaggcaggcacctggcaagggactggagtgggtcgcagtcatctcttatgacggctctaataaatactacgccgattccgtcaaaggccgctttacaataagccgcgataactctaaaaatacactttacctgcagatgaattccctccgtgccgaagacacagccgtgtactattgtgccaggagtacatcaggctcctactactacggaatggacgtatggggacagggaactacagtcacggttagttctgcatccggtggcggggggtctggcggtggcggcagtggggggggtggcagccaaagcgctttaactcaaccggcttccgtctccggtagcccggggcagtctatcactataagctgcactggcacatccagcgatgtgggtgattacaactatgtgtcttggtatcagcagcaccccgggaaggccccgaagttgatgatctatgatgtgtctaaccgcccatcaggagtgagcaaccgtttctctggatcaaaatcaggtaataccgcctccttgaccatctctggactgcaggctgaggacgaagctgagtactactgttcctcttacacgagctcctcaactttgttgtatgtgttcgggacaggcactaaggtaactgtattaggcggatctggatctcaccaccaccaccaccactga

SEQ ID NO: 359 - DNA, 840 bp, Artificial Sequence
  Synthetic construct
atgggatggtcatgtatcatcctttttctagtagcaactgcaaccggtgtacattcagaagctcagctggtcgagagtgggggaggcttggtgcagccgggccgctccctgaggctttcatgcgcagccagtggatttacgttcgatgattctgccatgcattgggtgagacaggctcctggaaagggcttggaatgggtgtcaggcatcagttggaattccggaaacgtggggtatgccgactccgtaaagggtcggttcactatctcacgtgataacgccaaaaattctctctacttgcagatgaactctctccgggcagaggacacggcactttactactgtactaaagctagccgctactgtagcagtaccatttgctactggaactggttcgacccatggggccagggcacattggttactgtgtcaagcgcgagcggagggggcggttctggcggcggtgggagtggtgggggaggctccgacattcaaatgacacagtcacccagctccttgagcgcctccgtgggagaccgcgtgactatcacctgtcgcgcctcccagtccatctcttcttacttgaactggtatcagcagaagcctggcaaggctcctaagcttctgatctatggggcatcttctctgcagactggcgttcccagtcgattttcaggctccggatcaggtacggattttactctcacaatacgctccctgcagccagaagatttcgcatcatactattgtcagcagtcttactcaacccccacattcgggggaggaacaaaagtggagattaaaggcggatctggatctcaccaccaccaccaccactga

SEQ ID NO: 360 - DNA, 840 bp, Artificial Sequence
  Synthetic construct
atgggatggtcatgtatcatcctttttctagtagcaactgcaaccggtgtacattcagaagtgcaattggtggagtccggggggggactcgtgaaacctggtgggagcttgcgactgtcctgcgccgcttctggcttcaccttttctggatatagcatgaattgggtgcggcaggcccccggcaagggcctggagtgggtgtcttccatttccagcagcagtagctatatatattatgctgacagcgtgaaaggcaggtttacaatttctcgggacaatgcaaagaattctctgtacttgcagatgaacagcctgagagctgaggataccgcagtctactactgcgctcggtggctgcaactgcgttccgactattactattttgggatggacgtatgggggcagggtactaccgtgactgtctcatctgcctccggcgggggaggaagcggaggcggaggaagtggtggcggagggtccgagatagttatgacccagtcccccgccactctgtcagtgtctcccggagagagagcaaccctgtcctgccgggcctcacaaagtgtgtcaaataaccttgcctggtaccaacacaagcccggtcaggcacctcgtcttcttatttatggagctagcaccagagcgactggcattccggcaaggttctccggctccggtagcggcacagaatttactctgacgatatcctctctgcaaagtgaagactttgcggtttatttttgccagcaatgctacaattggcccccatggactttcggacaggggaccaaggtggaaataaaaggcggatctggatctcaccaccaccaccaccactga

SEQ ID NO: 361 - DNA, 849 bp, Artificial Sequence
  Synthetic construct
atgggatggtcatgtatcatcctttttctagtagcaactgcaaccggtgtacattcacaagtgacactgaaagagagcggccccgtgctggtaaaaccgacggagaccttgacattgacttgtgcagtttcaggtttcagcttgtccaacgccaaaatgggcgtgagttggatacgccaaccacccggcaaagctcttgagtggctggcccatattttctctaatgatgagaaagcttattctactagtctgaaaactagactcacaatttcaaaggacacaagcaaaagccaagtcgtgctcaccgttaccaatatggaccccgtcgataccgccacgtactactgtgccaggattgtactgggagcctctgggacatatccgagtcctggatttgatccttggggtcagggcactctggtcacagtgtcttctgcatcaggcggaggaggctcaggcggcggcggctccggtggtggtgggtctcaaactgtggttacacaagagccaagcctgacggtctcacctggtggcactgttaccctgacatgtgcatcctctgcgggtgcggttacgtctgggtattatccgaattggttccagcagaaaccaggccaagcacccagagctctgatatacagtacagcgaataaacattcatggacactcgcgaggtttagcgggagtctccttggcgggaaagctgcacttacacttagcggcgtacagcctgaggatgaggccgagtattactgtttgctgtattacggcggcgcgtgggtgtttggcggcgggaccaaactgaccgtactgggcggatctggatctcaccaccaccaccaccactga

SEQ ID NO: 362 - DNA, 864 bp, Artificial Sequence
  Synthetic construct
atgggatggtcatgtatcatcctttttctagtagcaactgcaaccggtgtacattcacaagtccaattggtccaaagcggtgctgaagttaaaaaaccaggcgcctctgttaaggtttcttgtaaagcttcaggctatacatttacaagttacgatatcaattgggtgcgtcaggccaccgggcaaggtctggagtggatggggtggatgaaccctaactcaggcaacaccggttacgcccagaagttccaaggaagagtcacaatgacccgcaacacgtctattagcactgcctatatggagttgtcctctctcaggtctgaggataccgccctgtactactgtgcgcgggaagctcgctattttgattggatattcgagggctctgattactactattacggcatggacgtatggggccagggtactacagttaccgtttcctccgcttccgggggtggcggaagtggcggaggcggcagtggcggcggtggtagccagagcgctcttacacaacccccgagcgtttccggatctcccggccagtcagtgaccatcagttgcactggtacctctagtgacgttgggagctacaatagggtgtcatggtaccagcaacctcccggcaccgctcccaagctgatgatatacgaggtctccaataggccatccggagtgcctgatcggttttccggtagcaagagtggcaacaccgcctcattaacgatttccggcctgcaagctgaggatgaggccgactattactgctcttcctacacttcaagctcattacgagtttttgggggaggcaccaagttgaccgtgctcggcggatctggatctcaccaccaccaccaccactga

SEQ ID NO: 363 - DNA, 855 bp, Artificial Sequence
  Synthetic construct
atgggatggtcatgtatcatcctttttctagtagcaactgcaaccggtgtacattcacaggtgcagctcgttgaaagtggcggtggcgtcgtacagccagggcgctcactgaggctgtcttgcgccacctccgggttcacattcagcagcttcgccctgcattgggttagacaggctcccgggaaagggctcgagtgggtgaccgtgatcagtgacgacggcaataacaaatactatgtagactcagtgaaggggaggttcaccatctcccgggataactctaaaaataccctcttcctgcagatgaatagtcttcgagttgaggataccgccatttattactgcgctagagcttcttacaactctaattggagtattggggaatatttccgcgactggggccaggggactctggtgaccgtgtccagcgcaagcggcggcggggggtccgggggcggcgggtccggaggaggggggtcagatatagtgatgacacagtccccagattccctcgcagtctctctgggcgaaagagcaaccattaactgcaaaagttcacagtcactcctgtataccagtaacaacaagaattacctggcttggtatcagcagaagccaggccagcccccaaagcttcttatttactgggcatccacaagagagtctggcgtgcctgataggttctctggaagcggctcaggcacagatttcactttaacaattagcagcctccaagccgaggacgtcgctgtgtattactgccagcagtattactcccccccttggacctttggacagggtaccaaggtggaaatcaaaggcggatctggatctcaccaccaccaccaccactga

SEQ ID NO: 364 - DNA, 837 bp, Artificial Sequence
  Synthetic construct
atgggatggtcatgtatcatcctttttctagtagcaactgcaaccggtgtacattcacaactgcagctgcaagagtccggtccgggccttgtaaaaccttctgagacattatccctcacctgcactgtatccggaggttccatttcaagtacaacatactactgggggtggattcgacaaccaccgggaaaagggcttgagtggattgccagtatctactattcaggcagcacttactataacccctccctgaaatctagacttacagtttcagtggacaccagtaaaaatcaattttccttaaagctgtcttccgtgacggctgccgacacggcagtgtactactgtgcccggcagtggaaatggtttggagaagcatggtactttgacttatgggggagaggcacactcgtgaccgtctctagcgcctcaggaggcgggggatcagggggaggtggctccggtggcggcggtagtgagatcgtattaacacagagtccaggtaccctctccctttcccctggagaacgcgctagcctttcctgtcgagcctcccagtccgtgtcatcctcatacctcgcctggtaccagcaaaaacccgggcaggcgcctaggcttctgatctatggagccagttcacgcgccactgggatacctgatcgcttttctggtagcggcagcggaactgatttcacccttaccatatctcgattggaacccgaggacttcgctgtgtattattgccagcaatatggctcttccccctttaccttcggaggtggaaccaaggtggagatcaagggcggatctggatctcaccaccaccaccaccactga

SEQ ID NO: 365 - DNA, 831 bp, Artificial Sequence
  Synthetic construct
atgggatggtcatgtatcatcctttttctagtagcaactgcaaccggtgtacattcacaggtgcagttggtgcagagtggtgctgaggttaaaaaacctggaagcagcgtaaaggtgagctgcaaggcatcaggtggaacattctcttcttacgctataaattgggtgcgtcaagctcctggacagggactggaatggatgggcgggattattcccatctttggaacagcaaattacgcacagaaattccagggccgggtcacctttactgctgacgaaagtacctccaccgcctacatggaggtgagctctctcagatccgaggataccgctgtttattactgcgcccgaagttgtggcgattgctattccgcagacctggatttttggggacagggaaccctggtgactgtcagtagcgcctctggagggggggggtccggcggtggaggcagtgggggaggcggcagcgaaatcgtattaacacagtctcccgctactctttccctttctccaggcgagcgggccacacttagctgcagagccagccagtcaatttccagttttcttgcctggtatcagcagaaacctggccaagccccccgattgctgatatatgacgcgtcaaatcgagcgacaggcatccccgccagattttccggaagtggcagtggtactgactttaccctcacaatctccagtctggaacctgaggatttcgccgtgtattactgtcagcggagaagcaactggccgccatttacattcggccccggcacgaaagtagacatcaaaggcggatctggatctcaccaccaccaccaccactga

SEQ ID NO: 366 - DNA, 867 bp, Artificial Sequence
  Synthetic construct
atgggatggtcatgtatcatcctttttctagtagcaactgcaaccggtgtacattcagaagtgcagctcgtggagtccggaggaggggtagtccagcccggacgctccctccgcctgtcttgcgcagcatccggctttacattctctagttatgcaatgcactgggttcgccaagcacctggcaaaggcctggagtgggtggccgtgatctcatacgatggttccaataagtattacgccgatagcgttaagggaagattcaccataagccgggacaacactaagaacactttatatctccagatgaacagcttgcgcgctgaggacacagccgtttattactgtgcacgagacagcgagtactatgatatcctgactggatacctggcacctacccactattattactactacatggacgtctggggtaaagggaccacagtgactgtctcttccgcttctggtggtgggggatccggcggtggagggagtgggggcggcgggagcgaaatcgtgatgacccagtctccagcgaccctgtcagtgtcccccggcgagcgcgcaacattgtcctatcgcgcctcccagagcgtgtcctcaaacctcgcttggtatcaacagaaacccgggcaagccccacgtctcctgatctatggagcatcaacacgggcaaccgggatacccgccagattttctggatcagggagcggtacagagtttaccctcactatcagcagcttgcagagtgaggacttcgctgtgtactactgccaacagtacaacaactggccgcccgggacgttcggacaaggcaccaagctggagattaaaggcggatctggatctcaccaccaccaccaccactga

SEQ ID NO: 367 - DNA, 807 bp, Artificial Sequence
  Synthetic construct
atgggatggtcatgtatcatcctttttctagtagcaactgcaaccggtgtacattcagaagtccaactcgtggaatctggtcctgggcttgtaaagccaagtgagaccctgagcttgacttgtacagtgtctgggggatccatctcaagctactattggtcttggattaggcaacccccaggtaagaggctggaatggattgggtatatatactacagcggaagcaccaactacaacccttcattgaagtcccgggtcactatctccgtggatacgtctaaaaatcagttctcccttaaactgaatagtgtcactgctgctgacacagccgcctattactgtgcccgtggcttcgattactggggccaggggacccgagtcactgtgagtagtgcctccggcgggggaggtagcggtggtggtgggagcggtggcggaggaagcgacgtggtcatgacacagagccccgggacgctgtcactgagccccggcgagcgggctacactgagttgccgtgcttcccagaatgtatctagcagctatctggcctggtatcagcagaagcctggacaggcaccccgactgctcatttacggagccagttcccgggccaccgggatccctgacagattctccggatccggatcaggaacagactttacactcactatctcaagattagagcctgaggatttcgcagtctattattgtcagcagtatgggagtagccccctgttcactttcggcccaggaactaaggtggaaattaagggcggatctggatctcaccaccaccaccaccactga

SEQ ID NO: 368 - DNA, 837 bp, Artificial Sequence
  Synthetic construct
atgggatggtcatgtatcatcctttttctagtagcaactgcaaccggtgtacattcagaggtccagctcgtgcaaagcggagctgaggttaaaaagcctggatcctccgtgaaagtgtcatgcaaggcaagtgggggaacctttagcagctatgctataagctgggtgcggcaagccccaggccagggtctggaatggatgggggggatcatccccattttcggcaccaccaattatgcgcagaagtttcaaggcagggtgaccatcacaaccgacgagagcacttcaacggcttatatggagctttctagcttgcgatccgaggacactgccgtttattactgcgctagagtttatagctacgactccagtgggtactacttggagtactggggacagggaacccgggtgactgtttcctccgcctcaggcggggggggttcagggggcggaggaagcggaggcgggggaagcgagatcgttctgacacagtctcctgccacactgagcttatcccccggcgagcgagccacactctcatgtcgagcaagtcaaagcgtttcctcttatctcgcctggtaccagcagaaaccagggcaagccccacggctcctgatctacgacgctagcaatcgagccacgggcatcccagctcggtttagcgggtcaggtagcggaacagatttcaccttgacaatctcttctcttgaaccggaggacttcgcagtctactactgccagcaacgcagcaactggcctcccagcatcacttttggacaggggactcgattagagataaaaggcggatctggatctcaccaccaccaccaccactga

SEQ ID NO: 369 - DNA, 840 bp, Artificial Sequence
  Synthetic construct
atgggatggtcatgtatcatcctttttctagtagcaactgcaaccggtgtacattcagaggttcaattggtagaaagcggcggcggattggtgaaacccggtggctcactcagactgagctgtgctgccagtggatttaccttttcaaacgcatggatgagttgggtgcgacaagcacccggcaaaggcctcgaatgggtgggtaggattaagtccaagactgatggcggcaccactgattatgccgctcctgttaagggaaggttcactatttcaagagacgactcaaagaacaccctgtatttgcagatgaactcacttaaaaccgaagatacagcggtgtattattgtaccacagacagaggggatagctatggctactattattgcatggatgtttggggtaaaggtacaagagtgaccgtgagctccgctagtggtggtggcggatccggtggcggcgggtccggaggaggcgggtctcaactggtgctcacacaaccacctagtgtttctgtaagtcctggtcaaacagcgtctataacttgctctggtgacaaattgggcgataaatacgcgtgttggtaccaacaaaagccggggcaatctccagttctggtcatctatcaagattccaaacgaccaagtggcattcctgaacgctttagcggctccaattccgggaacactgcaacactgaccatcagcgggacacaggccatggatgaggcagattattactgtcaggcgtgggatagcagtacggccgtggtgttcggtggtggtactaaactgacggtccttggcggatctggatctcaccaccaccaccaccactga

SEQ ID NO: 370 - DNA, 846 bp, Artificial Sequence
  Synthetic construct
atgggatggtcatgtatcatcctttttctagtagcaactgcaaccggtgtacattcacaagtacagctcgtggaatccgggggcggcgtggtgcagcctgggcggagcctcagattgtcctgcgccgccagtggctttaccttctcctcatatgccatgcattgggtccgacaggccccaggcaaaggtcttgaatgggtcgccgtgataagttacgacgggtctaacaagtattacgctgattctgttaaagggaggtttacaatcagcagagacaattctaagaacacattgtatctgcagatgaactctttgcgagcggaggacactgccgtgtattactgcgcaagaagtgcatcaggctcatattatggagccttcgactactggggccagggaaccctcgtaaccgtgagcagcgcttccggaggagggggttccgggggcggcggatctggtggggggggatctcagactgtggtgacacagcctgcctcagtgtccggttctcccggacagtcaattaccatcagctgtactggcaccagctcagatgtcggcggatacaactatgtatcttggtatcagcaacatccagggaaggcccctaagctgatgatctatgacgttagcaatcgaccttccggagtgtccaaccgattttcagggagcaagtcaggcaacactgcctccttaactatatccggactccaggccgaagacgaagctgactattattgcagctcctatacctccagctcaacgcttggactctatgtattcggaaccgggactaaggttaccgtattaggcggatctggatctcaccaccaccaccaccactga

SEQ ID NO: 371 - DNA, 846 bp, Artificial Sequence
  Synthetic construct
atgggatggtcatgtatcatcctttttctagtagcaactgcaaccggtgtacattcacaagtacaattgcaagaatcaggaccaggactggttaaaccgagcgagactttgtcactcacatgtacagtgtcaggctatagtatcagttcagggtactattggggatggattcggcaaccacctggaaaaggactggaatggatcggatccatctatcattcaggaagcacttattacaatccatctttgaagagcagggtgacaatctctgttgatacatcaaagaatcaatttagcctcaagctgagttctgtgacagcggcagataccgcggtctattactgtgccagaactcctctctcccttagactgcggtataattggtattttgatctctggggtagaggcaccctcgttactgtaagcagtgcgagtggaggaggtggaagtggtggcggtgggagtggcggcggcggtagccaatcagttcttacccaaccaccatccgcctctggtactcccggtcaacgggttacaataagctgctccggcagcagcagcaacattggatctaacactgtcaactggtatcagcaacttcccgggacagccccaaagctgttgatctacagtaacaaccagaggccgtccggggtccccgacagatttagcggatctaaatccggtacatcagcatcccttgctatatcagggctgcagagtgaggatgaggctgactactactgtgcagcgtgggacgattcacttaacggaccagtattcggtggtggaaccaaactgaccgtcctcggcggatctggatctcaccaccaccaccaccactga

SEQ ID NO: 372 - DNA, 882 bp, Artificial Sequence
  Synthetic construct
atgggatggtcatgtatcatcctttttctagtagcaactgcaaccggtgtacattcacaagtgcaactggtccagagcggggccgaagtcaaaaaacctggtgcctccgtcaaagtgtcctgcaaagcgagcggctatacgttcacttcctacgacatcaattgggtgaggcaggccactggacaagggctggagtggatgggctggatgaaccccaacagcggcaacacagggtatgctcagaaattccagggccgagtcactatgacccggaacacatctatctccactgcatacatggaactgtcttccctcaggtcagaagacaccgctgtatactactgcgcccgcgcgaccacggattgctcctccaccagttgctggtcactcgatttctggtctggctactacacaggcgggcgggaaaagattttcgattgggggcagggcaccctggttacagtgtccagtgcaagtgggggtggaggcagtggaggggggggttctggaggaggtggtagcgagatcgtgctcacccagtcaccaggtactctcagcctcagtcctggagaaagggcaacgttgtcttgccgggcatctcagtccgtctcatcatcttaccttgcctggtaccagcagaagcctggtcaggccccccgcctcttaatctatggcgctagctctagggccacaggcataccggaccgattttctggcagtggttcaggtacagacttcaccctgactatcagccgtctcgagccagaggacttcgcggtctactactgtcagcagtatggcagtagtccaggaacatttggccaaggaaccaaggtggaaatcaagggcggatctggatctcaccaccaccaccaccactga

SEQ ID NO: 373 - DNA, 834 bp, Artificial Sequence
  Synthetic construct
atgggatggtcatgtatcatcctttttctagtagcaactgcaaccggtgtacattcagaggttcagttggtgcaatctggggccgaggttaaaaagcctggcgaatctctcaagatttcatgtaagggttcaggctacaggttcacgaattactggattgggtgggttcgccagatgcccggcaagggtctcgaatggatgggcattatttacccaggtgatagtgacacacgctacagcccctctttccagggccaggtcacaatctccgcagataagagcattaccacggcatatcttcagtggtcttcccttaaggcctcagatacagctatgtattactgtgcccggctctcagatcggtggtacagcccattcgatccatggggacaaggaactctggtcacagtgtcatcagcgtctgggggcgggggatctgggggcgggggttctggaggtggcggcagtcaggccgtggtcacacaggagcctagcctcaccgtatctcctggagggaccgtgaccttgacctgcggctcttccaccggggccgtgacatccggccactatccatattggttccagcagaaatccggtcaggcccccagaaccttaatttatgagactagtattaaacattcttggacccccgcccgtttcagcggatctctcctcggcgggaaggcagctctgaccctgtcaggcgcacagccagaagatgaggctgactactactgtctcctgagctactctggggctaggccggtgttcggcggggggaccaagctgactgttctgggcggatctggatctcaccaccaccaccaccactga

SEQ ID NO: 374 - DNA, 846 bp, Artificial Sequence
  Synthetic construct
atgggatggtcatgtatcatcctttttctagtagcaactgcaaccggtgtacattcagaggtccagcttgtggaatctggcgggggcttggtgcaaccgggcggctcattgcggctgtcctgtgccgctagcggcttcactgtgtcaagcaattacatgtcctgggtccggcaggcccccggcaagggcctggagtgggtatcagtgatctacagcgcaggctcaacttattatgccgatagcgtgaaaggacgctttagcatctcacgcgataaatcaaaaaataccctttatctccagatgaactcactcagggccgaagacactgccgtgtattactgtgcaaaggagggaggttctgggagtcttaggtactattattacggtatggacgtgtggggtcaggggaccaccgtcactgtgagtagcgccagcggaggcggaggatctgggggcggcgggagtgggggcggaggctcacagtccgtggttacacagcctcctagcgcttccggaactcccggccagcgggtgactataagttgctccgggtcctccagtaatatcgggtacaatatcgtgaattggtatcagcagttgccagggacggcccccaaactgctgatctattctaataaccagagacctagcggtgtacctgaccggtttagcgggagtaaatcaggcacgagcgccagtctgtccatctcagggctgcagtctgaggacgaggctgattattattgcgcagcctgggatgacagcttgaacgggtatgttttcgggacaggcacaaaagttacagtattaggcggatctggatctcaccaccaccaccaccactga

SEQ ID NO: 375 - DNA, 855 bp, Artificial Sequence
  Synthetic construct
atgggatggtcatgtatcatcctttttctagtagcaactgcaaccggtgtacattcagaagtccagctggtggagagcggagggggactggtgcagcccggtgggtcattgcggctgagctgcgcagcatccggcttcacgttttcttcatatgagatgaactgggtcaggcaagctccggggaaggggctggaatgggtgtcatatatttctagcagcggatcagccatctactacgctgattctgtgaaaggaaggttcacaatatcccgggacaacgcaaaaaacagcctttatctccagatgaactcccttagagtggaggacaccgccgtgtactattgcgcccgggaggctcggtcccggtacttcgactggcttccgtcctattattttgactattggggacagggaacactcgtcaccgtctctagcgcttccggaggcggcgggagcgggggagggggatctggcggggggggatctcagagcgcactcacgcagcccgccagcgtgtcagggtctccaggacagtcaataacgatctcctgcaccggaacctcatcagacattggcggatacaactatgtcagctggtaccagcaacatcccgggaaagccccaaagctgttgatctacgatgtctcaaacaggccatccggtgtaagcacccgcttctctggctcaaaatcaggaaataccgcgagccttaccatcagtgggttacaagccgaggatgaggctgattactattgttcatcctacacaagtagctcaactcacgtggtatttggcggggggacgaagctgacagtgctcggcggatctggatctcaccaccaccaccaccactga

SEQ ID NO: 376 - DNA, 837 bp, Artificial Sequence
  Synthetic construct
atgggatggtcatgtatcatcctttttctagtagcaactgcaaccggtgtacattcacaggtccagctgcagcagtggggagctggtctgctgaagcctagtgaaaccctgagtttaacttgcgcagtatacggcggctctttctctggccattattggtcctggattagacagccccctgggaaggggttggaatggatcggcgaaattaatcactcaggctctactaattacaatccctctctcaagtcccgagtcactatttctgtggatacttcaaagaatcagttctcactcaagctttcatcagttacagccgcagacactgcagtttactactgcgctaggccccctcaggcggctcggatacactattactattatatggatgtatggggcaaggggactaccgtgactgtttccagtgcatccggaggaggtggatcaggaggcggcggcagcgggggaggaggcagcgagatcgtaatgacccaaagtccggccacactctccgtcagccctggtgaacgtgccactttatcctgtcgcgccagtcagtcagtttccagtaatctggcctggtaccagcagaaacccggccaggccccaaggttactgatttatggcgcttctaccagagctacaggtatcccggcgcgcttttctggctctggctctggcaccgagtttaccctcacgataagcagcctccaatctgaggacttcgcagtgtattattgtcagcaatataactactggccaccattaaccttcggaggggggaccaaggtggagatcaaaggcggatctggatctcaccaccaccaccaccactga

SEQ ID NO: 377 - DNA, 858 bp, Artificial Sequence
  Synthetic construct
atgggatggtcatgtatcatcctttttctagtagcaactgcaaccggtgtacattcagaagtgcagctggtccagtccggtgcagaggtgaaaaagcctggtgagagccttaagatatcctgcaagggaagtggctactcattcacaagttattggatagattgggtacgtcagatgcctggaaaaggtttggagtggatgggcattatctatcccggggactcagatacacgttactctccttccttccaagggcaggtaaccatatcagccgacaagagtacctcaacagcatatctccagtggagctccttgaaagcaagcgataccgctatgtactattgtgctcggagaggtgaggcggccggaatatggtattttgacttgtggggacgcggaacactcgtgacagtgtcctctgcctcagggggcggaggctcaggcggcggcggaagtggaggcggcggctcccagccagtattgacacaacctccctcagcctccgccagcctcggagcgagcgtaacattaacgtgtacactgtccagcggatactccaactataaggtcgactggtatcagcagagaccggggaaaggtcctcgatttgtgatgagagtgggaacaggagggatagttggctctaaaggagacggaattccagacagattctcagtgttgggtagcggtttgaacagatacctgaccatcaaaaatattcaggaagaagacgagtccgattatcactgcggagctgatcatgggtctggcagtaattttgaatacgtcgtctttgggggcggcacaaagctgaccgttctgggcggatctggatctcaccaccaccaccaccactga

SEQ ID NO: 378 - DNA, 822 bp, Artificial Sequence
  Synthetic construct
atgggatggtcatgtatcatcctttttctagtagcaactgcaaccggtgtacattcagaggtccgtcttctggagtctggaggagggctcgtgcagccgggcggcagtcttcgactgtcctgcgctgcatctgggtttacattttctgactacgcaatgaactgggttcgacaggctccaggcaaggggctggagtgggtctcagccattagtgctaccggcgggtctaccttttatgcagacagcgtgaagggtcgattctccatatctagagacaatagcaaaaattccctcgtgcttcaaatgaacagtttacgagcagaagacaccgccgtatattactgtgccaagccctacggcatggacgtttgggggcaggggactactgtgaccgtaagcagcgcctcaggcggtggaggaagtgggggaggcggatctgggggtggcggatcacagagcgcactcacacagcccgcctccgttagcggcagccctggacaatctataacaattagttgtaccggtacttcctctgacgtggggggctataacttcgtgagttggcaccaacagcaccccggtaaggcccccaagaatatgatctacgatgtgagcaaacgcccttccggagttagcaaccgattttcaggaagcaaaagcggtaataccgcctccctcactatctctgggctgcatacagaagacgaggctgattactactgtagcagttatacttctacaagtacaccatgggtgttcggcggagggacaaaattgacagtactgggcggatctggatctcaccaccaccaccaccactga

SEQ ID NO: 379 - DNA, 837 bp, Artificial Sequence
  Synthetic construct
atgggatggtcatgtatcatcctttttctagtagcaactgcaaccggtgtacattcagaggtgcaattagtggagtccggcggaggtctcgtccagcccggtggttctctgaggttaagctgcgccgcaagtggctttaccttttcttcttattggatgaattgggtccgacaggctccggggaagggcctggaatgggtggcaaatatcaatcaggacggtggcgaaaagtactacgtagattctgtgcggggaagattcacaataagccgtgataacgctaagaatagcctctatctgcagatgaacagcctgcgcgctgaggatacggcagtttattattgcgcgcgcgacccatatgacctgtatggggactacggaggcacttttgattactggggacagggaacgttagttactgtctctagtgccagtgggggtggaggttcagggggcggaggttccggcggcggtggaagcgatatccagatgacccaatccccctcaacacttagtgcctctgtgggggatagggttacaatcacctgtagagctagccagagcatttccagctggctcgcctggtaccagcagaagccccggaaggcaccaaagctgctgatctataaagcatcaactctggaatcaggtgtgccaagtcggttttctggaagcggctccggcactgagttcactcttaccatatcttctctccaaccggacgattttgccacatactattgtcagcagtacaactcatacagcttgacttttggaggaggtactaaggtcgaaatcaagggcggatctggatctcaccaccaccaccaccactga

SEQ ID NO: 380 - DNA, 843 bp, Artificial Sequence
  Synthetic construct
atgggatggtcatgtatcatcctttttctagtagcaactgcaaccggtgtacattcacaggttcagctcgtcgaatcaggtggtggggtagttcagcccggacgcagtctgaggctgtcatgcgccgccagtggattcacattttcatcatacctgatgcactgggtgcgccaagcccccggtaaggggttggagtgggtggcagtgatttgggctaacggcaatcggtactacgccgacagtgtaaagggcaggttcacgatcagccgcgacatatccaaaaataccctgtacttacagatgaattcactgcgcgccgaggatactgctatgtattactgcgcacgagactactgcaatggagtgacatgtaactccaattattggggacagggaaccctcgtcactgtttccagtgccagcggagggggcgggtccggcggaggggggtctggaggcgggggctccgattttgtcatgacgcagtctccgggaagcttagcagtgagtctcggcgagagagctaccataaattgccgcagcagccagtctgtgctggacaattcttccaataaaaaccatctcgcatggcaccaacaaaaacccgggcaaccacctaagctcctgatttactgggcctctactcgcgagtcaggagtccctgacagatttagtggtagcggaagtggtactgatttcaccttgacaatctctagtctccaggccgaggacgttgcagtgtattattgccagcagtattactcttctcattggacattcggacaggggaccaaggtggaaatcaaaggcggatctggatctcaccaccaccaccaccactga

SEQ ID NO: 381 - DNA, 837 bp, Artificial Sequence
  Synthetic construct
atgggatggtcatgtatcatcctttttctagtagcaactgcaaccggtgtacattcacagctgcaactgcaagaaagcgggccagggttagttaaaccaagccaaacactttccttaacttgtactgtctccggcggctccatctcttctgggggttattactggagctggataagacagttgcctggcaaggggctggagtggatagggtatatatattatagcgggagtacttactacaaccctagcctgaagagcagggtgacaattagcgtggatacttccaagaaccagttctcactgaatctttcttccgtgacagcagccgacaccgccgtttattattgtgcccggcaacagctcgattattacgactccagtggatgtttcgattattggggtcagggcaccctggtgactgtttctagcgctagcggaggggggggctccggcggaggtggatctgggggaggaggaagcgatattgtgatgacacagagcccctccttcctgtcagctagtgtgggggaccgcgtgaccatcacttgtcgggcctcacagggaatatcatcctatctggcctggtatcagcagaaacccggaaaagctcctaagctgctgatttacgcagcctcaacgctccaatccggagtaccttcacgtttcagcgggtctggttccggcacggaattcacgctcacgatcagttcacttcagccggaggatttcgccacatactactgtcagcagctccacagctatccattgactttcggagggggcaccaaggtcgatattaagggcggatctggatctcaccaccaccaccaccactga

SEQ ID NO: 382 - DNA, 843 bp, Artificial Sequence
  Synthetic construct
atgggatggtcatgtatcatcctttttctagtagcaactgcaaccggtgtacattcagaagtgcaattgttggaaagtggtggtggactggttcagccgggtggctctctgaaactctcttgtgcggctagcgggtttacgttttcaagttacgcgatgtcctgggtccgtcaagctccaggaaagggactggaatgggtatcagcgatctctggatcaggttctaatacttactatgccgacagcgtgaaaggccggtttacaatttcccgggataatagtaagaataccttgtacttgcagatgaattcactgagagcagaagataccgcagtctactattgcgctaaaggacttcgaggccaacaactcgtaatcccaactgagtatttccagcattggggccaaggtactcttgtgactgtttcttcagcctcaggcggaggaggctctggtggcggcggttcaggtggggggggttccgatatagttatgactcaaagtcccggtacgctctctctcagccccggggaacgcgcaacccttagctgtcgtgccagtcaatctgtcagctcaagctacttggcctggtaccaacaaaagcccgggcaggcaccgcgccttctgatttatggcgcgagctctcgggccaccgggatcccagatcgattttctggcagcggttcaggcactgactttacgctcaccatatctaggctcgagcctgaagacttcgcagtatactattgccaacagtacggatctagcttgttgactttcggtggtgggactaaagtcgaaataaaaggcggatctggatctcaccaccaccaccaccactga

SEQ ID NO: 383 - DNA, 861 bp, Artificial Sequence
  Synthetic construct
atgggatggtcatgtatcatcctttttctagtagcaactgcaaccggtgtacattcagaagtgcagttggtggaatcaggaggagggctcgtgaagcctggaggcagtttacggcttagttgcgcagcatcaggctttaccttcagtagctattctatgaactgggtgcgccaggctccaggaaaaggactcgagtgggtgtcttctataagctcatccagctcatacatctactacgctgacagtgttaaagggcgcttcaccatttctagggacaacgcaaagaactctctgtacctccagatgaactccctcagagctgaagataccgctgtttactattgtgcccgggacagggaatcctacgatatattgacaggatactctatggagggctgtttcgactactggggtcaggggactcttgtgactgtgtcctctgccagtggcgggggcggcagcggcggtggaggtagcggtggtggagggagtcagctggttttaacccagtccccttccgccagtgctagcctgggcgcctctgtaaaactgacttgcaccctttcaagcggacatagctcttacgctattgcttggcatcaacagcagcctgagaagggccctcggtatttgatgaagctcaactcagacggaagtcattcaaaaggcgacgggatacccgaccgcttcagtggctccagttccggggcggagaggtatctgaccattagctctctgcagtcagaagacgaggcggattactattgccagacctggggaacgggcatcagagtcttcggtggcggaacgaagttaacagttctgggcggatctggatctcaccaccaccaccaccactga

SEQ ID NO: 384 - DNA, 864 bp, Artificial Sequence
  Synthetic construct
atgggatggtcatgtatcatcctttttctagtagcaactgcaaccggtgtacattcacaggtccaactcgttcagtctggagccgaggtcaagaagccgggagcgtctgtgaaggtgagctgcaaggcgtccgggtacacgtttacatcttatggaatcagctgggtccggcaagctccaggtcaaggactggaatggatggggtggattagcgcttacaacggcaacacaaattacgcccaaaagctgcaggggagggtaaccatgaccaccgatacttctacaagtacagcatatatggagctgagatcactgcggtcagacgatactgctgtgtattactgtgccagggtgacagtggaagctatctttggtgtagtgatcctgcccctcaaaaactggttcgatccatggggacagggcaccctggttacggtgtcttctgctagtggggggggtgggtcaggcggcgggggcagcggcgggggaggatcacagtctgtgctgacacagccacctagcgtgagcggtgctcctggacagcgggtgaccatctcctgcactggatcaagtagcaatattggtgcagggtatgatgtccattggtatcagcagctcccaggcactgctccgaagctgctgatctacggaaactcaaaccgcccatctggtgttcctgaccgattcagcggcagcaaatcaggaaccagcgccagccttgctataactggcctccaggcggaggatgaagccgattactattgccagtcttacgatagcagtcttactctttatgtgtttggcactgggaccaaagtgacagtccttggcggatctggatctcaccaccaccaccaccactga

SEQ ID NO: 385 - DNA, 840 bp, Artificial Sequence
  Synthetic construct
atgggatggtcatgtatcatcctttttctagtagcaactgcaaccggtgtacattcacaggtacaactggtgcaatccggtgctgaggtaaagaagcccggagcctccgtcaaagtgtcatgcaaggcgagcggtcacacctttactagctactacatgcactgggtcagacaagcaccaggacaaggtcttgaatggatgggaataatcaatccttctggtggatcaacgagctacgcacagaaatttcagggtagggtaaccatgacgagagatacctctacaagcacagtatacatggaactttcctcactccgctctgaagatacggctgtgtattattgcgctagggggcccgaaaggggaattgtaggtgccacagactacttcgactattggggacaaggaactctggtaaccgttagttctgctagcggcggaggcggatcaggcggaggaggctctggcggaggcggatcggaaatcgtccttacgcagtcaccggggaccctttctctgagcccaggtgagagggccacactttcttgccgagcgagtcaaagcgtttcttcctcctatttggcttggtatcagcaaaaaccaggacaagccccgaggctgcttatatatggggcttcttctagggctaccggaatcccggatcggttcagcggctctggatcaggtacggatttcacactgactattagccgacttgagccagaagactttgcggtatattactgccaacaatatgtgagtagcccctggacattcggacaaggaacgaaagtagagataaagggcggatctggatctcaccaccaccaccaccactga